(19) United States
(12) Patent Application Publication
(10) Pub. No.: US 2024/0100018 A1
(43) Pub. Date: Mar. 28, 2024

(54) COMPOUNDS FOR THE TREATMENT OF A DISEASE OR DISORDER, METHODS FOR IDENTIFYING SAID COMPOUNDS

(71) Applicants: UNIVERSITY OF PITTSBURGH-OF THE COMMONWEALTH SYSTEM OF HIGHER EDUCATION, Pittsburgh, PA (US); CEDARS-SINAI MEDICAL CENTER, Los Angeles, CA (US)

(72) Inventors: Mark E. SCHURDAK, Warrendale, PA (US); Andreas VOGT, Pittsburgh, PA (US); Andrew Michael STERN, Bradenton, FL (US); Douglass Lansing TAYLOR, Pittsburgh, PA (US); Fangyuan CHEN, Beijing (CN); Fen PEI, Medford, MA (US); Hongying CHENG, Pittsburgh, PA (US); Ivet BAHAR, Wexford, PA (US); Qingya SHI, Beijing (CN); Moshe ARDITI, Encino, CA (US)

(21) Appl. No.: 18/273,161

(22) PCT Filed: Jan. 24, 2022
(86) PCT No.: PCT/US2022/013449

§ 371 Date: Jul. 19, 2023

Related U.S. Application Data
(60) Provisional application No. 63/140,574, filed on Jan. 22, 2021.

(51) Int. Cl. A61K 31/397; A61K 31/137; A61K 31/352; A61K 31/436; A61K 31/4375; A61K 45/06; A61P 31/04

(52) U.S. Cl. CPC A61K 31/397; A61K 31/137; A61K 31/352; A61K 31/436; A61K 31/4375; A61K 45/06; A61P 31/04

(57) ABSTRACT

Disclosed herein are methods of identifying a compound for treating or preventing an infection with an infectious microbe, such as a coronavirus, in a subject in need thereof. Also disclosed herein are compounds and compositions identified by said methods, and methods of use thereof.

### CROSS-REFERENCE TO RELATED APPLICATIONS

This application claims the benefit of priority to U.S. Provisional Application No. 63/140,574, filed Jan. 22, 2021, which is hereby incorporated herein by reference in its entirety.

### STATEMENT OF GOVERNMENT SUPPORT

This invention was made with government support under grant numbers GM103712; DK119973; and DK117881 awarded by the National Institutes of Health. The government has certain rights in the invention.

### BACKGROUND

Covid-19 (coronavirus disease-2019) caused by SARS-CoV-2 (severe acute respiratory syndrome coronavirus (CoV) type 2 virus) has led to over 1.3 million deaths as of mid-November, 2020, due to its high contagiousness and therefore rapid spread. There is an urgent need to develop new therapeutics against Covid-19.

While efforts to target viral proteins are underway, an alternative strategy is to pursue host-targeted therapies. The host cell response is essential to enabling viral entry, endosomal escape, translation, replication, assembly, and release. Host cells are also naturally armed with antiviral programs, which, if properly induced, can constrain the in vivo viral spread within a canonical 4-7 day period, upon sufficient adaptive immunity development.

There is a need for the identification and development of drugs that interact with cell host proteins involved in viral infection and immune response to viral infection. There is a need for the identification and development of drugs that interact with cell host proteins involved in SARS-CoV-2 infection and that protect from SARS-CoV-2 hyperinflammation. There is a need for the identification and development of drugs that inhibit SARS-CoV-2 entry into cells.

The compounds, compositions, and methods disclosed herein address these and other needs.

### SUMMARY

In accordance with the purposes of the disclosed materials and methods, as embodied and broadly described herein, the disclosed subject matter, in one aspect, relates to compounds, compositions, and methods of identifying, making, and using compounds and compositions.

For example, disclosed herein are methods of identifying a compound for treating or preventing an infection with an infectious microbe in a subject in need thereof, the methods comprising:
a) obtaining transcriptomic data from cells infected with the infectious microbe,
b) identifying differentially expressed genes (DEGs),
c) characterizing host-targeted antimicrobial or anticytokine signature,
d) identifying compounds that stimulate the anti-microbial or -cytokine signature,
e) evaluating known and predicted targets of compounds identified in step d),
f) constructing an infection host response protein-protein interaction (PPI) network and modules,
g) prioritizing compounds based on network proximity analysis,
h) clustering of prioritized compounds associated with selected disease modules,
i) selecting representative compounds from each cluster for in vitro assays, and
j) analyzing the results of steps a-i to thereby identify the compound for treating or preventing the infection.

In some examples, the infectious microbe can comprise a coronavirus.

Also disclosed herein are methods of identifying a compound for treating or preventing a coronavirus infection in a subject in need thereof, the method comprising:
a) obtaining transcriptomic data from coronavirus infected cells,
b) identifying differentially expressed genes (DEGs),
c) characterizing host-targeted antiviral or anticytokine signature,
d) identifying compounds that stimulate the anti-viral or -cytokine signature,
e) evaluating known and predicted targets of compounds identified in step d),
f) constructing a coronavirus infection host response protein-protein interaction (PPI) network and modules,
g) prioritizing compounds based on network proximity analysis,
h) clustering of prioritized compounds associated with selected disease modules,
i) selecting representative compounds from each cluster for in vitro assays, and
j) analyzing the results of steps a-i to thereby identify the compound for treating or preventing the infection.

Also disclosed herein are compositions comprising the compound identified by any of the methods disclosed herein. Also disclosed herein are methods of treating a disease or disorder in a subject in need thereof, the method comprising administering to the subject a composition comprising a therapeutically effective amount of the composition comprising the compound identified by any of the methods disclosed herein.

Also disclosed herein are methods of treating or preventing a coronavirus infection in a subject in need thereof, comprising administering to the subject a therapeutically effective amount of a composition comprising a compound selected from the group consisting of: imipramine, salmeterol, hexylresorcinol, brompheniramine, ezetimibe, temsirolimus, linsitinib, torin-1, rottlerin, semaxanib, ipratropium, AS-605240, mefenamic acid, JNJ16259685, QL-XII-47, midodrine, olanzapine, trifluoperazine, fluphenazine, azelastine, chlorphenamine, clarithromycin, saracatinib, JAK3-Inhibitor-II, AZD-8055, CGP-60474, hexamethylene, loperamide, nifedipine, liothyronine, atorvastatin, triptolide, pirfenidone, isoliquiritigenin, rucaparib, berbamine, darinaparsin, taurodeoxycholic acid; derivatives thereof; and combinations thereof.

Also disclosed herein are methods of treating or preventing a coronavirus infection in a subject in need thereof, comprising administering to the subject a therapeutically effective amount of a composition comprising an antiviral compound and an anti-hyperinflammatory compound.

Also disclosed herein are pharmaceutical compositions for the treatment of a coronavirus infection in a subject in need thereof, wherein the pharmaceutical composition comprises a pharmaceutically acceptable excipient and a therapeutically effective amount of a composition comprising a compound selected from the group consisting of: imipramine, salmeterol, hexylresorcinol, brompheniramine, ezetimibe, temsirolimus, linsitinib, torin-1, rottlerin, semaxanib, ipratropium, AS-605240, mefenamic acid, JNJ16259685, QL-XII-47, midodrine, olanzapine, trifluoperazine, fluphenazine, azelastine, chlorphenamine, clarithromycin, saracatinib, JAK3-Inhibitor-II, AZD-8055, CGP-60474, hexamethylene, loperamide, nifedipine, liothyronine, atorvastatin, triptolide, pirfenidone, isoliquiritigenin, rucaparib, berbamine, darinaparsin, taurodeoxycholic acid; derivatives thereof; and combinations thereof

Also disclosed herein are pharmaceutical compositions for the treatment of coronavirus comprising a pharmaceutically acceptable excipient and a therapeutically effective amount of a composition comprising an antiviral compound and an anti-hyperinflammatory compound

Additional advantages of the disclosed devices and methods will be set forth in part in the description which follows, and in part will be obvious from the description. The advantages of the disclosed devices and methods will be realized and attained by means of the elements and combinations particularly pointed out in the appended claims. It is to be understood that both the foregoing general description and the following detailed description are exemplary and explanatory only and are not restrictive of the disclosed devices and methods, as claimed.

The details of one or more embodiments of the invention are set forth in the accompanying drawings and the description below. Other features, objects, and advantages of the invention will be apparent from the description and drawings, and from the claims.

### DESCRIPTION OF FIGURES

The accompanying figures, which are incorporated in and constitute a part of this specification, illustrate several aspects of the disclosure, and together with the description, serve to explain the principles of the disclosure.

FIG. 1. Workflow of the quantitative systems pharmacology approach for selecting compounds for experimental evaluation. Panel A: The RNA-seq data from SARS-CoV-2 infected A549 cells (Blanco-Melo D et al. bioRxiv, 2020, 10.1101/2020.03.24.004655) and ACE2-overexpressing A549 cells were used as input (Blanco-Melo D et al. Cell, 2020, 181, 1036-1045.e1039). Panel B: Up- and down-regulated differentially expressed genes (DEGs) were identified from these data using Wald test with false-discovery rate (FDR) default upper value of 0.05. Panel C: The antiviral gene signature (top) and anti-cytokine gene signature (bottom) were identified upon manual curation of GO enrichment results corresponding to the DEGs, using the QuickGO hierarchical annotation (Binns D et al. Bioinformatics 2009, 25, 3045-3046) (see FIG. 2-FIG. 5 for details). Panel D: Two sets of compounds or repurposable drugs that best reproduced the antiviral and anti-cytokine signatures were extracted from Cmap (Lamb J et al. Science, 2006, 313, 1929-1935; Subramanian A et al. Cell, 2017, 171, 1437-1452.e1417). Panel E: Known and predicted targets of these compounds were identified using QuartataWeb (Li H et al. Bioinformatics, 2020, 36, 3935-3937). Panel F: A host response network composed of four modules related to SARS-CoV-2 infection (called disease modules) was constructed. Panel G: The target of the compounds identified in Panel E and the disease modules in Panel F were subjected to network proximity analysis (Guney E et al. Nat Commun. 2016, 7, 10331) using BioSNAP lung PPI network, to prioritize 25 repurposable or investigational drugs for each module. This step has been performed for antiviral compounds only. Panel H and Panel I: The compounds were clustered based on the interaction patterns with their targets, using QuartataWeb. Representatives from each cluster (Panel H) and additional compounds identified by manual curation were selected for experimental testing (Panel I).

FIG. 2. Illustration of the 4-step pipeline for identifying the intrinsic antiviral signature in A549 cells 24 h after SARS-CoV-2 infection: (1) Identification of 100 upregulated and 20 downregulated genes; (2) GO enrichment analysis for up- and downregulated genes, respectively. The hierarchy of enriched GO terms was generated using QuickGO; (3) Classification of pro- or antiviral GO terms. Upregulated GO terms are classified as either proviral, antiviral, or ambiguous. Downregulated GO terms are all considered as anti-viral; (4) Gene selection for antiviral signature from the classified GO terms. Genes were included if they were antiviral or unknown.

FIG. 3. GO enrichment of up (left) and down (right) regulated genes. GO terms were filtered by size and overlapping genes as described in Materials and Methods. A total of 17 upregulated (Biological Process) and 13 downregulated (Cellular Component) genes are illustrated. P-values were derived from Fisher's one-tailed test and adjusted by Benjamini-Hochberg for multiple test correction.

FIG. 4. Change in the expression levels of 36 genes defining the host-targeted antiviral signature. Log₂ fold change at 24-h post-SARS-CoV-2 infection from A549 cells are shown.

FIG. 5. Change in the expression levels of 17 genes defining anti-cytokine signature; log₂ fold change at 24 h post-high SARS-CoV-2 infection from A549-ACE2 cells are shown.

FIG. 6. Identification of candidate compounds/drugs, their prioritization and final selection of a small set for experimental tests, illustrated for Dataset 1 (related to FIG. 1). The flow diagram depicts the number of compounds/drugs extracted at various stages, indicated by the Panels A-I (on the left) consistent with FIG. 1 panels A-I. The original analysis of transcriptomics data from A549 cells leads to 36 DEGs, whose antiviral signature screened against Cmap database identifies 263 candidate compounds. Comparison with Excelra DB shows those (10 of them) already listed therein. Of these 263 compounds, 168 have target information available and/or predictable in QuartataWeb (Li H et al. Bioinformatics, 2020, 36, 3935-3937)—an interface that utilizes as input DrugBank and STITCH database. Two different paths are then followed, for the respective subsets of 168 and 95 compounds. In the former case, the targets of these 168 compounds are subjected to network proximity analysis with respect to four disease modules in SARS-CoV-2-host interactome, using BioSNAP human lung PPI network; this analysis yields 64 compounds, which, upon clustering (using QuartataWeb) to select representatives, are reduced to 13 high-priority compounds. The latter set of 95 compounds are manually analyzed to select two compounds, leading to a total set of 15 high priority compounds that have been further investigated in experiments. The diagram depicts the protocol for antiviral compounds. In the case of anti-cytokine compounds, the same schema without panel G is adopted.

FIG. 7. Host cell proteins targeted by potential antiviral (Dataset 1) compounds/drugs, rank-ordered by their promiscuity. Promiscuity refers to the number of predicted compounds/drugs (also called chemicals) that target the protein. FIG. 7 lists the top 100 targets corresponding to Dataset 1 compounds/drugs. The ordinate lists the proteins, and the horizontal bars (abscissa) show the corresponding number of compounds.

FIG. 8. Host cell proteins targeted by potential anti-cytokine (Dataset 2) compounds/drugs, rank-ordered by their promiscuity. Promiscuity refers to the number of predicted compounds/drugs (also called chemicals) that target the protein. FIG. 8 lists the top 100 targets corresponding to Dataset 2 compounds/drugs. The ordinate lists the proteins, and the horizontal bars (abscissa) show the corresponding number of compounds.

FIG. 9. Prioritized compounds proposed to have potential antiviral activities and their involvement in different modules in the viral-host PPI network. 25 compounds/drugs were identified for each of the four modules, resulting in a total of 64 distinct repurposable drugs or investigational compounds, some participating in multiple modules. The entries in the heat map display the ranking, color-coded from red (highest) to blue (lowest). The ranking was based on the proximity of their targets to proteins belonging to the modules.

FIG. 10. Distribution of the same compounds/drugs in the four studied modules. Compounds belonging to selected intersections and to the viral entry module are listed. Those colored red have been experimentally tested. See the complete list in FIG. 9 and Table 9.

FIG. 11. Interaction-pattern-based clustering of top-ranking compounds from four modules. Results for 64 compounds (or chemicals) identified to yield closest proximity to four selected modules. The compounds are clustered based on their interaction patterns with their targets listed in QuartataWeb. 12 main clusters (clusters 1-8, 10, 11, 13 and 14, from left to right, delimited by yellow squares) contain two or more compounds each; 16 chemicals do not belong to any cluster. From each of cluster, up to two chemicals were selected based on their side effects and MOA.

FIG. 12. Interaction-pattern-based clustering of chemicals targeting immune response. Clustering of 163 chemicals proposed to modulate the immune response, based on anti-cytokine signature gene derived from infected A546-Ace2 cells. The chemicals are clustered based on their interaction patterns reported in DrugBank or STITCH. 20 main clusters were distinguish (marked by yellow squares) which contain two or more chemicals, and 35 additional chemicals that do not form clusters.

FIG. 13. Structure of ten chemicals tested for SARS-CoV-2 infection inhibitory activity in vitro. Structures of salmeterol, rottlerin, temsirolimus, torin-1, ezetimibe, brompheniramine, imipramine, linsitinib, hexylresorcinol, and semaxanib, selected for in vitro assays.

FIG. 14. Representative fluorescence images of Mock, SARS-CoV-2 infected (Control), and Salmeterol-treated wells analyzed with the Multiwavelength Cell Scoring application in MetaXpress. Grayscales of the images were adjusted to enable direct comparison of the relative levels of fluorescence among the treatments: Segmentation images show how cells were segmented and identified as spike positive. Purple, nuclei; cyan, spike. Scale bar, 100 μm.

FIG. 15. Suppression of SARS-CoV-2 infection by identified compounds. Vero-E6 cells were pretreated with compounds (salmeterol, rottlerin, temsirolimus, torin-1, or ezetimibe) for 1 h prior to SARS-CoV-2 inoculation. 48-h post-infection cells were fixed and labeled for SARS-CoV-2 S protein. Images are representative of five imaging fields in triplicate wells. Scale bar, 100 μm.

FIG. 16. Violin plots of Vero-E6 cells labeled for Spike protein. The Multiwavelength Cell Scoring algorithm in MetaXpress was used to determine the integrated fluorescent signal in individual cells as a measure of the amount of Spike protein within each cell. The plots show the population distribution of the integrated signal for all of the treatments. The Boxes in the plot show the interquartile range (IQR) with the top and bottom edges marking the 75^{th} and 25^{th} percentiles, respectively. The horizontal line in the box is the median value, and the whiskers are defined to be 1.5 IQR. The ordinate is a log scale. The effect of the treatment is assessed quantitatively by changes in the median signal level, and qualitatively by observing changes in the modes. The dashed line is 3 standard deviations above the mean signal in the Mock samples and is used as a cutoff to quantify the number of cells that are positive or negative for the Spike signal. The statistics table below the plots shows the number of cells counted in each treatment group and the median of the population.

FIG. 17. Pie charts showing the effect of treatment on preventing infection of Vero-E6 cells. The number of cells above and below the cutoffs for being positive for Spike were counted and the percent cells in each category were determined. All analyses were done in Tibco Spotfire.

FIG. 18. Dose-response curve for Nafamostat in the syncytia assay. Data are the aggregate of 8 independent biological repeats; where errors are shown they represent SD from matching concentrations in at least three experiments.

FIG. 19. Dose-response curve for Linsitinib in the syncytia assay. Data are the aggregate of 8 independent biological repeats; where errors are shown they represent SD from matching concentrations in at least three experiments.

FIG. 20. Dose-response curve for Hexylresorcinol in the syncytia assay. Data are the aggregate of 8 independent biological repeats; where errors are shown they represent SD from matching concentrations in at least three experiments.

FIG. 21. Dose-response curves for dec-RVKR-CMK in the syncytia assay. Data are the aggregate of 8 independent biological repeats; where errors are shown they represent SD from matching concentrations in at least three experiments.

FIG. 22. Dose-response curve for Bromopheniramine in the syncytia assay. Data are the aggregate of 8 independent biological repeats; where errors are shown they represent SD from matching concentrations in at least three experiments.

FIG. 23. Dose-response curve for Salmeterol in the syncytia assay. Data are the aggregate of 8 independent biological repeats; where errors are shown they represent SD from matching concentrations in at least three experiments.

FIG. 24. Quantification of syncytia formation in HEK293 cells treated with dec-RVKR-CMK relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars for n=1.

FIG. 25. Quantification of syncytia formation in HEK293 cells treated with brompheniramine relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars for n=1.

FIG. 26. Quantification of syncytia formation in HEK293 cells treated with hexylresorcinol relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars for n=1.

FIG. 27. Quantification of syncytia formation in HEK293 cells treated with imipramine relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars for n=1.

FIG. 28. Quantification of syncytia formation in HEK293 cells treated with linsitinib relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars for n=1.

FIG. 29. Quantification of syncytia formation in HEK293 cells treated with semaxanib relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars for n=1.

FIG. 30. Quantification of syncytia formation in HEK293 cells treated with ezetimibe relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars for n=1.

FIG. 31. Quantification of syncytia formation in HEK293 cells treated with salmeterol relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars for n=1.

FIG. 32. Representative images illustrating syncytia phenotype and compound activity in HEK293 cells. No spike, donor cells expressing GFP only. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 33. Representative images illustrating syncytia phenotype and compound activity in HEK293 cells treated with 100 μM DMSO. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 34. Representative images illustrating syncytia phenotype and compound activity in HEK293 cells treated with nafamostat (5.5 μM). Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 35. Representative images illustrating syncytia phenotype and compound activity in HEK293 cells treated with 100 μM dec-RVKR-CMK. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 36. Representative images illustrating syncytia phenotype and compound activity in HEK293 cells treated with 100 μM brompheniramine. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 37. Representative images illustrating syncytia phenotype and compound activity in HEK293 cells treated with 100 μM hexylresorcinol. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 38. Representative images illustrating syncytia phenotype and compound activity in HEK293 cells treated with 100 μM imipramine. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 39. Representative images illustrating syncytia phenotype and compound activity in HEK293 cells treated with linsitinib (25 μM). Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 40. Representative images illustrating syncytia phenotype and compound activity in HEK293 cells treated with semaxanib (50 μM). Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 41. Representative images illustrating syncytia phenotype and compound activity in HEK293 cells treated with 100 μM ezetimibe. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 42. Representative images illustrating syncytia phenotype and compound activity in HEK293 cells treated with 100 μM salmeterol. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm. No spike, donor cells expressing GFP only.

FIG. 43. Quantification of syncytia formation in Calu-3 cells treated with dec-RVKR-CMK relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars, n=1.

FIG. 44. Quantification of syncytia formation in Calu-3 cells treated with brompheniramine relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars, n=1.

FIG. 45. Quantification of syncytia formation in Calu-3 cells treated with hexylresorcinol relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars, n=1.

FIG. 46. Quantification of syncytia formation in Calu-3 cells treated with imipramine relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars, n=1.

FIG. 47. Quantification of syncytia formation in Calu-3 cells treated with linsitinib relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars, n=1.

FIG. 48. Quantification of syncytia formation in Calu-3 cells treated with semaxanib relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars, n=1.

FIG. 49. Quantification of syncytia formation in Calu-3 cells treated with ezetimibe relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars, n=1.

FIG. 50. Quantification of syncytia formation in Calu-3 cells treated with salmeterol relative to nafamostat. Numbers indicate p-values obtained by one-way ANOVA (non-matched, unpaired) with Dunnett's multiple comparisons test in Graph Pad Prism (v7.00) compared with vehicle control (dotted line). No p-value, p>0.05. Bars and errors represent the means±SD from multiple independent biological repeats, each performed in quadruplicate. No error bars, n=1.

FIG. 51. Representative images illustrating syncytia phenotype and compound activity in Calu-3 cells. No spike, donor cells expressing GFP only. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 52. Representative images illustrating syncytia phenotype and compound activity in Calu-3 cells treated with 100 μM DMSO. Upper panel, raw fluorescence micrograph; lower panel, images with CNT overlay. GFP positive objects that met the criteria for syncytia are colored purple; cellular aggregates that are not syncytia are shown in yellow. Scale bar, 100 μm.

FIG. 53. Representative images illustrating syncytia phenotype and compound activity in Calu-3 cells treated with nafamostat (5.5 μM). Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 54. Representative images illustrating syncytia phenotype and compound activity in Calu-3 cells treated with 100 μM dec-RVKR-CMK. Upper panel, raw fluorescence micrograph; lower panel, image with CNT overlay. GFP positive objects that met the criteria for syncytia are coloredpurp/e; cellular aggregates that are not syncytia are shown in yellow. Scale bar, 100 μm.

FIG. 55. Representative images illustrating syncytia phenotype and compound activity in Calu-3 cells treated with 100 μM brompheniramine. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 56. Representative images illustrating syncytia phenotype and compound activity in Calu-3 cells treated with 100 μM hexylresorcinol. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 57. Representative images illustrating syncytia phenotype and compound activity in Calu-3 cells treated with 100 μM imipramine. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 58. Representative images illustrating syncytia phenotype and compound activity in Calu-3 cells treated with linsitinib (25 μM). Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 59. Representative images illustrating syncytia phenotype and compound activity in Calu-3 cells treated with semaxanib (50 μM). Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 60. Representative images illustrating syncytia phenotype and compound activity in Calu-3 cells treated with 100 μM ezetimibe. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 61. Representative images illustrating syncytia phenotype and compound activity in Calu-3 cells treated with 100 μM salmeterol. Upper panel, raw fluorescence micrograph; lower panel, image with CNT overlay. GFP positive objects that met the criteria for syncytia are colored purple; cellular aggregates that are not syncytia are shown in yellow. Scale bar, 100 μm.

FIG. 62. The 36 genes of antiviral signature expression in A549 cells (same as FIG. 4).

FIG. 63. The 36 genes of antiviral signature expression in A549-ACE2 cells.

FIG. 64. The 17 genes of anti-inflammatory signature expression in A549-ACE2 cells (same as FIG. 5).

FIG. 65. The 17 genes of anti-inflammatory signature expression in A549 cells.

FIG. 66. Schematic representation of various stages of SARS-CoV-2 infection: viral entry, endosomal maturation, replication, translation, and accompanying cell signaling and regulation or immune responses, described in the main text. Mainly, SARS-CoV-2 spike binds the host receptor ACE2 (Hoffmann M et al. Cell, 2020, 181, 271-280) complexed with the amino acid transporter B⁰ AT1 (Yan R et al. Science, 2020, 367, 1444-1448). Proteolytic cleavages (e.g., by TMPRSS2) are essential to viral entry, including spike priming and membrane fusion, or lysosomal escape after endocytosis. PlKfyve is the main enzyme synthesizing PI(3,5)P₂ in early endosome (de Lartigue J et al. Traffic, 2009, 10, 883-893), and PI(3,5)P₂ regulates early-to-late endosome events. TPC2 is a major downstream effector of PI(3,5)P₂ (Li P et al. Trends Biochem Sci. 2019, 44, 110-124). Dominant pathways in four modules involved in SARS-CoV-2 infection are listed in the upper right boxes (see also Table 7). The diagram also shows selected drugs that have been identified and experimentally validated to inhibit or reduce SARS-2-CoV-2 infection (mainly viral entry) in highlighted in boxes (with red fonts).

FIG. 67. Subnet of PPIs between host cell proteins implicated in SARS-CoV-2 infection and those targeted by selected compounds. The sandy brown nodes and edges represent the proteins and interactions in the SARS-CoV-2 host response network; and in the background (transparent light blue nodes and edges) is the lung tissue-specific protein interactome. The relative size of each protein node is consistent with its degree (number of connections) in the PPI network. Thirteen compounds were identified as candidate repurposable or investigational drugs for host-targeted antiviral therapy (based on Dataset 1) and their connections to targets in host response network (as reported in DrugBank or STITCH) are shown by color-coded labels and connectors. Magenta nodes represent the compounds that predominantly inhibit viral entry; light green and red represent those against viral translation, replication, and immune response; and cyan nodes represent multifunctional compounds.

FIG. 68. Chemical structures of selected drugs displayed in FIG. 66 and FIG. 67 targeting various components of the viral-host interactome; see all tested drugs in FIG. 13.

### DETAILED DESCRIPTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an analog” includes mixtures of two or more such analogs, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

Values can be expressed herein as an “average” value. “Average” generally refers to the statistical mean value.

By “substantially” is meant within 5%, e.g., within 4%, 3%, 2%, or 1%.

It is understood that throughout this specification the identifiers “first” and “second” are used solely to aid the reader in distinguishing the various components, features, or steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

As used herein, microbes include, for example, bacteria, fungi, viruses, protozoa, etc.

As used herein, antimicrobials include, for example, antibacterials, antifungals, and antivirals. As used herein, “antimicrobial” refers to the ability to treat or control (e.g., reduce, prevent, treat, or eliminate) the growth of a microbe at any concentration. Similarly, the terms “antibacterial,” “antifungal,” and “antiviral” refer to the ability to treat or control the growth of bacteria, fungi, and viruses at any concentration, respectively.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., microbe population/infection). Similarly, “increase” or other forms of the word, such as “increasing” or “increase,” is meant raising of an event or characteristic. It is understood that in both cases this is typically in relation to some standard or expected value, in other words it is relative, but that it is not always necessary for the standard or relative value to be referred to. For example, “reduces tumor growth” means decreasing the amount of tumor cells relative to a standard or a control. For example, “reducing microbial infection” means reducing the spread of a microbial infection relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. “Prevent” does not require comparison to a control as it is typically more absolute than, for example, “reduce.” As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

As used herein, “treat” or other forms of the word, such as “treated” or “treatment” refers to obtaining beneficial or desired clinical results. Beneficial or desired clinical results include, but are not limited to, any one or more of: alleviating of one or more symptoms (such as viral spread), diminishing the extent of cancer or viral infection, stabilizing (i.e., not worsening) state of disease, preventing or delaying spread of the viral infection, delaying occurrence or recurrence of disease, delaying or slowing of disease progression, ameliorating the disease state, and remission (whether partial or total). For example, “treat” or other forms of the word, such as “treated” or “treatment,” can refer to administration of a composition or performing a method in order to reduce, prevent, inhibit, or eliminate a particular characteristic or event (e.g., microbe growth or survival). The term “control” is used synonymously with the term “treat.”

The term “therapeutically effective amount” refers to the amount of the composition used is of sufficient quantity to ameliorate one or more causes or symptoms of a disease or disorder. Such amelioration only requires a reduction or alteration, not necessarily elimination. In reference to viral infections, an effective amount comprises an amount sufficient to cure, palliate, ameliorate, stabilize, reverse, prevent, slow or delay the progression of the disease, pathological condition, or disorder. In some embodiments, an effective amount is an amount sufficient to delay development or infection. In some embodiments, an effective amount is an amount sufficient to prevent or delay occurrence and/or recurrence. An effective amount can be administered in one or more doses. In the case of a viral infection, the effective amount of the drug or composition may: cure viral infections, palliate or ameliorate symptoms associated with viral infections, stabilize to some extent and preferably stop viral replication, prevent viral infections or the onset of complications associated with viral infections, slow or delay the progression of viral replication.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

As used herein, the term “delivery” encompasses both local and systemic delivery. As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone.

Methods and Compositions

Disclosed herein are methods of identifying a compound for treating, preventing, or ameliorating a disease or disorder in a subject in need thereof. Also disclosed herein are methods for of treating, preventing, or ameliorating a disease or disorder in a subject in need thereof, the methods comprising administering to the subject a therapeutically effect amount of a compound, such as a compound identified by the methods disclosed herein, or a therapeutically effective amount of a composition (such as a pharmaceutical composition) comprising said compound.

For example, the compounds and compositions described herein or pharmaceutically acceptable salts thereof are useful for treating a disease or disorder in humans, e.g., pediatric and geriatric populations, and in animals, e.g., veterinary applications. The disclosed methods can optionally include identifying a patient who is or may be in need of treatment of a disease or disorder.

In some examples, the disease or disorder comprises an infection, such as with an infectious microbe (e.g., bacteria, virus, fungi, protozoa, etc.). In some examples, the disease or disorder comprises an infection with a coronavirus.

Examples of viruses include both DNA viruses and RNA viruses. Exemplary viruses can belong to the following non-exclusive list of families Adenoviridae, Arenaviridae, Astroviridae, Baculoviridae, Barnaviridae, Betaherpesvirinae, Birnaviridae, Bromoviridae, Bunyaviridae, Caliciviridae, Chordopoxvirinae, Circoviridae, Comoviridae, Coronaviridae, Cystoviridae, Corticoviridae, Entomopoxvirinae, Filoviridae, Flaviviridae, Fuselloviridae, Geminiviridae, Hepadnaviridae, Herpesviridae, Gammaherpesvirinae, Inoviridae, Iridoviridae, Leviviridae, Lipothrixviridae, Microviridae, Myoviridae, Nodaviridae, Orthomyxoviridae, Papovaviridae, Paramyxoviridae, Paramyxovirinae, Partitiviridae, Parvoviridae, Phycodnaviridae, Picornaviridae, Plasmaviridae, Pneumovirinae, Podoviridae, Polydnaviridae, Potyviridae, Poxviridae, Reoviridae, Retroviridae, Rhabdoviridae, Sequiviridae, Siphoviridae, Tectiviridae, Tetraviridae, Togaviridae, Tombusviridae, and Totiviridae.

Specific examples of viruses include, but are not limited to, Mastadenovirus, Adenovirus, Human adenovirus 2, Aviadenovirus, African swine fever virus, classical swine fever virus, arenavirus, Lymphocytic choriomeningitis virus, Ippy virus, Lassa virus, Arterivirus, Human astrovirus 1, Nucleopolyhedrovirus, Autographa californica nucleopolyhedrovirus, Granulovirus, Plodia interpunctella granulovirus, Badnavirus, Commelina yellow mottle virus, Rice tungro bacilliform, Barnavirus, Mushroom bacilliform virus, Aquabirnavirus, Infectious pancreatic necrosis virus, Avibirnavirus, Infectious bursal disease virus, Entomobirnavirus, Drosophila X virus, Alfamovirus, Alfalfa mosaic virus, Ilarvirus, Ilarvirus Subgroups 1-10, Tobacco streak virus, Bromovirus, Brome mosaic virus, Cucumovirus, Cucumber mosaic virus, Bhanja virus Group, Kaisodi virus, Mapputta virus, Okola virus, Resistencia virus, Upolu virus, Yogue virus, Bunyavirus, Anopheles A virus, Anopheles B virus, Bakau virus, Bunyamwera virus, Bwamba virus, C virus, California encephalitis virus, Capim virus, Gamboa virus, Guama virus, Koongol virus, Minatitlan virus, Nyando virus, Olifantsvlei virus, Patois virus, Simbu virus, Tete virus, Turlock virus, Hantavirus, Hantaan virus, Nairovirus, Crimean-Congo hemorrhagic fever virus, Dera Ghazi Khan virus, Hughes virus, Nairobi sheep disease virus, Qalyub virus, Sakhalin virus, Thiafora virus, Crimean-congo hemorrhagic fever virus, Phlebovirus, Sandfly fever virus, Bujaru complex, Candiru complex, Chilibre complex, Frijoles complex, Punta Toro complex, Rift Valley fever complex, Salehabad complex, Sandfly fever Sicilian virus, Uukuniemi virus, Uukuniemi virus, Tospovirus, Tomato spotted wilt virus, Calicivirus, Vesicular exanthema of swine virus, Capillovirus, Apple stem grooving virus, Carlavirus, Carnation latent virus, Caulimovirus, Cauliflower mosaic virus, Circovirus, Chicken anemia virus, Closterovirus, Beet yellows virus, Comovirus, Cowpea mosaic virus, Fabavirus, Broad bean wilt virus 1, Nepovirus, Tobacco ringspot virus, Coronavirus, Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, porcine reproductive and respiratory syndrome virus, Rat coronavirus, Turkey coronavirus, Rabbit coronavirus, Torovirus, Berne virus, Breda virus, Corticovirus, Alteromonas phage PM2, Pseudomonas Phage phi6, Deltavirus, Hepatitis delta virus, Hepatitis D virus, Hepatitis E virus, Dianthovirus, Carnation ringspot virus, Red clover necrotic mosaic virus, Sweet clover necrotic mosaic virus, Enamovirus, Pea enation mosaic virus, Filovirus, Marburg virus, Ebola virus, Ebola virus Zaire, Flavivirus, Yellow fever virus, Tick-borne encephalitis virus, Rio Bravo Group, Japanese encephalitis, Tyuleniy Group, Ntaya Group, Uganda S Group, Dengue Group, Modoc Group, Pestivirus, Bovine diarrhea virus, Hepatitis C virus, Furovirus, Soil-borne wheat mosaic virus, Beet necrotic yellow vein virus, Fusellovirus, Sulfobolus virus 1, Subgroup I, II, and III geminivirus, Maize streak virus, Beet curly top virus, Bean golden mosaic virus, Orthohepadnavirus, Hepatitis B virus, Avihepadnavirus, Alphaherpesvirinae, Simplexvirus, Human herpesvirus 1, Herpes Simplex virus-1, Herpes Simplex virus-2, Varicellovirus, Varicella-Zoster virus, Epstein-Barr virus, Human herpesvirus 3, Cytomegalovirus, Human herpesvirus 5, Muromegalovirus, Mouse cytomegalovirus 1, Roseolovirus, Human herpesvirus 6, Lymphocryptovirus, Human herpesvirus 4, Rhadinovirus, Ateline herpesvirus 2, Hordeivirus, Barley stripe mosaic virus, Hypoviridae, Hypovirus, Cryphonectria hypovirus 1-EP713, Idaeovirus, Raspberry bushy dwarf virus, Inovirus, Coliphage fd, Plectrovirus, Acholeplasma phage L51, Iridovirus, Chilo iridescent virus, Chloriridovirus, Mosquito iridescent virus, Ranavirus, Frog virus 3, Lymphocystivirus, Lymphocystis disease virus flounder isolate, Goldfish virus 1, Levivirus, Enterobacteria phage MS2, Allolevirus, Enterobacteria phage Qbeta, Lipothrixvirus, Thermoproteus virus 1, Luteovirus, Barley yellow dwarf virus, Machlomovirus, Maize chlorotic mottle virus, Marafivirus, Maize rayado fino virus, Microvirus, Coliphage phiX174, Spiromicrovirus, Spiroplasma phage 4, Bdellomicrovirus, Bdellovibrio phage MAC 1, Chlamydiamicrovirus, Chlamydia phage 1, T4-like phages, coliphage T4, Necrovirus, Tobacco necrosis virus, Nodavirus, Nodamura virus, Influenzavirus A, B and C, Thogoto virus, Polyomavirus, Murine polyomavirus, Papillomavirus, Rabbit (Shope) Papillomavirus, Paramyxovirus, Human parainfluenza virus 1, Morbillivirus, Measles virus, Rubulavirus, Mumps virus, Pneumovirus, Human respiratory syncytial virus, Partitivirus, Gaeumannomyces graminis virus 019/6-A, Chrysovirus, Penicillium chrysogenum virus, Alphacryptovirus, White clover cryptic viruses 1 and 2, Betacryptovirus, Parvovirinae, Parvovirus, Minute mice virus, Erythrovirus, B19 virus, Dependovirus, Adeno-associated virus 1, Densovirinae, Densovirus, Junonia coenia densovirus, Iteravirus, Bombyx mori virus, Contravirus, Aedes aegypti densovirus, Phycodnavirus, 1-Paramecium bursaria Chlorella NC64A virus group, Paramecium bursaria chlorella virus 1, 2-Paramecium bursaria Chlorella Pbi virus, 3-Hydra viridis Chlorella virus, Enterovirus, Poliovirus, Human poliovirus 1, Rhinovirus, Human rhinovirus 1A, Hepatovirus, Human hepatitis A virus, Cardiovirus, Encephalomyocarditis virus, Aphthovirus, Foot-and-mouth disease virus, Plasmavirus, Acholeplasma phage L2, Podovirus, Coliphage T7, Ichnovirus, Campoletis sonorensis virus, Bracovirus, Cotesia melanoscela virus, Potexvirus, Potato virus X, Potyvirus, Potato virus Y, Rymovirus, Ryegrass mosaic virus, Bymovirus, Barley yellow mosaic virus, Orthopoxvirus, Vaccinia virus, Parapoxvirus, Orf virus, Avipoxvirus, Fowlpox virus, Capripoxvirus, Sheep pox virus, Leporipoxvirus, Myxoma virus, Suipoxvirus, Swinepox virus, Molluscipoxvirus, Molluscum contagiosum virus, Yatapoxvirus, Yaba monkey tumor virus, Entomopoxviruses A, B, and C, Melolontha melolontha entomopoxvirus, Amsacta moorei entomopoxvirus, Chironomus luridus entomopoxvirus, Orthoreovirus, Mammalian orthoreoviruses, reovirus 3, Avian orthoreoviruses, Orbivirus, African horse sickness viruses 1, Bluetongue viruses 1, Changuinola virus, Corriparta virus, Epizootic hemarrhogic disease virus 1, Equine encephalosis virus, Eubenangee virus group, Lebombo virus, Orungo virus, Palyam virus, Umatilla virus, Wallal virus, Warrego virus, Kemerovo virus, Rotavirus, Groups A-F rotaviruses, Simian rotavirus SA11, Coltivirus, Colorado tick fever virus, Aquareovirus, Groups A-E aquareoviruses, Golden shiner virus, Cypovirus, Cypovirus types 1-12, Bombyx mori cypovirus 1, Fijivirus, Fijivirus groups 1-3, Fiji disease virus, Fijivirus groups 2-3, Phytoreovirus, Wound tumor virus, Oryzavirus, Rice ragged stunt, Mammalian type B retroviruses, Mouse mammary tumor virus, Mammalian type C retroviruses, Murine Leukemia Virus, Reptilian type C oncovirus, Viper retrovirus, Reticuloendotheliosis virus, Avian type C retroviruses, Avian leukosis virus, Type D Retroviruses, Mason-Pfizer monkey virus, BLV-HTLV retroviruses, Bovine leukemia virus, Lentivirus, Bovine lentivirus, Bovine immunodeficiency virus, Equine lentivirus, Equine infectious anemia virus, Feline lentivirus, Feline immunodeficiency virus, Canine immunodeficiency virus Ovine/caprine lentivirus, Caprine arthritis encephalitis virus, Visna/maedi virus, Primate lentivirus group, Human immunodeficiency virus 1, Human immunodeficiency virus 2, Human immunodeficiency virus 3, Simian immunodeficiency virus, Spumavirus, Human spuma virus, Vesiculovirus, Vesicular stomatitis virus, Vesicular stomatitis Indiana virus, Lyssavirus, Rabies virus, Ephemerovirus, Bovine ephemeral fever virus, Cytorhabdovirus, Lettuce necrotic yellows virus, Nucleorhabdovirus, Potato yellow dwarf virus, Rhizidiovirus, Rhizidiomyces virus, Sequivirus, Parsnip yellow fleck virus, Waikavirus, Rice tungro spherical virus, Lambda-like phages, Coliphage lambda, Sobemovirus, Southern bean mosaic virus, Tectivirus, Enterobacteria phage PRD1, Tenuivirus, Rice stripe virus, Nudaurelia capensis beta-like viruses, Nudaurelia beta virus, Nudaurelia capensis omega-like viruses, Nudaurelia omega virus, Tobamovirus, Tobacco mosaic virus (vulgare strain; ssp. NC82 strain), Tobravirus, Tobacco rattle virus, Alphavirus, Sindbis virus, Rubivirus, Rubella virus, Tombusvirus, Tomato bushy stunt, virus, Carmovirus, Carnation mottle virus, Turnip crinkle virus, Totivirus, Saccharomyces cerevisiae virus, Giardiavirus, Giardia lamblia virus, Leishmaniavirus, Leishmania brasiliensis virus 1-1, Trichovirus, Apple chlorotic leaf spot virus, Tymovirus, Turnip yellow mosaic virus, Umbravirus, Carrot mottle virus, Variola virus, Coxsackie virus, Dengue virus, Rous sarcoma virus, Zika virus, Lassa fever virus, Eastern Equine Encephalitis virus, Venezuelan equine encephalitis virus, Western equine encephalitis virus, St. Louis Encephalitis virus, Murray Valley fever virus, West Nile virus, Human T-cell Leukemia virus type-1, echovirus, norovirus, and feline calicivirus (FCV).

In some examples, the virus can comprise an influenza virus, a coronavirus, or a combination thereof. Examples of influenza viruses include, but are not limited to, Influenzavirus A (including the H1N1, H2N2, H3N2, H5N1, H7N7, H1N2, H9N2, H7N2, H7N3, H10N7, H7N9, and H6N1 serotypes), Influenzavirus B, Influenzavirus C, and Influenzavirus D. Examples of coronaviruses include, but are not limited to, avian coronavirus (IBV), porcine epidemic diarrhea virus (PEDV), porcine respiratory coronavirus (PRCV), porcine reproductive and respiratory syndrome (PRRS) virus, transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2), and middle east respiratory syndrome (MERS) coronavirus (CoV) (MERS-CoV). In some examples, the virus can comprise Severe Acute Respiratory Syndrome (SARS)-Coronavirus (CoV)-2 (SARS-CoV-2).

Specific examples of bacteria include, but are not limited to, Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, Salmonella Typhimurium, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, Brucella suis, Brucella melitensis, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, rickettsia, rickettsia prowazekii, rickettsia typhi, other Rickettsial species, Ehrlichia species, Staphylococcus aureus, Staphylococcus epidermidis, Streptococcus pneumoniae, Streptococcus pyogenes, Streptococcus agalactiae, Streptococcus uberis, Escherichia coli, Vibrio cholerae, Vibrio parahaemolyticus, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, Clostridium botulinum, Clostridium perfringens, other Clostridium species, Yersinia enterolitica, Yersinia pestis, other Yersinia species, Mycoplasma species, Bacillus anthracis, Bacillus abortus, other Bacillus species, Corynebacterium diptheriae, Corynebacterium bovis, Francisella tularensis, Chlamydophila psittaci, Campylocavter jejuni, Enterobacter aerogenes, Klebsiella pneumoniae, Klebsiella oxytoca, Proteus spp., Serratia marcescens, Trueperella pyogenes, and Vibria vulnificus.

Specific examples of fungi include, but are not limited to, Candida albicans, Cryptococcus neoformans, Histoplama capsulatum, Aspergillus niger, Aspergillus oryzae, Aspergillus fumigatus, Coccidiodes immitis, Paracoccidioides brasiliensis, Blastomyces dermitidis, Pneumocystis carinii, Penicillium marneffi, Alternaria alternate, coccidioides immitits, Fusarium oxysporum, Geotrichum candidum, and Histoplasma capsulatum.

Specific examples of parasites include, but are not limited to, Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

For example, disclosed herein are methods of identifying a compound for treating or preventing an infection with an infectious microbe in a subject in need thereof, the methods comprising:
a) obtaining transcriptomic data from cells infected with the infectious microbe,
b) identifying differentially expressed genes (DEGs),
c) characterizing host-targeted antimicrobial or anti-cytokine signature,
d) identifying compounds that stimulate the anti-microbial or -cytokine signature,
e) evaluating known and predicted targets of compounds identified in step d),
f) constructing an infection host response protein-protein interaction (PPI) network and modules,
g) prioritizing compounds based on network proximity analysis,
h) clustering of prioritized compounds associated with selected disease modules,
i) selecting representative compounds from each cluster for in vitro assays, and
j) analyzing the results of steps a-i to thereby identify the compound for treating or preventing the infection.

In some examples, the infectious microbe comprises a coronavirus.

Also disclosed herein are methods of identifying a compound for treating or preventing a coronavirus infection in a subject in need thereof, the methods comprising:
a) obtaining transcriptomic data from coronavirus infected cells,
b) identifying differentially expressed genes (DEGs),
c) characterizing host-targeted antiviral or anti-cytokine signature,
d) identifying compounds that stimulate the anti-viral or -cytokine signature,
e) evaluating known and predicted targets of compounds identified in step d),
f) constructing a coronavirus infection host response protein-protein interaction (PPI) network and modules,
g) prioritizing compounds based on network proximity analysis,
h) clustering of prioritized compounds associated with selected disease modules,
i) selecting representative compounds from each cluster for in vitro assays, and
j) analyzing the results of steps a-i to thereby identify the compound for treating or preventing the infection.

In some examples, the coronavirus comprises human coronavirus, SARS-CoV, SARS-CoV-2, or MERS-CoV.

The infected cells can, for example, comprise infected A549 cells, ACE2-overexpressing A549 cells, or a combination thereof.

In some examples, the differentially expressed genes are identified using Wald test with false-discovery rate (FDR) default upper value of 0.05.

In some examples, the host-targeted antimicrobial, antiviral, and/or anti-cytokine signature is/are characterized using manual curation of gene ontology (GO) enrichment results corresponding to the DEGs.

In some examples, the compounds that stimulate the antimicrobial, antiviral, and/or anti-cytokine signature are identified using Cmap.

In some examples, the known and predicted targets of compounds are evaluated using QuartataWeb.

The compounds can, for example, be prioritized based on network proximity analysis using the lung PPI network in BioSNAP.

In some examples, the in vitro assays can comprise viral inhibition or cell fusion (syncytia) assays.

In some examples, the methods can further comprise considering additional criteria such as drug development status, side effects, mechanism of action (MOA), and antiviral activities of the prioritized compounds in order to identify the compound for treating the infection.

Also disclosed herein are methods of treating or preventing a coronavirus infection in a subject in need thereof, comprising administering to the subject a therapeutically effective amount of a composition comprising a compound selected from the group consisting of: imipramine, salmeterol, hexylresorcinol, brompheniramine, ezetimibe, temsirolimus, linsitinib, torn-1, rottlerin, semaxanib, ipratropium, AS-605240, mefenamic acid, JNJ16259685, QL-XII-47, midodrine, olanzapine, trifluoperazine, fluphenazine, azelastine, chlorphenamine, clarithromycin, saracatinib, JAK3-Inhibitor-II, AZD-8055, CGP-60474, hexamethylene, loperamide, nifedipine, liothyronine, atorvastatin, triptolide, pirfenidone, isoliquiritigenin, rucaparib, berbamine, darinaparsin, taurodeoxycholic acid; derivatives thereof; and combinations thereof. In some examples, the composition comprises an antiviral compound, an anti-hyperinflammatory compound, or a combination thereof. In some examples, the coronavirus comprises human coronavirus, SARS-CoV, SARS-CoV-2, or MERS-CoV.

Also disclosed herein are methods of treating or preventing a coronavirus infection in a subject in need thereof, comprising administering to the subject a therapeutically effective amount of a composition comprising an antiviral compound and an anti-hyperinflammatory compound. In some examples, the coronavirus comprises human coronavirus, SARS-CoV, SARS-CoV-2, or MERS-CoV.

In some examples, the antiviral compound inhibits cell fusion or viral entry. In some examples, the antiviral compound comprises a histamine receptor antagonist, an acetylcholine receptor antagonist, a norepinephrine and serotonin reuptake inhibitor, an autophagy enhancer, a mTOR inhibitor, a PI3K inhibitor, an IGF-1- and insulin receptor inhibitor, a TB K1 activator through ARF1, an adrenergic receptor agonist, a VEGFR inhibitor, a local anesthetic, a cyclooxygenase inhibitor, a glutamate receptor antagonist, a Niemann-Pick Cl-like 1 protein antagonist, a cholesterol inhibitor, a cytoplasmic tyrosine protein kinase BMX inhibitor, a MAPK and protein kinase inhibitor, or a combination thereof. In some examples, the antiviral compound comprises: imipramine, salmeterol, hexylresorcinol, brompheniramine, ezetimibe, temsirolimus, linsitinib, torin-1, rottlerin, semaxanib, ipratropium, AS-605240, mefenamic acid, JNJ16259685, QL-XII-47; derivatives thereof; or a combination thereof. In some examples, the antiviral compound comprises: salmeterol, rottlerin, imipramine, linsitinib, hexylresorcinol, ezetimibe, brompheniramine; derivatives thereof; or a combination thereof. In some examples, the antiviral compound comprises salmeterol, linisitinib, imipramine, derivatives thereof, or a combination thereof. In some examples, the antiviral compound comprises salmeterol, linisitinib, imipramine, fluvoxamine, or a combination thereof. In some examples, the antiviral compound comprises an IGF-1R and/or insulin receptor inhibitor, such as linsitinib. In some examples, the antiviral compound comprises an adrenergic receptor agonist, such as salmeterol.

In some examples, the anti-hyperinflammatory compound comprises an adrenergic receptor agonist, a dopamine receptor antagonist, an autophagy enhancer, an autophagy dual modulator, a histamine receptor antagonist, a bacterial 50S ribosomal subunit inhibitor, an autophagy inhibitor, a SRC inhibitor, a JAK inhibitor, a mTOR inhibitor, a CDK inhibitor, a sodium/hydrogen antiport inhibitor, an opioid receptor agonist, a calcium channel blocker, a thyroid hormone stimulant, a HMGCR inhibitor, a RNA polymerase inhibitor, a TGFβ receptor inhibitor, an anti-fibrotic, a guanylate cyclase activator, a PARP inhibitor, a calmodulin antagonist, an apoptosis stimulant, a bile acid, or a combination thereof. In some examples, the anti-hyperinflammatory compound comprises midodrine, olanzapine, trifluoperazine, fluphenazine, azelastine, chlorphenamine, clarithromycin, saracatinib, JAK3-Inhibitor-II, AZD-8055, CGP-60474, hexamethylene, loperamide, nifedipine, liothyronine, atorvastatin, triptolide, pirfenidone, isoliquiritigenin, rucaparib, berbamine, darinaparsin, taurodeoxycholic acid, derivatives thereof, or a combination thereof. In some examples, the anti-hyperinflammatory compound elevates IFN signaling and/or suppresses cytokine pathways. In some examples, the anti-hyperinflammatory compound elevates IFN signaling and suppresses cytokine pathways.

In some examples, the composition comprises salmeterol, linsitinib, impramine, derivatives thereof, or a combination thereof, optionally in combination with one or more additional agents.

In some examples, the composition comprises salmeterol in combination with one or more additional agents. In some examples, the composition comprises salmeterol in combination with an RNA-dependent RNA polymerase inhibitor, a 3CL protease inhibitor, or a combination thereof. In some examples, the composition comprises salmeterol in combination with molnupiravir, paxlovid, or a combination thereof. In some examples, the composition comprises salmeterol, molnupiravir, and paxlovid.

In some examples, wherein the composition comprises linsitinib in combination with one or more additional agents.

In some examples, the composition comprises impramine or a derivative thereof in combination with one or more additional agents.

Also disclosed herein are pharmaceutical compositions comprising any of the compositions and/or compounds disclosed herein.

For example, also disclosed herein are pharmaceutical compositions comprising any of the compounds disclosed herein (e.g., a compound identified by any of the methods disclosed herein) and one more additional agents.

Also disclosed herein are compositions comprising the compound identified by any of the methods disclosed herein. In some examples, the composition further comprises a pharmaceutically acceptable excipient.

Also disclosed herein are pharmaceutical compositions comprises a pharmaceutically acceptable excipient and a therapeutically effective amount of any of the compositions disclosed herein.

In some examples, the compositions can further comprise one or more additional agents.

In some examples, the compound comprises imipramine, salmeterol, hexylresorcinol, brompheniramine, ezetimibe, temsirolimus, linsitinib, torin-1, rottlerin, semaxanib, ipratropium, AS-605240, mefenamic acid, JNJ16259685, QL-XII-47, midodrine, olanzapine, trifluoperazine, fluphenazine, azelastine, chlorphenamine, clarithromycin, saracatinib, JAK3-Inhibitor-II, AZD-8055, CGP-60474, hexamethylene, loperamide, nifedipine, liothyronine, atorvastatin, triptolide, pirfenidone, isoliquiritigenin, rucaparib, berbamine, darinaparsin, taurodeoxycholic acid; derivatives thereof; or a combination thereof. In some examples, the compound comprises imipramine, salmeterol, hexylresorcinol, brompheniramine, ezetimibe, temsirolimus, linsitinib, torin-1, rottlerin, semaxanib, ipratropium, AS-605240, mefenamic acid, JNJ16259685, QL-XII-47; derivatives thereof; or a combination thereof. In some examples, the compound comprises salmeterol, rottlerin, imipramine, linsitinib, hexylresorcinol, ezetimibe, brompheniramine; derivatives thereof; or a combination thereof. In some examples, the compound comprises salmeterol, linisitinib, imipramine, derivatives thereof, or a combination thereof. In some examples, the compound comprises salmeterol, linisitinib, imipramine, fluvoxamine, or a combination thereof.

Also disclosed herein are pharmaceutical compositions for the treatment of a coronavirus infection in a subject in need thereof, wherein the pharmaceutical composition comprises a pharmaceutically acceptable excipient and a therapeutically effective amount of a composition comprising a compound selected from the group consisting of: imipramine, salmeterol, hexylresorcinol, brompheniramine, ezetimibe, temsirolimus, linsitinib, torin-1, rottlerin, semaxanib, ipratropium, AS-605240, mefenamic acid, JNJ16259685, QL-XII-47, midodrine, olanzapine, trifluoperazine, fluphenazine, azelastine, chlorphenamine, clarithromycin, saracatinib, JAK3-Inhibitor-II, AZD-8055, CGP-60474, hexamethylene, loperamide, nifedipine, liothyronine, atorvastatin, triptolide, pirfenidone, isoliquiritigenin, rucaparib, berbamine, darinaparsin, taurodeoxycholic acid; derivatives thereof; and combinations thereof. In some examples, the composition comprises an antiviral compound, an anti-hyperinflammatory compound, or a combination thereof.

Also disclosed herein are pharmaceutical compositions for the treatment of coronavirus comprising a pharmaceutically acceptable excipient and a therapeutically effective amount of a composition comprising an antiviral compound and an anti-hyperinflammatory compound.

In some examples, the antiviral compound inhibits cell fusion or viral entry. In some examples, the antiviral compound comprises a histamine receptor antagonist, an acetylcholine receptor antagonist, a norepinephrine and serotonin reuptake inhibitor, an autophagy enhancer, a mTOR inhibitor, a PI3K inhibitor, an IGF-1- and insulin receptor inhibitor, a TB K1 activator through ARF1, an adrenergic receptor agonist, a VEGFR inhibitor, a local anesthetic, a cyclooxygenase inhibitor, a glutamate receptor antagonist, a Niemann-Pick Cl-like 1 protein antagonist, a cholesterol inhibitor, a cytoplasmic tyrosine protein kinase BMX inhibitor, a MAPK and protein kinase inhibitor, or a combination thereof. In some examples, the antiviral compound comprises: imipramine, salmeterol, hexylresorcinol, brompheniramine, ezetimibe, temsirolimus, linsitinib, torin-1, rottlerin, semaxanib, ipratropium, AS-605240, mefenamic acid, JNJ16259685, QL-XII-47; derivatives thereof; or a combination thereof. In some examples, the antiviral compound comprises: salmeterol, rottlerin, imipramine, linsitinib, hexylresorcinol, ezetimibe, brompheniramine; derivatives thereof; or a combination thereof. In some examples, the antiviral compound comprises salmeterol, linisitinib, imipramine, derivatives thereof, or a combination thereof. In some examples, the antiviral compound comprises salmeterol, linisitinib, imipramine, fluvoxamine, or a combination thereof. In some examples, the antiviral compound comprises an IGF-1R and/or insulin receptor inhibitor, such as linsitinib. In some examples, the antiviral compound comprises an adrenergic receptor agonist, such as salmeterol.

In some examples, the anti-hyperinflammatory compound comprises an adrenergic receptor agonist, a dopamine receptor antagonist, an autophagy enhancer, an autophagy dual modulator, a histamine receptor antagonist, a bacterial 50S ribosomal subunit inhibitor, an autophagy inhibitor, a SRC inhibitor, a JAK inhibitor, a mTOR inhibitor, a CDK inhibitor, a sodium/hydrogen antiport inhibitor, an opioid receptor agonist, a calcium channel blocker, a thyroid hormone stimulant, a HMGCR inhibitor, a RNA polymerase inhibitor, a TGFβ receptor inhibitor, an anti-fibrotic, a guanylate cyclase activator, a PARP inhibitor, a calmodulin antagonist, an apoptosis stimulant, a bile acid, or a combination thereof. In some examples, the anti-hyperinflammatory compound comprises midodrine, olanzapine, trifluoperazine, fluphenazine, azelastine, chlorphenamine, clarithromycin, saracatinib, JAK3-Inhibitor-II, AZD-8055, CGP-60474, hexamethylene, loperamide, nifedipine, liothyronine, atorvastatin, triptolide, pirfenidone, isoliquiritigenin, rucaparib, berbamine, darinaparsin, taurodeoxycholic acid, derivatives thereof, or a combination thereof. In some examples, the anti-hyperinflammatory compound elevates IFN signaling and/or suppresses cytokine pathways. In some examples, the anti-hyperinflammatory compound elevates IFN signaling and suppresses cytokine pathways.

In some examples, the composition comprises salmeterol, linsitinib, impramine, derivatives thereof, or a combination thereof, optionally in combination with one or more additional agents.

In some examples, the composition comprises salmeterol in combination with one or more additional agents. In some examples, the composition comprises salmeterol in combination with an RNA-dependent RNA polymerase inhibitor, a 3CL protease inhibitor, or a combination thereof. In some examples, the composition comprises salmeterol in combination with molnupiravir, paxlovid, or a combination thereof. In some examples, the composition comprises salmeterol, molnupiravir, and paxlovid.

In some examples, wherein the composition comprises linsitinib in combination with one or more additional agents.

In some examples, the composition comprises impramine or a derivative thereof in combination with one or more additional agents.

In some examples, the compositions can further comprise one or more additional agents.

Also disclosed herein are methods of treating a disease or disorder in a subject in need thereof comprising administering a therapeutically effective amount of any of the compositions (e.g., pharmaceutical compositions) as disclosed herein. In some examples, the disease or disorder comprises an infection, such as with an infectious microbe (e.g., bacteria, virus, fungi, protozoa, etc.). In some examples, the disease or disorder comprises an infection with a coronavirus. In some examples, the coronavirus comprises human coronavirus, SARS-CoV, SARS-CoV-2, or MERS-CoV.

The methods of treatment of the disease or disorder described herein can further include treatment with one or more additional agents. The one or more additional agents and the compounds as described herein can be administered in any order, including simultaneous administration, as well as temporally spaced order of up to several days apart. The methods can also include more than a single administration of the one or more additional agents and/or the compounds or compositions as described herein. The administration of the one or more additional agents and the compounds or compositions as described herein can be by the same or different routes. When treating with one or more additional agents, the compounds or compositions as described herein can be combined into a pharmaceutical composition that includes the one or more additional agents.

The one or more additional agents can, for example, comprise an anti-inflammatory agent, an antimicrobial agent, or a combination thereof. As used herein, antimicrobials include, for example, antibacterials, antifungals, and antivirals. Accordingly, in some examples, the methods can further include treatment with one or more additional antiviral agents, anti-inflammatory agents, or a combination thereof.

Examples of antimicrobial agents include, but are not limited to, alexidine, asphodelin A, atromentin, auranthine, austrocortilutein, austrocortirubin, azerizin, chlorbisan, chloroxine, cidex, cinoxacin, citreorosein, copper usnate, cupiennin, curvularin, DBNPA, dehydrocurvularin, desoxyfructo-serotonin, dichloroisocyanuric acid, elaiomycin, holtfreter's solution, malettinin, naphthomycin, neutrolin, niphimycin, nitrocefin, oxadiazoles, paenibacterin, proclin, ritiometan, ritipenem, silicone quaternary amine, stylisin, taurolidine, tirandamycin, trichloroisocyanuric acid, triclocarban, and combinations thereof.

Examples of antibacterials include, but are not limited to, acetoxycycloheximide, aciduliprofundum, actaplanin, actinorhodin, alazopeptin, albomycin, allicin, allistatin, allyl isothiocyanate, ambazone, aminocoumarin, aminoglycosides, 4-aminosalicylic acid, ampicillin, ansamycin, anthramycin, antimycin A, aphidicolin, aplasmomycin, archaeocin, arenicin, arsphenamine, arylomycin A2, ascofuranone, aspergillic acid, avenanthramide, avibactam, azelaic acid, bafilomycin, bambermycin, beauvericin, benzoyl peroxide, blasticidin S, bottromycin, brilacidin, caprazamycin, carbomycin, cathelicidin, cephalosporins, ceragenin, chartreusin, chromomycin A3, citromycin, clindamycin, clofazimine, clofoctol, clorobiocin, coprinol, coumermycin A1, cyclic lipopeptides, cycloheximide, cycloserine, dalfopristin, dapsone, daptomycin, debromomarinone, 17-dimethylaminoethylamino-17-demethoxygeldanamycin, echinomycin, endiandric acid C, enediyne, enviomycin, eravacycline, erythromycin, esperamicin, etamycin, ethambutol, ethionamide, (6S)-6-fluoroshikimic acid, fosfomycin, fosmidomycin, friulimicin, furazolidone, furonazide, fusidic acid, geldanamycin, gentamycin, gepotidacin, glycyciclines, glycyrrhizol, gramicidin S, guanacastepene A, hachimycin, halocyamine, hedamycin, helquinoline, herbimycin, hexamethylenetetramine, hitachimycin, hydramacin-1, isoniazid, kanamycin, katanosin, kedarcidin, kendomycin, kettapeptin, kidamycin, lactivicin, lactocillin, landomycin, landomycinone, lasalocid, lenapenem, leptomycin, lincosamides, linopristin, lipiarmycins, macbecin, macrolides, macromomycin B, maduropeptin, mannopeptimycin glycopeptide, marinone, meclocycline, melafix, methylenomycin A, methylenomycin B, monensin, moromycin, mupirocin, mycosubtilin, myriocin, myxopyronin, naphthomycin A, narasin, neocarzinostatin, neopluramycin, neosalvarsan, neothramycin, netropsin, nifuroxazide, nifurquinazol, nigericin, nitrofural, nitrofurantoin, nocathiacin I, novobiocin, omadacycline, oxacephem, oxazolidinones, penicillins, peptaibol, phytoalexin, plantazolicin, platensimycin, plectasin, pluramycin A, polymixins, polyoxins, pristinamycin, pristinamycin IA, promin, prothionamide, pulvinone, puromycin, pyocyanase, pyocyanin, pyrenocine, questiomycin A, quinolones, quinupristin, ramoplanin, raphanin, resistome, reuterin, rifalazil, rifamycins, ristocetin, roseophilin, salinomycin, salinosporamide A, saptomycin, saquayamycin, seraticin, sideromycin, sodium sulfacetamide, solasulfone, solithromycin, sparassol, spectinomycin, staurosporine, streptazolin, streptogramin, streptogramin B, streptolydigin, streptonigrin, styelin A, sulfonamides, surfactin, surotomycin, tachyplesin, taksta, tanespimycin, telavancin, tetracyclines, thioacetazone, thiocarlide, thiolutin, thiostrepton, tobramycin, trichostatin A, triclosan, trimethoprim, trimethoprim, tunicamycin, tyrocidine, urauchimycin, validamycin, viridicatumtoxin B, vulgamycin, xanthomycin A, xibornol, amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, vancomycin, and combinations thereof.

Examples of antifungals include, but are not limited to, abafungin, acibenzolar, acibenzolar-S-methyl, acrisorcin, allicin, aminocandin, amorolfine, amphotericin B, anidulafungin, azoxystrobin, bacillomycin, Bacillus pumilus, barium borate, benomyl, binapacryl, boric acid, bromine monochloride, bromochlorosalicylanilide, bupirimate, butenafine, candicidin, caprylic acid, captafol, captan, carbendazim, caspofungin, cerulenin, chloranil, chlormidazole, chlorophetanol, chlorothalonil, chloroxylenol, chromated copper arsenate, ciclopirox, cilofungin, cinnamaldehyde, clioquinol, copper(I) cyanide, copper(II) arsenate, cruentaren, cycloheximide, davicil, dehydroacetic acid, dicarboximide fungicides, dichlofluanid, dimazole, diphenylamine, echinocandin, echinocandin B, epoxiconazole, ethonam, falcarindiol, falcarinol, famoxadone, fenamidone, fenarimol, fenpropimorph, fentin acetate, fenticlor, filipin, fluazinam, fluopicolide, flusilazole, fluxapyroxad, fuberidazole, griseofulvin, halicylindramide, haloprogin, hamycin, hexachlorobenzene, hexachlorocyclohexa-2,5-dien-1-one, 5-hydroxy-2(5H)-furanone, iprodione, lime sulfur, mancozeb, maneb, melafix, metalaxyl, metam sodium, methylisothiazolone, methylparaben, micafungin, miltefosine, monosodium methyl arsenate, mycobacillin, myclobutanil, natamycin, beta-nitrostyrene, nystatin, paclobutrazol, papulacandin B, parietin, pecilocin, pencycuron, pentamidine, pentachloronitrobenzene, pentachlorophenol, perimycin, 2-phenylphenol, polyene antimycotic, propamocarb, propiconazole, pterulone, ptilomycalin A, pyrazophos, pyrimethanil, pyrrolnitrin, selenium disulfide, sparassol, strobilurin, sulbentine, tavaborole, tebuconazole, terbinafine, theonellamide F, thymol, tiabendazole, ticlatone, tolciclate, tolnaftate, triadimefon, triamiphos, tribromometacresol, 2,4,6-tribromophenol, tributyltin oxide, triclocarban, triclosan, tridemorph, trimetrexate, undecylenic acid, validamycin, venturicidin, vinclozolin, vinyldithiin, vusion, xanthene, zinc borate, zinc pyrithione, zineb, ziram, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, amphotericin b., and combinations thereof.

Examples of antivirals include, but are not limited to, afovirsen, alisporivir, angustific acid, angustifodilactone, alovudine, beclabuvir, 2,3-bis(acetylmercaptomethyl)quinoxaline, brincidofovir, dasabuvir, docosanol, fialuridine, ibacitabine, imiquimod, inosine, inosine pranobex, interferon, metisazone, miltefosine, neokadsuranin, neotripterifordin, ombitasvir, oragen, oseltamivir, pegylated interferon, podophyllotoxin, radalbuvir, semapimod, tecovirimat, telbivudine, theaflavin, tilorone, triptofordin C-2, variecolol, ZMapp, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, zidovudine. and combinations thereof.

Examples of suitable immunotherapeutic agents include, but are not limited to, alemtuzumab, cetuximab (ERBITUX), gemtuzumab, iodine 131 tositumomab, rituximab, trastuzamab (HERCEPTIN), and combinations thereof.

In some examples, the one or more additional agents can comprise an anti-inflammatory agent, such as steroidal and/or non-steroidal anti-inflammatory agents. Examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Examples of non-steroidal anti-inflammatory drugs include acetaminophen, aspirin, ibuprofen, naproxen, Celebrex, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

In some examples, the one or more additional agents comprises a nucleic acid. Particular nucleic acid examples include, but are not limited to, oligonucleotides, miRNA, saRNA, shRNA, siRNA, DNA, RNA, mRNA, cDNA, double stranded nucleic acid, single stranded nucleic acid, and so forth. In some examples, the nucleic acid encodes a protein or peptide, e.g. for therapeutic use.

In some examples, the one or more additional agents can comprise an RNA-dependent RNA polymerase inhibitor, a 3CL protease inhibitor, or a combination thereof.

In some examples, the one or more additional agents comprises molnupiravir, paxlovid, or a combination thereof.

In some examples, the one or more additional agents can comprise an antiviral agent(s) selected from the group consisting of abacavir, acyclovir, adefovir, amantadine, amprenavir, ampligen, arbidol, atazanavir, atripla, balapiravir, BCX4430/Galidesivir, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, GS-5734/remdesivir, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, zidovudine, and combinations thereof.

Effective amounts of a compound or composition described herein for treating a mammalian subject can, in some examples, be 1 microgram (μg) per kilogram (kg) of body weight of the subject per day (μg/kg/day) or more (e.g., 5 μg/kg/day or more, 10 μg/kg/day or more, 15 μg/kg/day or more, 20 μg/kg/day or more, 25 μg/kg/day or more, 30 μg/kg/day or more, 35 μg/kg/day or more, 40 μg/kg/day or more, 45 μg/kg/day or more, 50 μg/kg/day or more, 60 μg/kg/day or more, 70 μg/kg/day or more, 80 μg/kg/day or more, 90 μg/kg/day or more, 100 μg/kg/day or more, 125 μg/kg/day or more, 150 μg/kg/day or more, 175 μg/kg/day or more, 200 μg/kg/day or more, 225 μg/kg/day or more, 250 μg/kg/day or more, 300 μg/kg/day or more, 350 μg/kg/day or more, 400 μg/kg/day or more, 450 μg/kg/day or more, 500 μg/kg/day or more, 600 μg/kg/day or more, 700 μg/kg/day or more, 800 μg/kg/day or more, 900 μg/kg/day or more, 1 milligram (mg) per kilogram (kg) of body weight of the subject per day (mg/kg/day) or more, 5 mg/kg/day or more, 10 mg/kg/day or more, 15 mg/kg/day or more, 20 mg/kg/day or more, 25 mg/kg/day or more, 30 mg/kg/day or more, 35 mg/kg/day or more, 40 mg/kg/day or more, 45 mg/kg/day or more, 50 mg/kg/day or more, 60 mg/kg/day or more, 70 mg/kg/day or more, 80 mg/kg/day or more, 90 mg/kg/day or more, 100 mg/kg/day or more, 125 mg/kg/day or more, 150 mg/kg/day or more, 175 mg/kg/day or more, 200 mg/kg/day or more, 225 mg/kg/day or more, 250 mg/kg/day or more, 300 mg/kg/day or more, 350 mg/kg/day or more, 400 mg/kg/day or more, 450 mg/kg/day or more, 500 mg/kg/day or more, 600 mg/kg/day or more, 700 mg/kg/day or more, 800 mg/kg/day or more, or 900 mg/kg/day or more). In some examples, effective amounts of a compound or composition described herein for treating a mammalian subject can be 1000 milligrams (mg) per kilogram (kg) of body weight of the subject per day (mg/kg/day) or less (e.g., 900 mg/kg/day or less, 800 mg/kg/day or less, 700 mg/kg/day or less, 600 mg/kg/day or less, 500 mg/kg/day or less, 450 mg/kg/day or less, 400 mg/kg/day or less, 350 mg/kg/day or less, 300 mg/kg/day or less, 250 mg/kg/day or less, 225 mg/kg/day or less, 200 mg/kg/day or less, 175 mg/kg/day or less, 150 mg/kg/day or less, 125 mg/kg/day or less, 100 mg/kg/day or less, 90 mg/kg/day or less, 80 mg/kg/day or less, 70 mg/kg/day or less, 60 mg/kg/day or less, 50 mg/kg/day or less, 45 mg/kg/day or less, 40 mg/kg/day or less, 35 mg/kg/day or less, 30 mg/kg/day or less, 25 mg/kg/day or less, 20 mg/kg/day or less, 15 mg/kg/day or less, 10 mg/kg/day or less, 5 mg/kg/day or less, 1 mg/kg/day or less, 900 microgram (μg) per kilogram (kg) of body weight of the subject per day (μg/kg/day) or less, 800 μg/kg/day or less, 700 μg/kg/day or less, 600 μg/kg/day or less, 500 μg/kg/day or less, 450 μg/kg/day or less, 400 μg/kg/day or less, 350 μg/kg/day or less, 300 μg/kg/day or less, 250 μg/kg/day or less, 225 μg/kg/day or less, 200 μg/kg/day or less, 175 μg/kg/day or less, 150 μg/kg/day or less, 125 μg/kg/day or less, 100 μg/kg/day or less, 90 μg/kg/day or less, 80 μg/kg/day or less, 70 μg/kg/day or less, 60 μg/kg/day or less, 50 μg/kg/day or less, 45 μg/kg/day or less, 40 μg/kg/day or less, 35 μg/kg/day or less, 30 μg/kg/day or less, 25 μg/kg/day or less, 20 μg/kg/day or less, 15 μg/kg/day or less, 10 μg/kg/day or less, or 5 μg/kg/day or less).

Effective amounts of a compound or composition described herein for treating a mammalian subject can range from any of the minimum values described above to any of the maximum values described above. For example, effective amounts of a compound or composition described herein for treating a mammalian subject can include from 1 microgram (μg) per kilogram (kg) of body weight of the subject per day (mg/kg/day) to 1000 mg per kg of body weight of the subject per day (e.g., from 1 mg/kg/day to 1 mg/kg/day, from 1 mg/kg/day to 1000 mg/kg/day, from 1 μg/kg/day to 100 μg/kg/day, from 100 μg/kg/day to 1 mg/kg/day, from 1 mg/kg/day to 100 mg/kg/day, from 100 mg/kg/day to 1000 mg/kg/day, from 5 μg/kg/day to 1000 mg/kg/day, from 1 μg/kg/day to 900 mg/kg/day, from 5 μg/kg/day to 900 mg/kg/day, from 500 mg/kg/day to 500 mg/kg/day, from 1 to 100 mg/kg/day, or from 10 to 100 mg/kg/day). The doses can be acute or chronic. A broad range of disclosed composition dosages are believed to be both safe and effective.

It is understood, however, that the specific dose level for any particular subject will depend upon a variety of factors. Such factors include the age, body weight, general health, sex, and diet of the subject. Other factors include the time and route of administration, rate of excretion, drug combination, and the type and severity of the particular disease or disorder.

The methods, compounds, and compositions as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds or compositions as described herein are administered to a subject prior to onset (e.g., before obvious signs of the disease or disorder), during early onset (e.g., upon initial signs and symptoms of the disease or disorder), or after an established development of the disease or disorder. Prophylactic administration can occur for several days to years prior to the manifestation of symptoms of a disease or disorder. Therapeutic treatment involves administering to a subject a therapeutically effective amount of the compound or composition as described herein after the disease or disorder is diagnosed.

In certain embodiments, it is desirable to target a nanoparticle using a targeting moiety that is specific to a cell type and/or tissue type. In some embodiments, a nanoparticle may be targeted to a particular cell, tissue, and/or organ using a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′)2 fragments), single domain antibodies, camelid antibodies and fragments thereof, human antibodies and fragments thereof, monoclonal antibodies, and multispecific antibodies (e.g., bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42(5):439-62, 2003 and Abra et al., J. Liposome Res. 12:1-3, 2002.

The targeting moiety can target any known cell type, including, but not limited to, hepatocytes, colon cells, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells).

In some examples, the pharmaceutical composition is administered to a subject. In some examples, the subject is a mammal. In some examples, the mammal is a primate. In some examples, the mammal is a human. In some examples, the human is a patient.

In some examples, the disclosed compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The compositions can be conveniently presented in unit dosage form and prepared by any of the methods well known in the art of pharmacy.

Methods of Making

Also disclosed herein are methods of making any of the compounds or compositions disclosed herein.

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Katchem (Prague, Czech Republic), Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, Ill.), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical excipients disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Compositions, Formulations, Methods of Administration, and Kits

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable excipient in order to facilitate effective administration of the compound. The compositions used can also be in a variety of forms. These include, for example, solid, semisolid, and liquid dosage forms, such as tablets, pills, powders, liquid solutions or suspension, suppositories, injectable and infusible solutions, and sprays. The preferred form depends on the intended mode of administration and application. The compositions can also include conventional pharmaceutically-acceptable carriers and diluents which are known to those skilled in the art.

Examples of carriers or diluents for use with the compounds include ethanol, dimethyl sulfoxide, glycerol, alumina, starch, saline, and equivalent carriers and diluents. To provide for the administration of such dosages for the desired application, compositions disclosed herein can comprise between about 0.1% and 100% by weight of the total of one or more of the subject compounds based on the weight of the total composition including carrier or diluent.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art.

For the treatment of oncological disorders, the compounds or compositions disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds or compositions disclosed herein. For example, the compounds or compositions disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etopo side or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Pharmaceutical compositions disclosed herein suitable for injectable use include sterile aqueous solutions or dispersions. Furthermore, the compositions can be in the form of sterile powders for the extemporaneous preparation of such sterile injectable solutions or dispersions. In some examples, the final injectable form can be sterile and can be effectively fluid for easy syringability. In some examples, the pharmaceutical compositions can be stable under the conditions of manufacture and storage; thus, they can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical compositions disclosed herein can be in a form suitable for topical use such as, for example, an aerosol, cream, ointment, lotion, dusting powder, mouth washes, gargles, solution, tincture, and the like. In some examples, the compositions can be in a form suitable for use in transdermal devices. In some examples, it will be desirable to administer them topically to the skin as compositions, in combination with a dermatologically acceptable carrier, which can be a solid or a liquid. Compounds and agents and compositions disclosed herein can be applied topically to a subject's skin. These formulations can be prepared, utilizing any of the compounds disclosed herein or pharmaceutically acceptable salts thereof, via conventional processing methods.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Pharmaceutical compositions disclosed herein can be in a form suitable for rectal administration wherein the carrier is a solid. In some examples, the mixture forms unit dose suppositories. Suitable carriers include cocoa butter and other materials commonly used in the art. The suppositories can be conveniently formed by first admixing the composition with the softened or melted carriers) followed by chilling and shaping in molds.

In some examples, the pharmaceutical compositions disclosed herein can further comprise a propellant. Examples of propellants include, but are not limited to, compressed air, ethanol, nitrogen, carbon dioxide, nitrous oxide, hydrofluoroalkanes (HFA), 1,1,1,2,-tetrafluoroethane, 1,1,1,2,3,3,3-heptafluoropropane, and combinations thereof.

For administration by inhalation, the compounds or compositions can be delivered in the form of an aerosol spray from pressured container or dispenser which contains a suitable propellant or a nebulizer.

Also disclosed herein are pressurized containers comprising any of the compounds or compositions (e.g., pharmaceutical compositions) disclosed herein. Examples of containers include, but are not limited to, manual pump sprays, inhalers (e.g., meter-dosed inhalers, dry powder inhalers, etc.), and nebulizers (e.g., vibrating mesh nebulizers, jet nebulizers, ultrasonic wave nebulizers, etc.).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing any of the compounds disclosed herein, and/or pharmaceutically acceptable salts thereof, can also be prepared in powder or liquid concentrate form.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

In some examples, the kit further comprises at least one agent, wherein the compound and the agent are co-formulated.

In some examples, the compound and the agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—a Systems-Level Study Reveals Host-Targeted Repurposable Drugs Against SARS-CoV-2 Infection

Abstract. Understanding the mechanism of SARS-CoV-2 infection and identifying potential therapeutics are global imperatives. Using a quantitative systems pharmacology approach, a set of repurposable and investigational drugs were identified as potential therapeutics against COVID-19. These were deduced from the gene expression signature of SARS-CoV-2-infected A549 cells screened against Connectivity Map and prioritized by network proximity analysis with respect to disease modules in the viral-host interactome. Immuno-modulating compounds aiming at suppressing hyperinflammatory responses in severe COVID-19 patients were also identified based on the transcriptome of ACE2-overexpressing A549 cells. Experiments with Vero-E6 cells infected by SARS-CoV-2, as well as independent syncytia formation assays for probing ACE2/SARS-CoV-2 spike protein-mediated cell fusion using HEK293T and Calu-3 cells, showed that several predicted compounds had inhibitory activities. Among them, salmeterol, rottlerin, and mTOR inhibitors exhibited antiviral activities in Vero-E6 cells; imipramine, linsitinib, hexylresorcinol, ezetimibe, and brompheniramine impaired viral entry. These findings provide new paths for broadening the repertoire of compounds pursued as therapeutics against COVID-19.

Introduction. Coronavirus disease-2019 (COVID-19) caused by severe acute respiratory syndrome coronavirus (CoV) type 2 virus (SARS-CoV-2) has led to over 3 million deaths as of April 2021, and there is an urgent need to better understand the mechanisms of infection and the host cell response and to develop new therapeutics. Identification of repurposable drugs became a widespread approach for addressing current pharmacological challenges, including those faced by the current pandemic. Many compounds under clinical trials against SARS-CoV-2 are potentially repurposable drugs that target viral proteins (Esposito S et al. Infez Med. 2020, 28, 198-211; Tu Y-F et al. Int J Mot Sci. 2020, 21, 2657). While such efforts are worth pursuing, an alternative strategy is to discover host-targeted therapies. The focus herein is on the identification of repurposable compounds that modulate host cell responses, using a comprehensive, mechanism unbiased, and highly integrated systems-level approach.

The current quantitative systems pharmacology approach leverages recent progress in the field in an integrated computational/experimental framework (Stern A M et al. J Biomol Screen, 2016, 21, 521-534): One is the rigorous evaluation of the differentially expressed genes (DEGs) in SARS-CoV-2-infected cells, and the use of these DEG patterns for extracting from the Connectivity Map (CMap) database (Lamb J et al. Science, 2006, 313, 1929-1935; Subramanian A et al. Cell, 2017, 171, 1437-1452.e1417) candidate compounds/drugs that would reverse the infected cells' transcriptional program. Recent study showed, for example, the success of a CMap-based drug signature refinement approach for improving drug repositioning predictions (Iorio F et al. PLoS One, 2015, 10, e0139446). Herein, the transcriptome data from SARS-CoV-2-infected A549 (human adenocarcinomic alveolar basal epithelial) cells (Blanco-Melo D et al. bioRxiv, 2020, 10.1101/2020.03.24.004655) from lung tissue, as well as those of A549 cells overexpressing the host cell receptor angiotensin-converting enzyme 2 (ACE2) (Blanco-Melo D et al. Cell, 2020, 181, 1036-1045.e1039), were used. The latter ensures high multiplicity of infection and allows for observing the DEGs under severe infection.

Another important advance is the characterization of virus-host cell interactome for SARS-CoV-2 (Gordon D E et al. Nature, 2020, 583, 459-468) and knowledge of cell-specific protein-protein interaction (PPI) networks. These data, combined with network-based proximity analysis (Guney E et al. Nat Commun. 2016, 7, 10331), can help quantify the extent of interaction between the targets of each compound and the host cell proteins participating in the interactome with the virus. For example, Zhou et al. recently proposed 16 repurposable drugs using a network proximity analysis between drug targets in the human PPIs and host cell proteins associated with four human CoVs (SARS-CoV, MERS-CoV, HCoV-229E, and HCoV-NL63), the mouse MHV, and avian IBV, but not SARS-CoV-2 (Zhou Y et al. Cell Discov. 2020, 6, 14).

There is also access to increasingly larger databases on protein-target interactions and target-pathway mappings and interfaces, such as QuartataWeb webserver (Li H et al. Bioinformatics, 2020, 36, 3935-3937), that permit one to identify and/or predict drug-target associations and to bridge targets to cellular pathways completing chemical-target-pathway mappings.

Herein, the identification of 15 compounds is reported, including repurposable and investigational drugs, that are proposed to act against SARS-CoV-2 upon targeting the host cell machinery. In vitro assays conducted in Vero-E6 cells, HEK293T cells, and Calu-3 lung cancer cells for 10 of these prioritized compounds—six repurposable FDA-approved drugs (imipramine, salmeterol, hexylresorcinol, brompheniramine, ezetimibe, and temsirolimus) and four under development (linsitinib, torin-1, rottlerin, semaxanib)—demonstrated that several of them inhibited SARS-CoV-2 viral entry in a dose-dependent manner, with linsitinib being particularly effective. Additionally, 23 compounds are proposed for possible anti-hyperinflammatory (adjuvant) actions. These findings expand the repertoire of drugs/compounds that could be repurposed/developed for possible COVID-19 treatment.

Results

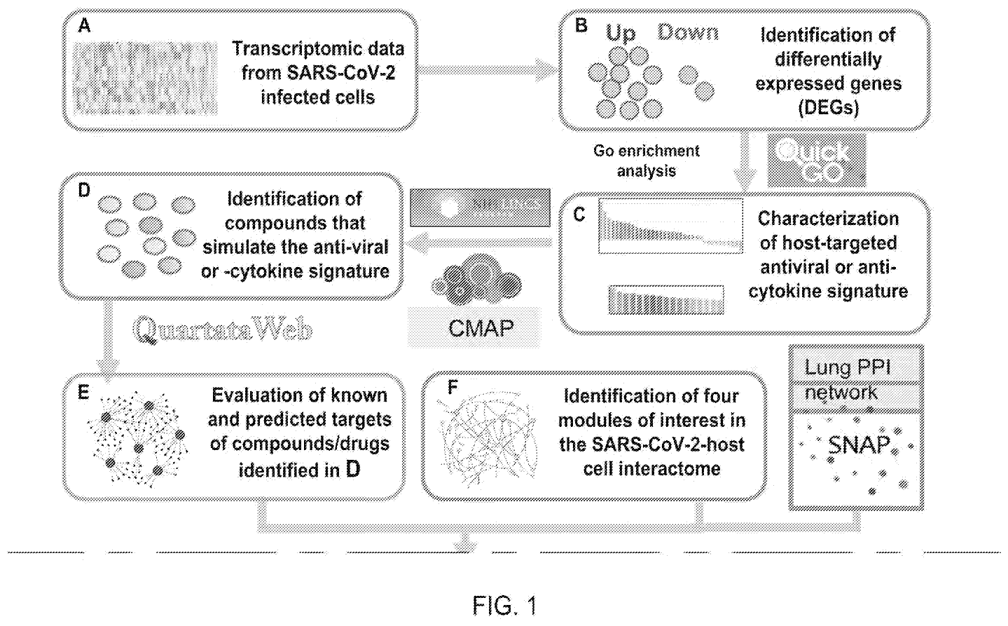

Overall workflow. FIG. 1 schematically describes the computational workflow adopted in the present study. As input, the RNA-seq data from SARS-CoV-2-infected A549 cells (Blanco-Melo D et al. bioRxiv, 2020, 10.1101/2020.03.24.004655) (referred to as Dataset 1), and those from SARS-CoV-2-infected A549 cells overexpressing ACE2 were used (shortly designated as A549-ACE2 cells) (Blanco-Melo D et al. Cell, 2020, 181, 1036-1045.e1039; tenOever B R et al. GSE147507, 2020, https://www.ncbi.nlm.nih.gov/geo/query/acc.cgi ?acc=GSE147507; Blanco-Melo D et al. bioRxiv, 2020, 10.1101/2020.03.24.004655) (referred to as Dataset 2). The corresponding DEGs were analyzed to construct antiviral and immuno-modulating (anti-inflammatory) gene signatures respectively, which were then used to predict optimal compounds/drugs that match those signatures using CMap (FIG. 1, panels A-D). Of note, the simple signature reversal approach, as utilized in many CMap studies and a recent study of SARS-CoV-2 (Duarte R R R et al. ChemRxiv, 2020, https://doi.org/10.26434/chemrxiv. 12148764. v12148761) is not applicable here, because part of the infection-induced signature promotes viral life cycle while another part reflects antiviral responses which should be promoted rather than suppressed. To address this point, 36 DEGs were selected from Dataset 1 and 17 DEGs were selected from Dataset 2, whose actions should be either reversed or promoted by CMap-deduced drugs/compounds, depending on their role in the host proteome, as will be presented in the next subsection.

Following the identification of the compounds or repurposable drugs expected to reverse the SARS-CoV-2 pathogenic (and not the host cell immunoprotective) effects (FIG. 1, panel D), a subset was prioritized following the network proximity analysis introduced by Guney et al. (Guney E et al. Nat Commun. 2016, 7, 10331) (FIG. 1, panels E-G). To this aim, the SARS-CoV-2-host interactome (Gordon D E et al. Nature, 2020, 583, 459-468) and the lung PPI network in the BioSNAP dataset were used (Zitnik M et al. BioSNAP datasets: Stanford biomedical network dataset collection. 2018, http://snapstanfordedu/biodata) (FIG. 1, panel F). First, four disease modules—viral entry, viral replication and translation, cell signaling and regulation, and immune response modules—were identified in the viral—host interactome; and then, the “distance” of each compound from each disease module was evaluated based on the proximity of the compounds' targets to the proteins belonging to the module using the lung PPI network in BioSNAP (FIG. 1, panel G).

The compounds “closest” to each module, called the prioritized compounds, were then analyzed and clustered based on their interaction patterns with targets using QuartataWeb (Li H et al. Bioinformatics, 2020, 36, 3935-3937), to select representatives from each cluster (FIG. 1, panel H). Additional criteria, such as drug development status, side effects, mechanism of action (MOA), and antiviral activities from databases and/or literature, were considered in making the final selections from among the cluster representatives for experimental tests and possible validation (FIG. 1, panel I). More specifics on the successive steps and outputs are provided below.

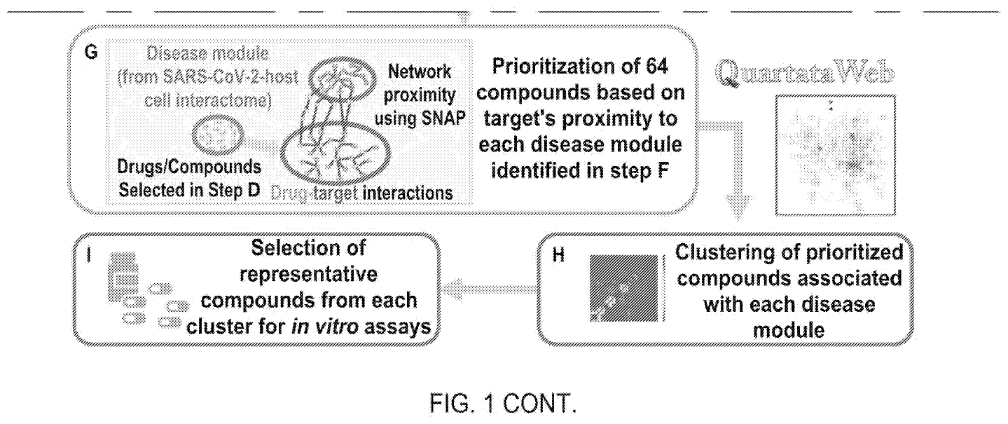

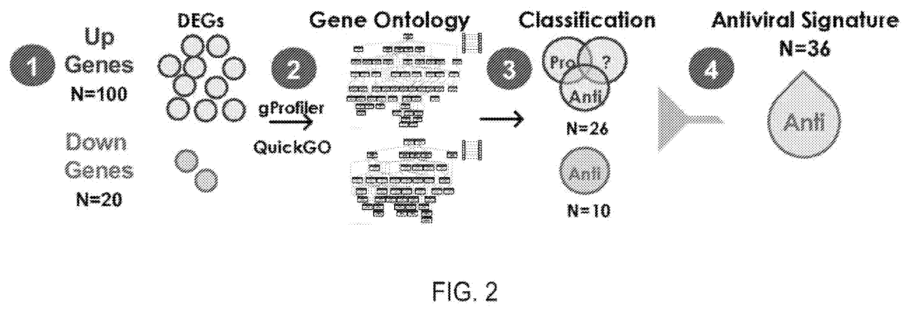

Antiviral and anti-inflammatory signatures derived from post-SARS-CoV-2 infection transcriptomics. 120 DEGs composed of 100 upregulated and 20 downregulated genes were identified by DESeq2 analysis (Love M I et al. Genome Biol. 2014, 15, 550) of the transcriptome of SARS-CoV-2-infected A549 cells (Dataset 1), using false-discovery rate (FDR) default upper value of 0.05 (FIG. 2 and Table 1).

FIG. 2-FIG. 5 show the antiviral and anti-cytokine signature derived from the post-SARS-CoV-2-infection transcriptome the respective A549 and A549-ACE2.

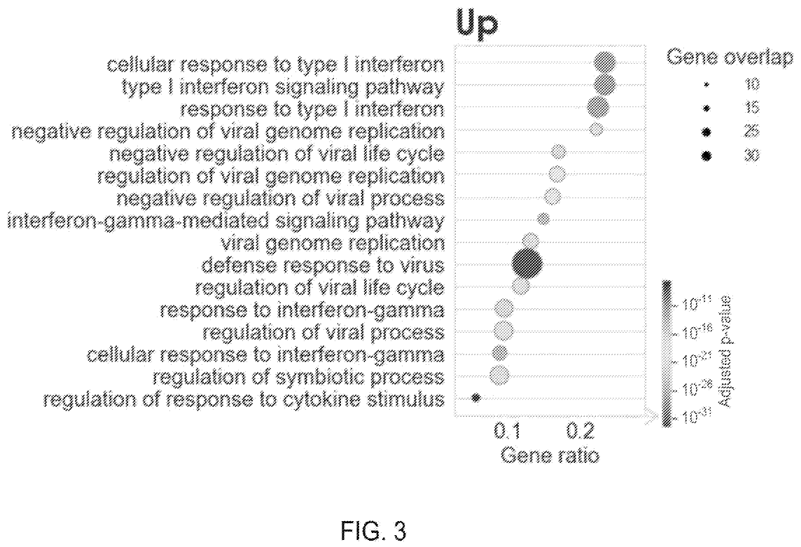

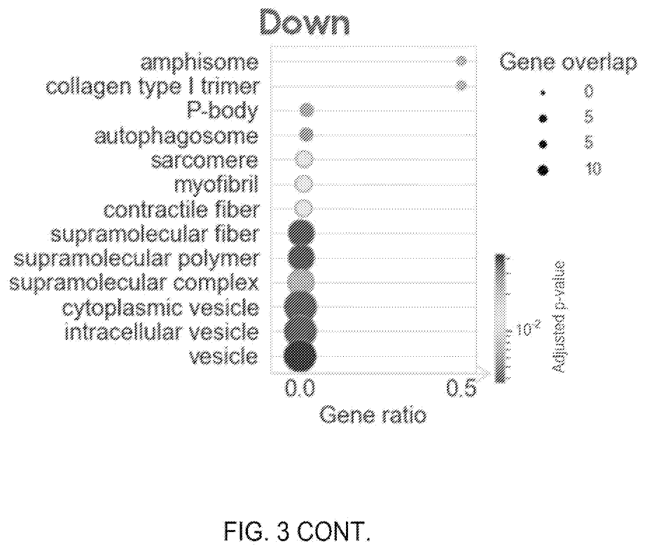

Gene Ontology (GO) (Ashburner M et al. Nat Genet. 2000, 25, 25-29; UniProt Consortium. Nucleic Acids Res. 2019, 47, D506-D515) enrichment analysis of the 100 upregulated genes showed that they were mainly involved in viral life cycle and some in early defensive immune responses mediated by interferons (IFNs; FIG. 3). Such early responses include viral translation inhibition, RNA degradation, RNA editing, or nitric oxide synthesis (Samuel C E. Clin Microbiol Rev. 2001, 14, 778-809). Nevertheless, the induction of interferon types I and III was relatively more “muted” in SARS-CoV-2-infected A549 cells compared to those of other respiratory viruses such as influenza A and respiratory syncytial virus (Blanco-Melo D et al. Cell, 2020, 181, 1036-1045.e1039).

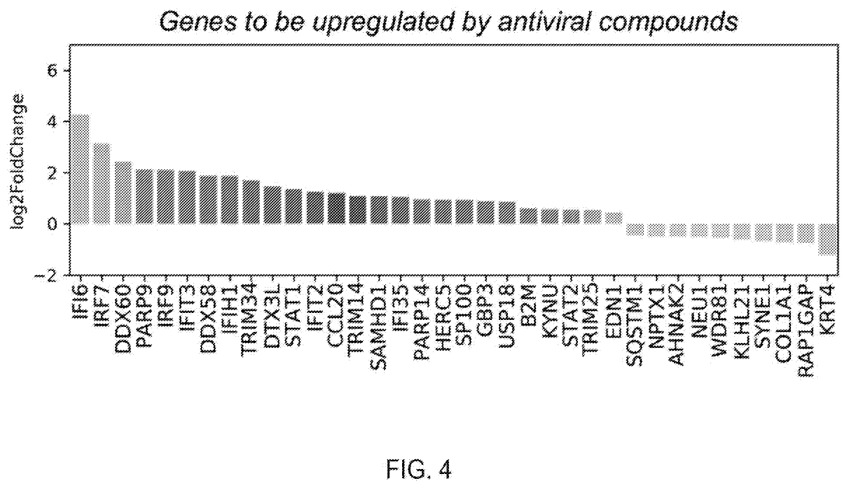

As to downregulated genes, they mainly comprised vesicle-related structures or endosomal events, including autophagosome formation for autophagic elimination of the virus (Kudchodkar S B et al. Rev Med Virol. 2009, 19, 359-378). Promoting autophagy showed potential in reducing MERS infection (Gassen N C et al. Nat Commun. 2019, 10, 5770) and thus down-regulation of this process might contribute to viral escape. In CMap applications to diabetes (Zhang M et al. PLoS One, 2015, 10, e0126082) and obesity (Liu J et al. Cell, 2015, 161, 999-1011), compounds that reverse the gene signature induced by the disease were selected. However, in SARS-CoV-2 infection, it is important to promote the adaptive immune response mediated by IFNs at early stage rather than blindly reversing the complete gene signature. Therefore, after overrepresentation analysis, and evaluation of the GO annotations associated with these genes as described in the Materials and Methods, 36 genes were selected to be upregulated (FIG. 4). These genes include (i) 26 genes upregulated in SARS-CoV-2 infected A549 cells, which are associated with viral defense and should be upregulated for antiviral activity, and (ii) 10 genes downregulated in A549 cells, associated with endocytic or vesicular processes, which should be reverted. Table 2 lists the corresponding gene products/proteins (left two columns). Table 3 provides information on their GO biological processes.

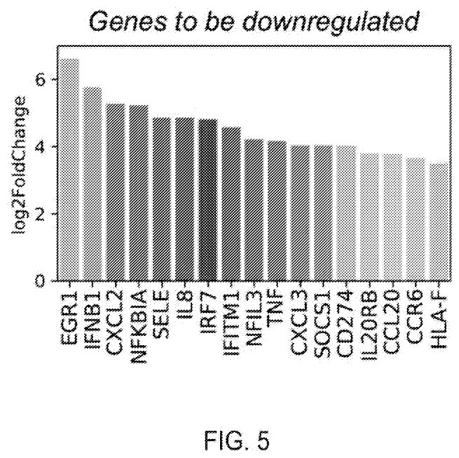

The A549-ACE2 cells (Dataset 2) repeatedly exhibited a more pronounced cytokine upregulation, along with IFN response insufficiency, compared to A549 cells. Based on this observation, immune-modulating therapies have been suggested (Blanco-Melo D et al. Cell, 2020, 181, 1036-1045.e1039). The most strongly upregulated 17 genes were selected (log₂ fold change of 3.5 or higher; see Materials and Methods), toward identifying compounds that would suppress the excessive inflammatory cytokine response in severe COVID-19 patients. This led to the anti-inflammatory (or anti-cytokine) signature shown in FIG. 5 composed of 17 genes to be downregulated (Table 2 right two columns). Table 4 list the corresponding proteins and their GO annotations.

TABLE 1
120 differentially expressed genes (DEGs) in
SARS-CoV-2-infected A549 cells.

| | | | |
|---|---|---|---|
| | | log₂ | |
| | | fold | |
| Gene name | Protein name | change | P_{adjusted} |

Upregulated 100 genes in SARS-CoV-2-infected A549 cells

| | | | |
|---|---|---|---|
| MX1 | IFN-induced | 5.20 | 1.16E−93 |
| | GTP-binding protein Mx1 | 4.59 | 6.80E−04 |
| IFI44 | IFN-induced protein 44 (Fragment) | | |
| IFIT1 | IFN-induced protein with | 4.43 | 6.18E−96 |
| | tetratricopeptide repeats 1 | | |
| | (IFIT-1) (IFN-induced 56 kDa | | |
| | protein) (IFI-56K) (P56) | | |
| IFI6 | IFNα-inducible protein 6 | 4.27 | 1.01E−145 |
| OAS2 | 2′-5′-oligoadenylate synthase 2 | 4.25 | 1.49E−06 |
| IFITM1 | IFN induced transmembrane | 4.22 | 3.12E−05 |
| | protein 1 (9-27), isoform | | |
| | CRA_a | | |
| ISG15 | Ubiquitin-like protein ISG15 | 3.80 | 6.21E−85 |
| | (Fragment) | | |
| IFI27 | IFNα-inducible protein 27, | 3.54 | 8.09E−14 |
| | mitochondrial (Fragment) | | |
| IRF7 | IFN regulatory factor 7, | 3.13 | 5.69E−43 |
| | isoform CRA_a | | |
| PTPRE | Receptor-type tyrosine-protein | 3.02 | 3.36E−10 |
| | phosphatase epsilon | | |
| | (Protein-tyrosine phosphatase | | |
| | epsilon) (R-PTP-epsilon) | | |
| | (EC 3.1.3.48) | | |
| OASL | 2′-5′-oligoadenylate | 2.53 | 8.17E−07 |
| | synthase-like protein (Fragment) | | |
| DDX60 | ATP-dependent RNA helicase DDX60 | 2.42 | 1.43E−25 |
| CMPK2 | Mitochondrial cytidine | 2.23 | 7.66E−03 |
| | monophosphate (UMP-CMP) | | |
| | kinase 2 (Fragment) | | |
| PARP9 | Protein mono-ADP-ribosyltransferase | 2.13 | 4.32E−43 |
| | PARP9 (Fragment) | | |
| IRF9 | IFN regulatory factor 9 | 2.11 | 6.73E−42 |
| IFIT3 | IFN-induced protein with | 2.07 | 1.88E−21 |
| | tetratricopeptide repeats 3 | | |
| | (IFIT-3) (CIG49) (ISG-60) | | |
| | (IFN-induced 60 kDa protein) | | |
| | (IFI-60K) (Retinoic acid-induced | | |
| | gene G protein) (P60) | | |
| | (RIG-G) | | |
| SAMD9L | Sterile α motif domain- | 1.97 | 5.51E−06 |
| | containing protein 9-like | | |
| | (Fragment) | | |
| DDX58 | Antiviral innate immune | 1.88 | 3.16E−19 |
| | response receptor RIG-I | | |
| IFIH1 | IFN-induced helicase C | 1.88 | 4.01E−15 |
| | domain-containing protein 1 | | |
| PARP10 | Poly [ADP-ribose] polymerase | 1.83 | 9.35E−07 |
| | (PARP) (EC 2.4.2 .-) | | |
| SAMD9 | Sterile a motif domain- | 1.79 | 1.76E−07 |
| | containing protein 9 (Fragment) | | |
| TRIM34 | Tripartite motif-containing | 1.71 | 2.07E−04 |
| | protein 34 (IFN-responsive | | |
| | finger protein 1) (RING | | |
| | finger protein 21) | | |
| HERC6 | Probable E3 ubiquitin-protein | 1.52 | 9.14E−14 |
| | ligase HERC6 | | |
| REC8 | Meiotic recombination protein | 1.51 | 1.81E−04 |
| | REC8 homolog (Fragment) | | |
| OAS1 | 2′-5′ oligoadenylate synthetase | 1.51 | 2.69E−33 |
| | 1 p49 isoform (Fragment) | | |
| DTX3L | E3 ubiquitin-protein ligase | 1.46 | 1.44E−21 |
| | DTX3L (EC 2.3.2.27) (B- | | |
| | lymphoma-and BAL-associated | | |
| | protein) (Protein deltex-3- | | |
| | like) (RING-type E3 | | |
| | ubiquitin transferase DTX3L; | | |
| | Rhysin-2) | | |
| HELZ2 | Helicase with zinc finger | 1.38 | 3.78E−17 |
| | domain 2 (ATP-dependent | | |
| | helicase PRIC285) | | |
| | (transcriptional coactivator) (PPAR-α- | | |
| | interacting complex protein | | |
| | 285) (PPAR-γ DNA-binding | | |
| | domain-interacting protein 1) | | |
| | (PDIP1) (Peroxisomal | | |
| | proliferator-activated receptor | | |
| | A-interacting complex 285 | | |
| | kDa protein | | |
| EIF2AK2 | eIF2AK2 protein | 1.36 | 3.59E−12 |
| STAT1 | Signal transducer and activator | 1.35 | 1.90E−28 |
| | of transcription 1 | | |
| | (Fragment) | | |
| OAS3 | 2′-5′-oligoadenylate | 1.35 | 3.29E−26 |
| | synthetase 3, 100 kDa, isoform | | |
| | CRA_a | | |
| IFI16 | Γ-IFN-inducible protein 16 | 1.35 | 4.30E−03 |
| PLSCR1 | Phospholipid scramblase (Fragment) | 1.27 | 4.77E−13 |
| IFIT2 | IFN-induced protein with | 1.25 | 1.68E−02 |
| | tetratricopeptide repeats 2 | | |
| SP110 | Sp110 nuclear body | 1.23 | 6.32E−08 |
| | protein (Fragment) | | |
| CCL20 | C-C motif chemokine 20 (Fragment) | 1.20 | 3.03E−06 |
| FGG | Fibrinogen y chain | 1.19 | 5.53E−03 |
| DDX60L | Putative ATP-dependent | 1.13 | 1.68E−05 |
| | RNA helicase DDX60 (EC | | |
| | 3.6.4.13) | | |
| CFB | Complement factor B (Fragment) | 1.12 | 7.26E−08 |
| TRIM14 | Tripartite motif-containing | 1.09 | 3.97E−11 |
| | 14, isoform CRA_c | | |
| IFIT5 | IFN-induced protein with | 1.09 | 3.90E−07 |
| | tetratricopeptide repeats 5 | | |
| | (IFIT-5) (Retinoic acid- | | |
| | and IFN-inducible 58 kDa | | |
| | protein) (P58) | | |
| SAMHD1 | Deoxynucleoside triphosphate | 1.08 | 5.81E−12 |
| | triphosphohydrolase | | |
| | SAMHD1 | | |
| PHF11 | PHD finger protein 11 (cDNA | 1.07 | 2.63E−03 |
| | FLJ56933, highly similar to | | |
| | Homo sapiens PHD finger | | |
| | protein 11 (PHF11), transcript | | |
| | variant 1, mRNA) | | |
| IFI35 | IFN-induced 35 kDa protein | 1.06 | 1.54E−08 |
| | (IFP 35) (Ifi-35) | | |
| LAP3 | Cytosol aminopeptidase (Fragment) | 1.03 | 2.82E−13 |
| CXCL5 | C-X-C motif chemokine 5 | 0.96 | 3.86E−08 |
| | (ENA-78(1-78)) (Epithelial- | | |
| | derived neutrophil-activating | | |
| | protein 78) (Small-inducible | | |
| | cytokine B5) [Cleaved into: | | |
| | ENA-78(8-78); ENA-78(9-78)] | | |
| PARP14 | Protein mono-ADP- | 0.96 | 4.02E−08 |
| | ribosyltransferase PARP14 | | |
| HERC5 | E3 ISG15—protein ligase | 0.94 | 3.70E−05 |
| | HERC5 (HECT and RLD | | |
| | domain- containing E3 | | |
| | ubiquitin protein ligase 5) | | |
| | (Fragment) | | |
| SP100 | Nuclear autoantigen Sp-100 | 0.94 | 1.23E−07 |
| | (Fragment) | | |
| BCL2A1 | Bcl-2-related protein A1 | 0.93 | 1.15E−02 |
| | (Bcl-2-like protein 5) (Bcl2-L-5) | | |
| | (Hemopoietic-specific early | | |
| | response protein) (Protein | | |
| | BFL-1) (Protein GRS) | | |
| IFITM3 | IFN-induced transmembrane protein 3 | 0.91 | 1.75E−03 |
| GBP3 | Guanylate-binding protein 3 | 0.89 | 1.26E−02 |
| USP18 | Ubl carboxyl-terminal | 0.86 | 1.21E−04 |
| | hydrolase 18 (EC 3.4.19.-) (43 kDa | | |
| | ISG15-specific protease) | | |
| | (hUBP43) (ISG15-specific- | | |
| | processing protease) | | |
| | (Ubl thioesterase 18) | | |
| CP | CP protein (Ceruloplasmin | 0.86 | 8.59E−04 |
| | (Ferroxidase)) (Ceruloplasmin | | |
| | (Ferroxidase), isoform CRA_a) | | |
| CFH | Complement factor H | 0.85 | 1.15E−02 |
| PARP12 | Poly (ADP-ribose) polymerase | 0.82 | 9.48E−05 |
| | family, member 12, | | |
| | isoform CRA_b (Zinc | | |
| | finger CCCH type domain | | |
| | containing 1) | | |
| STEAP1 | Six transmembrane epithelial | 0.81 | 9.43E−03 |
| | antigen of the prostate 1, | | |
| | isoform CRA_a | | |
| EHF | ETS homologous factor (Fragment) | 0.81 | 1.60E−02 |
| PTGS2 | Prostaglandin-endoperoxidase | 0.80 | 1.47E−03 |
| | synthase 2 (EC 1.14.99.1) | | |
| | (Fragment) | | |
| C1R | Complement C1r subcomponent | 0.80 | 4.43E−07 |
| SAT1 | Diamine acetyltransferase 1 | 0.78 | 1.89E−06 |
| BIVM- | BIVM-ERCC5 readthrough | 0.76 | 3.05E−02 |
| ERCC5 | (Fragment) | | |
| C19orf66 | Shiftless antiviral inhibitor | 0.76 | 1.03E−03 |
| | of ribosomal frameshifting | | |
| | protein (SFL) (SHFL) | | |
| | (IFN-regulated antiviral protein) | | |
| | (IRAV) (Repressor of yield | | |
| | of DENV protein) (RyDEN) | | |
| SNAP25 | Synaptosomal-associated | 0.76 | 2.62E−03 |
| | protein 25 (Fragment) | | |
| CXCL8 | Multifunctional fusion | 0.75 | 1.17E−06 |
| | protein [Includes: Interleukin-8 | | |
| | (IL-8) (C-X-C motif chemokine 8) | | |
| PDK4 | Protein-serine/threonine | 0.75 | 1.29E−02 |
| | kinase (EC 2.7.11.-) | | |
| PNPT1 | Polyribonucleotide | 0.74 | 9.53E−04 |
| | nucleotidyltransferase 1, mitochondrial | | |
| MMD | Monocyte to macrophage | 0.72 | 2.08E−02 |
| | differentiation factor | | |
| APOL6 | Apolipoprotein L, 6 | 0.71 | 3.42E−02 |
| | (Apolipoprotein L6) (cDNA | | |
| | FLJ38562 fis, clone | | |
| | HCHON2004002, similar to | | |
| | Apolipoprotein-L6) | | |
| C1S | Complement C1s subcomponent | 0.71 | 1.43E−06 |
| CXCL2 | C-X-C motif chemokine | 0.71 | 2.37E−04 |
| UBE2L6 | Ubiquitin/ISG15-conjugating | 0.71 | 3.07E−04 |
| | enzyme E2 L6 (Fragment) | | |
| NUCB2 | Nesfatin 1 (Fragment) | 0.70 | 3.56E−02 |
| APOL1 | Apolipoprotein L, 1, isoform CRA_b | 0.70 | 5.96E−03 |
| PLA2G4A | Cytosolic phospholipase | 0.70 | 1.93E−02 |
| | A2 (cPLA2) (Phospholipase A2 | | |
| | group IVA) | | |
| PAPPA- | Protein PAPPAS | 0.69 | 2.25E−02 |
| AS1 | (DIPLA1 antisense RNA 1) | | |
| TYMP | Thymidine phosphorylase isoform 2 | 0.66 | 1.33E−02 |
| FGA | Fibrinogen α chain | 0.66 | 9.95E−04 |
| PTPN12 | Tyrosine-protein phosphatase | 0.66 | 1.56E−02 |
| | non-receptor type 12 | | |
| FILIP1 | Filamin A interacting protein | 0.66 | 2.08E−02 |
| | 1, isoform CRA_c (Filamin- | | |
| | A-interacting protein 1) | | |
| ESF1 | ESF1 homolog | 0.64 | 3.36E−02 |
| NCOA7 | Nuclear receptor coactivator 7, | 0.63 | 2.58E−03 |
| | isoform CRA_c | | |
| CXCL3 | C-X-C motif chemokine 3 | 0.62 | 1.61E−02 |
| | (GRO-γ(1-73)) (Growth- | | |
| | regulated protein γ) (GRO-γ) | | |
| | (Macrophage inflammatory | | |
| | protein 2-β) (MIP2-β) | | |
| | [Cleaved into: GRO-γ(5-73)] | | |
| B2M | B-2-microglobulin | 0.61 | 1.17E−02 |
| TDRD7 | Tudor domain-containing | 0.61 | 3.92E−03 |
| | protein 7 (PCTAIRE2-binding | | |
| | protein) (Tudor repeat | | |
| | associator with PCTAIRE-2) (Trap) | | |
| C3 | Complement C3 (Fragment) | 0.61 | 1.47E−03 |
| PSIP1 | Alternative protein PSIP1 | 0.60 | 5.96E−03 |
| KTN1 | Kinectin 1 (Kinesin receptor), | 0.59 | 3.36E−02 |
| | isoform CRA_a (Kinectin 1 | | |
| | (Kinesin receptor), isoform CRA_b) | | |
| CXCL1 | Growth-regulated a protein | 0.59 | 2.00E−03 |
| | (C-X-C motif chemokine 1) | | |
| | (GRO-α(1-73)) (Melanoma | | |
| | growth stimulatory activity) | | |
| | (MGSA) (Neutrophil- | | |
| | activating protein 3) (NAP-3) | | |
| | [Cleaved into: GRO-α(4-73); | | |
| | GRO-α(5-73); GRO-α(6- | | |
| | 73)] | | |
| TCIM | Transcriptional and immune | 0.58 | 5.53E−03 |
| | response regulator (Thyroid | | |
| | cancer protein 1) (TC-1) | | |
| ADAR | Double-stranded RNA-specific | 0.57 | 1.92E−05 |
| | adenosine deaminase | | |
| | (fragment) | | |
| DKK1 | Dickkopf-like protein 1 | 0.57 | 1.24E−03 |
| KYNU | Kynureninase (Fragment) | 0.57 | 2.98E−02 |
| AREG | Amphiregulin | 0.57 | 2.09E−03 |
| LGALS3BP | Lectin galactoside-binding | 0.55 | 1.25E−02 |
| | soluble 3 binding protein | | |
| | isoform 1 (CRA_d) (Fragment) | | |
| CDH1 | Cadherin-1 | 0.55 | 2.88E−03 |
| STAT2 | Signal transducer and activator | 0.55 | 7.66E−03 |
| | of transcription 2 | | |
| HIF1A | Hypoxia-inducible factor 1, | 0.54 | 3.82E−02 |
| | a subunit (Basic helix-loop- | | |
| | helix transcription factor), | | |
| | isoform CRA_a | | |
| TRIM25 | E3 ubiquitin/ISG15 ligase TRIM25 | 0.53 | 7.01E−04 |
| HSP90B1 | Endoplasmin | 0.48 | 3.44E−02 |
| EDN1 | Endothelin-1 (Preproendothelin-1) | 0.45 | 2.50E−02 |
| | (PPET1) [Cleaved into: | | |
| | Endothelin-1 (ET-1); Big endothelin-1] | | |

Downregulated 20 genes in SARS-CoV-2-infected A549 cells

| | | | |
|---|---|---|---|
| SQSTM1 | Sequestosome-1 | −0.46 | 2.50E−02 |
| NPTX1 | Neuronal pentraxin-1 (NP1) | −0.51 | 2.25E−02 |
| | (Neuronal pentraxin I) (NP-I) | | |
| AHNAK2 | Protein AHNAK2 | −0.51 | 3.19E−03 |
| NEU1 | Sialidase-1 | −0.52 | 1.24E−02 |
| WDR81 | WD repeat-containing protein 81 | −0.53 | 2.50E−02 |
| FAM102A | Protein FAM102A | −0.57 | 2.34E−03 |
| | (Early estrogen-induced gene 1 | | |
| | protein) | | |
| DANCR | Uncharacterized protein | −0.59 | 1.84E−02 |
| | DANCR (Anti-differentiation | | |
| | ncRNA protein) (Small | | |
| | nucleolar RNA host gene protein | | |
| | 13) | | |
| NT5DC2 | 5′-nucleotidase domain- | −0.59 | 1.23E−02 |
| | containing protein 2 (Fragment) | | |
| MAP3K14 | Mitogen-activated protein | −0.59 | 1.44E−02 |
| | kinase kinase kinase 14 | | |
| | (Fragment) | | |
| KLHL21 | Kelch-like protein 21 | −0.61 | 5.58E−03 |
| LOC284454 | /(ncRNA) | −0.64 | 3.92E−02 |
| SYNE1 | Nesprin-1 | −0.68 | 2.34E−03 |
| COL1A1 | Collagen, type I, α 1, isoform CRA_a | −0.73 | 1.05E−03 |
| OSGIN1 | Oxidative stress-induced | −0.74 | 6.80E−04 |
| | growth inhibitor 1 (Fragment) | | |
| RAP1GAP | Rap1 GTPase-activating protein 1 | −0.74 | 6.04E−05 |
| NPIPB5 | Nuclear pore complex- | −0.83 | 1.06E−02 |
| | interacting protein family member | | |
| | B5 | | |
| UAP1L1 | UDP-N-acetylhexosamine | −0.84 | 4.55E−04 |
| | pyrophosphorylase-like protein | | |
| | 1 | | |
| NUPR1 | Nuclear protein 1 | −0.99 | 5.96E−03 |
| NECAB2 | N-terminal EF-hand calcium- | −1.12 | 2.50E−02 |
| | binding protein 2 (Fragment) | | |
| KRT4 | Keratin, type II cytoskeletal 4 | −1.23 | 1.84E−04 |

TABLE 2
Antiviral and anti-inflammatory signature genes derived from
SARS-CoV-2-infected cells.

| Antiviral signature | | Anti-inflammatory signature | |
|---|---|---|---|
| (based on A549 Cells) | | (A549-ACE2 cells) | |
| Gene^{a} | Protein^{b} | Gene^{c} | Protein^{b} |
| To-be-upregulated | | To-be-downregulated | |
| IFI6 | IFNα-inducible protein 6 | EGR1 | Early growth |
| | | | response |
| | | | protein |
| IRF7 | IFN regulatory factor | IFNB1 | Interferon beta |
| | 7, isoform CRA_a | | |
| DDX60 | ATP-dependent RNA | CXCL2 | C-X-C motif |
| | helicase DDX60 | | chemokine |
| PARP9 | Protein mono-ADP- | NFKBIA | NFκB inhibitor α |
| | ribosyltransferase | | |
| | PARP9 | | |
| IRF9 | IFN regulatory factor 9 | SELE | E-selectin |
| IFIT3 | IFN-induced protein | IL8 | Interleukin-8 |
| | with tetratricopeptide | | |
| | repeats 3 (Retinoic | | |
| | acid-induced gene G | | |
| | protein) (RIG-G) | | |
| DDX58 | Antiviral innate immune | IRF7 | IFN regulatory |
| | response receptor | | factor 7 |
| | RIG-I | | |
| IFIH1 | IFN-induced helicase | IFITM1 | IFN-induced |
| | C domain-containing | | transmembrane |
| | protein 1 | | protein 1 |
| TRIM34 | Tripartite motif-containing | NFIL3 | Nuclear factor |
| | protein 34 (IFN- | | interleukin- |
| | responsive finger | | 3-regulated |
| | protein 1) (RING finger | | prot |
| | protein 21) | | |
| DTX3L | E3 ubiquitin-protein | TNF | Tumor necrosis |
| | ligase DTX3L (EC | | factor |
| | 2.3.2.27) | | α |
| STAT1 | Signal transducer | CXCL3 | C-X-C motif |
| | and activator of | | chemokine 3 |
| | transcription 1 | | |
| IFIT2 | IFN-induced protein | SOCS1 | Suppressor |
| | with tetratricopeptide | | of cytokine |
| | repeats 2 | | signaling 1 |
| CCL20 | C-C motif chemokine | CD274 | Programmed |
| | 20 (Fragment) | | cell death |
| | | | 1 ligand 1 |
| TRIM14 | Tripartite motif- | IL20RB | Interleukin-20 |
| | containing 14, isoform | | receptor |
| | CRA_c | | subunit β |
| SAMHD1 | Deoxynucleoside | CCL20 | C-C motif |
| | triphosphate | | chemokine |
| | triphosphohydrolase | | 20 |
| IFI35 | IFN-induced 35 kDa protein | CCR6 | C-C chemokine |
| | (IFP 35) (Ifi-35) | | receptor type 6 |
| PARP14 | Protein mono-ADP- | PARP14 | HLA-F Human |
| | ribosyltransferase | | leukocyte |
| | | | antigen F |
| HERC5 | E3 ISG15—protein | | |
| | ligase HERC5 | | |
| | (Fragment) | | |
| SP100 | Nuclear autoantigen | | |
| | Sp-100 (Fragment) | | |
| GBP3 | Guanylate-binding protein 3 | | |
| USP18 | Ubl carboxyl-terminal | | |
| | hydrolase 18 | | |
| B2M | β2-microglobulin | | |
| KYNU | Kynureninase (Fragment) | | |
| STAT2 | Signal transducer | | |
| | and activator of | | |
| | transcription 2 | | |
| TRIM25 | E3 ubiquitin/ISG15 | | |
| | ligase TRIM25 | | |
| EDN1 | Endothelin-1 | | |
| | (Preproendothelin-1) (PPET1) | | |

| | |
|---|---|
| Gene^{d} | Protein^{b} |

To-be-upregulated

| | |
|---|---|
| SQSTM1 | Sequestosome-1 |
| AHNAK2 | Protein AHNAK2 |
| NPTX1 | Neuronal pentraxin-1 |
| | (NP1) |
| NEU1 | Sialidase-1 |
| WDR81 | WD repeat-containing |
| | protein 81 |
| KLHL21 | Kelch-like protein 21 |
| SYNE1 | Nesprin-1 |
| COL1A1 | Collagen, type I, α1, |
| | isoform CRA_a |
| RAP1GAP | Rap1 GTPase- |
| | activating protein 1 |
| KRT4 | Keratin, type II |
| | cytoskeletal 4 |

ªGenes observed to be upregulated in the transcriptome of A549 cells (Dataset 1).
^{b}Protein: gene product from UniProt Consortium (UniProt Consortium. Nucleic Acids Res. 2019, 47, D506-D515).
^{c}Genes observed to be upregulated in the transcriptome of ACE2-overexpressing A549 cells (Dataset 2); All genes are ordered by log₂ fold change in descending order. See Table 3 and Table 4 for the log₂ fold change values and associated GO biological processes or cellular components. See also FIG. 4 and FIG. 5 for the respective log₂ fold change profiles observed in SARS-CoV-2-infected-A549 and SARS-CoV-2-infected-A549-ACE2 cells.
^{d}Genes observed to be downregulated in the transcriptome of A549 cells (Dataset 1).

TABLE 3
Properties of the 36 DEGs that define the antiviral gene signature
derived from the transciptome of SARS-CoV-2-infected A549 cells.

| | | | | | | |
|---|---|---|---|---|---|---|
| | Gene | | log₂ fold | | GO | |
| Index | name | Protein name | Change | Database | Annotation | GO number |

Upregulated Genes That Should Be Upregulated By Small Molecules

| | | | | | | |
|---|---|---|---|---|---|---|
| 1 | IFI6 | IFNα-inducible | 4.27 | GO:BP | type I IFN | GO:0060337 |
| | | protein 6 | | | signaling pathway | |
| | | | | | defense response | GO:0051607 |
| | | | | | to virus | |
| | | | | | cellular response | GO:0071357 |
| | | | | | to type I IFN | |
| | | | | | response to type I IFN | GO:0034340 |
| 2 | IRF7 | IFN regulatory | 3.13 | GO:BP | cellular response | GO:0071346 |
| | | factor 7, isoform | | | to IFN-γ | |
| | | CRA_a | | | type I IFN | GO:0060337 |
| | | | | | signaling pathway | |
| | | | | | cellular response | GO:0071357 |
| | | | | | to type I IFN | |
| | | | | | response to type I IFN | GO:0034340 |
| | | | | | response to IFN-γ | GO:0034341 |
| | | | | | defense response | GO:0051607 |
| | | | | | to virus | |
| | | | | | regulation of response | GO:0060759 |
| | | | | | to cytokine stimulus | |
| | | | | | IFN-γ-mediated | GO:0060333 |
| | | | | | signaling pathway | |
| 3 | DDX60 | ATP-dependent | 2.42 | GO:BP | defense response | GO:0051607 |
| | | RNA helicase DDX60 | | | to virus | |
| 4 | PARP9 | Protein mono-ADP- | 2.13 | GO:BP | regulation of response | GO:0060759 |
| | | ribosyltransferase | | | to cytokine stimulus | |
| | | PARP9 (Fragment) | | | cellular response | GO:0071346 |
| | | | | | to IFN-γ | |
| | | | | | IFN-γ-mediated | GO:0060333 |
| | | | | | signaling pathway | |
| | | | | | response to IFN-γ | GO:0034341 |
| | | | | | defense response | GO:0051607 |
| | | | | | to virus | |
| 5 | IRF9 | IFN regulatory | 2.11 | GO:BP | response to type I IFN | GO:0034340 |
| | | factor 9 | | | cellular response | GO:0071346 |
| | | | | | to IFN-γ | |
| | | | | | response to IFN-γ | GO:0034341 |
| | | | | | IFN-γ-mediated | GO:0060333 |
| | | | | | signaling pathway | |
| | | | | | cellular response | GO:0071357 |
| | | | | | to type I IFN | |
| | | | | | type I IFN | GO:0060337 |
| | | | | | signaling pathway | |
| | | | | | defense response | GO:0051607 |
| | | | | | to virus | |
| 6 | IFIT3 | IFN-induced | 2.07 | GO:BP | cellular response | GO:0071357 |
| | | protein with | | | to type I IFN | |
| | | tetratricopeptide | | | defense response | GO:0051607 |
| | | repeats 3 | | | to virus | |
| | | | | | type I IFN | GO:0060337 |
| | | | | | signaling pathway | |
| | | | | | response to type I IFN | GO:0034340 |
| 7 | DDX58 | Antiviral innate | 1.88 | GO:BP | defense response | GO:0051607 |
| | | immune response | | | to virus | |
| | | receptor RIG-I | | | regulation of response | GO:0060759 |
| | | | | | to cytokine stimulus | |
| 8 | IFIH1 | IFN-induced | 1.88 | GO:BP | defense response | GO:0051607 |
| | | helicase C domain- | | | to virus | |
| | | containing protein 1 | | | regulation of response | GO:0060759 |
| | | | | | to cytokine stimulus | |
| 9 | TRIM34 | Tripartite motif- | 1.71 | GO:BP | defense response | GO:0051607 |
| | | containing protein | | | to virus | |
| | | 34 (IFN-responsive | | | response to IFN-γ | GO:0034341 |
| | | finger protein 1) | | | IFN-γ-mediated | GO:0060333 |
| | | (RING finger | | | signaling pathway | |
| | | protein 21) | | | cellular response | GO:0071346 |
| | | | | | to IFN-γ | |
| 10 | DTX3L | E3 ubiquitin-protein | 1.46 | GO:BP | defense response | GO:0051607 |
| | | ligase DTX3L | | | to virus | |
| 11 | STAT1 | Signal transducer | 1.35 | GO:BP | regulation of response | GO:0060759 |
| | | and activator of | | | to cytokine stimulus | |
| | | transcription 1 | | | type I IFN | GO:0060337 |
| | | (Fragment) | | | signaling pathway | |
| | | | | | defense response | GO:0051607 |
| | | | | | to virus | |
| | | | | | cellular response | GO:0071357 |
| | | | | | to type I IFN | |
| | | | | | negative regulation | GO:0048525 |
| | | | | | of viral process | |
| | | | | | response to type I IFN | GO:0034340 |
| | | | | | IFN-γ-mediated | GO:0060333 |
| | | | | | signaling pathway | |
| | | | | | response to IFN-γ | GO:0034341 |
| | | | | | regulation of symbiosis, | GO:0043903 |
| | | | | | encompassing mutualism | |
| | | | | | through parasitism | |
| | | | | | negative regulation of | GO:0043901 |
| | | | | | multi-organism process | |
| | | | | | cellular response | GO:0071346 |
| | | | | | to IFN-γ | |
| | | | | | regulation of | GO:0050792 |
| | | | | | viral process | |
| 12 | IFIT2 | IFN-induced | 1.25 | GO:BP | cellular response | GO:0071357 |
| | | protein with | | | to type I IFN | |
| | | tetratricopeptide | | | defense response | GO:0051607 |
| | | repeats 2 | | | to virus | |
| | | | | | type I IFN | GO:0060337 |
| | | | | | signaling pathway | |
| | | | | | response to type I IFN | GO:0034340 |
| 13 | CCL20 | C-C motif | 1.2 | GO:BP | response to IFN-γ | GO:0034341 |
| | | chemokine 20 | | | cellular response | GO:0071346 |
| | | (Fragment) | | | to IFN-γ | |
| 14 | TRIM14 | Tripartite motif- | 1.09 | GO:BP | regulation of | GO:0050792 |
| | | containing 14, | | | viral process | |
| | | isoform CRA_c | | | regulation of symbiosis, | GO:0043903 |
| | | | | | encompassing mutualism | |
| | | | | | through parasitism | |
| | | | | | negative regulation of | GO:0043901 |
| | | | | | multi-organism process | |
| | | | | | negative regulation | GO:0048525 |
| | | | | | of viral process | |
| 15 | SAMHD1 | Deoxynucleoside | 1.08 | GO:BP | cellular response | GO:0071357 |
| | | triphosphate | | | to type I IFN | |
| | | triphosphohydrolase | | | negative regulation of | GO:0043901 |
| | | SAMHD1 | | | multi-organism process | |
| | | | | | regulation of response | GO:0060759 |
| | | | | | to cytokine stimulus | |
| | | | | | response to type I IFN | GO:0034340 |
| | | | | | defense response | GO:0051607 |
| | | | | | to virus | |
| | | | | | type I IFN | GO:0060337 |
| | | | | | signaling pathway | |
| 16 | IFI35 | IFN-induced 35 | 1.06 | GO:BP | type I IFN | GO:0060337 |
| | | kDa protein (IFP | | | signaling pathway | |
| | | 35) (Ifi- 35) | | | cellular response | GO:0071357 |
| | | | | | to type I IFN | |
| | | | | | response to type I IFN | GO:0034340 |
| 17 | PARP14 | Protein mono-ADP- | 0.96 | GO:BP | regulation of response | GO:0060759 |
| | | ribosyltransferase | | | to cytokine stimulus | |
| | | PARP14 | | | IFN-γ-mediated | GO:0060333 |
| | | | | | signaling pathway | |
| | | | | | cellular response | GO:0071346 |
| | | | | | to IFN-γ | |
| | | | | | response to IFN-γ | GO:0034341 |
| | | | | | negative regulation of | GO:0043901 |
| | | | | | multi-organism process | |
| 18 | HERC5 | E3 ISG15-protein | 0.94 | GO:BP | defense response | GO:0051607 |
| | | ligase HERC5 | | | to virus | |
| | | (Fragment) | | | | |
| 19 | SP100 | Nuclear autoantigen | 0.94 | GO:BP | cellular response | GO:0071357 |
| | | Sp-100 (Fragment) | | | to type I IFN | |
| | | | | | type I IFN | GO:0060337 |
| | | | | | signaling pathway | |
| | | | | | response to type I IFN | GO:0034340 |
| | | | | | response to IFN-γ | GO:0034341 |
| | | | | | IFN-γ-mediated | GO:0060333 |
| | | | | | signaling pathway | |
| | | | | | cellular response | GO:0071346 |
| | | | | | to IFN-γ | |
| 20 | GBP3 | Guanylate-binding | 0.89 | GO:BP | response to IFN-γ | GO:0034341 |
| | | protein 3 | | | cellular response | GO:0071346 |
| | | | | | to IFN-γ | |
| | | | | | defense response | GO:0051607 |
| | | | | | to virus | |
| 21 | USP18 | Ubl carboxyl- | 0.86 | GO:BP | type I IFN | GO:0060337 |
| | | terminal hydrolase | | | signaling pathway | |
| | | 18 | | | cellular response | GO:0071357 |
| | | | | | to type I IFN | |
| | | | | | response to type I IFN | GO:0034340 |
| | | | | | regulation of response | GO:0060759 |
| | | | | | to cytokine stimulus | |
| 22 | B2M | B-2-microglobulin | 0.61 | GO:BP | response to IFN-γ | GO:0034341 |
| | | | | | cellular response | GO:0071346 |
| | | | | | to IFN-γ | |
| | | | | | IFN-γ-mediated | GO:0060333 |
| | | | | | signaling pathway | |
| 23 | KYNU | Kynureninase | 0.57 | GO:BP | response to IFN-γ | GO:0034341 |
| | | (Fragment) | | | | |
| 24 | STAT2 | Signal transducer | 0.55 | GO:BP | defense response | GO:0051607 |
| | | and activator of | | | to virus | |
| | | transcription 2 | | | response to type I IFN | GO:0034340 |
| | | | | | cellular response | GO:0071357 |
| | | | | | to type I IFN | |
| | | | | | type I IFN | GO:0060337 |
| | | | | | signaling pathway | |
| 25 | TRIM25 | E3 ubiquitin/ISG15 | 0.53 | GO:BP | cellular response | GO:0071346 |
| | | ligase TRIM25 | | | to IFN-γ | |
| | | | | | regulation of | GO:1903900 |
| | | | | | viral life cycle | |
| | | | | | response to IFN-γ | GO:0034341 |
| | | | | | negative regulation | GO:1903901 |
| | | | | | of viral life cycle | |
| | | | | | IFN-γ-mediated | GO:0060333 |
| | | | | | signaling pathway | |
| | | | | | regulation of | GO:0050792 |
| | | | | | viral process | |
| | | | | | negative regulation of | GO:0043901 |
| | | | | | multi-organism process | |
| | | | | | defense response | GO:0051607 |
| | | | | | to virus | |
| | | | | | negative regulation | GO:0048525 |
| | | | | | of viral process | |
| | | | | | regulation of symbiosis, | GO:0043903 |
| | | | | | encompassing mutualism | |
| | | | | | through parasitism | |
| 26 | EDN1 | Endothelin-1 | 0.45 | GO:BP | regulation of response | GO:0060759 |
| | | (Preproendothelin- | | | to cytokine stimulus | |
| | | 1) (PPET1) | | | response to IFN-γ | GO:0034341 |
| | | | | | cellular response | GO:0071346 |
| | | | | | to IFN-γ | |

Down regulated Genes That Should Be Upregulated By Small Molecules

| | | | | | | |
|---|---|---|---|---|---|---|
| 1 | SQSTM1 | Sequestosome-1 | −0.46 | GO:CC | supramolecular complex | GO:0099080 |
| | | | | | intracellular vesicle | GO:0097708 |
| | | | | | supramolecular fiber | GO:0099512 |
| | | | | | supramolecular polymer | GO:0099081 |
| | | | | | cytoplasmic vesicle | GO:0031410 |
| | | | | | myofibril | GO:0030016 |
| | | | | | sarcomere | GO:0030017 |
| | | | | | contractile fiber | GO:0043292 |
| | | | | | amphisome | GO:0044753 |
| | | | | | P-body | GO:0000932 |
| | | | | | autophagosome | GO:0005776 |
| | | | | | vesicle | GO:0031982 |
| 2 | AHNAK2 | Protein AHNAK2 | −0.51 | GO:CC | supramolecular complex | GO:0099080 |
| | | | | | intracellular vesicle | GO:0097708 |
| | | | | | cytoplasmic vesicle | GO:0031410 |
| | | | | | sarcomere | GO:0030017 |
| | | | | | supramolecular polymer | GO:0099081 |
| | | | | | supramolecular fiber | GO:0099512 |
| | | | | | myofibril | GO:0030016 |
| | | | | | contractile fiber | GO:0043292 |
| | | | | | vesicle | GO:0031982 |
| 3 | NPTX1 | Neuronal pentraxin- | −0.51 | GO:CC | cytoplasmic vesicle | GO:0031410 |
| | | 1 (NP1) (Neuronal | | | intracellular vesicle | GO:0097708 |
| | | pentraxin I) (NP-I) | | | vesicle | GO:0031982 |
| 4 | NEU1 | Sialidase-1 | −0.52 | GO:CC | cytoplasmic vesicle | GO:0031410 |
| | | | | | intracellular vesicle | GO:0097708 |
| | | | | | vesicle | GO:0031982 |
| 5 | WDR81 | WD repeat- | −0.53 | GO:CC | cytoplasmic vesicle | GO:0031410 |
| | | containing protein | | | vesicle | GO:0031982 |
| | | 81 | | | autophagosome | GO:0005776 |
| | | | | | intracellular vesicle | GO:0097708 |
| 6 | KLHL21 | Kelch-like protein | −0.61 | GO:CC | supramolecular polymer | GO:0099081 |
| | | 21 | | | cytoplasmic vesicle | GO:0031410 |
| | | | | | intracellular vesicle | GO:0097708 |
| | | | | | supramolecular complex | GO:0099080 |
| | | | | | vesicle | GO:0031982 |
| | | | | | supramolecular fiber | GO:0099512 |
| 7 | SYNE1 | Nesprin-1 | −0.68 | GO:CC | P-body | GO:0000932 |
| | | | | | myofibril | GO:0030016 |
| | | | | | contractile fiber | GO:0043292 |
| | | | | | supramolecular polymer | GO:0099081 |
| | | | | | sarcomere | GO:0030017 |
| | | | | | supramolecular complex | GO:0099080 |
| | | | | | supramolecular fiber | GO:0099512 |
| 8 | COL1A1 | Collagen, type I, α | −0.73 | GO:CC | vesicle | GO:0031982 |
| | | 1, isoform CRA_a | | | supramolecular fiber | GO:0099512 |
| | | | | | supramolecular polymer | GO:0099081 |
| | | | | | cytoplasmic vesicle | GO:0031410 |
| | | | | | intracellular vesicle | GO:0097708 |
| | | | | | supramolecular complex | GO:0099080 |
| | | | | | collagen type I trimer | GO:0005584 |
| 9 | RAP1GAP | Rap1 GTPase- | −0.74 | GO:CC | cytoplasmic vesicle | GO:0031410 |
| | | activating protein 1 | | | vesicle | GO:0031982 |
| | | | | | intracellular vesicle | GO:0097708 |
| 10 | KRT4 | Keratin, type II | −1.23 | GO:CC | supramolecular fiber | GO:0099512 |
| | | cytoskeletal 4 | | | supramolecular polymer | GO:0099081 |
| | | | | | supramolecular complex | GO:0099080 |

* Showing enrichment result from Gene Ontology Biological Process (GO:BP) and Cellular Component (GO:CC) databases with term size <300 genes and overlap size >10 genes.

TABLE 4
Properties of the 17 DEGs that define the anti-cytokine signature derived
from the transcriptome of SARS-CoV-2-infected A549-ACE2 cells.

| | Gene | | log₂ Fold | | | GO | |
|---|---|---|---|---|---|---|---|
| Index | name | Protein name | Change | P_{adjusted} | Database | Annotation | GO number |
| 1 | EGR1 | Early growth | 6.62 | 0.00E+00 | GO:BP | cytokine-mediated | GO:0019221 |
| | | response protein | | | | signaling pathway | |
| | | | | | | cellular response to | GO:0071345 |
| | | | | | | cytokine stimulus | |
| 2 | IFNB1 | Interferon β | 5.76 | 1.15E−28 | GO:BP | cytokine-mediated | GO:0019221 |
| | | | | | | signaling pathway | |
| | | | | | | cellular response to | GO:0071345 |
| | | | | | | cytokine stimulus | |
| | | | | | | regulation of response | GO:0032101 |
| | | | | | | to external stimulus | |
| | | | | | | positive regulation of | GO:0002684 |
| | | | | | | immune system process | |
| 3 | CXCL2 | C-X-C motif | 5.27 | 0.00E+00 | GO:BP | cytokine-mediated | GO:0019221 |
| | | chemokine | | | | signaling pathway | |
| | | | | | | cellular response to | GO:0071345 |
| | | | | | | cytokine stimulus | |
| 4 | NFKBIA | NFκB | 5.23 | 0.00E+00 | GO:BP | cytokine-mediated | GO:0019221 |
| | | inhibitorα | | | | signaling pathway | |
| | | | | | | cellular response to | GO:0071345 |
| | | | | | | cytokine stimulus | |
| | | | | | | regulation of response | GO:0032101 |
| | | | | | | to external stimulus | |
| 5 | SELE | E-selectin | 4.85 | 1.59E−25 | GO:BP | regulation of response | GO:0032101 |
| | | | | | | to external stimulus | |
| | | | | | | positive regulation of | GO:0002684 |
| | | | | | | immune system process | |
| 6 | IL8 | Multifunctional | 4.85 | 0.00E+00 | GO:BP | cytokine-mediated | GO:0019221 |
| | | fusion protein | | | | signaling pathway | |
| | | [Includes: | | | | cellular response to | GO:0071345 |
| | | Interleukin-8 | | | | cytokine stimulus | |
| | | | | | | regulation of response | GO:0032101 |
| | | | | | | to external stimulus | |
| | | | | | | positive regulation of | GO:0002684 |
| | | | | | | immune system process | |
| 7 | IRF7 | Interferon | 4.81 | 2.69E−97 | GO:BP | cytokine-mediated | GO:0019221 |
| | | regulatory | | | | signaling pathway | |
| | | factor 7 | | | | cellular response to | GO:0071345 |
| | | | | | | cytokine stimulus | |
| | | | | | | regulation of response | GO:0032101 |
| | | | | | | to external stimulus | |
| | | | | | | positive regulation of | GO:0002684 |
| | | | | | | immune system process | |
| 8 | IFITM1 | Interferon | 4.58 | 3.21E−47 | GO:BP | cytokine-mediated | GO:0019221 |
| | | induced | | | | signaling pathway | |
| | | transmembrane | | | | cellular response to | GO:0071345 |
| | | protein 1 | | | | cytokine stimulus | |
| 9 | NFIL3 | Nuclear factor | 4.22 | 0.00E+00 | GO:BP | cellular response to | GO:0071345 |
| | | interleukin-3- | | | | cytokine stimulus | |
| | | regulated protein | | | | | |
| 10 | TNF | Tumor necrosis | 4.16 | 3.70E−29 | GO:BP | cytokine-mediated | GO:0019221 |
| | | factor | | | | signaling pathway | |
| | | | | | | cellular response to | GO:0071345 |
| | | | | | | cytokine stimulus | |
| | | | | | | regulation of response | GO:0032101 |
| | | | | | | to external stimulus | |
| | | | | | | positive regulation of | GO:0002684 |
| | | | | | | immune system process | |
| 11 | CXCL3 | C-X-C motif | 4.03 | 0.00E+00 | GO:BP | cytokine-mediated | GO:0019221 |
| | | chemokine 3 | | | | signaling pathway | |
| | | | | | | cellular response to | GO:0071345 |
| | | | | | | cytokine stimulus | |
| 12 | SOCS1 | Suppressor of | 4.02 | 1.48E−16 | GO:BP | cytokine-mediated | GO:0019221 |
| | | cytokine | | | | signaling pathway | |
| | | signaling 1 | | | | cellular response to | GO:0071345 |
| | | | | | | cytokine stimulus | |
| | | | | | | regulation of response | GO:0032101 |
| | | | | | | to external stimulus | |
| | | | | | | positive regulation of | GO:0002684 |
| | | | | | | immune system process | |
| 13 | CD274 | Programmed cell | 4.01 | 1.20E−46 | GO:BP | positive regulation of | GO:0002684 |
| | | death 1 ligand 1 | | | | immune system process | |
| 14 | IL20RB | Interleukin-20 | 3.79 | 1.15E−34 | GO:BP | cytokine-mediated | GO:0019221 |
| | | receptor | | | | signaling pathway | |
| | | subunit β | | | | cellular response to | GO:0071345 |
| | | | | | | cytokine stimulus | |
| | | | | | | regulation of response | GO:0032101 |
| | | | | | | to external stimulus | |
| 15 | CCL20 | C-C motif | 3.77 | 0.00E+00 | GO:BP | cytokine-mediated | GO:0019221 |
| | | chemokine 20 | | | | signaling pathway | |
| | | | | | | cellular response to | GO:0071345 |
| | | | | | | cytokine stimulus | |
| | | | | | | positive regulation of | GO:0002684 |
| | | | | | | immune system process | |
| 16 | CCR6 | C-C chemokine | 3.66 | 1.60E−16 | GO:BP | cytokine-mediated | GO:0019221 |
| | | receptor type 6 | | | | signaling pathway | |
| | | | | | | cellular response to | GO:0071345 |
| | | | | | | cytokine stimulus | |
| | | | | | | regulation of response | GO:0032101 |
| | | | | | | to external stimulus | |
| | | | | | | positive regulation of | GO:0002684 |
| | | | | | | immune system process | |
| 17 | HLA-F | HLA-F | 3.49 | 3.59E−86 | GO:BP | cytokine-mediated | GO:0019221 |
| | | | | | | signaling pathway | |
| | | | | | | cellular response to | GO:0071345 |
| | | | | | | cytokine stimulus | |
| | | | | | | regulation of response | GO:0032101 |
| | | | | | | to external stimulus | |
| | | | | | | positive regulation of | GO:0002684 |
| | | | | | | immune system process | |

* Gene name: gene symbol of the DEGs. Protein name: protein name of corresponding genes from UniProt (UniProt Consortium. Nucleic Acids Res. 2019, 47, D506-D515) (Genes are ordered by log₂ fold change in descending order. see STAR*Methods for the definition of adjusted P. Showing enrichment results from GO Biological Process database with term size < 1,200 genes and overlap size >10 genes. Log₂ Fold Change: log₂ transformation of the fold change in gene expression level after viral infection; Database: Gene ontology (GO) database used. BP refers to biological process, CC refers to cellular component; Term: name of the gene set in the GO database; GO number: the index of the GO term.
* Genes are ordered by descending log₂ Fold Change.

Identification of antiviral and anti-cytokine compounds and corresponding targets. The compounds that best matched the antiviral and anti-cytokine signatures determined above were identified by screening each signature against the CMap database. Briefly, the Touchstone collection of perturbagen signatures from 3,000 compounds on six cell lines was searched to assign a Cmap connectivity score to each compound. The score is based on the similarity between the compound-induced gene signature in Cmap and the query/input signature, repeated separately for the antiviral and anti-cytokine signatures. This led to a set of 263 potentially antiviral compounds, and another of 275 potentially anti-cytokine compounds, using default thresholds in Cmap (see Materials and Methods), listed in Table 5 and Table 6, respectively. The compounds included twelve (chlorpromazine, apicidin, ribavirin, mycophenolate, entacapone, equilin, metformin, mercaptopurine, gemcitabine, mepacrine/quinacrine, daunorubicin, and valproic acid) listed in the COVID-19 drug repurposing database compiled by Excelra (Excelra, 2020 COVID-19 drug repurposing database. https://wwwexcelracom/covid-19-drug-repurposing-database/).

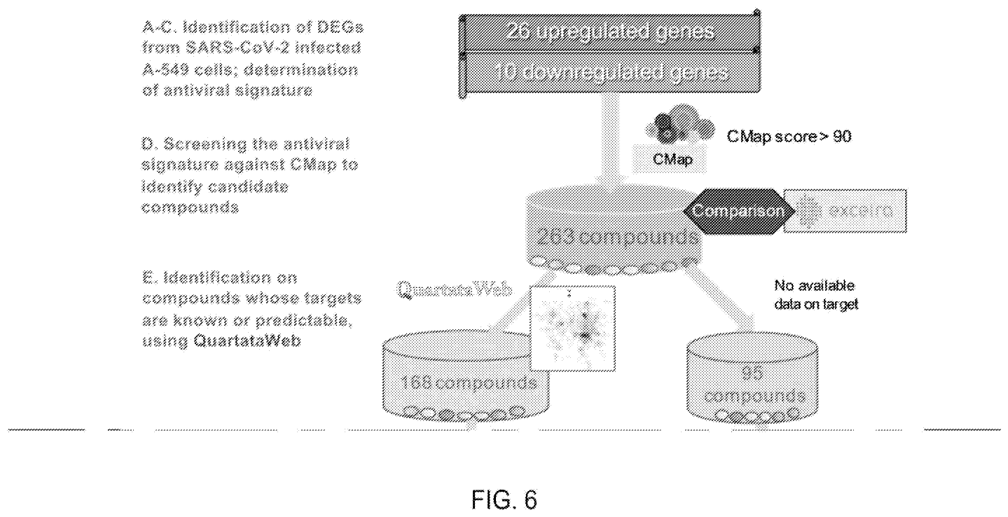

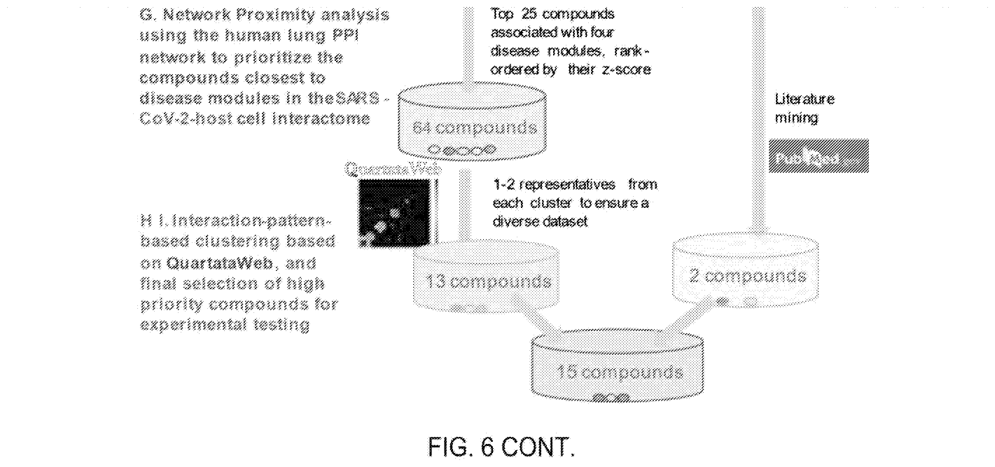

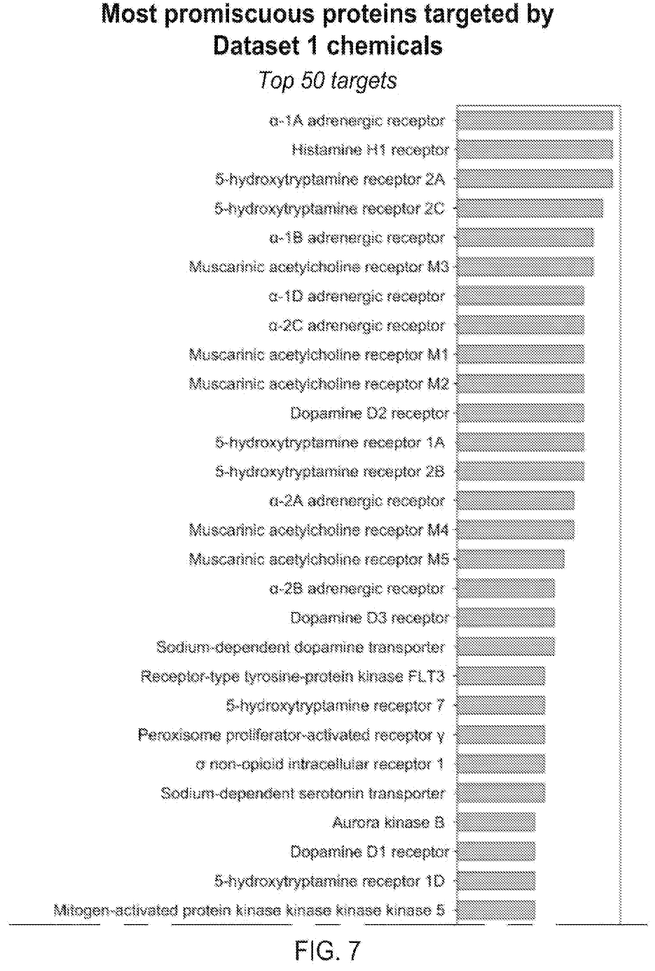

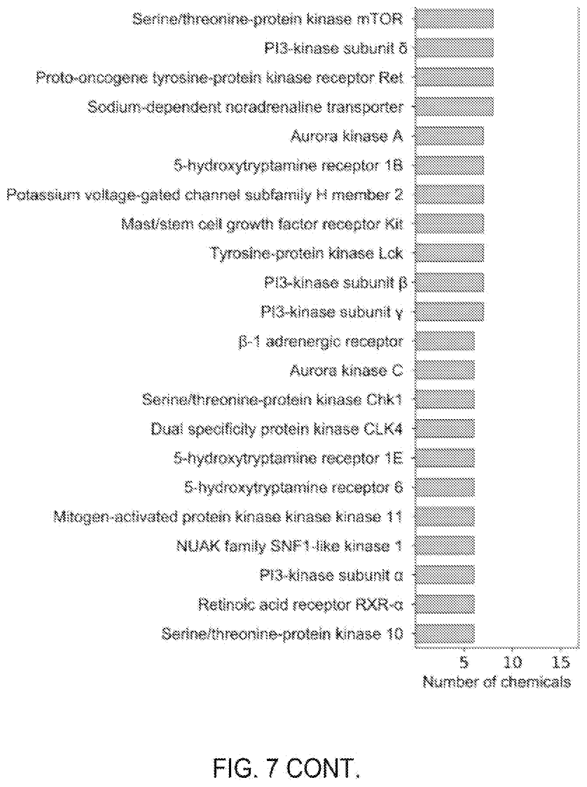

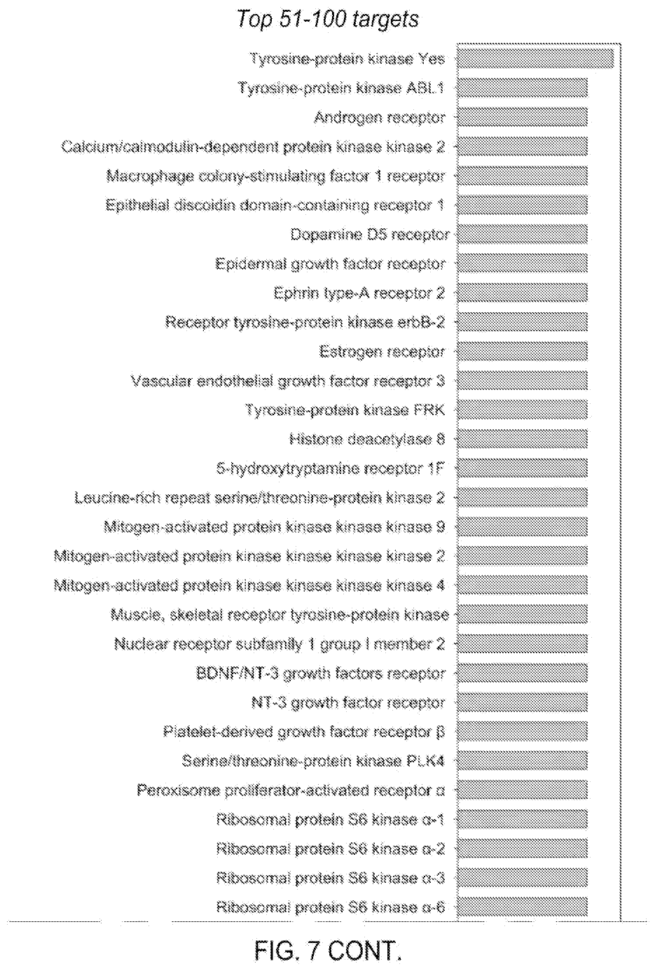

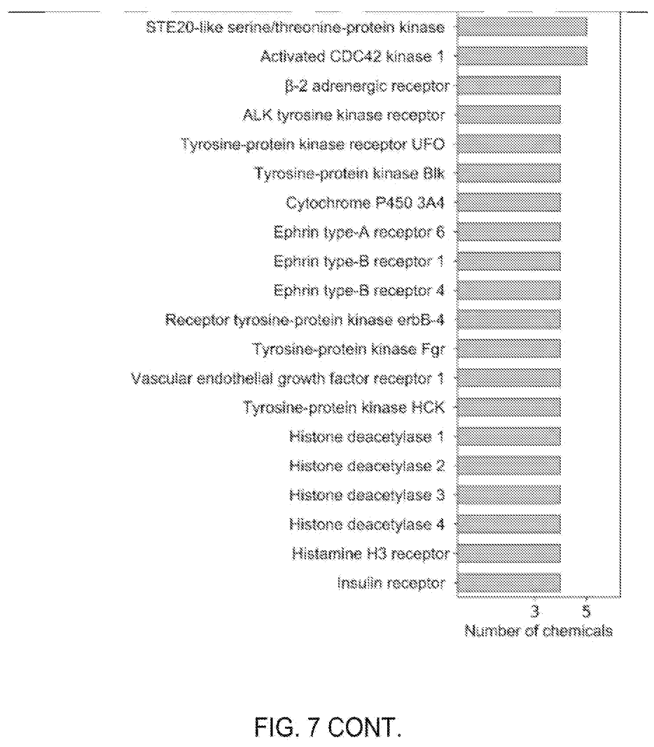

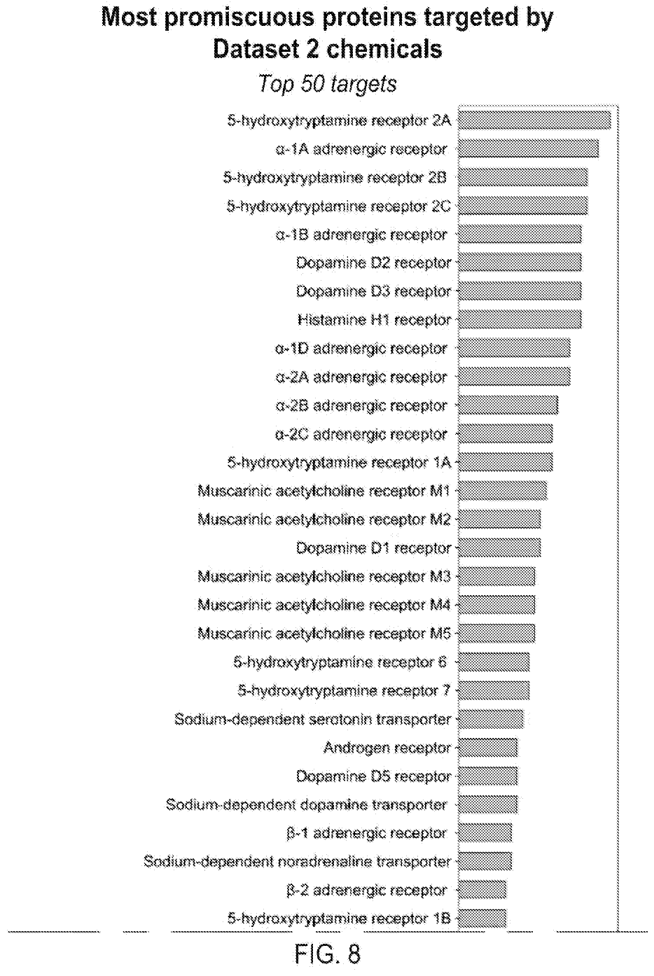

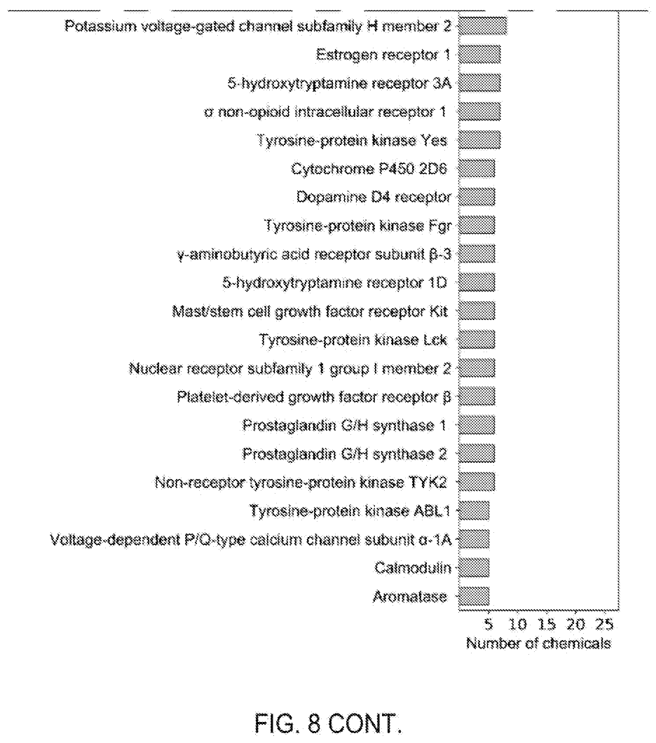

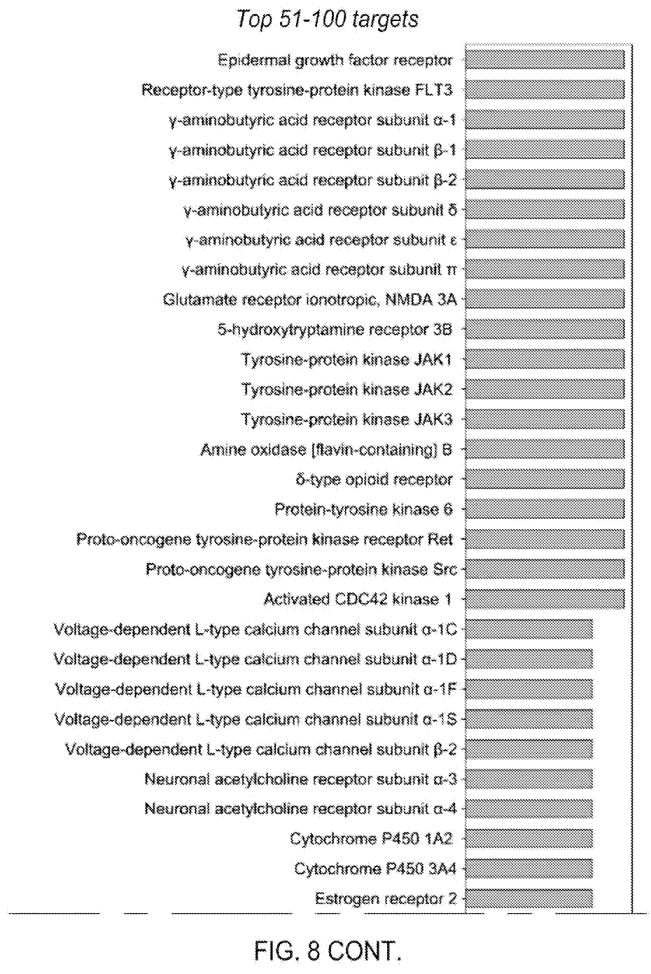

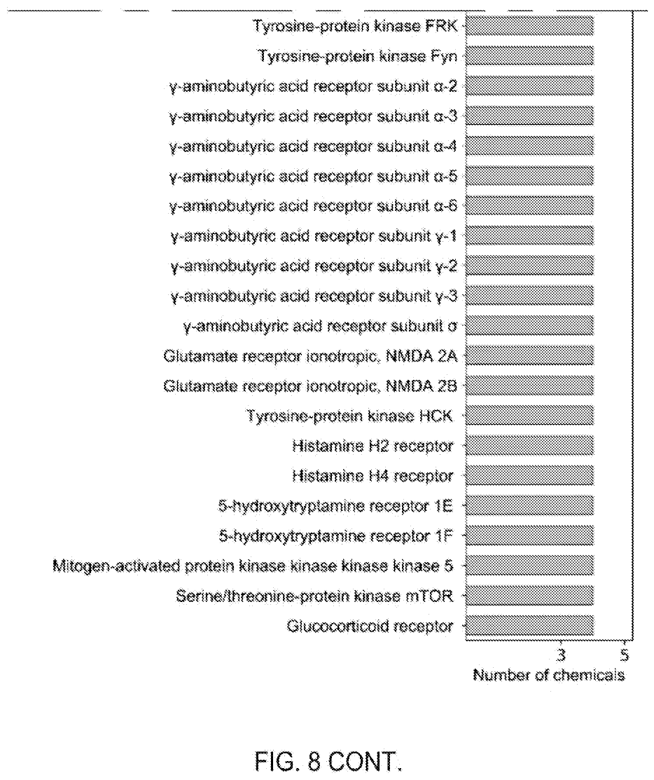

Of these two respective sets, 168 and 163 compounds were annotated in QuartataWeb (Li H et al. Bioinformatics, 2020, 36, 3935-3937), which provided information on the targets of these compounds using DrugBank-all (Wishart D S et al. Nucleic Acids Res. 2018, 46, D1074-D1082) and STITCH-experimental (Szklarczyk D et al. Nucleic Acids Res. 2016, 44, D380-384) data as input. The remaining compounds were “manually” analyzed based on existing literature, as schematically described in the FIG. 6 for Dataset 1. FIG. 7-FIG. 8 shows that the host cell proteins most frequently targeted by the candidate antiviral compounds were adrenergic receptor α1A (gene ADRA1A), serotonin receptor 2A (HTR2A), and histamine H1 receptor (HRH1). Incidentally, ADRA1A and HRH1 were also among the most upregulated genes in SARS-CoV-2 infected A549 cells (Emanuel W et al. bioRxiv, 2020, https://doi.org/10.1101/2020.05.05.079194). Elevated HRH1 can be associated with hyperinflammation (Thurmond R L et al. Nat Rev Drug Discov. 2008, 7, 41-53). Serotonin receptor 2A was maximally targeted by potential anti-cytokine compounds drawing attention to the impact on neurotransmission.

TABLE 5
263 compounds with potential antiviral activity against SARS-CoV-2
infected A549 cells and corresponding CMap scores (all >90).

| In- | | | CMap | Mechanism of |
|---|---|---|---|---|
| dex | Compound name | CMap ID | score | Action (MOA) |
| 1 | Salmeterol | BRD-A01320529 | 100.00 | Adrenergic |
| | | | | receptor agonist |
| 2 | Avrainvillamide- | BRD-A70731303 | 99.99 | nucleophosmin |
| | analog-5 | | | inhibitor |
| 3 | Terbutaline | BRD-A50157456 | 99.98 | Adrenergic |
| | | | | receptor agonist |
| 4 | Oxybutynin | BRD-A65013509 | 99.98 | Acetylcholine |
| | | | | receptor antagonist |
| 5 | Leflunomide | BRD-K78692225 | 99.98 | Dihydroorotate |
| | | | | dehydrogenase |
| | | | | inhibitor, PDGFR |
| | | | | receptor inhibitor |
| 6 | GS-39783 | BRD-K75478907 | 99.98 | GABA receptor |
| | | | | modulator |
| 7 | nTZDpa | BRD-K54708045 | 99.98 | PPAR receptor |
| | | | | agonist |
| 8 | n-arachidonyl- | BRD-K06024458 | 99.98 | cannabinoid |
| | GABA | | | receptor agonist |
| 9 | Salicin | BRD-K64614248 | 99.98 | Anti-inflammatory |
| 10 | SR-27897 | BRD-K35629949 | 99.98 | CCK receptor |
| | | | | antagonist |
| 11 | Thiotepa | BRD-K09631521 | 99.98 | Cytochrome |
| | | | | P450 inhibitor |
| 12 | Brazilin | BRD-A83326220 | 99.98 | Nitric oxide |
| | | | | production inhibitor |
| 13 | Stavudine | BRD-K93880783 | 99.98 | DNA directed |
| | | | | DNA polymerase |
| | | | | inhibitor, Reverse |
| | | | | transcriptase |
| | | | | inhibitor |
| 14 | Oxiconazole | BRD-K23369905 | 99.98 | Bacterial cell |
| | | | | wall synthesis |
| | | | | inhibitor |
| 15 | Mirtazapine | BRD-A64977602 | 99.98 | Adrenergic |
| | | | | receptor antagonist, |
| | | | | Serotonin receptor |
| | | | | antagonist |
| 16 | Liothyronine | BRD-K89152108 | 99.98 | Thyroid hormone |
| | | | | stimulant |
| 17 | Bergenin | BRD-A15034104 | 99.98 | Interleukin inhibitor |
| 18 | Chlorpromazine | BRD-K89997465 | 99.98 | Dopamine receptor |
| | | | | antagonist |
| 19 | Fludarabine | BRD-K66788707 | 99.96 | DNA synthesis |
| | | | | inhibitor, DNA |
| | | | | repair enzyme |
| | | | | inhibitor, Purine |
| | | | | antagonist |
| 20 | Iodophenpropit | BRD-K51918615 | 99.96 | Histamine receptor |
| | | | | antagonist |
| 21 | SA-94315 | BRD-K20197062 | 99.96 | Caspase inhibitor |
| 22 | L-733060 | BRD-K15791587 | 99.96 | Tachykinin |
| | | | | antagonist |
| 23 | Tetrahydro- | BRD-A67605442 | 99.96 | Nitric oxide (NO) |
| | biopterin | | | stimulant, NO |
| | | | | synthase stimulant, |
| | | | | Phenylalanine |
| | | | | 4-hydroxylase |
| | | | | stimulant |
| 24 | Duloxetine | BRD-K71103788 | 99.96 | Serotonin and |
| | | | | norepinephrine |
| | | | | reuptake inhibitor |
| 25 | PSB-36 | BRD-A70407468 | 99.96 | Adenosine receptor |
| | | | | antagonist |
| 26 | CGP-7930 | BRD-K65786282 | 99.95 | GABA receptor |
| | | | | positive allosteric |
| | | | | modulator |
| 27 | Ochratoxin-a | BRD-K39944607 | 99.95 | Phenylalanyl |
| | | | | tRNA synthetase |
| | | | | inhibitor |
| 28 | Alfacalcidol | BRD-K93433262 | 99.94 | Vitamin D receptor |
| | | | | agonist |
| 29 | Molsidomine | BRD-K35531059 | 99.94 | Guanylyl cyclase |
| | | | | activator |
| 30 | NGB-2904 | BRD-K05181084 | 99.94 | Dopamine receptor |
| | | | | antagonist |
| 31 | BH3I-1 | BRD-A38913120 | 99.93 | BCL inhibitor |
| 32 | Isotretinoin | BRD-K76723084 | 99.93 | Retinoid receptor |
| | | | | agonist |
| 33 | Lonidamine | BRD-K96670504 | 99.92 | Glucokinase |
| | | | | inhibitor |
| 34 | L-741626 | BRD-K05181463 | 99.91 | Dopamine receptor |
| | | | | antagonist |
| 35 | PG-9 | BRD-A70268693 | 99.91 | Acetylcholine |
| | | | | receptor agonist |
| 36 | GSK-1059615 | BRD-K06750613 | 99.90 | PI3K inhibitor |
| 37 | Cheno- | BRD-K18135438 | 99.90 | 11-β-HSD1 |
| | deoxycholic- | | | inhibitor, FXR |
| | acid | | | agonist |
| 38 | Edrophonium | BRD-K81128206 | 99.89 | Acetylcholinesterase |
| | | | | inhibitor |
| 39 | Nilotinib | BRD-K81528515 | 99.89 | ABL inhibitor, |
| | | | | BCR-ABL kinase |
| | | | | inhibitor |
| 40 | 5-nonyloxy- | BRD-K08219523 | 99.88 | Serotonin receptor |
| | tryptamine | | | agonist |
| 41 | Tribenoside | BRD-A60294240 | 99.88 | Anti-inflammatory, |
| | | | | Capillary |
| | | | | stabilizing agent |
| 42 | Meclozine | BRD-A50311610 | 99.85 | CAR agonist |
| 43 | Azathioprine | BRD-K32821942 | 99.85 | Dehydrogenase |
| | | | | inhibitor |
| 44 | HDAC3-selective | BRD-K29313308 | 99.84 | HDAC inhibitor |
| 45 | MRS-1845 | BRD-A32949107 | 99.84 | Calcium channel |
| | | | | blocker |
| 46 | KU-55933 | BRD-K25311561 | 99.84 | ATM kinase inhibitor |
| 47 | Arecaidine | BRD-K63792901 | 99.84 | Acetylcholine |
| | | | | receptor agonist |
| 48 | Cortisone | BRD-A54487287 | 99.83 | Glucocorticoid |
| | | | | receptor agonist |
| 49 | Torin-1 | BRD-K40175214 | 99.80 | MTOR inhibitor, |
| | | | | PI3K inhibitor |
| 50 | ENMD-2076 | BRD-K68488863 | 99.79 | FLT3 inhibitor, |
| | | | | VEGFR inhibitor, |
| | | | | Aurora kinase |
| | | | | inhibitor |
| 51 | Aspirin | BRD-K11433652 | 99.79 | Cyclooxygenase |
| | | | | inhibitor |
| 52 | AS-703026 | BRD-K89014967 | 99.74 | MEK inhibitor |
| 53 | Razoxane | BRD-K07265709 | 99.74 | Chelating agent, |
| | | | | Topoisomerase |
| | | | | inhibitor |
| 54 | Ibuprofen | BRD-A17655518 | 99.72 | Cyclooxygenase |
| | | | | inhibitor, NFkB |
| | | | | pathway inhibitor |
| 55 | Demeclocycline | BRD-A75368507 | 99.71 | Bacterial 30S |
| | | | | ribosomal subunit |
| | | | | inhibitor |
| 56 | KU-0063794 | BRD-K67566344 | 99.71 | MTOR inhibitor |
| 57 | Desoxy- | BRD-A75402480 | 99.69 | Mineralocorticoid |
| | corticosterone | | | receptor agonist |
| 58 | Imipramine | BRD-K38436528 | 99.65 | Norepinephrine |
| | | | | and Serotonin |
| | | | | transporter inhibitor |
| 59 | AS-605240 | BRD-K41895714 | 99.63 | PI3K inhibitor |
| 60 | OSI-027 | BRD-K94294671 | 99.62 | MTOR inhibitor |
| 61 | PD-0325901 | BRD-K49865102 | 99.59 | MEK inhibitor, |
| | | | | MAPK inhibitor, |
| | | | | Protein kinase |
| | | | | inhibitor |
| 62 | PKCβ-inhibitor | BRD-K89687904 | 99.59 | PKC inhibitor |
| 63 | Fostamatinib | BRD-K20285085 | 99.58 | SYK inhibitor |
| 64 | Semaxanib | BRD-K63504947 | 99.57 | VEGFR inhibitor |
| 65 | SB-216641 | BRD-K30867024 | 99.57 | Serotonin receptor |
| | | | | antagonist |
| 66 | Rescinnamine | BRD-K52930707 | 99.53 | ACE inhibitor |
| 67 | Tolazoline | BRD-K46211610 | 99.53 | Adrenergic receptor |
| | | | | antagonist |
| 68 | Dactolisib | BRD-K12184916 | 99.51 | MTOR inhibitor, |
| | | | | PI3K inhibitor, |
| | | | | Protein kinase |
| | | | | inhibitor |
| 69 | GSK-1904529A | BRD-K04833372 | 99.50 | IGF-1 inhibitor, |
| | | | | IGF-1R inhibitor, |
| | | | | Insulin receptor |
| | | | | ligand |
| 70 | ML-9 | BRD-K68402494 | 99.44 | Myosin light chain |
| | | | | kinase inhibitor |
| 71 | UNC-0321 | BRD-K74236984 | 99.42 | Histone lysine |
| | | | | methyltransferase |
| | | | | inhibitor |
| 72 | Pizotifen | BRD-K75958195 | 99.41 | Serotonin receptor |
| | | | | antagonist |
| 73 | Pterostilbene | BRD-K92870997 | 99.40 | Cyclooxygenase |
| | | | | inhibitor, PPAR |
| | | | | receptor agonist |
| 74 | VER-155008 | BRD-K32330832 | 99.39 | HSP inhibitor |
| 75 | PIK-90 | BRD-K99818283 | 99.35 | PI3K inhibitor |
| 76 | Panobinostat | BRD-K02130563 | 99.34 | HDAC inhibitor |
| 77 | Oleoyl- | BRD-K66956375 | 99.31 | Cannabinoid |
| | ethanolamide | | | receptor agonist, |
| | | | | Glucose dependent |
| | | | | insulinotropic |
| | | | | receptor agonist, |
| | | | | Potassium channel |
| | | | | blocker, PPAR |
| | | | | receptor agonist |
| 78 | TC-2559 | BRD-K67352070 | 99.30 | Acetylcholine |
| | | | | receptor agonist |
| 79 | Mosapride | BRD-A39052811 | 99.26 | Serotonin receptor |
| | | | | agonist |
| 80 | Dapsone | BRD-K62363391 | 99.21 | Bacterial antifolate |
| 81 | BAY-36-7620 | BRD-K54704028 | 99.20 | Glutamate receptor |
| | | | | antagonist |
| 82 | Emetine | BRD-A25687296 | 99.20 | Protein synthesis |
| | | | | inhibitor |
| 83 | Arecaidine | BRD-K23922020 | 99.19 | Acetylcholine |
| | | | | receptor agonist |
| 84 | Apicidin | BRD-K64606589 | 99.18 | HDAC inhibitor |
| 85 | Niacin | BRD-K61993165 | 99.16 | NAD precursor |
| | | | | with lipid lowering |
| | | | | effect, vitamin B |
| 86 | Entinostat | BRD-K77908580 | 99.16 | HDAC inhibitor |
| 87 | Elesclomol | BRD-K82135108 | 99.15 | Oxidative stress |
| | | | | inducer |
| 88 | BMY-14802 | BRD-A15435692 | 99.14 | Sigma receptor |
| | | | | antagonist |
| 89 | Proxymetacaine | BRD-K79116891 | 99.14 | Sodium channel |
| | | | | blocker |
| 90 | Zamifenacin | BRD-K80451230 | 99.14 | Acetylcholine |
| | | | | receptor antagonist |
| 91 | Anandamide | BRD-K78280988 | 99.12 | Cannabinoid |
| | | | | receptor agonist |
| 92 | Temsirolimus | BRD-A62025033 | 99.10 | MTOR inhibitor |
| 93 | Desmethyl- | BRD-K10042277 | 99.07 | Acetylcholine |
| | clozapine | | | receptor agonist |
| 94 | QL-XII-47 | BRD-U86922168 | 99.03 | BTK inhibitor, |
| | | | | Cytoplasmic |
| | | | | tyrosine protein |
| | | | | kinase BMX |
| | | | | inhibitor |
| 95 | PI-103 | BRD-K67868012 | 99.03 | MTOR inhibitor, |
| | | | | PI3K inhibitor |
| 96 | Eugenol | BRD-K32977963 | 99.01 | Androgen receptor |
| | | | | antagonist |
| 97 | SKF-81297 | BRD-A09828896 | 98.98 | Dopamine receptor |
| | | | | agonist |
| 98 | 2-aminopurine | BRD-K35128472 | 98.97 | Serine/threonine |
| | | | | kinase inhibitor |
| 99 | GBR-12783 | BRD-K92015269 | 98.97 | Dopamine uptake |
| | | | | inhibitor |
| 100 | Mephenytoin | BRD-A83937277 | 98.96 | Hydantoin |
| | | | | antiepileptic |
| 101 | Terfenadine | BRD-A06352418 | 98.89 | Histamine receptor |
| | | | | antagonist |
| 102 | KIN001-127 | BRD-A29901043 | 98.89 | ITK inhibitor |
| 103 | GBR-12935 | BRD-K50135270 | 98.88 | Dopamine uptake |
| | | | | inhibitor |
| 104 | Flavanone | BRD-A07824748 | 98.74 | 11-β-HSD1 inhibitor |
| 105 | VU-0366037-2 | BRD-K39823328 | 98.73 | Glutamate receptor |
| | | | | modulator |
| 106 | Ioxaglic-acid | BRD-K79124250 | 98.68 | Radiopaque medium |
| 107 | FR-122047 | BRD-K30990140 | 98.65 | Cyclooxygenase |
| | | | | inhibitor |
| 108 | WZ-3146 | BRD-K73293050 | 98.61 | EGFR inhibitor |
| 109 | Droxinostat | BRD-K11558771 | 98.60 | HDAC inhibitor |
| 110 | Cimaterol | BRD-A65440446 | 98.56 | Adrenergic |
| | | | | receptor agonist |
| 111 | SCH-23390 | BRD-K45435259 | 98.48 | Dopamine |
| | | | | receptor antagonist |
| 112 | Ribavirin | BRD-A96255180 | 98.45 | Antiviral |
| 113 | Mycophenolate- | BRD-K92428153 | 98.41 | Dehydrogenase |
| | mofetil | | | inhibitor, |
| | | | | Hydroxycarboxylic |
| | | | | acid receptor |
| | | | | agonist, |
| | | | | Immunosuppressant, |
| | | | | Inosine |
| | | | | monophosphate |
| | | | | dehydrogenase |
| | | | | inhibitor, Inositol |
| | | | | monophosphatase |
| | | | | inhibitor |
| 114 | Linsitinib | BRD-K08589866 | 98.37 | IGF-1 inhibitor |
| 115 | LY-288513 | BRD-K24675965 | 98.28 | CCK receptor |
| | | | | antagonist |
| 116 | Wiskostatin | BRD-A18579359 | 98.25 | Neural Wiskott- |
| | | | | Aldrich syndrome |
| | | | | protein inhibitor |
| 117 | AG-879 | BRD-K59469039 | 98.23 | Angiogenesis |
| | | | | inhibitor, Tyrosine |
| | | | | kinase inhibitor, |
| | | | | VEGFR inhibitor |
| 118 | BIX-01338 | BRD-K26863634 | 98.21 | Histone lysine |
| | | | | methyltransferase |
| | | | | inhibitor |
| 119 | Arcyriaflavin-a | BRD-K72726508 | 98.20 | CDK inhibitor |
| 120 | AY-9944 | BRD-K03642198 | 98.15 | Hedgehog pathway |
| | | | | modulator |
| 121 | NU-7026 | BRD-K09537769 | 98.15 | DNA dependent |
| | | | | protein kinase |
| | | | | inhibitor, MTOR |
| | | | | inhibitor, PI3K |
| | | | | inhibitor |
| 122 | GW-9662 | BRD-K93258693 | 98.08 | PPAR receptor |
| | | | | antagonist |
| 123 | APHA- | BRD-K74733595 | 98.07 | HDAC inhibitor |
| | compound-8 | | | |
| 124 | Mefenamic-acid | BRD-K92778217 | 98.00 | Cyclooxygenase |
| | | | | inhibitor |
| 125 | Heliomycin | BRD-K64517075 | 98.00 | ATP synthase |
| | | | | inhibitor |
| 126 | PP-30 | BRD-K30677119 | 97.98 | RAF inhibitor |
| 127 | NVP-TAE684 | BRD-K50140147 | 97.96 | ALK inhibitor |
| 128 | Ropivacaine | BRD-K50938786 | 97.64 | Sodium channel |
| | | | | blocker |
| 129 | MK-5108 | BRD-K53665955 | 97.59 | Aurora kinase |
| | | | | inhibitor |
| 130 | Ciclacillin | BRD-K89046952 | 97.54 | Bacterial cell |
| | | | | wall synthesis |
| | | | | inhibitor |
| 131 | Sulfafurazole | BRD-K50859149 | 97.43 | Bacterial antifolate |
| 132 | Dephostatin | BRD-K60274257 | 97.43 | Tyrosine phosphatase |
| | | | | inhibitor |
| 133 | Entacapone | BRD-K83636919 | 97.31 | Catechol O |
| | | | | methyltransferase |
| | | | | inhibitor |
| 134 | Oxfendazole | BRD-A33447119 | 97.21 | Anthelmintic |
| 135 | Rottlerin | BRD-K03816923 | 97.11 | MAP kinase |
| | | | | inhibitor, Protein |
| | | | | kinase inhibitor |
| 136 | Desipramine | BRD-K60762818 | 97.10 | Tricyclic |
| | | | | antidepressant |
| 137 | Perospirone | BRD-K85503079 | 97.10 | Dopamine and |
| | | | | serotonin receptors' |
| | | | | antagonist |
| 138 | Pimozide | BRD-K01292756 | 97.10 | Dopamine receptor |
| | | | | antagonist |
| 139 | Ceforanide | BRD-K37848908 | 97.00 | Penicillin binding |
| | | | | protein inhibitor |
| 140 | Equilin | BRD-K04046242 | 96.99 | Estrogen receptor |
| | | | | agonist |
| 141 | SB-590885 | BRD-K01253243 | 96.95 | RAF inhibitor |
| 142 | LY-2140023 | BRD-K49519144 | 96.93 | Glutamate receptor |
| | | | | agonist |
| 143 | Glipizide | BRD-K12219985 | 96.92 | Sulfonylurea |
| 144 | Moracizine | BRD-K21548250 | 96.91 | Sodium channel |
| | | | | blocker |
| 145 | Kavain | BRD-A75455249 | 96.82 | Calcium channel |
| | | | | modulator, |
| | | | | Sodium channel |
| | | | | blocker |
| 146 | Wortmannin | BRD-A11678676 | 96.81 | PI3K inhibitor |
| 147 | Decitabine | BRD-K79254416 | 96.67 | DNA |
| | | | | methyltransferase |
| | | | | inhibitor |
| 148 | Metformin | BRD-K79602928 | 96.61 | Insulin sensitizer |
| 149 | Eicosatrienoic- | BRD-K63913457 | 96.58 | Vasodilator |
| | acid | | | |
| 150 | Raloxifene | BRD-K63828191 | 96.58 | Estrogen receptor |
| | | | | antagonist, |
| | | | | Selective estrogen |
| | | | | receptor |
| | | | | modulator (SERM) |
| 151 | Ezetimibe | BRD-A41519720 | 96.57 | Niemann-Pick |
| | | | | C1-like 1 protein |
| | | | | antagonist, |
| | | | | Cholesterol |
| | | | | inhibitor |
| 152 | NBI-27914 | BRD-K61177364 | 96.54 | CRF receptor |
| | | | | antagonist |
| 153 | RS-67333 | BRD-K46142322 | 96.52 | Serotonin receptor |
| | | | | partial agonist |
| 154 | BMS-191011 | BRD-K95609758 | 96.46 | Potassium channel |
| | | | | activator |
| 155 | H-7 | BRD-A55756846 | 96.39 | PKA inhibitor |
| 156 | VU-0404997-2 | BRD-A34208323 | 96.39 | Glutamate receptor |
| | | | | modulator |
| 157 | Cisapride | BRD-K06895174 | 96.23 | Serotonin receptor |
| | | | | agonist |
| 158 | Y-134 | BRD-K94832621 | 96.20 | Estrogen receptor |
| | | | | antagonist |
| 159 | Metrizamide | BRD-A45543382 | 95.98 | Radiopaque medium |
| 160 | Dydrogesterone | BRD-K68620903 | 95.94 | Progesterone receptor |
| | | | | agonist |
| 161 | Altrenogest | BRD-A27554692 | 95.93 | Progestogen hormone |
| 162 | Homosalate | BRD-A34751532 | 95.90 | HSP inducer |
| 163 | Bosutinib | BRD-K99964838 | 95.89 | ABL inhibitor, |
| | | | | BCR-ABL kinase |
| | | | | inhibitor, SRC |
| | | | | inhibitor |
| 164 | Puromycin | BRD-A28970875 | 95.88 | Protein synthesis |
| | | | | inhibitor |
| 165 | Methimazole | BRD-K54416256 | 95.71 | Antithyroid |
| 166 | ALW-II-38-3 | BRD-K68191783 | 95.60 | Ephrin inhibitor |
| 167 | SN-38 | BRD-A36630025 | 95.51 | Topoisomerase |
| | | | | inhibitor |
| 168 | Ipratropium | BRD-A05352148 | 95.50 | Acetylcholine |
| | | | | receptor antagonist |
| 169 | TGX-221 | BRD-A41692738 | 95.47 | PI3K inhibitor |
| 170 | Homo- | BRD-K76674262 | 95.43 | Protein synthesis |
| | harringtonine | | | inhibitor |
| 171 | Metergoline | BRD-A30435184 | 95.42 | Dopamine receptor |
| | | | | agonist, |
| | | | | Serotonin receptor |
| | | | | antagonist |
| 172 | WZ-4-145 | BRD-U25771771 | 95.41 | EGFR inhibitor |
| 173 | Mercaptopurine | BRD-K91601245 | 95.33 | Immunosuppressant, |
| | | | | Protein |
| | | | | synthesis inhibitor, |
| | | | | Purine |
| | | | | antagonist |
| 174 | Calmidazolium | BRD-A98283014 | 95.28 | Calcium channel |
| | | | | blocker, |
| | | | | Calmodulin |
| | | | | antagonist |
| 175 | Mesna | BRD- | 95.21 | Antioxidant |
| | | M40783228 | | |
| 176 | SDZ-205-557 | BRD-K15868788 | 95.20 | Serotonin receptor |
| | | | | antagonist |
| 177 | Procyclidine | BRD-A31800922 | 95.16 | Acetylcholine |
| | | | | receptor antagonist |
| 178 | Amiodarone | BRD-K17561142 | 95.15 | Potassium channel |
| | | | | blocker |
| 179 | Midodrine | BRD-A79981887 | 95.14 | Adrenergic receptor |
| | | | | agonist |
| 180 | Mepireserpate | BRD-A71765365 | 95.11 | Catecholamine |
| | | | | depleting |
| | | | | sympatholytic |
| 181 | SA-792728 | BRD-K20755323 | 95.06 | Sphingosine kinase |
| | | | | inhibitor |
| 182 | Brompheniramine | BRD-A68723818 | 94.90 | Histamine receptor |
| | | | | antagonist |
| 183 | Sumatriptan | BRD-K50938287 | 94.83 | Serotonin receptor |
| | | | | agonist |
| 184 | Gemcitabine | BRD-K15108141 | 94.82 | Ribonucleotide |
| | | | | reductase inhibitor |
| 185 | JAK3-Inhibitor-II | BRD-K52850071 | 94.81 | JAK inhibitor |
| 186 | CHEMBL- | BRD-K59962020 | 94.79 | NFkB pathway |
| | 374350 | | | inhibitor |
| 187 | Vorinostat | BRD-K81418486 | 94.70 | HDAC inhibitor |
| 188 | Dipyridamole | BRD-K86301799 | 94.48 | Phosphodiesterase |
| | | | | inhibitor |
| 189 | JNJ-16259685 | BRD-K64670467 | 94.47 | Glutamate |
| | | | | receptor antagonist |
| 190 | VU-0415374-1 | BRD-K83010055 | 94.46 | Glutamate receptor |
| | | | | modulator |
| 191 | Pidorubicine | BRD-K04548931 | 94.42 | Topoisomerase |
| | | | | inhibitor |
| 192 | KU-C103443N | BRD-A81402010 | 94.22 | CDC inhibitor, |
| | | | | Rho associated |
| | | | | kinase inhibitor |
| 193 | Dichlorobenzamil | BRD-K12906962 | 94.16 | Sodium/calcium |
| | | | | exchange inhibitor |
| 194 | Mepacrine | BRD-A45889380 | 94.14 | Cytokine production |
| | | | | inhibitor, |
| | | | | NFkB pathway |
| | | | | inhibitor, TP53 |
| | | | | activator |
| 195 | E-4031 | BRD-K41713976 | 93.96 | Potassium |
| | | | | channel blocker |
| 196 | Narciclasine | BRD-K06792661 | 93.94 | Coflilin |
| | | | | signaling pathway |
| | | | | activator, LIM |
| | | | | kinase activator, |
| | | | | Rho associated |
| | | | | kinase activator |
| 197 | Mesoridazine | BRD-A14395271 | 93.80 | Dopamine |
| | | | | receptor antagonist |
| 198 | Tranylcypromine | BRD-A43974575 | 93.79 | Monoamine |
| | | | | oxidase inhibitor |
| 199 | Lypressin | BRD-K93331255 | 93.76 | Vasopressin |
| | | | | receptor agonist |
| 200 | Reserpine | BRD-K95921201 | 93.69 | Vesicular |
| | | | | monoamine |
| | | | | transporter |
| | | | | inhibitor |
| 201 | Abiraterone | BRD-K55301415 | 93.64 | 17, 20 lyase |
| | | | | inhibitor, Androgen |
| | | | | biosynthesis |
| | | | | inhibitor, |
| | | | | Cytochrome |
| | | | | P450 inhibitor, |
| | | | | Steroid sulfatase |
| | | | | inhibitor |
| 202 | I-OMe-AG-538 | BRD-K35377380 | 93.59 | IGF-1 inhibitor |
| 203 | Somatostatin | BRD-K14681867 | 93.48 | Somatostatin |
| | | | | receptor agonist |
| 204 | Splitomycin | BRD-K27710560 | 93.37 | SIRT inhibitor |
| 205 | AM-281 | BRD-K59419204 | 93.34 | Cannabinoid |
| | | | | receptor antagonist |
| 206 | Sphingosine | BRD-K62959606 | 93.32 | Ceramidase inhibitor |
| 207 | Hydroxy- | BRD-A36707673 | 93.30 | LXR agonist |
| | cholesterol | | | |
| 208 | TPCA-1 | BRD-K51575138 | 93.29 | IKK inhibitor |
| 209 | FGIN-1-27 | BRD-K09778810 | 93.26 | Inositol |
| | | | | monophosphatase |
| | | | | inhibitor |
| 210 | Trichostatin-a | BRD-K68202742 | 93.06 | HDAC inhibitor, |
| | | | | CDK activator, |
| | | | | ID1 inhibitor |
| 211 | Hexylresorcinol | BRD-K99946902 | 92.99 | Local anesthetic |
| 212 | Epicatechin | BRD-K50660797 | 92.96 | Bacterial DNA |
| | | | | gyrase inhibitor, |
| | | | | Cyclooxygenase |
| | | | | inhibitor, DNA |
| | | | | polymerase inhibitor |
| 213 | RS-17053 | BRD-K76840893 | 92.95 | Adrenergic |
| | | | | receptor antagonist |
| 214 | NSC-663284 | BRD-K03109492 | 92.78 | CDC inhibitor |
| 215 | L-165041 | BRD-K40656405 | 92.55 | PPAR receptor |
| | | | | agonist |
| 216 | ML-7 | BRD-K93201660 | 92.46 | Myosin light chain |
| | | | | kinase inhibitor |
| 217 | Alisertib | BRD-K75295174 | 92.44 | Aurora kinase |
| | | | | inhibitor |
| 218 | GR-127935 | BRD-K11911061 | 92.37 | Serotonin receptor |
| | | | | antagonist |
| 219 | Clobenpropit | BRD-K71430621 | 92.36 | Histamine receptor |
| | | | | antagonist |
| 220 | NNC-55-0396 | BRD-K78122587 | 92.31 | T-type calcium |
| | | | | channel blocker |
| 221 | Barasertib | BRD-K63923597 | 92.27 | Aurora kinase |
| | | | | inhibitor |
| 222 | Benidipine | BRD-A35519318 | 92.25 | Calcium channel |
| | | | | blocker |
| 223 | Sertraline | BRD-K82036761 | 92.24 | Serotonin |
| | | | | receptor antagonist |
| 224 | ZM-447439 | BRD-K72703948 | 92.16 | Aurora kinase |
| | | | | inhibitor |
| 225 | BIBX-1382 | BRD-K70914287 | 92.15 | EGFR inhibitor, |
| | | | | Tyrosine kinase |
| | | | | inhibitor |
| 226 | Immethridine | BRD-K49519092 | 92.15 | Histamine receptor |
| | | | | agonist |
| 227 | MR-16728 | BRD-A30590053 | 92.15 | Acetylcholine |
| | | | | release enhancer, |
| | | | | Acetylcholine |
| | | | | release stimulant |
| 228 | Heraclenol | BRD-A77050075 | 92.11 | Vitamin K antagonist |
| 229 | NU-7441 | BRD-K00337317 | 92.10 | DNA dependent |
| | | | | protein kinase |
| | | | | inhibitor, |
| | | | | P-glycoprotein |
| | | | | inhibitor |
| 230 | Hyoscyamine | BRD-K40530731 | 91.85 | Acetylcholine |
| | | | | receptor antagonist |
| 231 | m- | BRD-K36965586 | 91.76 | Serotonin receptor |
| | chlorophenyl- | | | agonist |
| | biguanide | | | |
| 232 | Prostaglandin-b2 | BRD-K82865713 | 91.75 | cAMP inhibitor |
| 233 | BML-ST330 | BRD-A77118605 | 91.59 | Phospholipase |
| | | | | inhibitor |
| 234 | STO-609 | BRD-K52620403 | 91.57 | Calmodulin |
| | | | | antagonist |
| 235 | Tyrphostin- | BRD-K14441456 | 91.50 | EGFR inhibitor |
| | AG-556 | | | |
| 236 | Corynanthine | BRD-K06467078 | 91.46 | Adrenergic receptor |
| | | | | antagonist |
| 237 | PD-102807 | BRD-A89337244 | 91.31 | Acetylcholine |
| | | | | receptor antagonist |
| 238 | Norgestrel | BRD-A50928468 | 91.25 | Progesterone |
| | | | | receptor agonist |
| 239 | Telmisartan | BRD-K73999723 | 91.25 | Angiotensin |
| | | | | receptor antagonist |
| 240 | BMY-45778 | BRD-K84895041 | 91.22 | IP1 prostacyclin |
| | | | | receptor agonist |
| 241 | Dihydrosamidin | BRD-K63945320 | 91.19 | Phospholipase |
| | | | | inhibitor, Nitric |
| | | | | oxide production |
| | | | | inhibitor, platelet |
| | | | | activating factor |
| | | | | receptor |
| | | | | antagonist |
| 242 | HG-6-64-01 | BRD-U37049823 | 91.09 | RAF inhibitor |
| 243 | KUC104502N | BRD-K24538644 | 91.06 | — |
| 244 | Formestane | BRD-A31801025 | 91.00 | Aromatase inhibitor |
| 245 | BRD-K64835161 | BRD-K64835161 | 90.93 | — |
| 246 | M2-PK-activator | BRD-K80672993 | 90.89 | — |
| 247 | Cetraxate | BRD-K48932581 | 90.81 | Mucus protecting |
| | | | | agent |
| 248 | Terbinafine | BRD-K68132782 | 90.76 | Fungal squalene |
| | | | | epoxidase |
| | | | | inhibitor |
| 249 | Phospho- | BRD-K68873215 | 90.69 | Phosphodiesterase |
| | diesterase-V- | | | inhibitor |
| | inhibitor-II | | | |
| 250 | Ponalrestat | BRD-K68332390 | 90.68 | Aldose reductase |
| | | | | inhibitor |
| 251 | Phenytoin | BRD-K55930204 | 90.67 | Hydantoin |
| | | | | antiepileptic |
| 252 | Phylloquinone | BRD-A55815733 | 90.64 | Vitamin K, Γ |
| | | | | carboxylase enzyme |
| 253 | AZD-8055 | BRD-K69932463 | 90.64 | MTOR inhibitor |
| 254 | PHA-665752 | BRD-K95435023 | 90.53 | c-Met inhibitor |
| 255 | PD-184352 | BRD-K05104363 | 90.52 | MEK inhibitor |
| 256 | RU-28318 | BRD-A92585442 | 90.43 | Cytochrome P450 |
| | | | | inhibitor |
| 257 | Fenoldopam | BRD-A50684349 | 90.38 | Dopamine receptor |
| | | | | agonist |
| 258 | Camptothecin | BRD-A30437061 | 90.32 | Topoisomerase |
| | | | | inhibitor |
| 259 | Tretinoin | BRD-K06926592 | 90.31 | Retinoid receptor |
| | | | | agonist, Retinoid |
| | | | | receptor ligand |
| 260 | Metixene | BRD-A33711280 | 90.16 | Acetylcholine |
| | | | | receptor antagonist |
| 261 | Tetra- | BRD-A43940795 | 90.12 | Serotonin release |
| | hydropalmatine | | | inhibitor |
| 262 | YM-976 | BRD-K12932420 | 90.09 | Phosphodiesterase |
| | | | | inhibitor |
| 263 | Alaproclate | BRD-A14966924 | 90.03 | Serotonin receptor |
| | | | | antagonist |

TABLE 6
275 compounds with CMap scores <−90, which can
potentially elicit anti-cytokine activity against
hyperinflammation in SARS-CoV-2-infected A549-ACE2 cells.

| In- | Compound | | CMap | Mechanism of |
|---|---|---|---|---|
| dex | name | CMap ID | score | Action (MOA) |
| 1 | n-(3-acetami- | BRD-K61217870 | −100 | Glutamate |
| | dophenyl)-3- | | | receptor |
| | chloro- | | | antagonist |
| | benzamide | | | |
| 2 | BI-78D3 | BRD-K73982490 | −99.98 | JNK inhibitor |
| 3 | Xaliproden | BRD-K88358234 | −99.98 | Serotonin |
| | | | | receptor agonist |
| 4 | Rhamnetin | BRD-K37206356 | −99.98 | HDAC inhibitor |
| 5 | MR-16728 | BRD-A30590053 | −99.98 | Acetylcholine |
| | | | | release enhancer |
| | | | | or stimulant |
| 6 | Palonosetron | BRD-K08924299 | −99.98 | Serotonin |
| | | | | receptor antagonist |
| 7 | Clarithromycin | BRD-K49668410 | −99.98 | Bacterial 50S |
| | | | | ribosomal subunit |
| | | | | inhibitor |
| 8 | SAL-1 | BRD-K40213712 | −99.98 | Adenosine |
| | | | | receptor antagonist |
| 9 | Nimodipine | BRD-A58048407 | −99.98 | Calcium channel |
| | | | | blocker |
| 10 | Isoliquiritigenin | BRD-K33583600 | −99.98 | Guanylate cyclase |
| | | | | activator |
| 11 | Cyclazosin | BRD-A37837077 | −99.98 | Adrenergic |
| | | | | receptor antagonist |
| 12 | Eicosatetray- | BRD-K06080977 | −99.98 | Cyclooxygenase |
| | noic-acid | | | inhibitor, |
| | | | | Lipoxygenase |
| | | | | inhibitor |
| 13 | Oxantel | BRD-K66019333 | −99.96 | Anthelmintic |
| 14 | Nor- | BRD-A11135865 | −99.96 | Opioid receptor |
| | binaltorphimine | | | antagonist |
| 15 | PCA-4248 | BRD-A29289453 | −99.95 | Platelet activating |
| | | | | factor |
| | | | | receptor |
| | | | | antagonist |
| 16 | Ketanserin | BRD-K49671696 | −99.95 | Serotonin receptor |
| | | | | antagonist |
| 17 | Tyrphostin- | BRD-K03670461 | −99.94 | EGFR inhibitor |
| | AG-82 | | | |
| 18 | Azelastine | BRD-A68888262 | −99.94 | Histamine receptor |
| | | | | antagonist |
| 19 | Diethyl- | BRD-K45330754 | −99.93 | Estrogen receptor |
| | stilbestrol | | | agonist |
| 20 | Raltegravir | BRD-K05658747 | −99.93 | HIV integrase |
| | | | | inhibitor |
| 21 | KI-16425 | BRD-A25569250 | −99.93 | Lysophosphatidic |
| | | | | acid receptor |
| | | | | antagonist |
| 22 | Pyroxamide | BRD-K11663430 | −99.93 | HDAC inhibitor |
| 23 | Maprotiline | BRD-K03319035 | −99.93 | Norepinephrine |
| | | | | reuptake |
| | | | | inhibitor, Tricyclic |
| | | | | antidepressant |
| 24 | Reserpic-acid | BRD-K32755366 | −99.91 | Norepinephrine |
| | | | | transporter |
| | | | | inhibitor |
| 25 | Fostamatinib | BRD-K20285085 | −99.91 | SYK inhibitor |
| 26 | Y-27632 | BRD-K44084986 | −99.91 | Rho associated |
| | | | | kinase inhibitor |
| 27 | Dexketoprofen | BRD-K43764301 | −99.9 | Cyclooxygenase |
| | | | | inhibitor |
| 28 | EMF-bca1-60 | BRD-K68437527 | −99.9 | caspase inhibitor |
| 29 | Fluphenazine | BRD-K55127134 | −99.9 | Dopamine receptor |
| | | | | antagonist |
| 30 | Gabazine | BRD-K93280214 | −99.89 | GABA receptor |
| | | | | antagonist |
| 31 | α-estradiol | BRD-A60070924 | −99.88 | Estrogen receptor |
| | | | | agonist |
| 32 | Benzydamine | BRD-K76133116 | −99.88 | Membrane integrity |
| | | | | inhibitor, |
| | | | | Prostanoid receptor |
| | | | | antagonist, |
| | | | | Prostanoid receptor |
| | | | | inhibitor |
| 33 | Navitoclax | BRD-K82746043 | −99.88 | BCL inhibitor |
| 34 | Nifurtimox | BRD-A00100033 | −99.86 | DNA inhibitor |
| 35 | Thenoyltri- | BRD-K00959089 | −99.86 | Chelating agent |
| | fluoroacetone | | | |
| 36 | NVP-AUY922 | BRD-K41859756 | −99.86 | HSP inhibitor |
| 37 | BRD- | BRD-K64835161 | −99.85 | NA |
| | K64835161 | | | |
| 38 | Atorvastatin | BRD-U88459701 | −99.85 | HMGCR (HMG |
| | | | | CoA reductase) |
| | | | | inhibitor |
| 39 | Securinine | BRD-A25775766 | −99.82 | GABA receptor |
| | | | | antagonist, |
| | | | | TP53 activator |
| 40 | Nikkomycin | BRD-A74771556 | −99.82 | Chitin inhibitor |
| 41 | Zuclopenthixol | BRD-K28761384 | −99.78 | Dopamine receptor |
| | | | | antagonist |
| 42 | Temozolomide | BRD-K32107296 | −99.78 | DNA alkylating |
| | | | | agent |
| 43 | HY-11007 | BRD-K97056771 | −99.77 | BCR-ABL kinase |
| | | | | inhibitor |
| 44 | Salsolinol | BRD-K99595596 | −99.74 | Monoamine |
| | | | | oxidase inhibitor, |
| | | | | Tyrosine hydroxylase |
| | | | | inhibitor |
| 45 | SCH-28080 | BRD-K55748775 | −99.73 | ATPase inhibitor |
| 46 | Retinol | BRD-K13927029 | −99.72 | Retinoid receptor |
| | | | | ligand |
| 47 | SB-216763 | BRD-K59184148 | −99.72 | Glycogen synthase |
| | | | | kinase |
| | | | | inhibitor |
| 48 | YS-035 | BRD-K06208435 | −99.71 | Calcium channel |
| | | | | blocker |
| 49 | Bisoprolol | BRD-A89175223 | −99.69 | Adrenergic receptor |
| | | | | antagonist |
| 50 | Carteolol | BRD-A42167015 | −99.62 | Adrenergic receptor |
| | | | | antagonist |
| 51 | TER-14687 | BRD-A33833419 | −99.62 | Inhibitor of |
| | | | | translocation of |
| | | | | PKCq in T cells |
| 52 | Selegiline | BRD-K86434416 | −99.59 | Monoamine |
| | | | | oxidase inhibitor |
| 53 | Triptolide | BRD-A13122391 | −99.58 | RNA polymerase |
| | | | | inhibitor |
| 54 | Lisuride | BRD-K88871508 | −99.57 | Dopamine receptor |
| | | | | agonist |
| 55 | Topiramate | BRD-K29653726 | −99.57 | Carbonic |
| | | | | anhydrase inhibitor, |
| | | | | Glutamate receptor |
| | | | | antagonist, |
| | | | | Kainate receptor |
| | | | | antagonist |
| 56 | Berbamine | BRD-K50464341 | −99.55 | Calmodulin antagonist |
| 57 | Hexamethyl- | BRD-K40990712 | −99.53 | Sodium/hydrogen |
| | eneamiloride | | | antiport |
| | | | | inhibitor |
| 58 | MW-STK33- | BRD-K64310881 | −99.52 | Potassium channel |
| | 3B | | | activator |
| 59 | MLN-4924 | BRD-K67844266 | −99.5 | Nedd activating |
| | | | | enzyme |
| | | | | inhibitor |
| 60 | EHNA | BRD-K27450477 | −99.48 | Adenosine |
| | | | | deaminase inhibitor |
| 61 | Chlor- | BRD-K59058766 | −99.46 | Dopamine receptor |
| | prothixene | | | antagonist |
| 62 | DUP-697 | BRD-K06221026 | −99.45 | Cyclooxygenase |
| | | | | inhibitor |
| 63 | Chlor- | BRD-A04553218 | −99.45 | Histamine receptor |
| | phenamine | | | antagonist |
| 64 | NAS-181 | BRD-A23683907 | −99.38 | Serotonin receptor |
| | | | | antagonist |
| 65 | Linsitinib | BRD-K08589866 | −99.37 | IGF-1 inhibitor, |
| | | | | insulin |
| | | | | inhibitor, ARF6 |
| | | | | and TBK1 |
| | | | | activator |
| 66 | YC-1 | BRD-K60476892 | −99.34 | Guanylyl cyclase |
| | | | | activator |
| 67 | Olanzapine | BRD-K18895904 | −99.32 | Dopamine |
| | | | | receptor/serotonin |
| | | | | receptor antagonist |
| 68 | Orantinib | BRD-K91696562 | −99.3 | FGFR, VEGFR, |
| | | | | PDGFR |
| | | | | inhibitor |
| 69 | Phenelzine | BRD-K87024524 | −99.3 | Monoamine |
| | | | | oxidase inhibitor |
| 70 | TGX-221 | BRD-A41692738 | −99.29 | PI3K inhibitor |
| 71 | Latrepirdine | BRD-K55703048 | −99.29 | Glutamate receptor |
| | | | | antagonist |
| 72 | PU-H71 | BRD-K36529613 | −99.28 | HSP inhibitor |
| 73 | Bromocriptine | BRD-A69960130 | −99.24 | Dopamine receptor |
| | | | | agonist |
| 74 | Syrosingopine | BRD-K14200658 | −99.21 | Vesicular monoamine |
| | | | | transporter inhibitor |
| 75 | UNC-0321 | BRD-K74236984 | −99.21 | Histone lysine |
| | | | | methyltransferase |
| | | | | inhibitor |
| 76 | BRD- | BRD-A80383043 | −99.19 | Glutamate |
| | A80383043 | | | receptor agonist |
| | | | | and/or antagonist |
| 77 | Trifluoperazine | BRD-K89732114 | −99.17 | Dopamine |
| | | | | receptor antagonist |
| 78 | AQ-RA741 | BRD-K81729199 | −99.16 | Acetylcholine receptor |
| | | | | antagonist |
| 79 | Bromfenac | BRD-K47679368 | −99.14 | Cyclooxygenase |
| | | | | inhibitor |
| 80 | Oxaprozin | BRD-K25394294 | −99.14 | Cyclooxygenase |
| | | | | inhibitor |
| 81 | CGS-20625 | BRD-K68103045 | −99.12 | Benzodiazepine |
| | | | | receptor |
| | | | | agonist, GABA |
| | | | | benzodiazepine |
| | | | | site receptor |
| | | | | partial agonist |
| 82 | Rucaparib | BRD-K88560311 | −99.11 | PARP inhibitor |
| 83 | o-3M3FBS | BRD-K46384212 | −99.09 | phospholipase |
| | | | | activator |
| 84 | L-655240 | BRD-K89402695 | −99.07 | Thromboxane receptor |
| | | | | antagonist |
| 85 | L-750667 | BRD-K28806945 | −99.04 | Dopamine receptor |
| | | | | antagonist |
| 86 | Daunorubicin | BRD-K43389675 | −98.99 | RNA synthesis |
| | | | | inhibitor, |
| | | | | Topoisomerase |
| | | | | inhibitor |
| 87 | Profenamine | BRD-A16311756 | −98.98 | Butyrylcholinesterase |
| | | | | inhibitor, |
| | | | | Cholinergic receptor |
| | | | | antagonist |
| 88 | Saracatinib | BRD-K19540840 | −98.97 | SRC inhibitor |
| 89 | Trazodone | BRD-K70778732 | −98.96 | Adrenergic receptor |
| | | | | antagonist, |
| | | | | Serotonin receptor |
| | | | | antagonist, |
| | | | | Serotonin reuptake |
| | | | | inhibitor |
| 90 | Valproic-acid | BRD-K41260949 | −98.93 | HDAC inhibitor |
| 91 | Medetomidine | BRD-A66563878 | −98.93 | Adrenergic receptor |
| | | | | agonist |
| 92 | Piperine | BRD-K59522102 | −98.92 | Monoamine oxidase |
| | | | | inhibitor |
| 93 | Pyrazinamide | BRD-K28667793 | −98.83 | Fatty acid synthase |
| | | | | inhibitor |
| 94 | Fraxidin | BRD-K66944906 | −98.83 | Carbonic anhydrase |
| | | | | inhibitor |
| 95 | Larixinic-acid | BRD-K40619305 | −98.78 | Compound that |
| | | | | interacts with |
| | | | | metal centers |
| 96 | Midodrine | BRD-A79981887 | −98.74 | Adrenergic receptor |
| | | | | agonist |
| 97 | XAV-939 | BRD-K12762134 | −98.73 | Tankyrase inhibitor |
| 98 | AICA- | BRD-A67373739 | −98.7 | AMPK activator |
| | ribonucleotide | | | |
| 99 | PIK-90 | BRD-K99818283 | −98.66 | PI3K inhibitor |
| 100 | PNU-22394 | BRD-K16551401 | −98.63 | Serotonin receptor |
| | | | | agonist |
| 101 | AY-9944 | BRD-K03642198 | −98.62 | Hedgehog pathway |
| | | | | modulator |
| 102 | Gavestinel | BRD-K49890030 | −98.59 | Glutamate receptor |
| | | | | antagonist |
| 103 | Foliosidine | BRD-A49734948 | −98.54 | Plant alkaloid |
| 104 | Naftopidil | BRD-A01787639 | −98.48 | Adrenergic receptor |
| | | | | antagonist |
| 105 | GDC-0941 | BRD-K52911425 | −98.38 | PI3K inhibitor |
| 106 | Clonidine | BRD-K98530306 | −98.37 | Adrenergic receptor |
| | | | | agonist |
| 107 | CGP-54626 | BRD-A55369275 | −98.35 | GABA receptor |
| | | | | antagonist |
| 108 | Tosyllysyl- | BRD-K10136726 | −98.32 | Chymotrypsin |
| | chloromethyl- | | | inhibitor |
| | ketone | | | |
| 109 | Indatraline | BRD-K01649396 | −98.31 | Norepinephrine |
| | | | | transporter |
| | | | | inhibitor |
| 110 | 9-methyl- | BRD-K14696368 | −98.28 | NFkB pathway |
| | 5H-6-thia-4,5- | | | inhibitor |
| | diaza-chrysene- | | | |
| | 6,6-dioxide | | | |
| 111 | NNC-05-2090 | BRD-K85015012 | −98.27 | GAT inhibitor, |
| | | | | GABA uptake |
| | | | | inhibitor |
| 112 | 3-matida | BRD-A87125127 | −98.19 | Glutamate receptor |
| | | | | antagonist |
| 113 | Phenothiazine | BRD-K59597909 | −98.17 | Dopamine receptor |
| | | | | antagonist |
| 114 | Piribedil | BRD-K47936004 | −98.16 | Dopamine receptor |
| | | | | agonist |
| 115 | Mebeverine | BRD-A09467419 | −98.09 | Acetylcholine |
| | | | | receptor |
| | | | | antagonist |
| 116 | GANT-58 | BRD-K64451768 | −98.06 | GLI antagonist |
| 117 | RITA | BRD-K00317371 | −98.03 | MDM inhibitor |
| 118 | Pirenperone | BRD-K25224017 | −98.01 | Serotonin receptor |
| | | | | antagonist |
| 119 | KIN001-244 | BRD-K09186807 | −98.01 | Phosphoinositide |
| | | | | dependent |
| | | | | kinase inhibitor |
| 120 | Ozagrel | BRD-K19525698 | −97.97 | Thromboxane |
| | | | | synthase inhibitor |
| 121 | PP-30 | BRD-K30677119 | −97.96 | RAF inhibitor |
| 122 | CNQX | BRD-K53545112 | −97.96 | Glutamate receptor |
| | | | | antagonist |
| 123 | STO-609 | BRD-K52620403 | −97.91 | Calmodulin antagonist |
| 124 | Loperamide | BRD-K61250553 | −97.74 | Opioid receptor |
| | | | | agonist |
| 125 | Dichloroacetic- | BRD-K13664374 | −97.69 | Pyruvate |
| | acid | | | dehydrogenase kinase |
| | | | | inhibitor |
| 126 | PI-103 | BRD-K67868012 | −97.66 | MTOR inhibitor, |
| | | | | PI3K inhibitor |
| 127 | Metoclopramide | BRD-K75641298 | −97.54 | Dopamine |
| | | | | receptor and |
| | | | | serotonin receptor |
| | | | | antagonist |
| 128 | Spironolactone | BRD-K90027355 | −97.54 | Mineralocorticoid |
| | | | | receptor |
| | | | | antagonist |
| 129 | Loratadine | BRD-K82795137 | −97.51 | Histamine receptor |
| | | | | antagonist |
| 130 | Pirfenidone | BRD-K96862998 | −97.45 | TGF β receptor |
| | | | | inhibitor |
| 131 | ICI-89406 | BRD-A03359064 | −97.34 | Adrenergic receptor |
| | | | | antagonist |
| 132 | Clebopride | BRD-K17294426 | −97.31 | Dopamine receptor |
| | | | | antagonist |
| 133 | Prostaglandin- | BRD-K04010869 | −97.3 | HSP inducer, |
| | a1 | | | NFkB pathway |
| | | | | inhibitor |
| 134 | Butylparaben | BRD-K08287586 | −97.27 | DNA synthesis |
| | | | | inhibitor |
| 135 | Testosterone | BRD-A48720949 | −97.13 | androgen receptor |
| | | | | agonist |
| 136 | WZ-4002 | BRD-K72420232 | −97.11 | EGFR inhibitor |
| 137 | PLX-4720 | BRD-K16478699 | −97.01 | RAF inhibitor |
| 138 | Darinaparsin | BRD-K35723520 | −97.01 | Apoptosis stimulant |
| 139 | PTB1 | BRD-K16554956 | −96.94 | AMPK activator |
| 140 | Alosetron | BRD-K46742498 | −96.93 | Serotonin receptor |
| | | | | antagonist |
| 141 | U-99194 | BRD-K70281171 | −96.87 | Dopamine receptor |
| | | | | antagonist |
| 142 | Otenzepad | BRD-A00520476 | −96.85 | Acetylcholine |
| | | | | receptor |
| | | | | antagonist |
| 143 | Fursultiamine | BRD-A71157293 | −96.84 | Vitamin B |
| 144 | Piperacetazine | BRD-K16277217 | −96.79 | Dopamine receptor |
| | | | | antagonist |
| 145 | SD-169 | BRD-K91904471 | −96.77 | p38 MAPK inhibitor |
| 146 | Liothyronine | BRD-K89152108 | −96.77 | Thyroid hormone |
| | | | | stimulant |
| 147 | Mepacrine | BRD-A45889380 | −96.77 | Cytokine production |
| | | | | inhibitor, |
| | | | | NFkB pathway |
| | | | | inhibitor, TP53 |
| | | | | activator |
| 148 | Nicotine | BRD-K05395900 | −96.71 | Acetylcholine |
| | | | | receptor agonist |
| 149 | TG-101348 | BRD-K12502280 | −96.66 | FLT3 inhibitor, |
| | | | | JAK inhibitor |
| 150 | Quinpirole | BRD-A85280935 | −96.54 | Dopamine receptor |
| | | | | agonist |
| 151 | Mafenide | BRD-K30649484 | −96.53 | Carbonic anhydrase |
| | | | | inhibitor |
| 152 | Dephostatin | BRD-K60274257 | −96.5 | Tyrosine phosphatase |
| | | | | inhibitor |
| 153 | Cisapride | BRD-K06895174 | −96.49 | Serotonin receptor |
| | | | | agonist |
| 154 | Dicyclo- | BRD-K81521265 | −96.48 | Epoxide hydolase |
| | hexylurea | | | inhibitor |
| 155 | m- | BRD-K36965586 | −96.47 | Serotonin receptor |
| | chlorophenyl- | | | agonist |
| | biguanide | | | |
| 156 | Auraptene | BRD-K85013741 | −96.45 | Nitric oxide |
| | | | | production inhibitor |
| 157 | Alprenolol | BRD-A00993607 | −96.44 | Adrenergic |
| | | | | receptor antagonist |
| 158 | TPCA-1 | BRD-K51575138 | −96.39 | IKK inhibitor |
| 159 | Sertraline | BRD-K82036761 | −96.37 | Serotonin receptor |
| | | | | antagonist |
| 160 | AC-55649 | BRD-K93176058 | −96.37 | Retinoid receptor |
| | | | | agonist |
| 161 | CDK1-5- | BRD-K87932577 | −96.35 | CDK inhibitor, |
| | inhibitor | | | Glycogen |
| | | | | synthase kinase |
| | | | | inhibitor |
| 162 | D-64406 | BRD-K27665173 | −96.25 | PDGFR receptor |
| | | | | inhibitor |
| 163 | Fipronil | BRD-A50675702 | −96.24 | GABA gated |
| | | | | chloride channel |
| | | | | blocker |
| 164 | RO-25-6981 | BRD-K51541829 | −96.14 | Ionotropic |
| | | | | glutamate receptor |
| | | | | antagonist, Monamine |
| | | | | transporter modulator |
| 165 | BIIB021 | BRD-K51967704 | −96.11 | HSP inhibitor |
| 166 | AZD-6482 | BRD-K58772419 | −96.1 | PI3K inhibitor |
| 167 | EMD-386088 | BRD-K47659338 | −96.09 | Serotonin receptor |
| | | | | agonist |
| 168 | CITCO | BRD-K53263234 | −96.07 | CAR agonist |
| 169 | Exemestane | BRD-A73741725 | −95.97 | Aromatase inhibitor |
| 170 | GR-206 | BRD-K00184207 | −95.95 | Aryl hydrocarbon |
| | | | | receptor |
| | | | | ligand |
| 171 | Dasatinib | BRD-K49328571 | −95.91 | BCR-ABL kinase |
| | | | | inhibitor, |
| | | | | Ephrin inhibitor, |
| | | | | KIT inhibitor, |
| | | | | PDGFR receptor |
| | | | | inhibitor, SRC |
| | | | | inhibitor, |
| | | | | Tyrosine kinase |
| | | | | inhibitor |
| 172 | MDM2- | BRD-K84987553 | −95.89 | MDM inhibitor |
| | inhibitor | | | |
| 173 | Aminomethyl- | BRD-A28318179 | −95.8 | Nitric oxide |
| | transferase | | | synthase inhibitor |
| 174 | BRL-52537 | BRD-A37347161 | −95.7 | Opioid receptor |
| | | | | agonist |
| 175 | Amoxapine | BRD-K02265150 | −95.68 | Norepinephrine |
| | | | | reuptake |
| | | | | inhibitor |
| 176 | RO-08-2750 | BRD-K00486786 | −95.68 | NGF binding inhibitor |
| 177 | Flutamide | BRD-K28307902 | −95.68 | Androgen receptor |
| | | | | antagonist |
| 178 | DMBI | BRD-K96084870 | −95.63 | PDGFR receptor |
| | | | | inhibitor, |
| | | | | VEGFR inhibitor |
| 179 | Carmoxirole | BRD-K82484965 | −95.61 | Dopamine receptor |
| | | | | agonist |
| 180 | Tauro- | BRD-K33572481 | −95.59 | Bile acid |
| | deoxycholic- | | | |
| | acid | | | |
| 181 | Bupropion | BRD-A05186015 | −95.57 | Dopamine uptake |
| | | | | inhibitor |
| 182 | Chlor- | BRD-K86595100 | −95.49 | Benzodiazepine |
| | diazepoxide | | | receptor agonist |
| 183 | Roscovitine | BRD-K07691486 | −95.37 | CDK inhibitor |
| 184 | ALW-II-38-3 | BRD-K68191783 | −95.33 | Ephrin inhibitor |
| 185 | Ornidazole | BRD-A42759514 | −95.22 | Antiprotozoal |
| 186 | Iodophenpropit | BRD-K51918615 | −95.18 | Histamine receptor |
| | | | | antagonist |
| 187 | Prima-1-met | BRD-K49456190 | −95.18 | thioredoxin inhibitor |
| 188 | EI-247 | BRD-K32710582 | −95.17 | IGF-1 inhibitor |
| 189 | MK-2206 | BRD-K68065987 | −95.16 | AKT inhibitor |
| 190 | BMS-299897 | BRD-K02950022 | −95.14 | γ secretase inhibitor |
| 191 | Promazine | BRD-K06980535 | −95.11 | Dopamine receptor |
| | | | | antagonist |
| 192 | CGP-60474 | BRD-K79090631 | −95.09 | CDK inhibitor |
| 193 | PF-04217903 | BRD-K73319509 | −95.06 | c-Met inhibitor |
| 194 | Pantoprazole | BRD-A22380646 | −95.02 | ATPase inhibitor |
| 195 | Norgestimate | BRD-A04756508 | −94.95 | Progesterone receptor |
| | | | | agonist |
| 196 | Mead- | BRD-K09764130 | −94.82 | Cannabinoid receptor |
| | ethanolamide | | | agonist |
| 197 | CL-82198 | BRD-K00675675 | −94.73 | Metalloproteinase |
| | | | | inhibitor |
| 198 | HLI-373 | BRD-K17349619 | −94.73 | MDM inhibitor |
| 199 | Nifedipine | BRD-K96354014 | −94.71 | Calcium channel |
| | | | | blocker |
| 200 | Sildenafil | BRD-K50128260 | −94.71 | Phosphodiesterase |
| | | | | inhibitor |
| 201 | ICI-199441 | BRD-K73290745 | −94.63 | Opioid receptor |
| | | | | agonist |
| 202 | Ipsapirone | BRD-K90574421 | −94.61 | Serotonin receptor |
| | | | | agonist |
| 203 | Milrinone | BRD-K67080878 | −94.49 | Phosphodiesterase |
| | | | | inhibitor |
| 204 | Cotinine | BRD-K94144010 | −94.35 | Nicotine metabolite |
| 205 | Etilefrine | BRD-A09925278 | −94.23 | Adrenergic receptor |
| | | | | agonist |
| 206 | Thio- | BRD-K08619574 | −94.19 | Dopamine receptor |
| | properazine | | | antagonist |
| 207 | Acadesine | BRD-A95696820 | −94.15 | AMPK activator |
| 208 | Danazol | BRD-A92537424 | −94.09 | Estrogen receptor |
| | | | | antagonist, |
| | | | | Progesterone |
| | | | | receptor agonist |
| 209 | z-prolyl-p | BRD-K60174629 | −94.04 | Prolyl endopeptidase |
| | rolinal | | | inhibitor |
| 210 | Pravastatin | BRD-K60511616 | −94.03 | HMGCR inhibitor |
| 211 | PP-2 | BRD-K95785537 | −94.01 | SRC inhibitor |
| 212 | BRD- | BRD-K63784565 | −94.01 | Topoisomerase |
| | K63784565 | | | inhibitor |
| 213 | Geldanamycin | BRD-A19500257 | −93.9 | HSP inhibitor |
| 214 | Verapamil | BRD-A09533288 | −93.88 | Calcium channel |
| | | | | blocker |
| 215 | Amylocaine | BRD-A09062839 | −93.70 | Local anesthetic |
| 216 | Anagrelide | BRD-K62200014 | −93.38 | Phosphodiesterase |
| | | | | inhibitor |
| 217 | JAK3- | BRD-K52850071 | −93.37 | JAK inhibitor |
| | Inhibitor-II | | | |
| 218 | Felbamate | BRD-K99107520 | −93.35 | Glutamate receptor |
| | | | | antagonist |
| 219 | BP-554 | BRD-K45479396 | −93.33 | Serotonin receptor |
| | | | | agonist |
| 220 | Dicycloverine | BRD-K68507560 | −93.3 | Acetylcholine receptor |
| | | | | antagonist |
| 221 | Nicorandil | BRD-K97752965 | −93.29 | Nitric oxide donor, |
| | | | | Potassium |
| | | | | channel activator |
| 222 | SCH-442416 | BRD-K46469693 | −93.27 | Adenosine receptor |
| | | | | antagonist |
| 223 | Carpindolol | BRD-A15530910 | −93.19 | Adrenergic receptor |
| | | | | antagonist, |
| | | | | serotonin receptor |
| | | | | antagonist |
| 224 | VU-0420363-1 | BRD-K59633790 | −92.96 | SARS coronavirus |
| | | | | 3C-like |
| | | | | protease inhibitor |
| 225 | Oxybutynin | BRD-A65013509 | −92.9 | Acetylcholine receptor |
| | | | | antagonist |
| 226 | SA-792541 | BRD-K68143200 | −92.8 | CDC inhibitor |
| 227 | Dipropyl-5ct | BRD-K32645441 | −92.74 | Serotonin receptor |
| | | | | agonist |
| 228 | Ticlopidine | BRD-K00603606 | −92.72 | Purinergic receptor |
| | | | | antagonist |
| 229 | SDZ-WAG- | BRD-A31007383 | −92.71 | Adenosine receptor |
| | 994 | | | agonist |
| 230 | Meprylcaine | BRD-K65417056 | −92.69 | Local anesthetic |
| 231 | Cycloserine | BRD-K87226815 | −92.58 | Bacterial cell |
| | | | | wall synthesis |
| | | | | inhibitor |
| 232 | KIN001-127 | BRD-A29901043 | −92.54 | ITK inhibitor |
| 233 | Enrofloxacin | BRD-K76534306 | −92.54 | Bacterial DNA |
| | | | | gyrase inhibitor |
| 234 | Alverine | BRD-K89055274 | −92.52 | Muscle relaxant |
| 235 | Bepridil | BRD-A91008255 | −92.5 | Calcium channel |
| | | | | or L-type Ca⁺⁺ |
| | | | | channel blocker |
| 236 | Nefazodone | BRD-K90789829 | −92.39 | Adrenergic inhibitor, |
| | | | | Norepinephrine |
| | | | | reuptake |
| | | | | inhibitor, Serotonin |
| | | | | receptor |
| | | | | antagonist, |
| | | | | Serotonin reuptake |
| | | | | inhibitor |
| 237 | PSB-11 | BRD-K10177585 | −92.33 | Adenosine receptor |
| | | | | antagonist |
| 238 | Acetyl- | BRD-U01690642 | −92.32 | Isoprenylated protein |
| | geranyl- | | | methylation inhibitor |
| | cysteine | | | |
| 239 | Estradiol | BRD-A18917088 | −92.31 | Contraceptive |
| | | | | agent, Estrogen |
| | | | | receptor agonist |
| 240 | Seco- | BRD-K91733562 | −92.2 | Antioxidant |
| | isolariciresinol | | | |
| 241 | Prostaglandin | BRD-K09436313 | −92.13 | Prostanoid receptor |
| | | | | antagonist |
| 242 | Alfuzosin | BRD-A09056319 | −92.11 | Adrenergic receptor |
| | | | | antagonist |
| 243 | Oxybenzone | BRD-K59037100 | −92.1 | Lipase inhibitor |
| 244 | KIN001-220 | BRD-K53561341 | −91.98 | Aurora kinase |
| | | | | inhibitor |
| 245 | AZD-8055 | BRD-K69932463 | −91.85 | MTOR inhibitor |
| 246 | Toltrazuril | BRD-K64514229 | −91.83 | Antiprotozoal |
| 247 | Mepyramine | BRD-K97564742 | −91.79 | Histamine receptor |
| | | | | antagonist |
| 248 | Edaravone | BRD-K35458079 | −91.74 | Nootropic agent |
| 249 | FIT | BRD-K17896185 | −91.6 | Opioid receptor |
| | | | | agonist |
| 250 | Dopamine | BRD-K43887077 | −91.51 | Dopamine receptor |
| | | | | agonist |
| 251 | Tolterodine | BRD-K54316499 | −91.49 | Acetylcholine receptor |
| | | | | antagonist |
| 252 | L-BSO | BRD-A47706533 | −91.44 | Glutathione transferase |
| | | | | inhibitor |
| 253 | Dinoprostone | BRD-K26521938 | −91.34 | Prostanoid receptor |
| | | | | agonist |
| 254 | GR-144053 | BRD-K12120659 | −91.31 | Integrin antagonist |
| 255 | O-2050 | BRD-K02590140 | −91.29 | Cannabinoid receptor |
| | | | | antagonist |
| 256 | bis-tyrphostin | BRD-K32906660 | −91.25 | EGFR inhibitor |
| 257 | ITE | BRD-K60298136 | −91.16 | Aryl hydrocarbon |
| | | | | receptor |
| | | | | agonist |
| 258 | Nevirapine | BRD-K15502390 | −91.15 | Reverse transcriptase |
| | | | | inhibitor |
| 259 | GR-235 | BRD-K26674531 | −91.08 | Estrogen receptor |
| | | | | agonist, FXR |
| | | | | antagonist, |
| | | | | Progesterone |
| | | | | receptor agonist |
| 260 | Latrunculin-b | BRD-A19248578 | −91.08 | Actin polymerization |
| | | | | inhibitor, |
| | | | | Unidentified |
| | | | | pharmacological |
| | | | | activity |
| 261 | AR- | BRD-K40892394 | −91.08 | Nitric oxide synthase |
| | C133057XX | | | inhibitor |
| 262 | Temefos | BRD-K51805276 | −90.87 | Cholinesterase |
| | | | | inhibitor |
| 263 | Ilomastat | BRD-K51662849 | −90.81 | Matrix |
| | | | | metalloprotease |
| | | | | inhibitor |
| 264 | SID-26681509 | BRD-K08417745 | −90.75 | Cathepsin inhibitor |
| 265 | Formestane | BRD-A31801025 | −90.66 | Aromatase inhibitor |
| 266 | Iproniazid | BRD-K88568253 | −90.6 | Monoamine oxidase |
| | | | | inhibitor |
| 267 | Buphenine | BRD-A36267905 | −90.46 | Adrenergic receptor |
| | | | | agonist |
| 268 | Desipramine | BRD-K60762818 | −90.43 | Tricyclic |
| | | | | antidepressant |
| 269 | Tyrphostin-46 | BRD-K60184833 | −90.39 | Tyrosine kinase |
| | | | | inhibitor |
| 270 | RS-67506 | BRD-K50018155 | −90.29 | Serotonin |
| | | | | receptor partial |
| | | | | agonist |
| 271 | BRD- | BRD-K34437622 | −90.15 | Thymidylate |
| | K34437622 | | | synthase inhibitor |
| 272 | Rotenonic-acid | BRD-K34330170 | −90.11 | Retinoid receptor |
| | | | | antagonist |
| 273 | PIK-75 | BRD- | −90.1 | DNA protein |
| | | M16762496 | | kinase inhibitor, |
| | | | | PI3K inhibitor |
| 274 | Zacopride | BRD-A65615053 | −90.01 | Serotonin receptor |
| | | | | antagonist |
| 275 | Etifenin | BRD-K63979671 | −90.00 | Compound used |
| | | | | in hepatobiliary |
| | | | | scans of the liver |

Classification of host proteins implicated in SARS-CoV-2 infection in four modules. A set of 348 SARS-CoV-2-related host cell proteins composed of 332 proteins identified by Gordon et al., plus 16 reportedly involved in SARS-CoV-2 life cycle, were considered (Gordon D E et al. Nature, 2020, 583, 459-468; de Lartigue J et al. Traffic, 2009, 10, 883-893; Li P et al. Trends Biochem Sci. 2019, 44, 110-124; Hoffmann M et al. Cell, 2020, 181, 271-280; Ou X et al. Nat Commun. 2020, 11, 1620).

The 332 host cell proteins were identified by mass spectrometry upon expressing 26 of 29 SARS-CoV-2 proteins (non-structural proteins Nsp1-16, spike [S], envelop [E], membrane [M], nucleocapsid [N], and nine open reading frames [Orfs]), individually in HEK293T cells (Gordon D E et al. Nature, 2020, 583, 459-468). Comparison of the viral-human interactomes for SARS-CoV-2, SARS-CoV, and MERS-CoV (Gordon D E et al. Science 2020, 370, 1181) revealed that 14.7% of the SARS-CoV-2 host proteins were not among those detected in SARS-CoV-1 or MERSCoV interactomes, underscoring the significance of utilizing the viral/host interactome specific to SARS-CoV-2.

The additional 16 proteins are the receptor ACE2, the proteases transmembrane protease serine 2 (TMPRSS2), cathepsin B, and cathepsin L, as well as several cell signaling and regulation proteins (interleukin 6 [IL6] receptor, myeloid differentiation primary response 88 [MyD88], MAP kinase 1, protein kinase B [AKT1], mammalian target of rapamycin [mTOR], nuclear factor of activated T cells cytoplasmic 1 [NFATC1], nuclear factor κB subunit 1 [NFκB1], STAT3, ADAM metallopeptidase domain 17 [ADAM17], phosphatidylinositol 3-kinase catalytic subunit α [PIK3CA], phosphatidylinositol 3-phosphate 5-kinase [PlKfyve], and the two-pore channel 2 [TPC2]).

In order to better assess the involvement of these 348 host cell proteins in different phases of SARS-CoV-2 infection, they were mapped onto their KEGG pathways (243 pathways) and four functional modules (viral entry, viral replication and translation, host cell regulation and signaling, and immune response) were identified based on their KEGG annotations. This led to 27, 45, 27, and 32 proteins in the respective modules (see Table 7 and Table 8). Several proteins were shared between these modules, such that their union contained 103 host proteins. For example, MAPK and PI3KAKT-mTOR signaling pathways regulate CoV replication and translation (Zumla A et al. Nat Rev Drug Discov. 2016, 15, 327-347), in addition to mediating the immune response (Prompetchara E et al. Asian Pac J Allergy Immunol. 2020, 38, 1-9). Some proteins distinguished in a recent CRISPR screen (Daniloski Z et al. Cell 2021, 184, 1-14), including the Ras-related protein Rab-7A (RAB7A), and subunits of the ATPase vacuolar pump (ATP6AP1 and ATP6V1A) and intracellular cholesterol transporter (NPC2) are also noted in Table 7.

TABLE 7
Four modules mediating host cell response during SARS-CoV-2 infection,
corresponding pathways, and proteins.ª

| Module | | Gene names of the host cell |
|---|---|---|
| (# of | KEGG | proteins involved in |
| proteins) | pathways | the module |
| Viral Entry | Endocytosis, | SCARB1; ATP6AP1; AP3B1; NPC2; ITGB1; |
| (27 | lysosome | RAB8A; AP2A2; PIKFYVE; RHOA; |
| proteins) | pathway | RAB10; ACE2; AP2M1; ATP6V1A; |
| | | RNF41; CHMP2A; CTSB; WASHC4; |
| | | TMPRSS2; RAB7A; GLA; |
| | | SPART; CTSL; PPT1; ARF6; RAB5C; |
| | | NEU1; TPC2 |
| Viral | DNA | NUP62; ERLEC1; NUP214; |
| replication | replication, | EIF4E2; RPL36; |
| & | RNA | LMAN2; EXOSC3; NUP54; WFS1; PRIM2; |
| translation | transport, | SRP72; SIL1; UPF1; |
| (45 | RNA | SELENOS; POLA1; NUP88; |
| proteins) | degradation, | OS9; HYOU1; RAE1; RBX1; |
| | protein | EXOSC2; MRPS2; NUP98; PSMD8; |
| | processing | NGLY1; NUP58; ERO1B; EDEM3; |
| | in ER and | MRPS5; PRIM1; NUP210; ELOC; |
| | protein export | SRP54; ELOB; UGGT2; EXOSC5; IMPDH2; |
| | | PABPC4; EXOSC8; POLA2; SRP19; SLU7; |
| | | CUL2; MOGS; PABPCI |
| Regulation | Ras signaling, | IL6R; PIK3CA; RAB8A; |
| and | autophagy, | RALA; MTOR; TBK1; |
| signaling | AMPK | AKT1; NFKB1; GNG5; EIF4E2; PRKAR2A; |
| (27 | signaling, | RAB2A; MYD88; ATP6V1A; |
| proteins) | mTOR | COL6A1; RAB14; |
| | signaling, | MAPK1; RHOA; RAB10; |
| | PI3K-AKT | PRKAR2B; ITGB1; |
| | signaling, | GNB1; ARF6; RAB5C; ECSIT; PRKACA; |
| | and insulin | NFATC1 |
| | signaling | |
| Immune | Toll-like | MYD88; MAPK1; STAT3; |
| response | receptor-, | CUL2; HMOX1; |
| (32 | chemokine-, | ELOB; RIPK1; IL17RA; |
| proteins) | RIG-like | CSNK2B; MTOR; |
| | receptor-, | INHBE; PRKACA; GNB1; |
| | B cell | NLRX1; ERC1; |
| | receptor-, | RHOA; GDF15; TBK1; IL6R; |
| | NF-kB-, | AKT1; CSNK2A2; |
| | TCR-, | GNG5; NFATC1; TBKBP1; PIK3CA; |
| | and HIF-1- | CTSB; BX1; NFKB1; ELOC; |
| | signaling | REIF4E2; PLAT; ARF6 |
| | pathways | |

^{a}See Table 8 for the full names of the proteins whose gene codes are listed in column 3. Genes corresponding to some key proteins targeted by the proposed compounds/drug and/or mentioned in the text are written in bold face, including: ARF6 (ADP ribosylation factor 6); ATP6AV1A (ATPase H + transporting V1 subunit A); TBK1 (TANK-binding kinase 1); PRKACA (protein kinase CAMP-activated catalytic subunit α, or the catalytic subunit α of protein kinase A (PKA); RAB7A (Ras-related protein Rab-7A; RHOA (recombinant human RhoA); CTSL and CTSB (cathepsin L and B).

TABLE 8
Composition of four modules mediating host cell response during
SARS-CoV-2 infection.

| Module (# | Dominant | Host cell proteins (gene names) |
|---|---|---|
| of proteins) | pathways/processes | in the module |
| Viral entry | Endocytosis, | Scavenger receptor class B |
| (27 | lysosome | member 1 (SCARB1); V- |
| proteins) | pathway | type proton ATPase subunit |
| | | S1 (ATP6AP1); AP-3 |
| | | complex subunit β-1 (AP3B1); |
| | | NPC intracellular |
| | | cholesterol transporter 2 |
| | | (NPC2); integrin β-1 |
| | | (ITGB1); Ras-related protein |
| | | Rab-8A (RAB8A); AP-2 |
| | | complex subunit a-2 (AP2A2); |
| | | 1-phosphatidylinositol |
| | | 3-phosphate 5-kinase |
| | | (PIKFYVE); transforming |
| | | protein RhoA (RHOA); |
| | | Ras-related protein Rab-10 |
| | | (RAB10); angiotensin- |
| | | converting enzyme 2 (ACE2); |
| | | AP-2 complex subunit mu |
| | | (AP2M1); V-type proton |
| | | ATPase catalytic subunit A |
| | | (ATP6V1A); E3 ubiquitin- |
| | | protein ligase NRDP1 |
| | | (RNF41); charged |
| | | multivesicular body protein |
| | | 2a (CHMP2A); cathepsin |
| | | B (CTSB); WASH complex |
| | | subunit 4 (WASHC4); |
| | | TMPRSS2; Ras-related protein |
| | | Rab-7a (RAB7A); α- |
| | | galactosidase A (GLA); |
| | | spartin (SPART); cathepsin |
| | | L1 (CTSL); palmitoyl-protein |
| | | thioesterase 1 (PPT1); |
| | | ADP-ribosylation factor 6 |
| | | (ARF6); Ras-related protein |
| | | Rab-5C (RAB5C); sialidase-1 |
| | | (NEU1); two pore Ca²⁺ |
| | | channel protein 2 (TPC2) |
| Viral | DNA | Nuclear pore glycoprotein |
| replication | replication, | p62 (NUP62); endoplasmic |
| and | RNA transport, | reticulum lectin 1 (ERLEC1); |
| translation | RNA | nuclear pore complex |
| (45 | degradation, | protein Nup214 (NUP214); |
| proteins) | protein | eukaryotic translation |
| | processing | initiation factor 4E type 2 |
| | in ER | (EIF4E2); 60S ribosomal |
| | protein export | protein L36 (RPL36); vesicular |
| | | integral-membrane |
| | | protein VIP36 (LMAN2); |
| | | exosome complex |
| | | component RRP40 (EXOSC3); |
| | | nucleoporin p54 |
| | | (NUP54); wolframin (WFS1); |
| | | DNA primase large |
| | | subunit (PRIM2); signal |
| | | recognition particle subunit |
| | | SRP72 (SRP72); nucleotide |
| | | exchange factor SIL1 |
| | | (SIL1); regulator of nonsense |
| | | transcripts 1 (UPF1); |
| | | selenoprotein S (SELENOS); |
| | | DNA polymerase α |
| | | catalytic subunit (POLA1); |
| | | nuclear pore complex |
| | | protein Nup88 (NUP88); protein |
| | | OS-9 (OS9); hypoxia |
| | | up-regulated protein 1 |
| | | (HYOU1); mRNA export factor |
| | | (RAE1); E3 ubiquitin-protein |
| | | ligase RBX1 (RBX1); |
| | | exosome complex component |
| | | RRP4 (EXOSC2); |
| | | mitochondrial small ribosomal |
| | | subunit protein uS2m |
| | | (MRPS2); nuclear pore |
| | | complex protein Nup98-Nup96 |
| | | (NUP98); 26S proteasome |
| | | non-ATPase regulatory |
| | | subunit 8 (PSMD8); peptide-N |
| | | (4)-(N-acetyl-β- |
| | | glucosaminyl)asparagine |
| | | amidase (NGLY1); |
| | | nucleoporin p58 (NUP58); |
| | | ERO1-like protein β |
| | | (ERO1B); ER degradation- |
| | | enhancing α-mannosidase- |
| | | like protein 3 (EDEM3); |
| | | mitochondrial small |
| | | ribosomal subunit protein |
| | | uS5m (MRPS5); DNA |
| | | primase small subunit |
| | | (PRIM1); nuclear pore |
| | | membrane glycoprotein |
| | | 210 (NUP210); elongin-C |
| | | (ELOC); signal recognition |
| | | particle 54 kDa protein |
| | | (SRP54); elongin-B (ELOB); UDP- |
| | | glucose:glycoprotein |
| | | glucosyltransferase 2 (UGGT2); |
| | | exosome complex component |
| | | RRP46 (EXOSC5); |
| | | inosine-5′-monophosphate |
| | | dehydrogenase 2 |
| | | (IMPDH2); polyadenylate- |
| | | binding protein 4 |
| | | (PABPC4); exosome complex |
| | | component RRP43 |
| | | (EXOSC8); DNA polymerase |
| | | α subunit B (POLA2); |
| | | signal recognition particle |
| | | 19 kDa protein (SRP19); |
| | | pre-mRNA-splicing factor |
| | | SLU7 (SLU7); cullin-2 |
| | | (CUL2); mannosyl- |
| | | oligosaccharide glucosidase |
| | | (MOGS); polyadenylate- |
| | | binding protein 1 (PABPC |
| Regulation | Ras signaling, | Interleukin-6 receptor subunit |
| and | autophagy, | α (IL6R); PIK3CA; Ras- |
| signaling | AMPK | related protein Rab-8A |
| (27 | signaling, | (RAB8A); Ras-related protein |
| proteins) | mTOR | Ral-A (RALA); mTOR; |
| | signaling, PI3K- | serine/threonine-protein |
| | AKT | kinase TBK1 (TBK1); |
| | signaling and | AKT1; NFkB1; guanine |
| | insulin signaling | nucleotide-binding protein |
| | | G (I)/G (S)/G (O) subunit γ- |
| | | 5 (GNG5); eukaryotic |
| | | translation initiation factor 4E |
| | | type 2 (EIF4E2); cAMP- |
| | | dependent protein kinase type |
| | | II-α regulatory subunit |
| | | (PRKAR2A); Ras-related |
| | | protein Rab-2A (RAB2A); |
| | | MYD88; V-type proton |
| | | ATPase catalytic subunit A |
| | | (ATP6V1A); collagen α-1 |
| | | (VI) chain (COL6A1); |
| | | Ras-related protein Rab-14 |
| | | (RAB14); MAPK1; RHOA; |
| | | RAB10; cAMP- |
| | | dependent protein kinase type |
| | | II-β regulatory subunit |
| | | (PRKAR2B); integrin |
| | | β-1 (ITGB1); guanine |
| | | nucleotide-binding protein |
| | | G (I)/G (S)/G (T) subunit β- |
| | | 1 (GNB1); ARF6; RAB5C; |
| | | evolutionarily conserved |
| | | signaling intermediate in |
| | | Toll pathway (ECSIT); |
| | | CAMP-dependent protein |
| | | kinase catalytic subunit α |
| | | (PRKACA); NFATC1 |
| Immune | Interferon-, TLR-, | Myeloid differentiation primary |
| response | chemokine-, | response protein |
| (32 | NFkB-, | MyD88 (MYD88); MAPK1; |
| proteins) | RIG-like | STAT3; cullin-2 (CUL2); |
| | receptor-, | heme oxygenase 1 (HMOX1); |
| | B-cell | ELOB; receptor- |
| | receptor-, T | interacting serine/threonine- |
| | cell receptor- | protein kinase 1 (RIPK1); |
| | and | interleukin-17 receptor A |
| | HIF-1-signaling | (IL17RA); casein kinase II |
| | | subunit β (CSNK2B); serine/ |
| | | threonine-protein kinase |
| | | mTOR (MTOR); inhibin β |
| | | E chain (INHBE); cAMP- |
| | | dependent protein kinase |
| | | catalytic subunit α |
| | | (PRKACA); guanine nucleotide- |
| | | binding protein G |
| | | (I)/G (S)/G (T) subunit β-1 |
| | | (GNB1); NLR family |
| | | member X1 (NLRX1); ELKS/ |
| | | Rab6-interacting/CAST |
| | | family member 1 (ERC1); RHOA; |
| | | growth/differentiation |
| | | factor 15 (GDF15); |
| | | serine/threonine-protein kinase |
| | | TBK1 (TBK1); IL6R; |
| | | AKT1; casein kinase II |
| | | subunit α (CSNK2A2); |
| | | guanine nucleotide-binding |
| | | protein G (I)/G (S)/G (O) |
| | | subunit γ-5 (GNG5); NFATC1; |
| | | TANK-binding kinase |
| | | 1-binding protein 1 (TBKBP1); |
| | | PIK3CA; cathepsin B |
| | | (CTSB); E3 ubiquitin-protein |
| | | ligase RBX1 (RBX1); |
| | | NFkB1; ELOC; eukaryotic |
| | | translation initiation factor |
| | | 4E type 2 (EIF4E2); tissue-type |
| | | plasminogen activator |
| | | (PLAT); ARF6 |

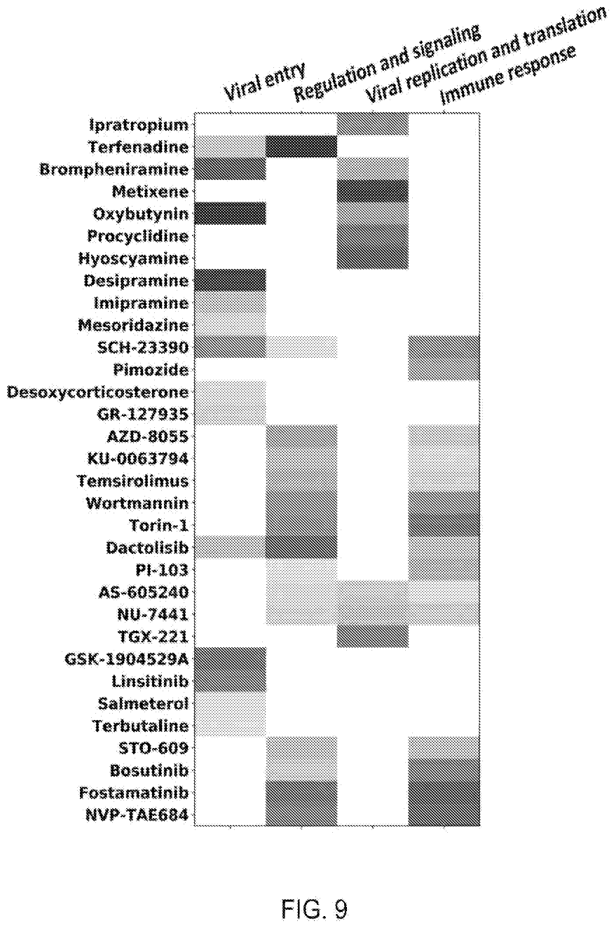

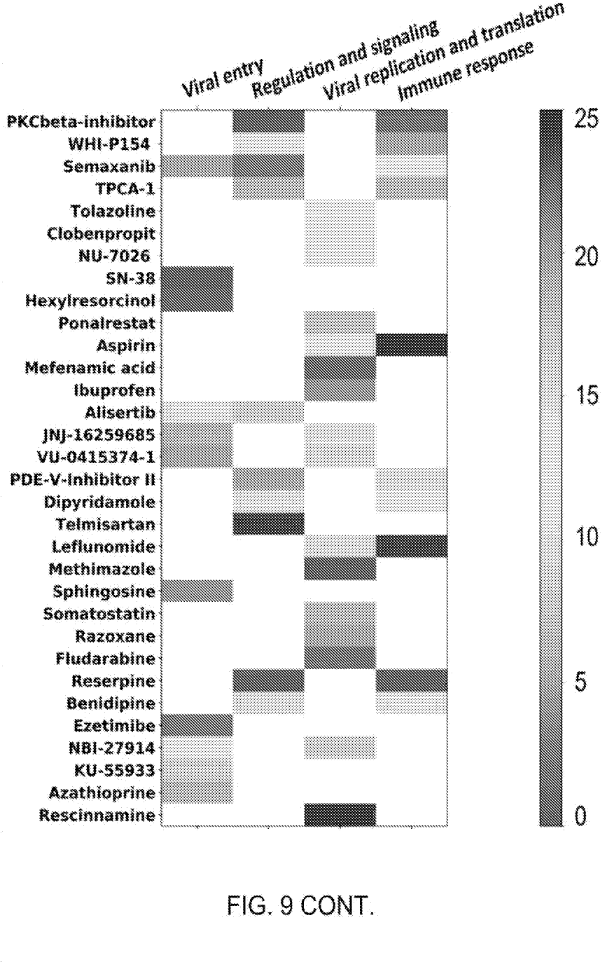

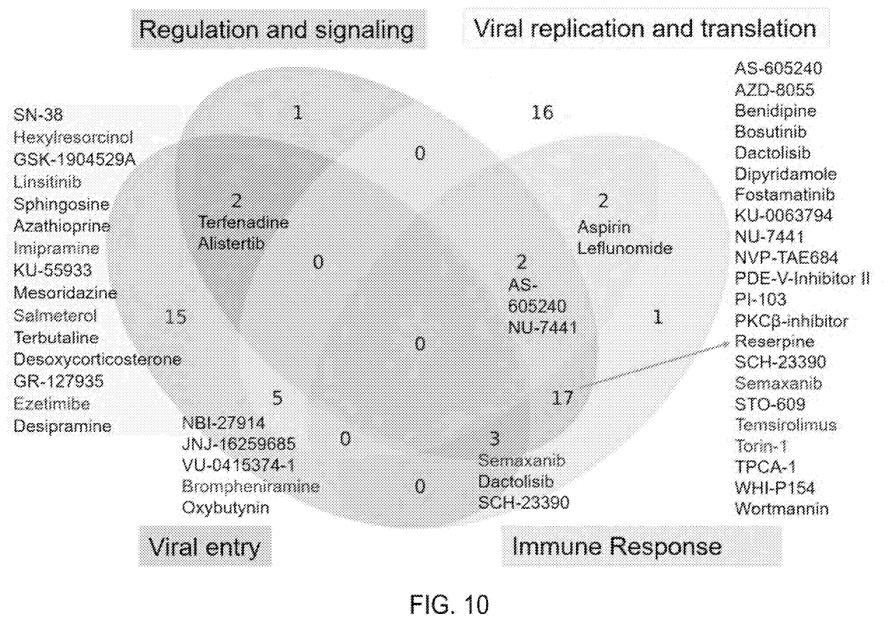

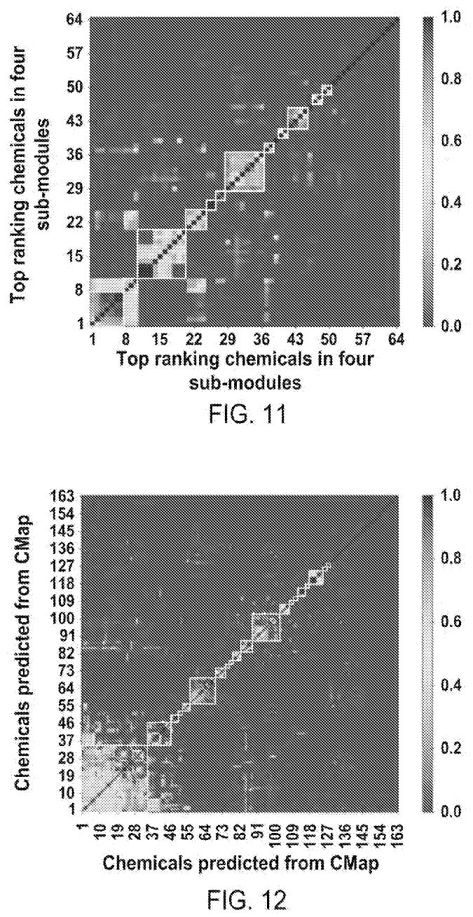

Prioritization of candidate compounds proposed to have antiviral effects. As a measure of the potential antiviral effect of the compounds deduced from the computational analysis, the proximity of their targets to each disease module were calculated. Specifically, the distance between the targets of each compound and the proteins belonging to each module were evaluated using the lung-specific PPI network from BioSNAP (Zitnik M et al. BioSNAP datasets: Stanford biomedical network dataset collection. 2018, http://snapstanfordedu/biodata) and network proximity analysis (Guney E et al. Nat Commun. 2016, 7, 10331) (see Materials and Methods). Top-ranking 25 compounds were selected for each module (FIG. 9 and Table 9) leading to a set of 64 distinct compounds in the union of four modules (FIG. 10). FIG. 9-FIG. 10 show the identification and classification of prioritized potentially antiviral compounds. Clustering of these based on their interaction patterns with target proteins (using QuartataWeb) led to 12 clusters (FIG. 11 and Table 10) containing 48 of the compounds; the remaining 16 exhibited unique interaction patterns. Up to two representatives were selected from each cluster and further evaluated (manually) with literature-based evidence including their MOAs, side effects, availability, and antiviral evidence if any, to generate a reduced set of 13 high-priority compounds, listed in Table 11. In addition, after manual evaluation of 95 compounds that lack data in DrugBank and STITCH, two investigational compounds, rottlerin, and QL-XII-47, with respective CMap scores of 97.11 and 99.03, were added to the high-priority list (see FIG. 6).

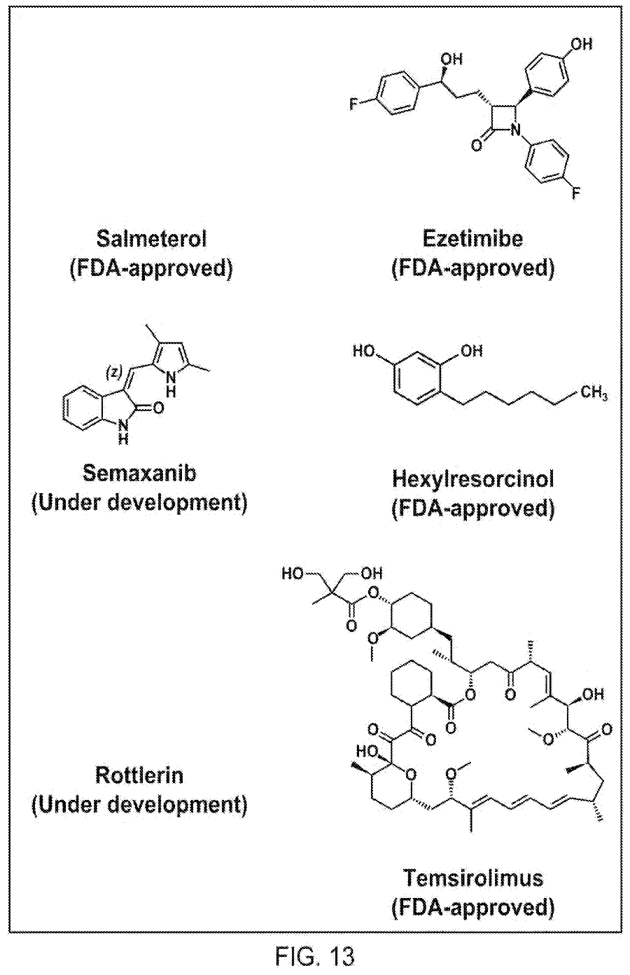

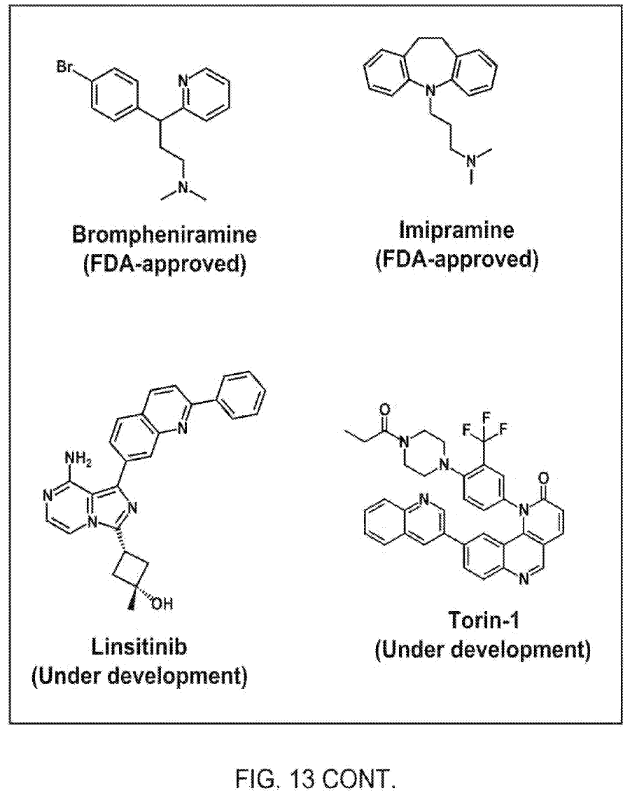

The final set of 15 compounds that are proposed to have antiviral activities (Table 11) contains eight FDA-approved (repurposable) drugs and seven under investigation. Ten of these have been tested in in vitro assays (indicated by asterisks in Table 11; and labeled in red in FIG. 10). FIG. 13 displays the corresponding chemical structures.

TABLE 9
Top-ranking 64 compounds involved in four
disease modules, rank-ordered by the proximity
of the corresponding targets to the disease modules (*)

| Viral entry | | | Viral replication & translation | | |
|---|---|---|---|---|---|
| In- | Compound | | In- | Compound | |
| dex | name | z-score | dex | name | z-score |
| 1 | SN-38 | −2.97E+00 | 1 | Methimazole | −3.93E+00 |
| 2 | Hexylresorcinol | −1.87E+00 | 2 | Mefenamic acid | −2.62E+00 |
| 3 | GSK-1904529A | −1.79E+00 | 3 | Fludarabine | −2.50E+00 |
| 4 | Linsitinib | −1.79E+00 | 4 | TGX-221 | −1.87E+00 |
| 5 | Sphingosine | −1.48E+00 | 5 | Ibuprofen | −1.50E+00 |
| 6 | Semaxanib | −1.09E+00 | 6 | Razoxane | −1.38E+00 |
| 7 | Azathioprine | −1.08E+00 | 7 | Somatostatin | −8.50E−01 |
| 8 | Imipramine | −8.18E−01 | 8 | NU-7441 | −8.39E−01 |
| 9 | KU-55933 | −7.96E−01 | 9 | AS-605240 | −7.83E−01 |
| 10 | Mesoridazine | −7.43E−01 | 10 | Leflunomide | −7.80E−01 |
| 11 | Alisertib | −7.13E−01 | 11 | NU-7026 | −6.66E−01 |
| | | | | (LY-293646) | |
| 12 | Salmeterol | −6.89E−01 | 12 | Aspirin | −6.62E−01 |
| 13 | Terbutaline | −6.89E−01 | 13 | Tolazoline | −4.83E−01 |
| 14 | NBI-27914 | −6.56E−01 | 14 | Clobenpropit | −4.83E−01 |
| 15 | Desoxy- | −6.45E−01 | 15 | JNJ-16259685 | −4.79E−01 |
| | corticosterone | | | | |
| 16 | GR-127935 | −6.45E−01 | 16 | VU-0415374-1 | −4.79E−01 |
| 17 | Terfenadine | −4.98E−01 | 17 | NBI-27914 | −4.79E−01 |
| 18 | Dactolisib | −4.57E−01 | 18 | Ponalrestat | −4.72E−01 |
| 19 | JNJ-16259685 | −4.48E−01 | 19 | Brompheniramine | −2.93E−01 |
| 20 | VU-0415374-1 | −4.48E−01 | 20 | Oxybutynin | −2.44E−01 |
| 21 | SCH-23390 | −4.46E−01 | 21 | Ipratropium | −2.44E−01 |
| 22 | Ezetimibe | −4.42E−01 | 22 | Procyclidine | −2.44E−01 |
| 23 | Brompheniramine | −4.39E−01 | 23 | Hyoscyamine | −2.44E−01 |
| 24 | Desipramine | −4.03E−01 | 24 | Metixene | −2.44E−01 |
| 25 | Oxybutynin | −3.61E−01 | 25 | Rescinnamine | −2.28E−01 |
| Cell signaling & regulation | | | Immune response | | |
| In- | Compound | | In- | Compound | |
| dex | name | z-score | dex | name | z-score |
| 1 | PKCβ-inhibitor | −4.10E+00 | 1 | Fostamatinib | −5.16E+00 |
| 2 | Dactolisib | −3.91E+00 | 2 | NVP-TAE684 | −4.47E+00 |
| 3 | Fostamatinib | −3.48E+00 | 3 | PKCβ-inhibitor | −4.37E+00 |
| 4 | NVP-TAE684 | −3.24E+00 | 4 | Bosutinib | −3.59E+00 |
| 5 | Wortmannin | −3.19E+00 | 5 | Wortmannin | −3.29E+00 |
| 6 | PDE-V- | −2.83E+00 | 6 | WHI-P154 | −3.04E+00 |
| | Inhibitor II | | | (JAK3- | |
| | | | | Inhibitor-II) | |
| 7 | TPCA-1 | −2.56E+00 | 7 | TPCA-1 | −3.04E+00 |
| 8 | STO-609 | −2.50E+00 | 8 | Dactolisib | −2.88E+00 |
| 9 | Bosutinib | −2.39E+00 | 9 | NU-7441 | −2.77E+00 |
| 10 | Benidipine | −2.37E+00 | 10 | PDE-V- | −2.68E+00 |
| | | | | Inhibitor II | |
| 11 | Dipyridamole | −2.22E+00 | 11 | Benidipine | −2.32E+00 |
| 12 | SCH-23390 | −2.00E+00 | 12 | Dipyridamole | −2.06E+00 |
| 13 | PI-103 | −1.92E+00 | 13 | Semaxanib | −2.06E+00 |
| 14 | WHI-P154 | −1.88E+00 | 14 | AS-605240 | −1.89E+00 |
| | (JAK3- | | | | |
| | Inhibitor-II) | | | | |
| 15 | AS-605240 | −1.88E+00 | 15 | KU-0063794 | −1.87E+00 |
| 16 | NU-7441 | −1.81E+00 | 16 | Temsirolimus | −1.87E+00 |
| 17 | Alisertib | −1.80E+00 | 17 | AZD-8055 | −1.87E+00 |
| 18 | KU-0063794 | −1.79E+00 | 18 | STO-609 | −1.84E+00 |
| 19 | Temsirolimus | −1.79E+00 | 19 | PI-103 | −1.81E+00 |
| 20 | AZD-8055 | −1.79E+00 | 20 | Pimozide | −1.79E+00 |
| 21 | Torin-1 | −1.72E+00 | 21 | SCH-23390 | −1.70E+00 |
| 22 | Semaxanib | −1.66E+00 | 22 | Torin-1 | −1.69E+00 |
| 23 | Reserpine | −1.54E+00 | 23 | Reserpine | −1.65E+00 |
| 24 | Telmisartan | −1.48E+00 | 24 | Leflunomide | −1.58E+00 |
| 25 | Terfenadine | −1.36E+00 | 25 | Aspirin | −1.40E+00 |
| 18 | KU-0063794 | −1.79E+00 | 18 | STO-609 | −1.84E+00 |

*Bold and italicized compounds/drugs have been experimentally tested. z-score is a measure of proximity, the lower scores indicating closer proximity. 25 compounds with the lowest proximity score are listed for each module. Several compounds participate in multiple modules resulting in 64 distinct compounds in the listed four modules.

TABLE 10
Grouping of 64 potentially antiviral compounds/drugs into clusters
based on their interaction patterns with their targets (*).

| Cluster | | | PubChem | DrugBank |
|---|---|---|---|---|
| index | Index | Compound name | ID | ID |
| 1 | 1 | Ipratropium | 657309 | DB00332 |
| | 2 | Terfenadine | 5405 | DB00342 |
| | 3 | Brompheniramine | 6834 | DB00835 |
| | 4 | Metixene | 4167 | DB00340 |
| | 5 | Oxybutynin | 4634 | DB01062 |
| | 6 | Procyclidine | 4919 | DB00387 |
| | 7 | Hyoscyamine | 154417 | DB00424 |
| 2 | 8 | Desipramine | 2995 | DB01151 |
| | 9 | Imipramine | 3696 | DB00458 |
| | 10 | Mesoridazine | 4078 | DB00933 |
| | 11 | SCH-23390 | 5018 | NA |
| | 12 | Pimozide | 16362 | DB01100 |
| | 13 | Desoxycorticosterone | 6166 | NA |
| | 14 | GR-127935 | 107780 | NA |
| 3 | 15 | AZD-8055 | 25262965 | DB12774 |
| | 16 | KU-0063794 | 16736978 | NA |
| | 17 | Temsirolimus | 6918289 | DB06287 |
| 4 | 18 | Wortmannin | 312145 | DB08059 |
| | 19 | Torin-1 | 49836027 | NA |
| | 20 | Dactolisib | 11977753 | NA |
| | 21 | PI-103 | 9884685 | NA |
| | 22 | AS-605240 | 5289247 | DB04769 |
| | 23 | NU-7441 | 11327430 | NA |
| | 24 | TGX-221 | 9907093 | NA |
| 5 | 25 | GSK-1904529A | 25124816 | NA |
| | 26 | Linsitinib | 11640390 | NA |
| 6 | 27 | Salmeterol | 5152 | DB00938 |
| | 28 | Terbutaline | 5403 | DB00871 |
| 7 | 29 | STO-609 | 3467590 | NA |
| | 30 | Bosutinib | 5328940 | DB06616 |
| | 31 | Fostamatinib | 11671467 | DB12010 |
| | 32 | NVP-TAE684 | 16038120 | NA |
| | 33 | PKCβ-inhibitor | 6419755 | NA |
| | 34 | WHI-P154 (JAK3-Inhibitor-II) | 3795 | NA |
| | 35 | Semaxanib | 5329098 | NA |
| | 36 | TPCA-1 | 9903786 | NA |
| 8 | 37 | Tolazoline | 5504 | DB00797 |
| | 38 | Clobenpropit | 2790 | NA |
| 9 | 39 | NU-7026 (LY-293646) | 9860529 | NA |
| 10 | 40 | SN-38 | 104842 | DB05482 |
| | 41 | Hexylresorcinol | 3610 | DB11254 |
| 11 | 42 | Ponalrestat | 5278 | NA |
| | 43 | Aspirin | 2244 | DB00945 |
| | 44 | Mefenamic acid | 4044 | DB00784 |
| | 45 | Ibuprofen | 3672 | DB01050 |
| 12 | 46 | Alisertib | 24771867 | DB05220 |
| 13 | 47 | JNJ-16259685 | 11313361 | NA |
| | 48 | VU-0415374-1 | 46869940 | NA |
| 14 | 49 | PDE-V-Inhibitor II | 9844109 | NA |
| | 50 | Dipyridamole | 3108 | DB00975 |
| 15 | 51 | Telmisartan | 65999 | DB00966 |
| 16 | 52 | Leflunomide | 3899 | DB01097 |
| 17 | 53 | Methimazole | 1349907 | DB00763 |
| 18 | 54 | Sphingosine | 5280335 | DB03203 |
| 19 | 55 | Somatostatin | 16129681 | DB09099 |
| 20 | 56 | Razoxane | 30623 | NA |
| 21 | 57 | Fludarabine | 657237 | DB01073 |
| 22 | 58 | Reserpine | 5770 | DB00206 |
| 23 | 59 | Benidipine | 656668 | DB09231 |
| 24 | 60 | Ezetimibe | 150311 | DB00973 |
| 25 | 61 | NBI-27914 | 176157 | NA |
| 26 | 62 | KU-55933 | 5278396 | NA |
| 27 | 63 | Azathioprine | 2265 | DB00993 |
| 28 | 64 | Rescinnamine | 5280954 | DB01180 |

*13 compounds bolded and italicized are prioritized after the analysis

TABLE 11
High-priority compounds with potential antiviral effects based on Dataset 1 (*).
Prioritized compounds based on Cmap scores and Network Proximity Ranks

| Drug/ | | Disease | | Description/ | |
|---|---|---|---|---|---|
| compoundª | Status | module | Rank^{b} | MOAs | Ref. |
| Brompheniramine* | FDA-approved | Viral entry | 23 | Histamine | Gwaltney Jr JM et |
| | | | | receptor | al. Clin Infect Dis. |
| | | | | antagonist | 1997, 25, 1188- |
| | | | | | 1194 |
| Ipratropium | FDA-approved | Viral | 21 | Acetylcholine | Barnes PJ. Am J |
| | | replication | | receptor | Med. 2004, |
| | | and | | antagonist | 117(Suppl 12A), |
| | | translation | | | 24S-32S |
| Imipramine* | FDA-approved | Viral entry | 8 | Norepinephrine | Shchors K et al. |
| | | | | and serotonin | Cancer Cell, 2015, |
| | | | | reuptake | 28, 456-471); |
| | | | | inhibitor, | Wichit S et al. Sci |
| | | | | autophagy | Rep. 2017, 7, 3145; |
| | | | | enhancer | Plenge P et al. Nat |
| | | | | | Commun. 2020, 11, |
| | | | | | 1491 |
| Temsirolimus* | FDA-approved | Immune | 16 | mTOR | Di Benedetto F et al. |
| | | response | | inhibitor, | Transplantation |
| | | Regulation | 19 | autophagy | 2010, 89, 733-738; |
| | | and | | enhancer | Soliman A et al. Exp |
| | | signaling | | | Clin Transplant, |
| | | | | | 2013, 11, 408-411; |
| | | | | | Bergmann L et al. |
| | | | | | Expert Rev |
| | | | | | Anticancer Ther. |
| | | | | | 2014, 14, 9-21; |
| | | | | | Kindrachuk J et al. |
| | | | | | Antimicrob Agents |
| | | | | | Chemother. 2015, |
| | | | | | 59, 1088-1099 |
| Torin-1* | Investigational | Immune | 22 | mTOR | Clippinger AJ et al. J |
| | | response | | inhibitor, PI3K | Virol. 2011, 85, |
| | | Regulation | 21 | inhibitor, | 3930-3939); |
| | | and | | autophagy | Bergmann L et al. |
| | | signaling | | enhancer | Expert Rev |
| | | | | | Anticancer Ther. |
| | | | | | 2014, 14, 9-21 |
| AS-605240 | Investigational | Regulation | 15 | PI3K inhibitor, | Azzi J et al. Diabetes |
| | | and | | autophagy | 2012, 61, 1509- |
| | | signaling | | enhancer | 1518 |
| | | Viral | 9 | | |
| | | replication | | | |
| | | and | | | |
| | | translation | | | |
| | | Immune | 14 | | |
| | | response | | | |
| Linsitinib* | Investigational | Viral entry | 4 | IGF-1-and | Mulvihill MJ et al. |
| | | | | insulin receptor | Future Med Chem. |
| | | | | inhibitor, TBK1 | 2009, 1, 1153- |
| | | | | activator | 1171; Sparrer KMJ |
| | | | | through ARF1 | et al. Nat Microbiol. |
| | | | | | 2017, 2, 1543-1557 |
| Salmeterol* | FDA-approved | Viral entry | 12 | β2 Adrenergic | Medigeshi GR et al. |
| | | | | receptor | Antimicrob Agents |
| | | | | agonist, | Chemother. 2016, |
| | | | | autophagy | 60, 6709-6718 |
| | | | | enhancer | |
| Semaxanib* | Investigational | Viral entry | 6 | VEGFR | O'Donnell A et al. |
| | | Regulation | 22 | inhibitor | Br J Cancer, 2005, |
| | | and | | | |
| | | signaling | | | |
| | | Immune | 13 | | 93, 876-883 |
| | | response | | | |
| Hexylresorcinol* | FDA-approved | Viral entry | 2 | Local anesthetic | Wilson CO et al. |
| | | | | | Textbook of organic |
| | | | | | medicinal and |
| | | | | | pharmaceutical |
| | | | | | chemistry, 1966, 5th |
| | | | | | edn. Philadelphia, |
| | | | | | PA: Lippincott |
| Mefenamic acid | FDA-approved | Viral repl | 2 | Cyclooxygenase | Rothan HA et al. |
| | | and | | inhibitor | Antiviral Res. 2016, |
| | | translation | | | 127,50-56 |
| JNJ16259685 | Investigational | Viral entry | 19 | Glutamate | Lavreysen H et al. |
| | | | | receptor | Neuropharmacology, |
| | | | | antagonist | 2004, 47, 961-972 |
| Ezetimibe* | FDA-approved | Viral entry | 22 | Niemann-Pick | Osuna-Ramos JF et |
| | | Regulation | 9 | C1-like 1 | al. Antiviral Res. |
| | | and | | protein | 2018, 160, 151-164 |
| | | signaling | | antagonist, | |
| | | | | cholesterol | |
| | | | | inhibitor, | |
| | | | | autophagy | |
| | | | | enhancer | |

Additional prioritized compounds (based on Cmap scores and literature)

| Drug/compoundª | Status | Description/MOAs | Ref. |
|---|---|---|---|
| QL-XII-47 | Investigational | Cytoplasmic tyrosine protein | de Wispelaere M et |
| | | kinase BMX inhibitor | al. J Biol Chem. |
| | | | 2020, 295, 1694- |
| | | | 1703 |
| Rottlerin* | Investigational | MAPK and protein kinase | Lama Z et al. |
| | | inhibitor, autophagy enhancer | Antiviral Res. |
| | | | 2019, 168, 51-60 |

ªThose tested in experiments are indicated by asterisks in the first column.
^{b}Rank refers to the proximity to the module in the third column, the lower the better.

Prioritization of candidate compounds proposed to have anti-inflammatory effects. A similar interaction pattern-based clustering of the 163 compounds predicted to potentially have anti-cytokine effect (among the high Cmap-scoring 275; see Table 6) led to 20 clusters of two or more compounds based on compound-protein interaction patterns, while 35 compounds were left as singletons (FIG. 12 and Table 12). Nineteen high-priority compounds representative of these clusters in addition to 5 singletons were selected. Furthermore, literature search of the remaining 112 potentially anti-inflammatory compounds for which no target data were available in DrugBank and STITCH, led to three additional candidate compounds. The resulting set of 27 potentially anti-inflammatory/cytokine compounds is presented in Table 13.

Table 13 contains 15 FDA-approved drugs and 12 compounds under investigation. Of note, two of the compounds under investigation (JAK3-Inhibitor-II and AZD-8055; in boldface) also belong to the 64 top-ranking compounds based on Dataset 1; and one, mepacrine/quinacrine, is listed in the Excelra COVID-19 drug repurposing database (https://wwwexcelracom/covid-19-drug-repurposing-database/). Another investigational drug in the list, PCA4248, is a platelet-activating factor (PAF) receptor antagonist (Fernandez-Gallardo S et al. J Pharmacol Exp Ther. 1990, 255, 34-39), and its utility against COVID-19 (e.g., for preventing coagulation or blood clots) is to be explored, as well as those of the two His receptor antagonists azelastine and chlorphenamine, identified here. Recent study draws attention to the possible repurposing of PAF receptor antagonists and His receptor antagonists against hyperinflammation and microthromboses in COVID-19 patients (Demopoulos C et al. BioFactors 2020, 46, 927-933).

Among approved drugs, pirfenidone is known to inhibit furin (Burghardt I et al. Biochem Biophys Res Commun. 2007, 354, 542-547), a human protease involved in the cleavage of the viral spike glycoprotein into S1 and S2 subunits (like TMPRSS2). Spike cleavage is essential to activate the S1 fusion trimer for viral entry. Pirfenidone combined with melatonin has been pointed out to be a promising therapy for reducing cytokine storm in COVID-19 patients (Artigas L et al. PloS One 2020, 15, e0240149). Finally, Table 13 also contains two approved cyclooxygenase inhibitors, oxaprozin and dexketoprofen, known as non-steroidal anti-inflammatory drugs (NSAIDs) (Miller L G. Clin Pharm. 1992, 11, 591-603; Moore R A et al. Clin Pharm. 2008, 8, 11).

TABLE 12
Grouping of 163 potential modulators of hyperinflammatory response
into clusters based on their interaction patterns with their targets (*).

| Cluster | | | PubChem | DrugBank |
|---|---|---|---|---|
| index | Index | Compound name | ID | ID |
| 1 | 1 | Chlorprothixene | 667467 | DB01239 |
| | 2 | Olanzapine | 4585 | DB00334 |
| | 3 | Amoxapine | 2170 | DB00543 |
| | 4 | Desipramine | 2995 | DB01151 |
| | 5 | Trifluoperazine | 5566 | DB00831 |
| | 6 | Maprotiline | 4011 | DB00934 |
| | 7 | Promazine | 4926 | DB00420 |
| | 8 | Ipsapirone | 56971 | NA |
| | 9 | Trazodone | 5533 | DB00656 |
| | 10 | Nefazodone | 4449 | DB01149 |
| | 11 | Zuclopenthixol | 5311507 | DB01624 |
| | 12 | Cisapride | 2769 | DB00604 |
| | 13 | Fluphenazine | 3372 | DB00623 |
| | 14 | Lisuride | 28864 | DB00589 |
| | 15 | Bromocriptine | 31101 | DB01200 |
| | 16 | Piribedil | 4850 | DB12478 |
| | 17 | Thioproperazine | 9429 | DB01622 |
| | 18 | Quinpirole | 54562 | NA |
| | 19 | Pirenperone | 4847 | NA |
| | 20 | Ketanserin | 3822 | DB12465 |
| | 21 | Latrepirdine | 197033 | DB11725 |
| | 22 | Cyclazosin | 132266 | NA |
| | 23 | Midodrine | 4195 | DB00211 |
| | 24 | Nor-binaltorphimine | 5480230 | NA |
| | 25 | EMD-386088 | 10131112 | NA |
| | 26 | Dopamine | 681 | DB00988 |
| | 27 | Azelastine | 2267 | DB00972 |
| | 28 | Loratadine | 3957 | DB00455 |
| | 29 | Verapamil | 2520 | DB00661 |
| | 30 | Naftopidil | 4418 | DB12092 |
| | 31 | Clonidine | 2803 | DB00575 |
| | 32 | Alfuzosin | 2092 | DB00346 |
| | 33 | Clebopride | 2780 | DB13511 |
| | 34 | U-99194 | 5626 | NA |
| 2 | 35 | Mepyramine | 4992 | DB06691 |
| | 36 | Chlorphenamine | 2725 | DB01114 |
| | 37 | Dicycloverine | 3042 | DB00804 |
| | 38 | Oxybutynin | 4634 | DB01062 |
| | 39 | Otenzepad | 107867 | NA |
| | 40 | Tolterodine | 443879 | DB01036 |
| | 41 | Profenamine | 3290 | DB00392 |
| | 42 | Xaliproden | NA | DB06393 |
| | 43 | Alverine | 3678 | DB01616 |
| | 44 | Bupropion | 444 | DB01156 |
| | 45 | Indatraline | 126280 | NA |
| | 46 | Sertraline | 68617 | DB01104 |
| 3 | 47 | Alprenolol | 2119 | DB00866 |
| | 48 | ICI-89406 | 123686 | NA |
| | 49 | Bisoprolol | 2405 | DB00612 |
| | 50 | Carteolol | 2583 | DB00521 |
| 4 | 51 | Clarithromycin | 84029 | DB01211 |
| | 52 | Bepridil | 2351 | DB01244 |
| 5 | 53 | Fipronil | 3352 | NA |
| | 54 | CGS-20625 | 163844 | NA |
| | 55 | Topiramate | 5284627 | DB00273 |
| | 56 | Chlordiazepoxide | 2712 | DB00475 |
| 6 | 57 | Saracatinib | 10302451 | DB11805 |
| | 58 | HY-11007 | 5311510 | NA |
| | 59 | Dasatinib | 3062316 | DB01254 |
| | 60 | Fostamatinib | 11671467 | DB12010 |
| | 61 | TG-101348 (Fedratinib) | 16722836 | DB12500 |
| | 62 | PLX-4720 | 24180719 | DB06999 |
| | 63 | TPCA-1 | 9903786 | NA |
| | 64 | DMBI | 5353593 | NA |
| | 65 | Orantinib | 5329099 | DB12072 |
| | 66 | D-64406 | 5330535 | NA |
| | 67 | WZ-4002 | 44607530 | NA |
| | 68 | bis-tyrphostin | 5329255 | NA |
| | 69 | JAK3-Inhibitor-II | 3795 | NA |
| 7 | 70 | TGX-221 | 9907093 | NA |
| | 71 | AZD-6482 | 44137675 | DB14980 |
| | 72 | GDC-0941 (Pictilisib) | 17755052 | DB11663 |
| | 73 | PI-103 | 9884685 | NA |
| | 74 | AZD-8055 | 25262965 | DB12774 |
| 8 | 75 | Roscovitine (Seliciclib) | 160355 | DB06195 |
| | 76 | CGP-60474 | 644215 | NA |
| 9 | 77 | Milrinone | 4197 | DB00235 |
| | 78 | Anagrelide | 2182 | DB00261 |
| 10 | 79 | Hexamethylene | 1794 | NA |
| | 80 | FIT | 84008 | NA |
| | 81 | Loperamide | 3955 | DB00836 |
| | 82 | BRL-52537 | 6603740 | NA |
| 11 | 83 | Nicotine | 89594 | DB00184 |
| | 84 | Zacopride | 108182 | NA |
| | 85 | Metoclopramide | 4168 | DB01233 |
| | 86 | m-chlorophenylbiguanide | 1354 | NA |
| | 87 | Palonosetron | 6337614 | DB00377 |
| | 88 | Alosetron | 2099 | DB00969 |
| 12 | 89 | Flutamide | 3397 | DB00499 |
| | 90 | Danazol | 28417 | DB01406 |
| | 91 | Norgestimate | 6540478 | DB00957 |
| | 92 | Oxybenzone | 4632 | DB01428 |
| | 93 | α-estradiol | 68570 | NA |
| | 94 | Diethylstilbestrol | 448537 | DB00255 |
| | 95 | Estradiol | 5757 | DB00783 |
| | 96 | Testosterone | 6013 | DB00624 |
| | 97 | Ticlopidine | 5472 | DB00208 |
| | 98 | Nifedipine | 4485 | DB01115 |
| | 99 | Nimodipine | 4497 | DB00393 |
| | 100 | Spironolactone | 5833 | DB00421 |
| | 101 | Liothyronine | 5920 | DB00279 |
| | 102 | CITCO | 9600409 | NA |
| 13 | 103 | Piperine | 638024 | DB12582 |
| | 104 | Phenelzine | 3675 | DB00780 |
| | 105 | Iproniazid | 3748 | DB04818 |
| | 106 | Selegiline | 26757 | DB01037 |
| | 107 | Auraptene | 1550607 | NA |
| 14 | 108 | Atorvastatin | 60823 | DB01076 |
| | 109 | Pravastatin | 54687 | DB00175 |
| 15 | 110 | Exemestane | 60198 | DB00990 |
| | 111 | Formestane | 11273 | DB08905 |
| 16 | 112 | Felbamate | 3331 | DB00949 |
| | 113 | Cycloserine | 6234 | DB00260 |
| | 114 | Gavestinel | 6450546 | DB06741 |
| | 115 | RO-25-6981 | 6604887 | NA |
| 17 | 116 | EHNA | 3206 | NA |
| | 117 | SCH-442416 | 10668061 | NA |
| 18 | 118 | Bromfenac | 60726 | DB00963 |
| | 119 | DUP-697 | 3177 | NA |
| | 120 | Dexketoprofen | 667550 | DB09214 |
| | 121 | Oxaprozin | 4614 | DB00991 |
| | 122 | Phenothiazine | 7108 | DB11447 |
| | 123 | Triptolide | 107985 | DB12025 |
| | 124 | Pyrazinamide | 1046 | DB00339 |
| 19 | 125 | SCH-28080 | 108137 | NA |
| | 126 | Pantoprazole | 4679 | DB00213 |
| 20 | 127 | BIIB021 | 16736529 | DB12359 |
| | 128 | Geldanamycin | 5288382 | DB02424 |
| 21 | 129 | STO-609 | 3467590 | NA |
| 22 | 130 | Mafenide | 3998 | DB06795 |
| 23 | 131 | AR-C133057XX | 9797857 | DB07002 |
| 24 | 132 | Buphenine (Nylidrin) | 4567 | DB06152 |
| 25 | 133 | AICA-ribonucleotide | 65110 | DB01700 |
| 26 | 134 | Valproic acid | 3121 | DB00313 |
| 27 | 135 | Iodophenpropit | 3035746 | NA |
| 28 | 136 | Retinol (Vitamin A) | 445354 | DB00162 |
| 29 | 137 | Sildenafil | 5212 | DB00203 |
| 30 | 138 | MK-2206 | 24964624 | NA |
| 31 | 139 | KI-16425 | 10367662 | NA |
| 32 | 140 | Dinoprostone | 5280360 | DB00917 |
| 33 | 141 | BI-78D3 | 2747117 | NA |
| 34 | 142 | Y-27632 | 448042 | DB08756 |
| 35 | 143 | SB-216763 | 176158 | NA |
| 36 | 144 | Linsitinib | 11640390 | NA |
| 37 | 145 | PF-04217903 | 17754438 | DB12848 |
| 38 | 146 | Mepacrine | 237 | DB01103 |
| 39 | 147 | Dicyclohexylurea | 4277 | NA |
| 40 | 148 | Pirfenidone | 40632 | DB04951 |
| 41 | 149 | Dichloroacetic acid | 6597 | DB08809 |
| 42 | 150 | MLN-4924 (Pevonedistat) | 16720766 | DB11759 |
| 43 | 151 | Thenoyltrifluoroacetone | 5601 | DB04795 |
| 44 | 152 | Navitoclax | 24978538 | DB12340 |
| 45 | 153 | Daunorubicin | 30323 | DB00694 |
| 46 | 154 | UNC-0321 | 46901937 | NA |
| 47 | 155 | Isoliquiritigenin | 638278 | DB03285 |
| 48 | 156 | PCA-4248 | 4698 | NA |
| 49 | 157 | Pyroxamide | 4996 | DB12847 |
| 50 | 158 | Rucaparib | 9931954 | DB12332 |
| 51 | 159 | Ilomastat | 132519 | DB02255 |
| 52 | 160 | Latrunculin-b | 6436219 | DB08080 |
| 53 | 161 | Nicorandil | 47528 | DB09220 |
| 54 | 162 | Z-prolyl-prolinal | 122623 | DB03535 |
| 55 | 163 | BRD-K63784565 | 97226 | DB12385 |

* The 24 compounds bolded and italicized are prioritized based on the cluster analysis

TABLE 13
Compounds proposed to help attenuate hyperinflammation based on Dataset 2.

| | | | |
|---|---|---|---|
| Drug/ | | Description/ | |
| Compound | Status | MOAs | Ref. |

Compounds extracted from Cmap and prioritized after QuartataWeb cluster analysis

| | | | |
|---|---|---|---|
| Midodrine | FDA-approved | Adrenergic receptor | Josset L et al. PLOS One, 2010, |
| | | agonist | 5, e13169 |
| Olanzapine | | Dopamine receptor | Altschuler EL et al. Med |
| | | antagonist, | Hypotheses 2020, 141, 109774 |
| | | autophagy enhancer | |
| Trifluoperazine | | Dopamine receptor | Ochiai H et al. Antiviral Res. |
| | | antagonist, | 1991, 15, 149-160 |
| | | autophagy dual- | |
| | | modulator | |
| Fluphenazine | | Dopamine receptor | Otreba M et al. Eur J |
| | | antagonist, | Pharmacol. 2020, 887, 173553 |
| | | autophagy enhancer | |
| Azelastine | | Dopamine receptor | Konrat R et al. bioRxiv, 2020, |
| | | antagonist, His | https://doi.org/10.1101/ |
| | | receptor antagonist | 2020.09.15.296228 |
| Chlorphenamine | | Histamine receptor | Xu W et al. Front Microbiol. |
| | | antagonist | 2018, 9, 2643 |
| Clarithromycin | | Bacterial 50S | Yamaya M et al. Eur Respir J. |
| | | ribosomal subunit | 2012, 40, P4364; Pani A et al. |
| | | inhibitor autophagy | Int J Antimicrob Agents, 2020, |
| | | inhibitor | 56, 106053 |
| Saracatinib | Investigational | SRC inhibitor | Shin JS et al. Viruses, 2018, 10, |
| | | | 283 |
| JAK3-Inhibitor-II | | JAK inhibitor | Schwartz DM et al. Nat Rev |
| | | | Drug Discov. 2017, 17, 843- |
| | | | 862 |
| AZD-8055 | | mTOR inhibitor, | Jiang Q et al. Cancer Res. |
| | | autophagy enhancer | 2011, 71, 4074-4084 |
| CGP-60474 | | CDK inhibitor | He B et al. F1000Research |
| | | | 2020, 9, 609 |
| Mepacrine/ | | Cytokine | Dermawan JKT et al. Mol |
| Quinacrine | | production | Cancer Ther. 2014, 14, 2203- |
| | | inhibitor, NFκB | 2214 |
| | | inhibitor | |
| Hexamethylene | | Sodium/hydrogen | Wilson L et al. Virology, 2006, |
| | | antiport inhibitor | 353, 294-306 |
| Loperamide | FDA-approved | Opioid receptor | Shen L et al. J Virol. 2019, 93, |
| | | agonist, autophagy | e00023-e119 |
| | | enhancer | |
| Nifedipine | | Calcium channel | Liu W et al. Life Sci. 2009, 85, |
| | | blocker, autophagy | 235 -240; Straus MR et al. |
| | | enhancer | bioRxiv, 2020, |
| | | | https://doi.org/10.1101/ |
| | | | 2020.07.21.214577 |
| Liothyronine | | Thyroid hormone | CN103705497B |
| | | stimulant | |
| Atorvastatin | | HMGCR inhibitor, | Episcopio D et al. FASEB J. |
| | | autophagy enhancer | 2019, 33, 9516-9525 |
| Triptolide | Investigational | RNA polymerase | Chaparala S et al. Preprints, |
| | | inhibitor, TNF-α | 2020, https://doi.org/10.20944/ |
| | | inhibitor | preprints202009.0459.v1 |
| Pirfenidone | FDA-approved | TGFβ receptor | Ferrara F et al. Eur J Clin |
| | | inhibitor, furin | Pharmacol. 2020, 76, 1615- |
| | | inhibitor, anti- | 1618 |
| | | fibrotic, autophagy | |
| | | enhancer | |
| Oxaprozin | | Cyclooxygenase | Miller LG. Clin Pharm. 1992, |
| | | inhibitor, NSAID | 11,591-603 |
| | | (non-steroidal anti- | |
| | | inflammatory drug) | |
| Dexketoprofen | | Cyclooxygenase | Moore RA et al. Clin Pharm. |
| | | inhibitor, NSAID | 2008, 8, 11 |
| Isoliquiritigenin | Investigational | Guanylate cyclase | Traboulsi H et al. Antimicrob |
| | | activator, | Agents Chemother. 2015, 59, |
| | | autophagy enhancer | 6317-6327 |
| PCA-4248 | | Platelet activating | Fernandez-Gallardo S et al. J |
| | | factor (PAF) | Pharmacol Exp Ther. 1990, |
| | | receptor antagonist | 255, 34-39 |
| Rucaparib | FDA-approved | PARP inhibitor, | Guo T et al. Nat Microbiol. |
| | | autophagy enhancer | 2019, 4, 1872-1884 |

Compounds extracted from Cmap and prioritized by literature search

| | | | |
|---|---|---|---|
| Berbamine | Investigational | Calmodulin | Huang L et al. Res Sq. 2020, |
| | | antagonist, | https://doi.org/10.21203/ |
| | | autophagy inhibitor | rs.3.rs-30922/v1 |
| Darinaparsin | | Apoptosis stimulant | Chowdhury T et al. Infect |
| | | | Disord Drug Targets 2020, 21, |
| | | | 608-618 |
| Taurodeoxycholic | | Bile acid | Li N et al. Sci Bull (Beijing), |
| acid | | | 2019, 64: 180-188 |

The three drugs/compounds in boldface are also predicted as antiviral drugs based on Dataset 1, listed in Table 11.

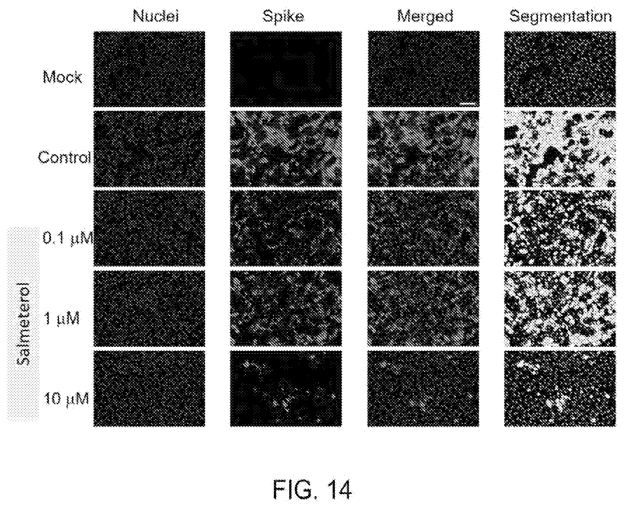

Testing the SARS-CoV-2 inhibitory properties of prioritized compounds in in vitro assays. First, five compounds (salmeterol, rottlerin, temsirolimus, torin-1, and ezetimibe) were selected from the list of 15 prioritized compounds described in Table 11 for a proof of concept in vitro evaluation of their anti-SARS-CoV-2 potential. FIG. 14-FIG. 17 show the suppression of SARS-CoV-2 infection by identified compounds.

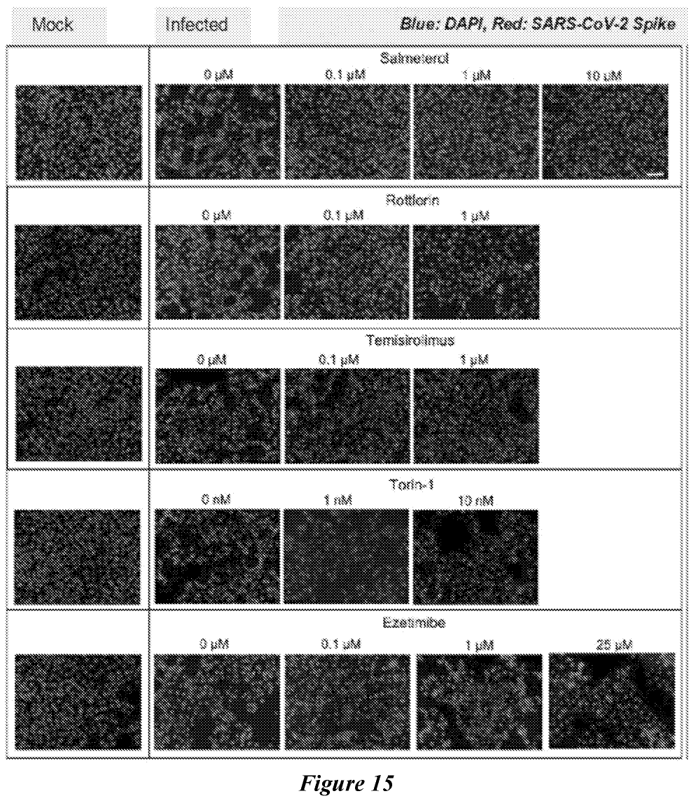

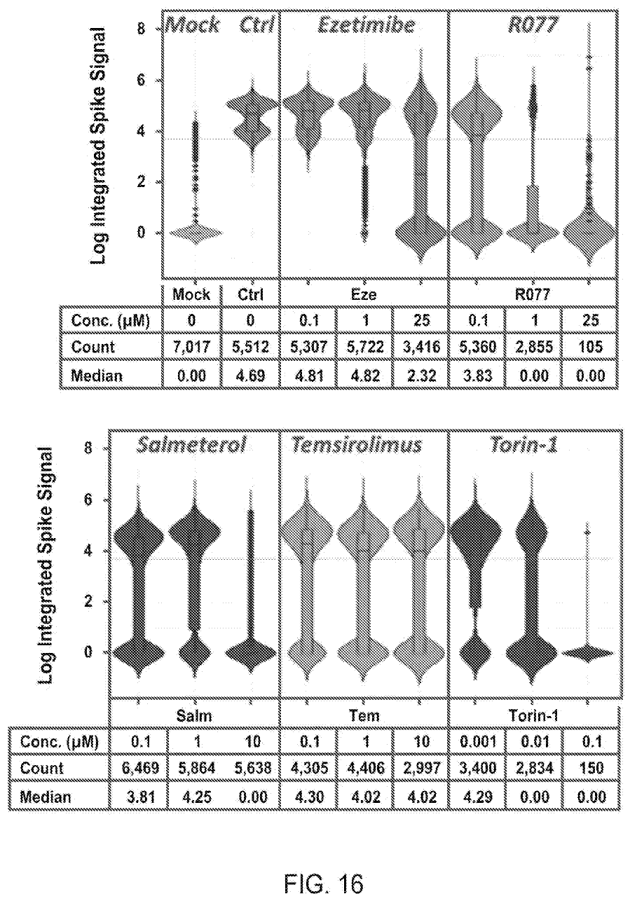

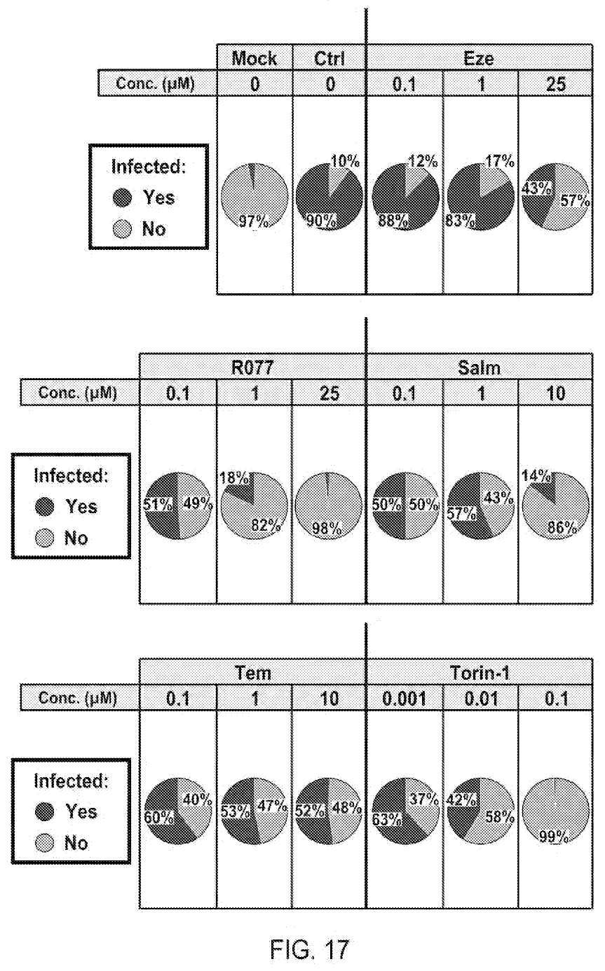

A SARS-CoV-2 infectious cell culture system (FIG. 14 and FIG. 15) where host Vero-E6 cells were pretreated with compounds (salmeterol, rottlerin (R077), temsirolimus, torin-1, or ezetimibe) for 1 h prior to SARS-CoV-2 inoculation was used. After 48-h post-infection, cells were fixed and fluorescently labeled for SARS-CoV-2 S protein and immunofluorescence was performed to assess viral infection (SARS-CoV-2 S protein; FIG. 14 and FIG. 15). Images were analyzed for spike-positive cells using the Multiwavelength Cell Scoring algorithm in MetaXpress. Representative mock and vehicle control images and their segmentation are shown in FIG. 14. Violin plots describing the distribution of the log integrated spike for each cell in the untreated and treated samples are shown in FIG. 16 along with complementary pie charts indicating the percent of cells positive for spike protein (FIG. 17). In the untreated controls, a bimodal distribution of spike-positive cells was evident, indicating the presence of two infected cell populations with one expressing more spike protein per cell than the other (FIG. 16). Salmeterol at 0.1 and 1 μM reduced the median of the spike-expressing population and showed a preferential antiviral effect for the lower spike-expressing subpopulation (FIG. 16). At 10 μM, salmeterol exhibited a greater antiviral effect on the entire population, although some (˜14%) spike-positive cells were evident (FIG. 16). Qualitatively similar results to salmeterol were obtained with rottlerin and the mTOR inhibitors, Temsirolimus, and Torin-1, although dose-limiting toxicity as evidenced by reduced cell count prevented a determination of a more complete antiviral effect on the higher-spike protein-expressing subpopulation in torin-1- and R077-treated cells (FIG. 16). Ezetimibe reduced spike protein-expressing populations only at the highest concentration studied (25 μM), where a reduction in cell numbers was also observed.

Next, cell fusion assays were used as a proxy for ACE2/SARS-CoV-2-mediated viral entry. Prioritized compounds predicted to potentially act as viral entry blockers, i.e., imipramine, brompheniramine, linsitinib, semaxanib, and hexylresorcinol, in addition to salmeterol and ezetimibe from the above set were focused on (see Table 11). The cell fusion assay, first described by Simmons et al. (Simmons G et al. Proc Natl Acad Sci USA, 2004, 101, 4240-4245), detects host-cell-spike interactions on a shorter time scale than the viral infection assay and has been used by several groups to investigate the mechanisms of cell entry of SARS-CoV-1, such as endosomal and protease involvement including TMPRSS2 (Matsuyama S et al. Proc Natl Acad Sci USA 2005, 102, 12543-12547; Matsuyama S et al. J Virol. 2010, 84, 12658-12664). More recently, the assay has also been used to investigate SARS-CoV-2-mediated cell entry (Ou X et al. Nat Commun. 2020, 11, 1620). The assay is based on the principle that susceptible host cells (“acceptors”) fuse with spike-expressing “donor” cells, forming large cell fusion constructs (syncytia), which can be quantified by fluorescence imaging.

This assay was implemented in a high-content, 384-well microplate format using HEK293T cells, which are not susceptible to viral infection unless transfected with ACE2 and TMPRSS2, and Calu-3 lung cancer cells, which possess the replete machinery for spike-mediated viral infection (Hoffmann M et al. Cell, 2020, 181, 271-280). HEK293T cells transfected with ACE2 and TMPRSS2 or native Calu-3 cells were incubated with donor cells co-expressing green fluorescent protein (GFP) and SARS-CoV-2 spike, and syncytia formation monitored by following GFP over time by fluorescence microscopy. After a 4-h incubation, syncytia were quantified by high-content analysis. Cell fusion was dependent on the presence of SARS-CoV-2 spike as donor cells expressing only GFP did not form syncytia.

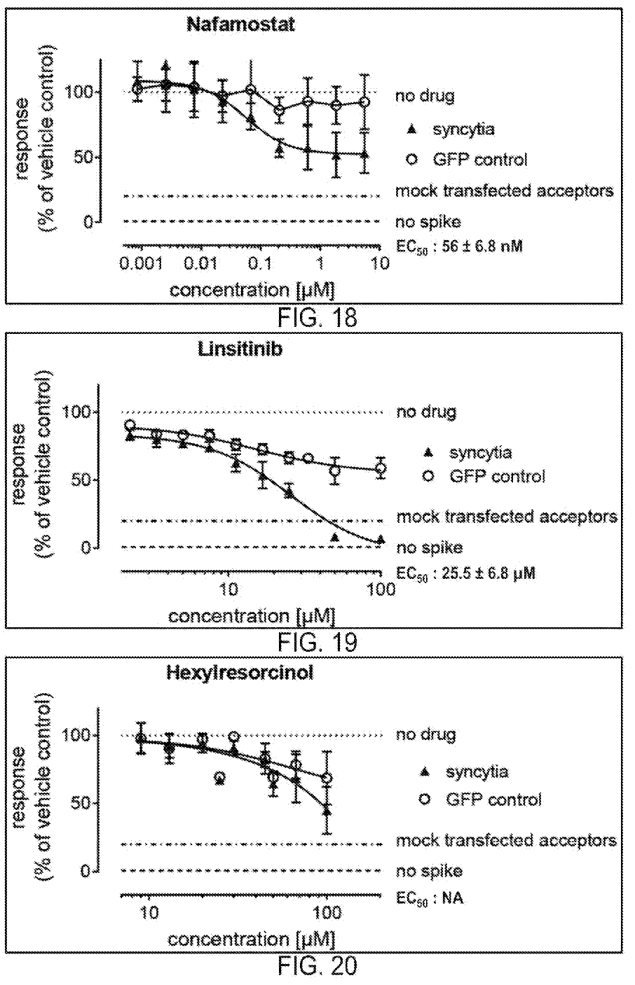

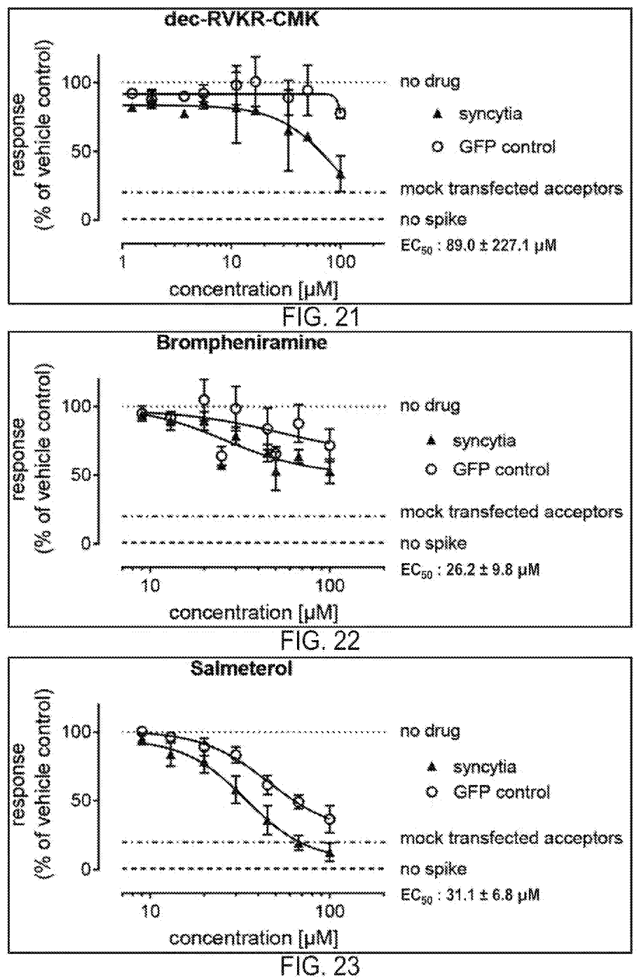

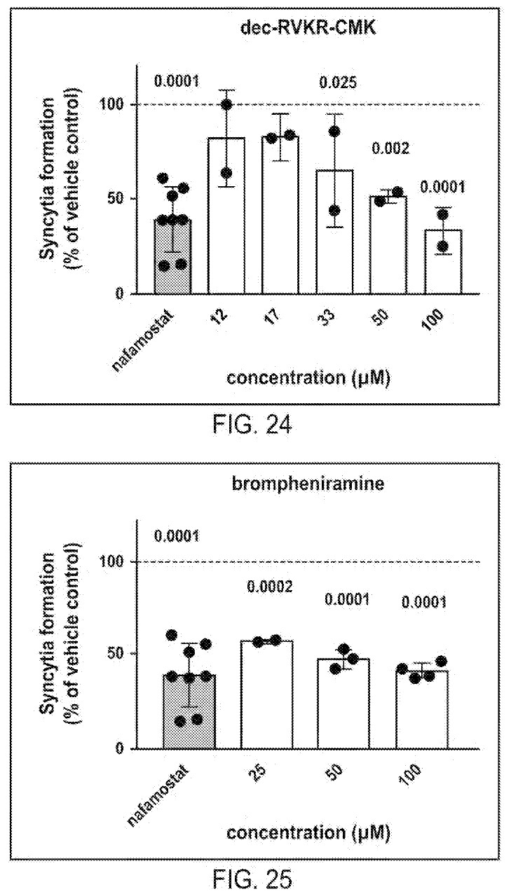

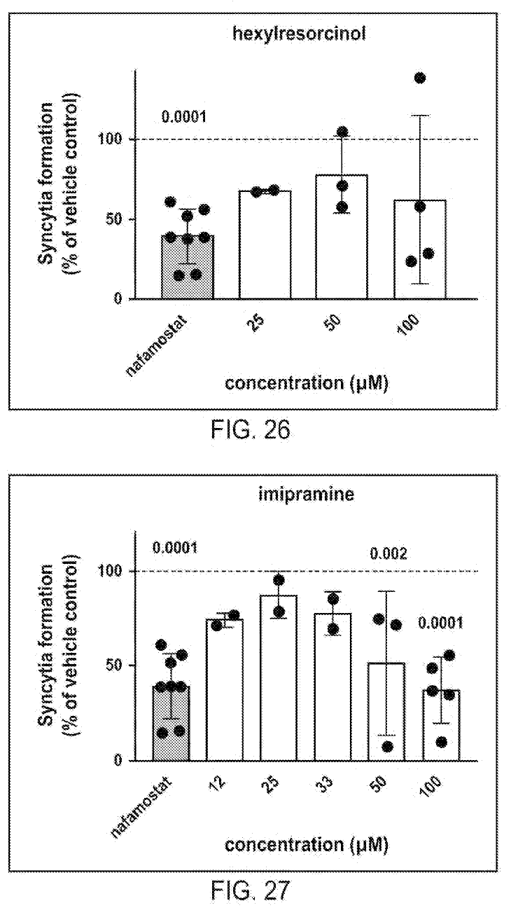

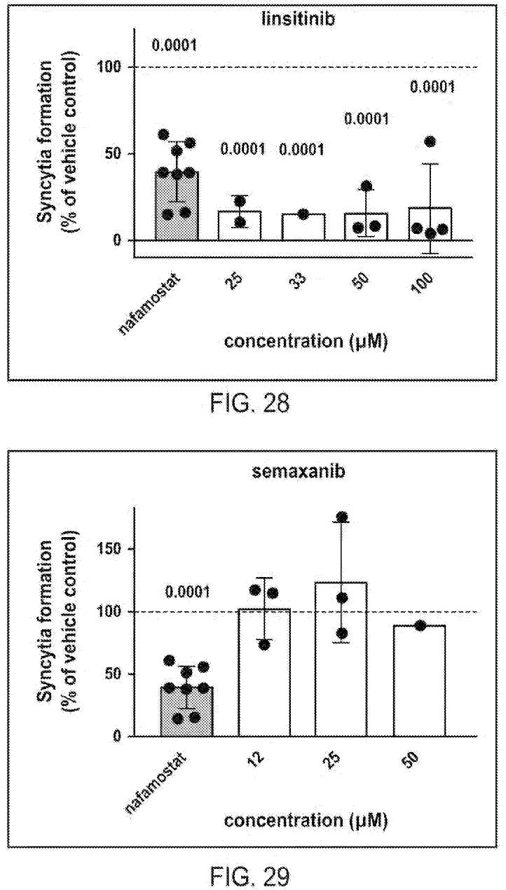

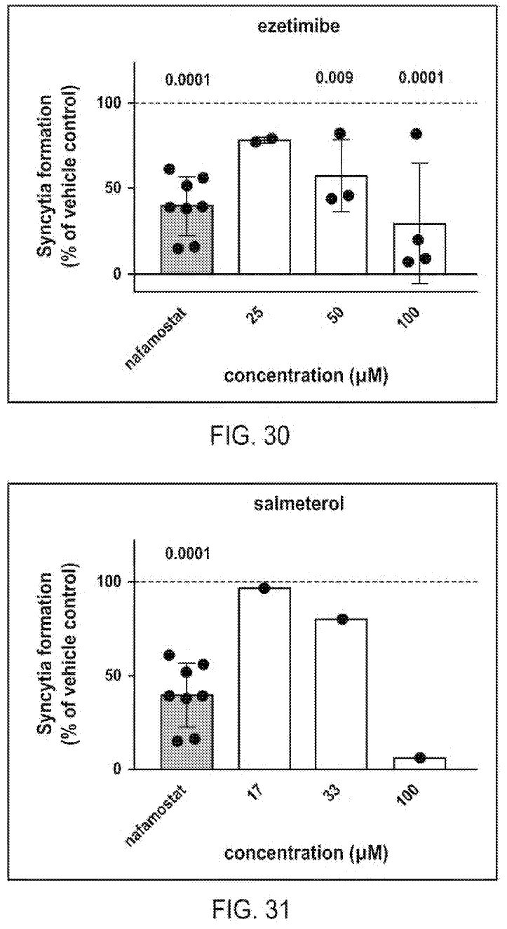

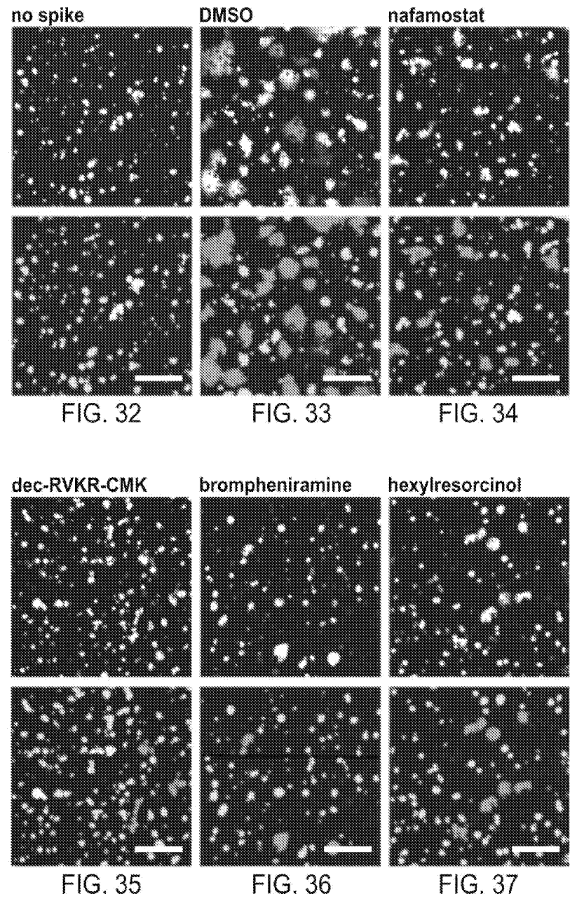

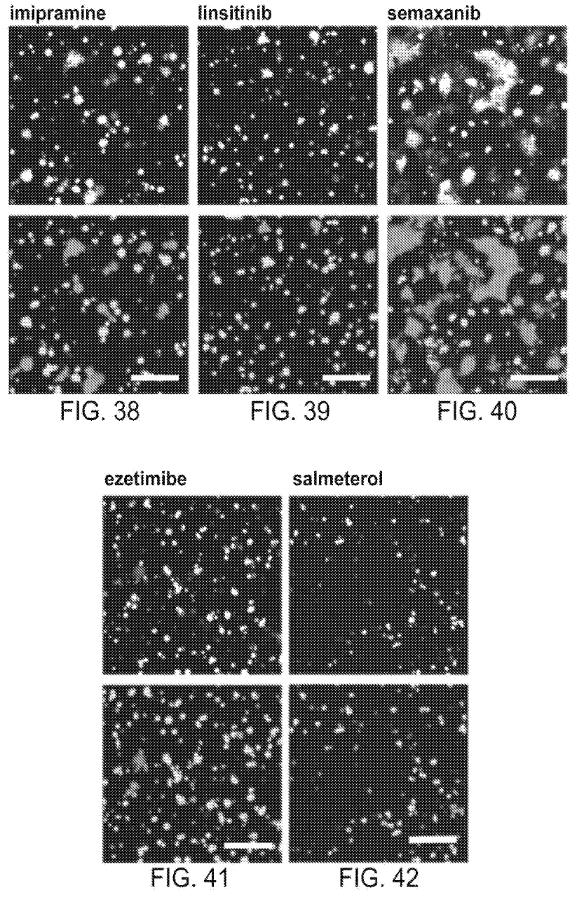

Quantification of syncytia formation in HEK293 cells is shown in FIG. 24-FIG. 31 (related to FIG. 18-FIG. 23). HEK293 acceptor cells transfected with or without ACE2 and TMPRSS2 were seeded in 384 well plates, pretreated with 7-point gradients of test compounds for 1-2 h, and co-cultured for 4 hours with HEK293 donor cells expressing SARS-CoV-2 spike and GFP, or donor cells expressing GFP only (no spike). Images of GFP-positive objects were acquired on a confocal high-content imager and analyzed for syncytia formation and total GFP as a measure of cytotoxicity, using a CNT algorithm as described in the Methods Section. Representative images illustrating syncytia phenotype and compound activity in HEK293 cells are shown in FIG. 32-FIG. 42. Images are shown at the 100 μM condition except nafamostat (5.5 μM), semaxanib (50 μM), and linsitinib (25 μM). Upper panels, raw fluorescence micrographs; lower panels, images with CNT overlay. GFP positive objects that met the criteria for syncytia are colored purple; cellular aggregates that are not syncytia are shown in yellow.

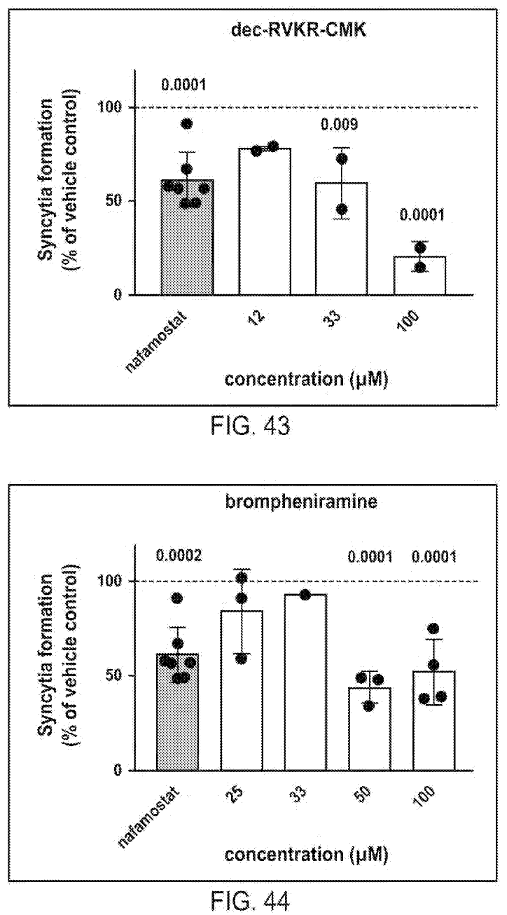

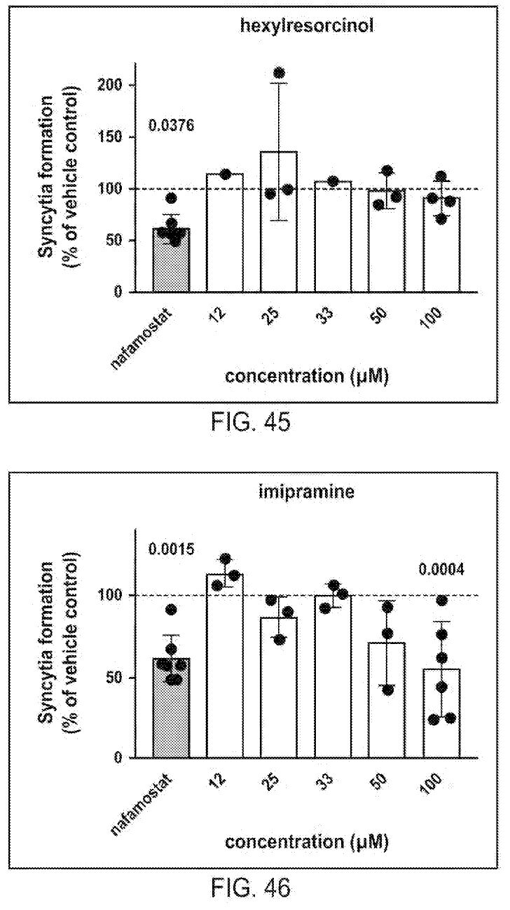

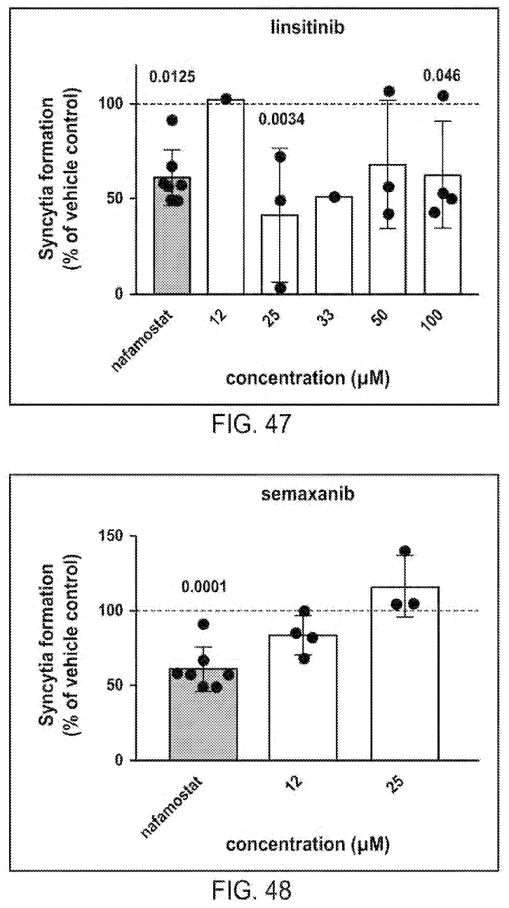

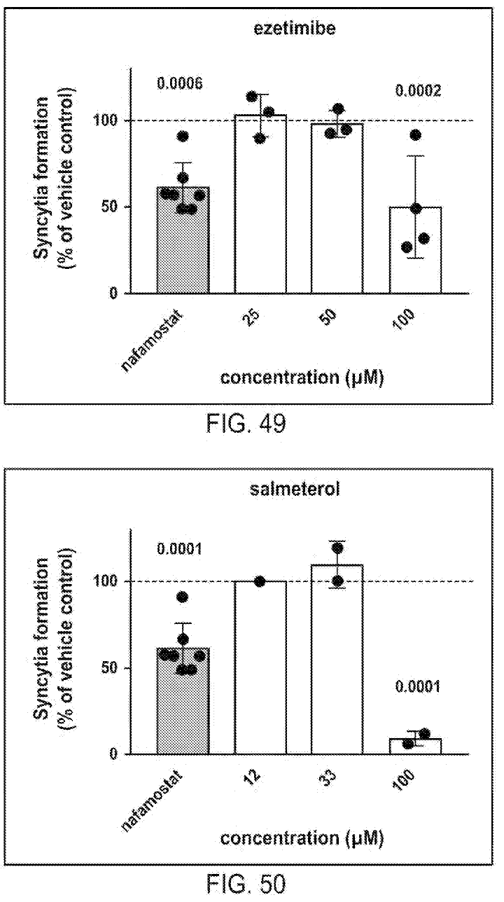

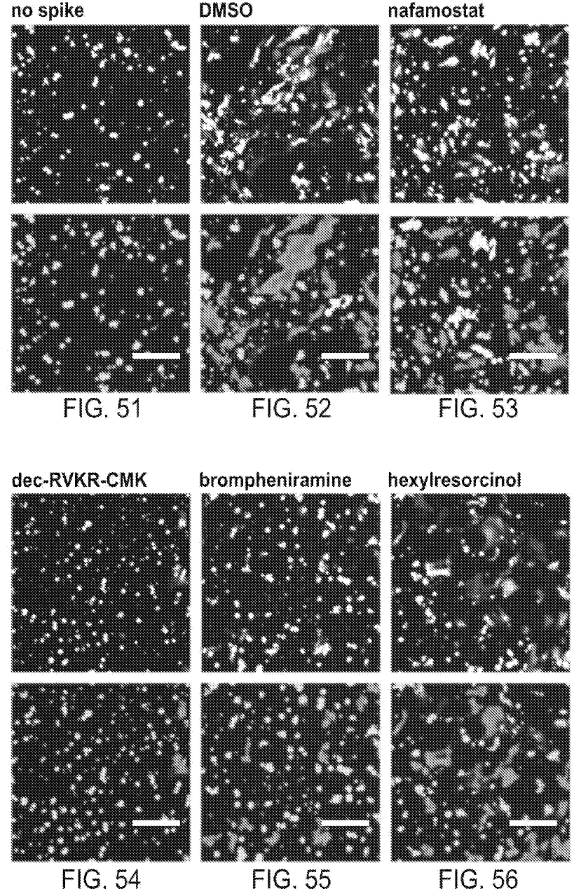

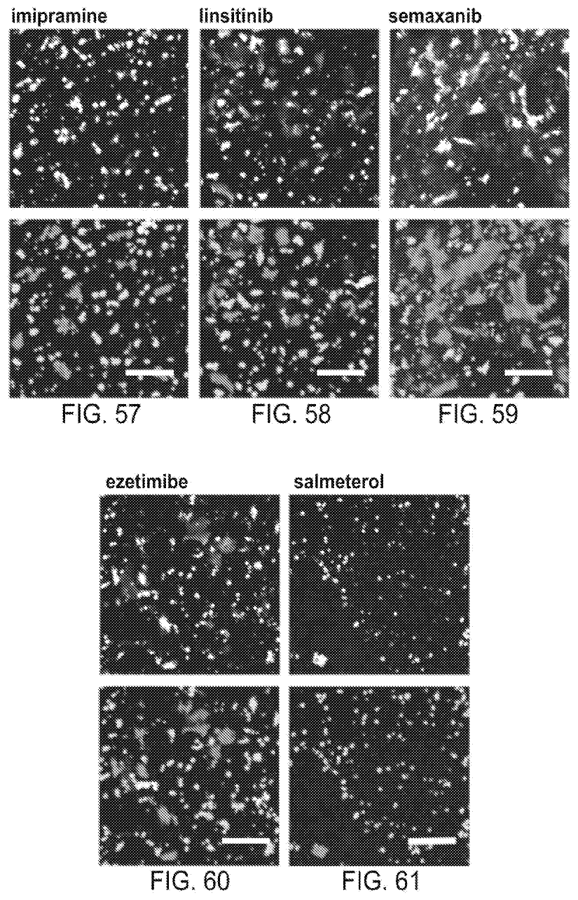

Quantification of syncytia formation in Calu-3 cells is shown in FIG. 43-FIG. 50. Calu-3 acceptor cells were seeded in 384 well plates, pretreated with compounds for 1-2 h, and co-cultured for 4 hours with HEK293 donor cells expressing SARS-CoV-2 spike and GFP. Images of GFP-positive objects were acquired on a confocal high-content imager and analyzed for syncytia formation using a CNT algorithm as described in the Methods. Representative images illustrating syncytia phenotype and compound activity in Calu-3 cells are shown in FIG. 51-FIG. 61. Images are shown at the 100 μM condition except nafamostat (5.5 μM), semaxanib (50 μM), and linsitinib (25 μM). Upper panels, raw fluorescence micrographs; lower panels, images with CNT overlay. GFP positive objects that met the criteria for syncytia are colored purple; cellular aggregates that are not syncytia are shown in yellow.

In a preliminary screen of seven computationally predicted compounds and two serine protease inhibitor positive controls (dec-RVKR-CMK and nafamostat), pretreatment with dec-RVKR-CMK and nafamostat prevented syncytia formation (FIG. 24-FIG. 42 and FIG. 43-FIG. 61), consistent with the involvement of those enzymes in spike-mediated viral entry (Ozden S et al. J Blot Chem. 2008, 283, 21899-21908; Matsuyama S et al. J Virol. 2018, 92, e00683-18; Hoffmann M et al. Cell, 2020, 181, 271-280). Notably, nafamostat, a potent wide spectrum serine protease inhibitor, has recently been found to inhibit the membrane fusion of SARS-CoV-2 at 15-fold higher efficiency than camostat mesylate (Hoffmann M et al. Antimicrob Agents Chemother. 2020, 64, e00754-00720). Dec-RVKR-CMK inhibits not only the enzymatic activity of furin but also those of cathepsin L, cathepsin B, trypsin, papain, and TMPRSS2 (Matsuyama S et al. J Virol. 2018, 92, e00683-18). With the exception of semaxanib, all predicted compounds/drugs inhibited cell fusion to some extent, although some did so only at high concentrations (FIG. 24-FIG. 42 and FIG. 43-FIG. 61).

Both agents that prevented viral infection in the experiments with Vero-E6 cells (salmeterol and ezetimibe), also had inhibitory activity in the cell fusion assay, although salmeterol was at least two orders of magnitude less potent in the cell fusion assay, and ezetimibe was inactive at the highest concentration tested in the viral infection assay, suggesting that their antiviral activity might not originate from an interference in viral entry, but other effects such as enhancement of autophagy, as discussed below. The most potent agent was the insulin-like growth factor 1 receptor (IGF1R) inhibitor, linsitinib. Inhibitor effects were qualitatively conserved in Calu-3 cells but generally more pronounced in transfected HEK293T cells (FIG. 24-FIG. 42 and FIG. 43-FIG. 61). The one exception was the furin inhibitor dec-RVKR-CMK, which was similarly potent in both cell types but with a seemingly larger maximal magnitude of inhibition in Calu-3 cells, suggesting it inhibited other cellular pathways in addition to viral entry.

Then, full dose-response curves were performed in HEK293 cells with selected compounds (linsitinib, brompheniramine, hexylresorcinol, and salmeterol), together with cytotoxicity assessments to test whether inhibition of syncytia formation could merely be a result of cell loss.

HEK293 acceptor cells transfected with or without ACE2 and TMPRSS2 were seeded in 384-well plates, pretreated with 7-point gradients of compounds for 1-2 h, and co-cultured for 4 h with HEK293 donor cells expressing SARS-CoV-2 spike and GFP, or donor cells expressing GFP only (no spike). Images of GFP-positive objects were acquired on a confocal high-content imager and analyzed for syncytia formation and integrated GFP area (total GFP) as a measure of cytotoxicity, using a CNT algorithm as described in the Materials and Methods.

Nafamostat, dec-RVKR-CMK, linsitinib, and to a lesser extent, brompheniramine, showed dose-responsive inhibition of syncytia formation that did not mirror cell loss (FIG. 18-FIG. 23). For example, linsitinib induced complete inhibition of cell fusion, whereas only partial cell loss was observed with a flattening of its dose-response curve. This quantitative and qualitative difference between the two dose-response curves suggests that the observed cell loss is likely to be an epiphenomenon, and not causing the inhibition of syncytia formation. In contrast, hexylresorcinol and salmeterol showed partial and full responses, respectively, on syncytia formation that were mirrored by cell loss (FIG. 18-FIG. 23). Further studies are required to determine in this assay with these particular drugs if cell loss (i) precedes inhibition of cell fusion thereby representing a nonspecific mechanism for preventing syncytia formation or (ii) is a specific result of inhibition of syncytia formation.

### Discussion

Utility of the computational pipeline for identifying repurposable drugs. Presented herein are the results from a computation-driven approach for identifying repurposable drugs or new compounds that comply with the antiviral or anti-cytokine signatures derived from SARSCoV-2-infected cells. The overall analysis was driven by the RNAseq data from SARS-CoV-2-infected A549 cells and A549-ACE2 cells, as well as a SARS-CoV-2-host PPI network, toward gaining a system-level understanding of the key players in the host cell that are involved in SARS-CoV-2 infection and identifying potential modulators of these key players. The extensive study led to 15 potentially antiviral and 23 potentially immune-modulatory compounds (Table 11 and Table 13). The assays conducted to test ten of the proposed antiviral compounds pointed to several repurposable drugs or investigational compounds that could be pursued for lead development against SARS-CoV-2 infection. Among them, salmeterol exhibited particularly strong inhibitory activities in Vero-E6 cells infected by SARS-CoV-2 and linsitinib substantially reduced spike-protein-dependent syncytia formation (viral entry) in engineered HEK293T cells.

Recent studies point to the utility of computational systems pharmacology approaches for identifying repurposable drugs against SARS-CoV-2 (Beck B R et al. Comput Struct Biotechnol J. 2020, 18, 784-790; Gordon D E et al. Nature, 2020, 583, 459-468; Riva L et al. Nature, 2020, 586, 113-119; Singh T U et al. Pharmacol Rep. 2020, 72, 1479-1508; Zhou Y et al. Cell Discov. 2020, 6, 14; Zhou Y et al. PLoS Biol. 2020, 18, e3000970; Zhou Y et al. Lancet Digit Health, 2020, 2, e667-676). Of note is the work of Zhou et al., where repurposable drugs against SARS-CoV-2 were identified by evaluating the proximity of targets of known drugs to human proteins engaged in the human-CoV-host cell interactome (Zhou Y et al. Cell Discov. 2020, 6, 14). This type of network proximity analysis, originally introduced by Guney et al. (Guney E et al. Nat Commun. 2016, 7, 10331), is also used here, but in a different context, mainly for prioritizing the candidate compounds/drugs that have been already identified from the DEG patterns of SARS-CoV-2 infected cells and corresponding Cmap signatures. In contrast, Zhou et al. used gene set enrichment data (from MERS-CoV and SARS-CoV-infected cells) and Cmap gene-drug signatures for validating their predicted drugs (Zhou Y et al. Cell Discov. 2020, 6, 14). Another important component unique to this analysis is the use of the interface QuartataWeb that allows for identifying drug-target associations, and for evaluating and classifying the pathways implicated in the disease modules deduced from the SARS-CoV-2-specific virus-host interactome (Gordon D E et al. Science 2020, 370, 1181; Gordon D E et al. Nature, 2020, 583, 459-468) and assessing the mechanisms of action. QuartataWeb was further used to cluster the selected compounds based on their mechanisms of action and select representatives from each cluster to obtain a sufficiently diverse set for experimental testing. Thus, this study differs from that of Zhou et al. (Zhou Y et al. Cell Discov. 2020, 6, 14) in the overall design of the computational protocol, the types of data used as input, as well as the output analyses for compound selection, prioritization, and validation, while both studies utilize state-of-the-art methods (network proximity analysis) and resources (e.g., Cmap library) at different steps of the workflow.

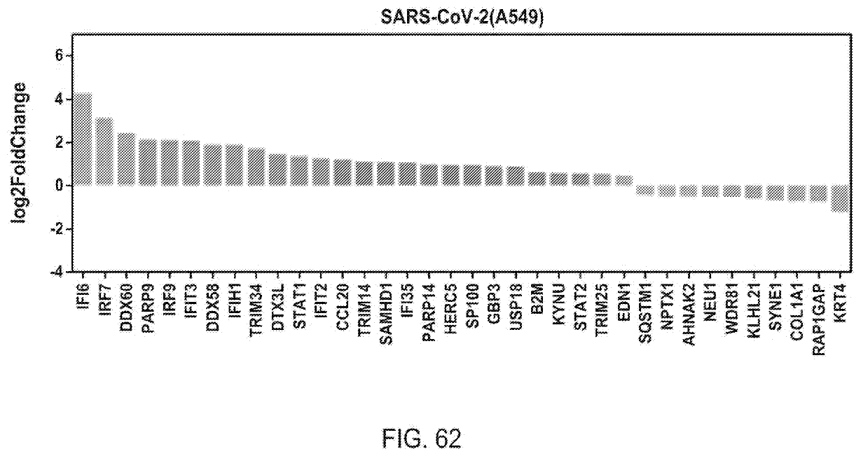

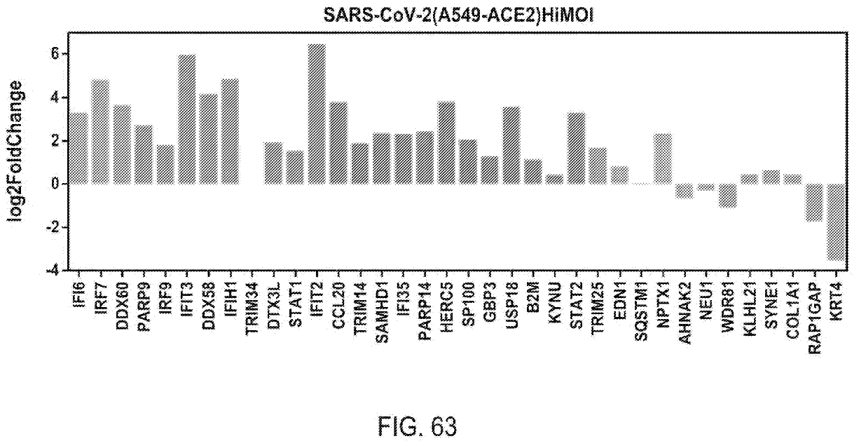

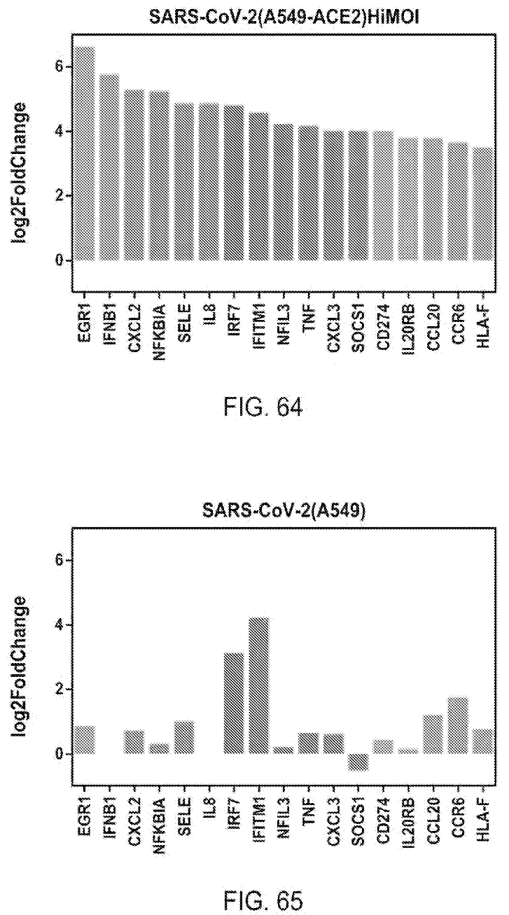

Unlike influenza A and respiratory syncytial virus, the host immune defensive reactions of SARS-CoV-2 were significantly muted unless ACE2 was overexpressed (Blanco-Melo D et al. bioRxiv, 2020, 10.1101/2020.03.24.004655). FIG. 62-FIG. 65 show a comparison of the behavior of A546 and A546-ACE2 cells vis-à-vis the expression levels of the genes that have been adopted for defining antiviral and anticytokine signatures. Cross-examination of the expression levels of the 17 anticytokine signature genes in A549 cells showed that most of these genes could not be clearly distinguished in those cells, i.e., their upregulation was specific to A549-ACE2 cells (compare FIG. 64 and FIG. 65), whereas the 36 genes that define the antiviral signature exhibited a comparable expression pattern in A549-ACE2 cells (see FIG. 62 and FIG. 63). These observations support the robustness of the antiviral signature on the one hand, and the utility of A549-ACE2 cells for detecting genes implicated in hyperinflammatory responses, on the other.

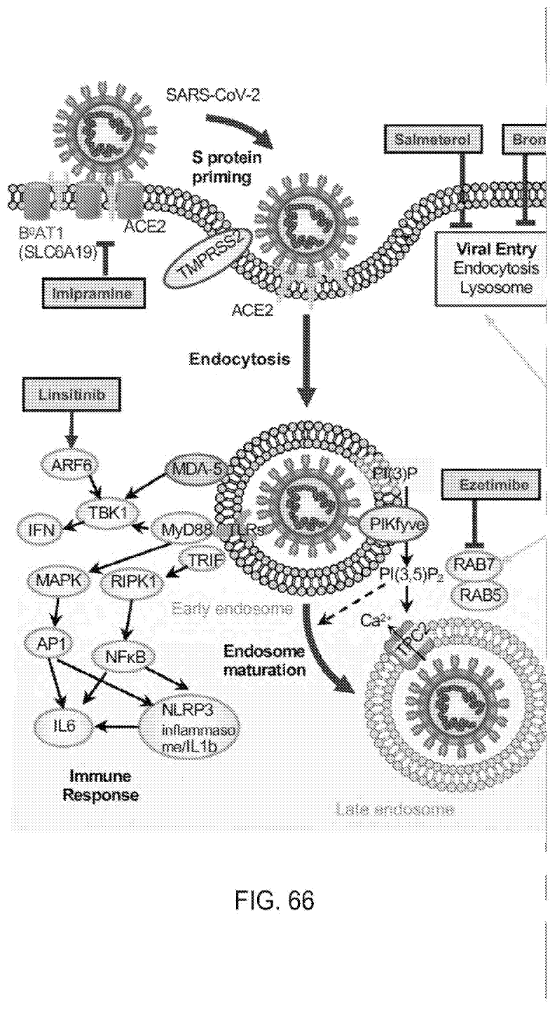

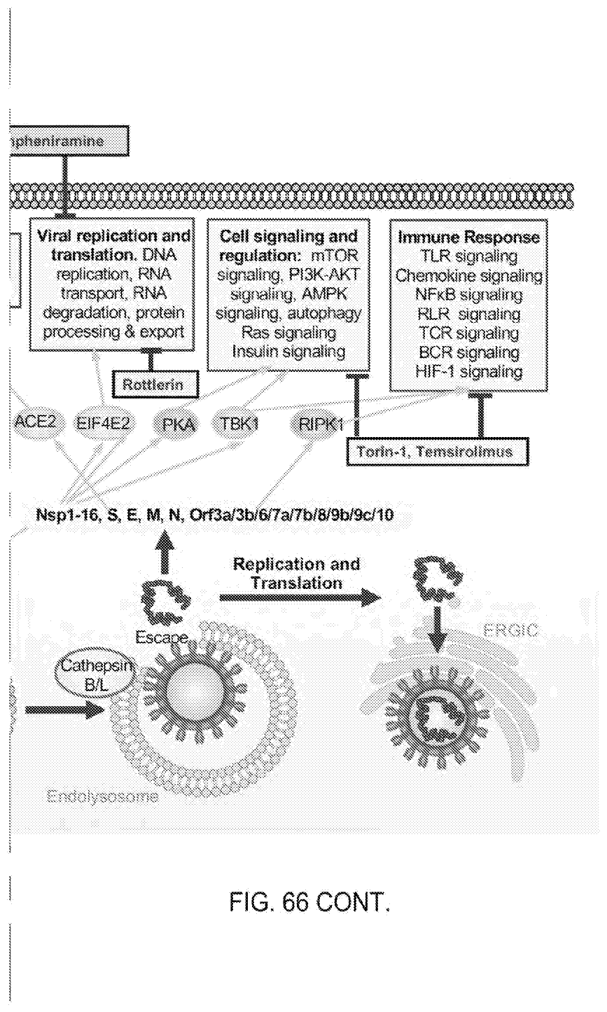

Potential mechanisms of action of drug candidates. Two types of in vitro assays were performed with ten predicted repurposable or investigational drug candidates most of which are proposed to be implicated in viral entry: linsitinib, imipramine, ezetimibe, hexylresorcinol, brompheniramine, salmeterol, semaxanib, rottlerin, temsirolimus, and torin-1. Viral entry is used here in a broad sense including (i) the fusion between viral and host cell membrane (involving ACE2 and B⁰ AT1 on the host cell membrane, and facilitated by host cell proteases such as TMPRSS2 and furin) and (ii) endosomal processes mediating the endocytosis of the virus and its release from the vesicles. The latter involves many signaling and regulatory proteins including those activated by the immune response, in addition to proteases such as cathepsins, as schematically depicted in FIG. 66. The two experimental assays were chosen to complement each other: the viral infection assay recapitulates the entire virus infection process, whereas the syncytia assay addresses a specific, defined mechanism in viral entry, namely fusion of the virus with the host cell, which is mediated by interaction of viral spike protein with the host cell receptor (ACE2), and facilitated by host cell proteases.

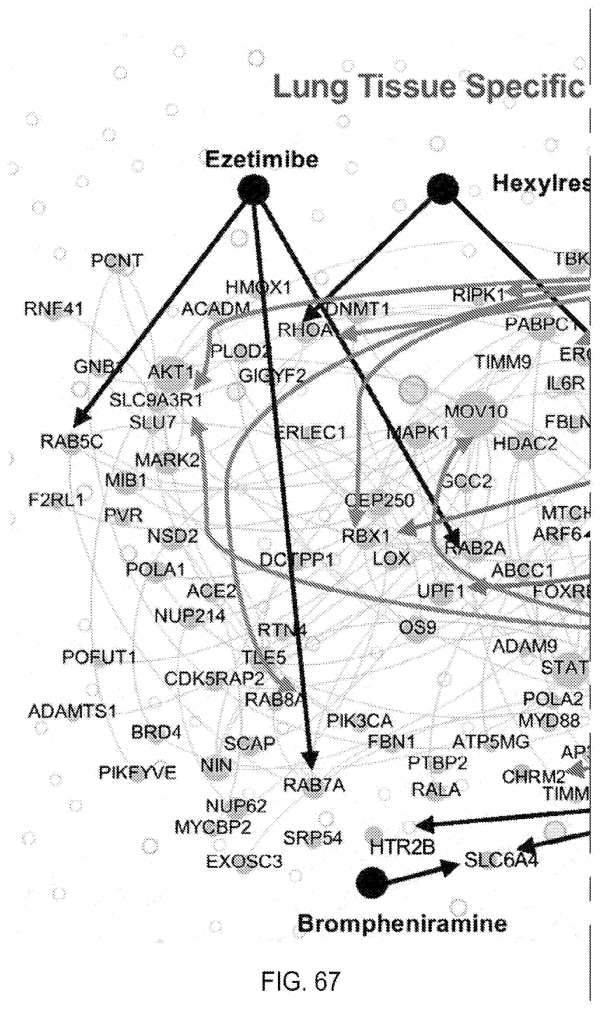

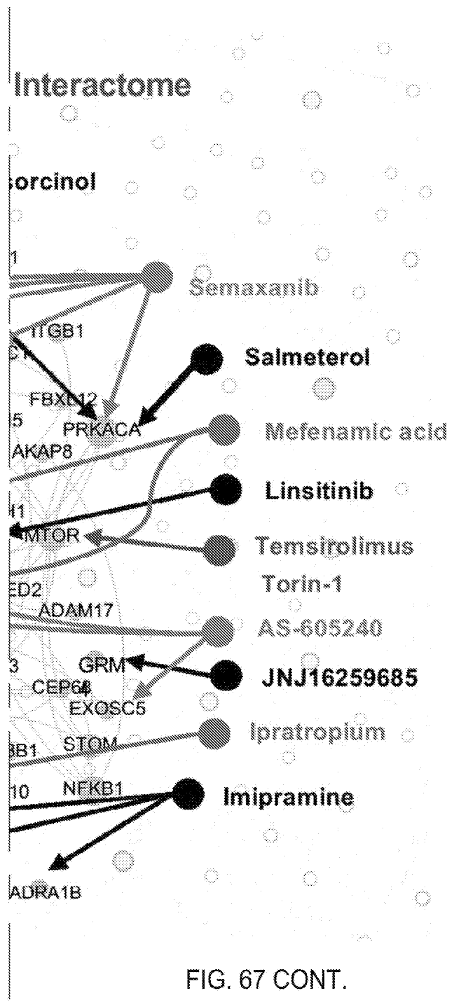

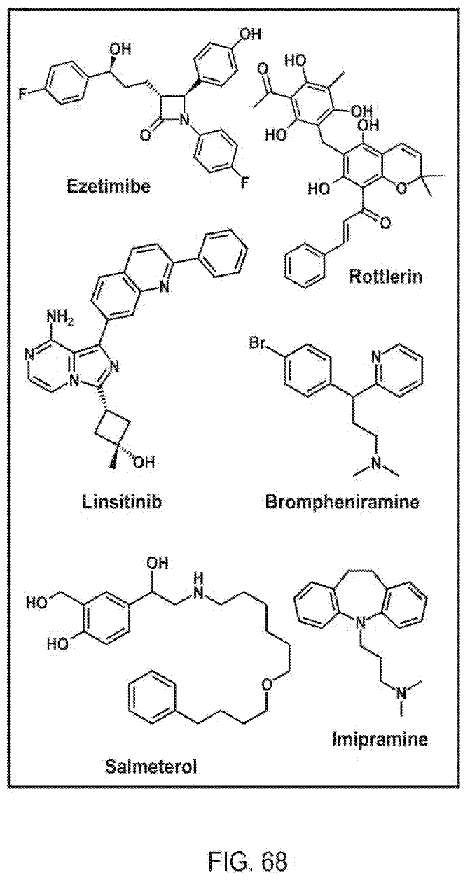

Below the experimental results for the tested compounds are discussed in the light of their CMap scores, the similarities between their interaction patterns (as indicated by the clusters in FIG. 7-FIG. 8), the involvement of their targets in the host cell PPI network or disease modules (FIG. 9-FIG. 10) with reference to lung-tissue interactome (FIG. 67), and relevant findings from previous work. The discussion begins with compounds/drugs implicated in viral entry, as the focus of current tests (FIG. 68).

Linsitinib. Linsitinib showed the highest inhibitory activity without overt cytotoxicity in the spike-induced syncytia formation assay that specifically measures viral entry. It is interesting to note that its proximity rank to the viral entry module (rank 4) was one of the highest among all tested compounds. Linsitinib is an IGF-1R and insulin receptor inhibitor (Mulvihill M J et al. Future Med Chem. 2009, 1, 1153-1171) currently under investigation for various types of cancer due to its ability to prevent tumor cell proliferation and induce tumor cell apoptosis (Fassnacht M et al. Lancet Oncol. 2015, 16, 426-435). The analysis also indicated that it targets the insulin receptor, which interacts with ADP ribosylation factor 6 (ARF6), a binding partner of SARS-CoV-2 endonuclease nsp15 (Gordon D E et al. Nature, 2020, 583, 459-468). As listed in Table 7, ARF is involved in multiple modules. Notably, the ubiquitination of the ARF domain of TRIM23 is essential for mediating virus-induced autophagy, an antiviral defense mechanism, via activation of TANK-binding kinase 1 (TBK1) (Sparrer K M J et al. Nat Microbiol. 2017, 2, 1543-1557). Therefore, it is proposed that its possible MOA is activation of TBK1 that promotes autophagy (see FIG. 66). While linsitinib was selected as a potential antiviral compound, it was also identified as an anti-inflammatory compound with a very high (−99.37) CMap score (Table 6), in strong support of its selection as a high priority compound. In this context, the EC50 for linsitinib was 25 μM in the cell fusion assay that may not be disparate from the reported Cₘₐₓ of 5-10 μM in patients (Macaulay V M et al. Clin Cancer Res. 2016, 22, 2897-2907). Since several IGF1/InsR inhibitors are available, this class of compounds is well suited for structure-activity studies. Such a study is particularly relevant, since CMap can implicitly account for structure-dependent non-canonical modes of antiviral activity that can differ among members of a particular drug class.

Imipramine. Imipramine, an FDA-approved tricyclic antidepressant (Gillman P K. Br J Pharmacol. 2007, 151, 737-748), has been also reported to inhibit Chikungunya virus fusion (entry) (Wichit S et al. Sci Rep. 2017, 7, 3145). It was distinguished by a high network proximity ranking (8^{th}) in viral entry module (Table 11). Notably, imipramine is a high-affinity allosteric inhibitor of serotonin transporter (SLC6A4) (Plenge P et al. Nat Commun. 2020, 11, 1491). Importantly, ACE2 is anchored into the host membrane through close association with the amino acid transporter, B⁰ AT1 (see FIG. 66). B⁰ AT1 is structurally homologous to serotonin transporter, sharing the LeuT fold typical of this family of sodium-coupled neurotransmitter transporters (Cheng M H et al. Nat Struct Mot Biol. 2019, 26, 545-556). Thus, imipramine is likely to also target B⁰ AT1, which may impair the ACE2-spike interaction, hence the observed inhibitory effect. In addition, imipramine has been reported to promote autophagy (Shchors K et al. Cancer Cell, 2015, 28, 456-471), and this could be another (indirect) mechanism for alleviating SARS-CoV-2 infection.

Brompheniramine. Brompheniramine is an FDA-approved drug known as a first-generation antihistamine drug, for treating common colds and allergic rhinitis (Simons F E et al. J Allergy Clin Immunol. 1982, 70, 458-464). It shares a similar mode of action with imipramine, also targeting serotonin transporter. In this study, brompheniramine was indicated to be highly related to SARS-CoV-2 entry (ranked 23^{rd} in the viral entry module). Both imipramine and brompheniramine inhibited syncytia formation, consistent with their hypothesized interaction with membrane-anchored ACE2.

Salmeterol. Salmeterol had the highest CMap score for inducing the antiviral signature, and very high (network) proximity to the viral entry module. It is canonically used as a bronchial smooth muscle relaxant in asthma and COPD, as a long-acting 02-adrenergic receptor ((32-AR) agonist. COPD has been shown to be associated with increased expression of ACE2 (Leung J M et al. Eur Respir J. 2020, 55, 2000688), and a recent study on the effects of inhaled corticosteroids (ICS) on the bronchial epithelial cell expression of SARS-CoV-2-related genes in COPD patients demonstrated that a treatment with ICS in combination with salmeterol/fluticasone propionate decreased the expression of ACE2 and ADAM17 (Milne S et al. medRxiv, 2020, https://doi.org/10.1101/2020.08.19.20178368). It is also noted that β2-AR interacts with the PKA catalytic subunit α (Cα; encoded by PRKACA), which promotes autophagy-mediated degradation (Lizaso A et al. Autophagy, 2013, 9, 1228-1243). Salmeterol has been reported to induce autophagy as a potential mechanism of inhibiting Dengue virus in vitro (Medigeshi G R et al. Antimicrob Agents Chemother. 2016, 60, 6709-6718). The observed inhibitory effect in Vero-E6 cells (FIG. 14-FIG. 17), which were not borne out by syncytia formation experiments with either HEK293T or Calu-3 cells, except at high concentration (FIG. 18-FIG. 23), is consistent with activities unrelated to viral entry, such as an innate immune response stimulation or autophagy enhancement.

Ezetimibe. Ezetimibe, an FDA-approved lipid-lowering drug (Kosoglou T et al. Clin Pharmacokinet. 2005, 44, 467-494), has a distinct MOA via the sterol transporter Niemann-Pick C1-Like 1 (Nutescu E A et al. Pharmacotherapy, 2003, 23, 1463-1474). It targets sterol O-acyltransferase 1 (SOAT1) in the ER, which, in turn, interacts with the Ras proteins encoded by RAB5C, RAB2A, and RAB7A, implicated in early-to-late endosomal maturation. These proteins bind SARS-CoV-2 nsp7 (Gordon D E et al. Nature, 2020, 583, 459-468). Loss of RAB7A (see FIG. 66 and FIG. 67) has been shown to reduce viral entry by altering endosomal trafficking and sequestering ACE2 inside cells (Daniloski Z et al. Cell 2021, 184, 1-14). Finally, ezetimibe was also reported to interfere with the entry and replication of Dengue virus (Osuna-Ramos J F et al. Antiviral Res. 2018, 160, 151-164). Herein, ezetimibe inhibited both viral infection and cell fusion. Its lower potency in the cell fusion assay is consistent with multiple mechanisms in addition to the dominant effect on viral entry, as described above.

Hexylresorcinol. Hexylresorcinol ranked 2^{nd} in the viral entry module. It is a FDA-approved over-the-counter product with anesthetic, antiseptic, and anthelmintic properties often used for upper respiratory irritations such as sore throat (Wilson C O et al. Textbook of organic medicinal and pharmaceutical chemistry, 1966, 5th edn. Philadelphia, PA: Lippincott). It has sodium channel blocking effects and interacts with transglutaminase 2, a substrate of two SARS-CoV-2-related host proteins RhoA and PKA Cα. It also showed potential action against respiratory virus parainfluenza type 3 and cytomegalovirus (Shephard A et al. Antiviral Res. 2015, 123, 158-162). Yet, the in vitro cell fusion assay herein suggests that virus-host cell interactions may not be major contributors to its reported antiviral activities.

Rottlerin. Rottlerin (R077), a natural polyphenolic compound, has been reported to inhibit influenza replication as an inhibitor of PKC (Hoffmann H H et al. Antiviral Res. 2008, 80, 124-134), and the translation of rabies virus circle by reducing intracellular ATP contents (Lama Z et al. Antiviral Res. 2019, 168, 51-60). It may have neuroprotective effects by its anti-oxidative and anti-inflammatory action in the central nervous system (Lee T H et al. J Neuroinflammation 2020, 17, 177). Rottlerin inhibited viral infection but dose-limiting toxicity prevented a detailed analysis of viral entry vs. infection.

Temsirolimus and torin-1. Temsirolimus and torin-1 are indicated to inhibit the protein kinase mTOR (Bergmann L et al. Expert Rev Anticancer Ther. 2014, 14, 9-21). The temsirolimus metabolite, sirolimus, as well as mTOR inhibitor rapamycin, are among the 128 approved drugs listed in the Excelra COVID-19 Drug Repurposing Database (https://wwwexcelracom/covid-19-drug-repurposing-database/). The PI3K-AKT-mTOR signaling pathway provides a cross-protective immunity against viral infection, especially against the influenza viruses (Lehrer S. World Acad Sci J. 2020, 2, 1), and has been recognized to regulate the translation and replication of coronaviruses (Zumla A et al. Nat Rev Drug Discov. 2016, 15, 327-347). mTOR inhibitors induce autophagy, which has been attributed to the inhibition of MERS-CoV (Gassen N C et al. Nat Commun. 2019, 10, 5770). Temsirolimus is currently FDA-approved for treating renal cell carcinoma (Miao H et al. J Virol. 2010, 84, 6687-6698). It has been reported to inhibit MERS-CoV infection (Kindrachuk J et al. Antimicrob Agents Chemother. 2015, 59, 1088-1099). Torin-1 inhibits both mTORC1/2 complexes with IC₅₀ values between 2 and 10 nM and therefore was used at 1-10 and 100 nM levels and was toxic at 100 nM. Further studies will be required to determine the relative antiviral effects of these mTOR inhibitors in the context of their intrinsic dose-limiting toxicity.

Semaxanib. Semaxanib a tyrosine kinase inhibitor, under development as a cancer therapeutic (O'Donnell A et al. Br J Cancer, 2005, 93, 876-883), did not exhibit any inhibitory activity, despite its involvement in multiple modules.

Compounds targeting immune response. Immunopathology of COVID-19 is longitudinally dynamic, individually diverse, more unique than other respiratory viral infections, and potentially detrimental when uncontrolled. It features lack of interferon response, lymphopenia, and overwhelming inflammatory activation-especially in severe stages or patients with poor prognosis (Blanco-Melo D et al. Cell, 2020, 181, 1036-1045.e1039; Liu J et al. EBioMedicine, 2020, 55, 102763; Ong E Z et al. Cell Host Microbe 2020, 27, 879-882; Zhou F et al. Lancet, 2020, 395, 1054-1062). Anti-cytokine therapeutics inhibiting IL-1 (NCT04324021, NCT0436281), IL-6 (NCT04320615, NCT04315298), TNF-α (Feldmann M et al. Lancet. 2020, 395, 1407-1409), or the broad-spectrum immune response by glucocorticoids (Lu S et al. Ann Transl Med. 2020, 8, 627) are currently investigated. Stemming from transcriptomic response following infection in A549-ACE2, inducers that both elevate IFN signaling while suppressing cytokine pathways were the aim. The resulting compounds (Table 13), interestingly, included His receptor antagonists and TNFα inhibitors as expected, while also containing candidates such as PAF receptor antagonists, NFκB, SRC, JAK, and mTOR inhibitors, and neurological drugs blocking ion channels or neurotransmitter receptors. These results reveal the complexity of immune transcriptome modulation, involving heterogeneous states of multiple components and their coupled dynamics.

Autophagy enhancement as a possible mechanism to exploit in combination therapies. The present analysis showed that certain autophagy-related vesicle pathways were downregulated, especially in the SARS-CoV-2-infected A549-ACE2 cells, which could be a potential escape mechanism from the immune system, as lysosomal digestion serves as an intrinsic antiviral program. These observations point to the opportunity of discovering drugs that exploit systems-level host response, i.e., stimulate autophagic response while suppressing hyperinflammatory responses. A recurrent pattern in several candidate compounds was indeed their involvement in autophagy enhancement. These include antidepressants as well as compounds repurposed to eliminate aggregates in the central nervous system, lung, or liver, such as trifluoperazine, fluphenazine (Table 13), and others (salmeterol and imipramine) that exhibited inhibitory activity in these experiments. Microglial autophagy has been recently pointed out to be essential for recovery from neuroinflammation (Berglund R et al. Sci Immunol. 2020, 5, eabb5077). In general, the role of autophagy in viral infection remains context-dependent, and both pathogen-destroying or viral-promoting effects have been reported (Maier H J et al. Viruses, 2012, 4, 3440-3451), whereas inducing autophagy has markedly reduced MERS-CoV replication (Gassen N C et al. Nat Commun. 2019, 10, 5770). The effectiveness of selected autophagy enhancers observed here support their further investigation, at least in combination therapy, against COVID-19.

Summary and Conclusions. The compounds prioritized here targeted system-level modules, rather than individual targets. Beyond the urgent need for repurposing, these drugs can also be exploited as mechanistic probes to enhance understanding of SARS-CoV-2 pathogenicity and drug resistance and provide a systems framework for developing combination therapies.

Comparison with earlier work showed that there are only nine compounds (apicidin, daunorubicin, entacapone, loratadine, metformin, mycophenolic acid, ribavirin, verapamil, and valproic acid) shared between the predictions herein and the recently reported 69 repurposable drugs (Gordon D E et al. Nature, 2020, 583, 459-468). Given the little overlap with the drugs currently under clinical trials against SARS-CoV-2, the current findings may help complement the global COVID-19 drug discovery pipeline.

While a systems-level approach was adopted herein, it should also be noted that the viral-host cell interactions that mediate viral entry and endosomal transitions, and on accompanying cell signaling and regulation events and immune response, were focused on, in line with the assays conducted for probing viral entry. Events at the nucleus relevant to viral replication and translation play an equally important role, as evidenced by recent genome-wide CRISPR screens in Vero-E6 cells (Wei J et al. Cell, 2021, 184, 1-16), which identified many proviral genes involved in chromatin regulation, histone modification, or epigenetic regulation. Compounds that target these specific pathways/processes, such as those involving the ubiquitous nuclear protein HMGB1 and the SWI/SNF chromatin remodeling complex (Wei J et al. Cell, 2021, 184, 1-16) or the upregulation of cholesterol biosynthesis (Daniloski Z et al. Cell 2021, 184, 1-14), are yet to be determined.

Materials and Methods

Evaluation of host-targeted antiviral and anti-hyperinflammatory signature from post-SARS-CoV-2 infection transcriptomics. The up- and downregulated gene list of A549 cells (human lung cancer) after 24 h of SARS-CoV-2 infection was obtained from GSE147507, and the corresponding DEGs were acquired from the DESeq2 result from the original publication with FDR adjusted P-value smaller than 0.05. This resulted in 100 upregulated and 20 downregulated genes listed in Table 1. Overrepresentation analysis was performed using gProfiler (Raudvere U et al. Nucleic Acids Res. 2019, 47, W191-W198) with GO database (Carbon S et al. Nucleic Acids Res. 2019, 47, D330-D338) for up- or downregulated genes, respectively, using Benjamini-Hochberg multiple test correction with a threshold of 0.05. Examination of the GO Biological Process (GO-BP) and GO cellular components (GO-CC) data for up- or downregulated genes resulted in 319 GO-BP and 13 GO-CC terms. The number of enriched upregulated terms was reduced by retaining those associated with no more than 300 genes, and not fewer than 10 overlapping genes, resulting in 16 GO terms (see column 6 in Table 3). Downregulated terms were all kept. The enriched GO terms were organized and visualized with quickGO and classified as antiviral, proviral, or ambiguous. Those genes that defined the “antiviral signature” were obtained by merging the up- (innate immune response) or down-(intracellular vesicle) regulated antiviral genes and excluding proviral (viral genome replication) components. Genes classified as proviral or ambiguous were not included in the antiviral signature.

The resulting signature (composed of 36 genes) was used to screen for compounds/drugs in the L1000 database (Subramanian A et al. Cell, 2017, 171, 1437-1452.e1417) which elicit a response that best matches the antiviral signature, reflected by their sufficiently high Cmap connectivity scores, at https://clue.io/query. CMap scores range from −100 to 100, the two limits representing the least and the most similar compound-induced gene signatures, compared to the input antiviral signature. Compounds with top scores (in the suggested default range of 90-100) were selected for further analysis.

For the construction of anti-hyperinflammation signature, cytokine-related events (to be suppressed) were focused on by overlapping the GO cytokine response gene set (GO:0034097) with the upregulated genes (adjusted P-value <0.05) from A549-ACE2-infected cells with high MOI of SARS-CoV-2 (GSE147507). A final candidate set of 17 genes at the 0.05 upper quantile of log₂ fold change were selected (see Table 4). This set of 17 genes was used as the upregulated gene input in Cmap screening within the L1000 database, and the 275 compounds with lowest connectivity scores (varying from −90 to −100), showing strongest opposing effect, were selected.

Identification of known compound-target interactions. The compound-target interaction search engine QuartataWeb (Li H et al. Bioinformatics, 2020, 36, 3935-3937), which integrates STITCH (version 5) (Szklarczyk D et al. Nucleic Acids Res. 2016, 44, D380-384) and DrugBank (version 5.1.7) (Wishart D S et al. Nucleic Acids Res. 2018, 46, D1074-D1082), was used to identify targets for compounds obtained from Cmap prediction. Specifically, all compound-target interactions recorded in DrugBank and the compound-target interactions with experimental confidence score no <0.4 in STITCH were integrated for further analysis. As a result, 1,800 known interactions between 168 compounds and 746 targets were retrieved, while no targets were identified for the remaining 95 compounds.

Prioritizing the predicted compounds using their network proximity. The basic idea of network proximity (Guney E et al. Nat Commun. 2016, 7, 10331) is to evaluate the significance of the network distance between a compound and a given disease module in the interactome. The methodology assumes that a compound is effective if it targets proteins within or in the immediate vicinity of a disease module. In this case, the human lung protein-protein interactome was extracted from the Biomedical Network Dataset Collection BioSNAP (Zitnik M et al. BioSNAP datasets: Stanford biomedical network dataset collection. 2018, http://snapstanfordedu/biodata). Five viral-related modules were defined, each containing a set (S) of pre-defined proteins derived from the host proteins implicated in SARS-CoV-2 infection (see the Results). For each compound, the set (T) of targets were determined using QuartataWeb in the human lung PPI network. The proteins in sets S and T were connected via paths of zero or more intermediate protein nodes. Then the distance between these targets and the pre-defined proteins from each viral-related module were evaluated, in the human lung PPI network, as the average shortest distance path between the respective nodes s and t belonging to the sets S and T, as:

$$d ( S , T ) = \frac{1}{‖ T ‖} {\sum }_{t ∈ T} {min}_{s ∈ S} d ( s , t )$$

Then, a reference distance distribution was constructed, corresponding to the expected distance between the disease module proteins and a randomly selected groups of proteins in the network, with the same size and degree of distribution as drug targets in the network. This procedure was repeated 1,000 times, and the mean and standard deviation of the reference distance distribution were used to calculate a z-score by converting the observed distance to a normalized distance. Each compound was assigned a z-score with respect to each disease module, a lower z-score meaning that its targets were closer to the disease module, or the compound would be more effective. The z-scores were evaluated using the toolbox package developed by Guney et al. (Guney E et al. Nat Commun. 2016, 7, 10331). Note that the network proximity provides a relative measure, the absolute value of which depends on the disease and application. In the current application to four disease modules, a uniform cutoff for the z-score was not selected. Instead, the top 25 compounds from each module were selected to include a set of compounds with diverse MOAs.

Compound clustering by means of interaction-pattern-based similarities. Top-ranking compounds were clustered by evaluating the similarities between the interaction patterns of these compounds vis-à-vis their known targets compiled in DrugBank and STITCH. Specifically, each compound i was assigned a vector uᵢ, the elements of which were the confidence score for the compound-target interaction (0 if there is no known interaction). Then, the interaction-pattern-based similarities between compound i and j were evaluated by calculating cosine distance between vector uᵢ and vector uⱼ using the similarity metric s=1−(uᵢ·uⱼ)/(|uᵢ||uⱼ|).

In vitro viral inhibition assays. SARS-CoV-2 viral assays were performed in UCLA BSL3 high containment facility. Vero-E6 [VERO C1008 (ATCC #CRL-1586™)] cells were obtained from ATCC and cultured at 37° C. with 5% CO₂ in EMEM growth media with 10% fetal bovine serum and 100 units/ml penicillin. SARS-CoV-2 Isolate USA-WA1/2020 was obtained from BEI Resources of National Institute of Allergy and Infectious Diseases (NIAID). Temsirolimus (CAS 162635-04-3), Ezetimibe (CAS 163222-33-1), Salmeterol (CAS 89365-50-4), and Torin-1 (CAS 1222998-36-8) were purchased from Selleckchem. Rottlerin (CAS 82-08-6) was purchased from TOCRIS. Vero-E6 cells were plated in 96-well plates (5×10³ cells/well) and pretreated with compounds (in triplicate, at indicated concentrations) for 1 h prior to addition of SARS-CoV-2 (MOI 0.1). After 48-h post-infection (hpi) the cells were fixed with methanol for 30-60 min in −20° C. Cells were washed three times with PBS and permeabilized using blocking buffer (0.3% Triton X-100, 2% BSA, 5% Goat Serum, 5% Donkey Serum in 1×PBS) for 1 h at room temperature.

Subsequently, cells were incubated with anti-SARS-CoV-2 Spike antibody (Sino Biological, 40150-R007, 1:200) at 4° C. overnight. Cells were then washed three times with PBS and incubated with Goat anti-mouse IgG Secondary Antibody, Alexa Fluor 555 (Fisher Scientific PIA32790, 1:1,000) for 1 h at room temperature. Nuclei were stained with DAPI (40,6-Diamidino-2-Phenylindole, Dihydrochloride; Life Technologies) at a dilution of 1:5,000 in PBS for 10 min. Cells were analyzed by fluorescence microscopy. Five images per well were quantified for each condition. The Multiwavelength Cell Scoring module in MetaXpress (Molecular Devices, Sunnyvale, CA) was used to measure the total integrated fluorescence spike signal in each cell. Histograms of the log of the integrated intensities were plotted in Spotfire (Tibco, Palo Alto, CA). A cutoff value of three standard deviations of the total integrated signal from the mock samples was established, above which cells were considered to have a positive spike signal, and thus be infected. The number of infected cells was divided by the total number of cells in each treatment group to determine the percent of infected cells after treatment.

Cell Fusion (Syncytia) Assay

Cell culture. HEK293T cells (ATCC CRL-3216) were maintained at 37° C. in a humidified incubator with a 5% CO₂ atmosphere. Cells were cultured in Dulbecco's modified Eagle medium (DMEM, Gibco 11965092) supplemented with 10% fetal bovine serum (FBS, Corning 35010CV), 1% penicillin-streptomycin (Cytiva HyClone SV30010), and 1% L-glutamine (Cytiva HyClone SH3003401). A cell bank of defined passage was established, and cells were propagated for no more than 15 passages in culture. A cell bank of Calu-3 cells (ATCC HTB-55) from cells maintained in DMEM as recommended by ATCC was established at early passage. Because Calu-3 cells grew very slowly in DMEM, for experiments cells were switched to Roswell Park Memorial Institute (RPMI) 1640 (Cytiva HyClone SH30027.01), which provided much better growth conditions. All cell lines were routinely tested for mycoplasma infection and passaged no more than 10 times from ATCC authenticated stocks.

Reagents. Expression plasmids for human ACE2, TMPRSS2, and HA-tagged SARS-CoV-2 spike were a gift from Stefan Pohlmann (Hoffmann M et al. Cell, 2020, 181, 271-280). Dec-RVKR-CMK (furin inhibitor-1) was from EMD Millipore (344930). Imipramine hydrochloride, Salmeterol, and Brompheniramine were from AK Scientific (J10511, K-590, and M-1266, respectively). Hexylresorcinol, Semaxanib (SU-5416), Ezetimibe, and Linsitinib (OSI-906) were from TargetMol (T0314, T2064, T1593, and T6017, respectively).

Transfection of cells for syncytia assay. On the day of experiments, acceptor cells were transfected with mammalian expression plasmids for ACE2 and TMPRSS2 using FuGene6 (Roche) at a 1:3 DNA-to-reagent ratio with 22 ng DNA per well (30 μl) of a 384-well plate. 4,000 cells were plated in collagen-coated microplates (Greiner 781956) and centrifuged at 500 g for 1 min. Donor cells were transfected under the same conditions with expression plasmids for eGFP or eGFP plus SARS-CoV-2 spike protein and plated in T-25 flasks (3 ml). Both donor and acceptor cells were incubated for 3 days at 37° C. Calu-3 cells were left untransfected and seeding density was 8,000 cells/well in RPMI.

Cell treatment for High Content Screening. On the day of co-culture, acceptor cells were pretreated for 1-2 h with vehicle or test agents; compounds were dissolved in DMSO and diluted into complete DMEM to a 3× concentration of the highest desired concentration in the assay. The resulting solutions were serially diluted on a 96-well plate into DMEM containing 3% DMSO. Fifteen microliter of the resulting gradients were transferred to cells using a Biomek 2000 liquid handler (Beckman Coulter) in duplicate to yield quadruplicate measurements for each concentration of test agents. The final concentration of DMSO in the assay was 1%. Each plate contained 80 wells of vehicle controls, 16 wells of mock-transfected acceptor cells, and 16 wells of ACE2/TMPRSS2 transfected acceptor cells incubated with GFP-only expressing donor cells (no spike).

Syncytia assay co-culture, imaging, and analysis. Donor cells were dislodged from their flasks with non-enzymatic cell dissociation buffer (Thermo Fisher 13151014) after two gentle washes with PBS. GFP-positive cells were counted in a hemocytometer. 2,000 GFP-positive cells in 15 μl DMEM were added to acceptor cells, plates centrifuged at 500 g for 1 min, and syncytia formation monitored. After 4 h cells were imaged live in the GFP channel (Ex485/Em525 nm) on a Molecular Devices ImageXpress Ultra or a Perkin Elmer OPERA Phenix Plus High Content Screening (HCS) reader using a 20× objective. Four fields were acquired per well. Images were uploaded to Definiens Developer (Ver 6, Definiens AG, Germany) and analyzed by a custom Cognition Network Technology (CNT) ruleset that separated individual cells, cell aggregates, and syncytia based on size, intensity, and texture of GFP expressing objects. The final parameters used for plotting were the percentage of GFP-positive area covered by syncytia relative to the total area covered by GFP-positive objects, and the total GFP-positive area as a surrogate for cell number. Data were averaged from the four imaging fields and normalized to vehicle-treated controls. Data from multiple independent experiments were pooled and analyzed by one-way ANOVA followed by Dunnett's multiple comparisons test. Dose-response data were fitted to a four-parameter logistic equation in GraphPad Prism (Ver. 7).

Data availability. The data and codes generated during the study are available at: https://github.com/Hannah-Qingya/Covid19_systems-level_analysis. The QuartataWeb server that is online accessible at http://quartata.csb.pitt.edu/ was also used.

### Example 2—Approach for the Discovery of Repurposed Drugs and Compounds for Treatment Against SARS-CoV-2 Infection

Disclosed herein are strategies for repurposing existing drugs and identifying new compounds for the treatment of SARS-CoV-2 infection

Summary: Covid-19 (Coronavirus Disease-2019) caused by SARS-CoV-2 (Severe Acute Respiratory Syndrome Coronavirus (CoV) type 2 virus) has led to over 1.3 million deaths as of mid-November 2020, due to its high contagiousness (basic reproductive number (R0) of 2.0-2.5) and therefore rapid spread, compared to the SARS-CoV (R0=1.7-1.9) or the Middle East respiratory syndrome (MERS) (R0=0.7) of the same coronavirus subfamily; and mortality rates in the ranges 4-28%, 3.6-30% and 60-65% have been reported for the three respective CoVs. A large number of vaccine- and drug-candidates under preclinical or clinical trials (191 vaccine candidates and 319 drug candidates) have been reported as of September 2020; with two vaccine trials, Pfizer/BioNTech and Moderna, now showing 95% success rate. Yet, no drugs have been FDA-approved to date, apart from remdesivir, an RNA-dependent RNA polymerase (RdRP) inhibitor that inhibits SARS-CoV and MERS-CoV, as a repurposable drug. Current practices such as the use of corticosteroids, such as dexamethasone, or intravenous immunoglobulin (IVIG) are supportive (for alleviating or preventing hyperinflammatory complications) rather than therapeutic according to the Centers for Disease Control (CDC) guidelines. There is an urgent need to develop new therapeutics against Covid-19.

While efforts to target viral proteins are underway, an alternative strategy is to pursue host-targeted therapies. The host cell response is essential to enabling viral entry, endosomal escape, translation, replication, assembly, and release. Host cells are also naturally armed with antiviral programs, which, if properly induced, can constrain the in vivo viral spread within a canonical 4-7 day period, upon sufficient adaptive immunity development. Herein, the focus was on the identification of compounds that modulate host cell responses, using a comprehensive, mechanism unbiased, and highly integrated systems-level approach. An important component of this type of study is knowledge of networks of protein-protein interactions (PPIs) in the host cell, or disease modules that play a role at various stages of viral infection.

Using transcriptome data from SARS-CoV-2-infected A549 (human adenocarcinomic alveolar basal epithelial) cells from lung tissue, and A549 cells overexpressing the host cell receptor angiotensin-converting enzyme 2 (ACE2), CMap analysis was employed to predict targets involved with SARS-CoV-2 infection, and the QuartataWeb server, developed for compound-target-pathway mapping, was utilized to predict drugs/compounds that would modulate those targets. This is a Quantitative Systems Pharmacology (QSP) approach that considers systems-level effects originating from the promiscuity of drugs and/or the pleiotropy of targets. Rather than a limited drug repurposing effort that merely preserves target class across indications, the focus herein was on a comprehensive unbiased virus-infected cell phenotype (manifested as a DEG signature) that reflects emergent virally driven cellular networks and connects these to drugs that can be repurposed without the need for molecular information.

Using this approach, 38 priority candidate compounds were identified (including repurposable and investigational drugs) that target the host system comprised of 15 compounds with potentially antiviral actions (Table 14) and 23 with possible anti-hyperinflammatory (adjuvant) actions (Table 15). Ten belonging to the first group, including six that are FDA-approved (imipramine, salmeterol, hexylresorcinol, brompheniraamine, ezetimibe, and temsirolimus) and four that are under development (linsitinib, torin-1, rottlerin, semaxanib), have been selected for in vitro assays with different types of cell lines (Vero-E6 cells, HEK293T cells and Calu-3 lung cancer cells). Several of these drug/compounds inhibited SARS-CoV-2 infection in a dose-dependent manner with salmeterol and linsitinib being particularly effective. These findings expand the repertoire of drugs/compounds that could be repurposed/developed for possible Covid-19 treatment as either single drugs or drug combinations.

Overall, a QSP workflow for predicting drugs and compounds that interact cell host proteins involved in viral infection and immune response to viral infection based on transcriptomic profiles was conceived and established (FIG. 1). The workflow was applied and drugs/compounds that interact with cell host proteins involved in SARS-CoV-2 infection (Table 14 and Table 16) and that would elicit anti-cytokine activity to protect from SARS-CoV-2 hyperinflammation were predicted therefrom (Table 15 and Table 17). A subset of the predicted drugs/compounds were experimentally tested and drugs/compounds that inhibit SARS-CoV-2 entry into cells were identified (FIG. 17 and FIG. 24-FIG. 31).

The approach established here is a combination of computational and systems biology analyses for the identification of cellular targets and mechanism associated with viral infection and the identification of compounds and repurposable drugs to modulate infection. The focus on host targets enables a new series of previously unrecognized targets with the potential for inhibition of SARS-CoV-2 infections. This approach can be more efficient than traditional screening approaches for identifying drugs/compounds to move forward into the clinical testing. Compounds were identified herein that were previously unknown to have potential for the inhibition of SARS-CoV-2 infection. Combinations of the predicted drugs may increase the efficacy.

TABLE 14
Prioritized potential anti-viral compounds and repurposable drugs

| | | | | Viral | |
|---|---|---|---|---|---|
| Drug/Compound | | Disease | Description/ | Entry | Syncytia |
| Name | Status | module | MOAs | Assay | Assay |
| Brompheniramine | FDA-approved | Viral entry | Histamine | Not | Active |
| | | | receptor | Tested | |
| | | | antagonist | | |
| Ezetimibe | FDA-approved | Viral entry, | Niemann-Pick | Active | Active |
| | | Regulation | C1-like 1 protein | | |
| | | and signaling | antagonist, | | |
| | | | cholesterol | | |
| | | | inhibitor, | | |
| | | | autophagy | | |
| | | | enhancer | | |
| Hexylresorcinol | FDA-approved | Viral entry | Local anesthetic | Not | Active |
| | | | | Teste | |
| Imipramine | FDA-approved | Viral entry | Norepinephrine | Not | Active |
| | | | and serotonin | Tested | |
| | | | reuptake | | |
| | | | inhibitor, | | |
| | | | autophagy | | |
| | | | enhancer | | |
| Ipratropium | FDA-approved | Viral | Acetylcholine | Not | Not |
| | | replication | receptor | Tested | Tested |
| | | and | antagonist | | |
| | | translation | | | |
| Mefenamic acid | FDA-approved | Viral repl and | Cyclooxygenase | Not | Not |
| | | translation | inhibitor | Tested | Tested |
| Salmeterol | FDA-approved | Viral entry | Adrenergic | Active | Active |
| | | | receptor agonist, | | |
| | | | autophagy | | |
| | | | enhancer | | |
| Temsirolimus | FDA-approved | Immune | mTOR inhibitor, | Active | Not |
| | | response, | autophagy | | Tested |
| | | Regulation | enhancer | | |
| | | and signaling | | | |
| AS-605240 | Investigational | Viral | PI3K inhibitor, | Not | Not |
| | | replication | autophagy | Tested | Tested |
| | | and | enhancer | | |
| | | translation, | | | |
| | | Regulation | | | |
| | | and signaling, | | | |
| | | Immune | | | |
| | | response | | | |
| JNJ16259685 | Investigational | Viral entry | Glutamate | Not | Not |
| | | | receptor | Tested | Tested |
| | | | antagonist | | |
| Linsitinib | Investigational | Viral entry | IGF-1-and | Not | Active |
| | | | insulin receptor | Tested | |
| | | | inhibitor, TBK1 | | |
| | | | activator through | | |
| | | | ARF1 | | |
| QL-XII-47 | Investigational | XXX | Cytoplasmic | Not | Not |
| | | | tyrosine protein | Tested | Tested |
| | | | kinase BMX | | |
| | | | inhibitor | | |
| Rottlerin | Investigational | XXX | MAPK and | Active | Not |
| | | | protein kinase | | Tested |
| | | | inhibitor, | | |
| | | | autophagy | | |
| | | | enhancer | | |
| Semaxanib | Investigational | Viral entry, | VEGFR inhibitor | Not | Active |
| | | Regulation | | Tested | |
| | | and signaling, | | | |
| | | Immune | | | |
| | | response | | | |
| Torin-1 | Investigational | Immune | mTOR inhibitor, | Active | Not |
| | | response, | PI3K inhibitor, | | Tested |
| | | Regulation | autophagy | | |
| | | and | enhancer | | |
| | | signaling | | | |

TABLE 15
Prioritized potential anti-hyperimmune compounds and repurposable drugs.

| Drug/Compound | | |
|---|---|---|
| Name | Status | Description/MOAs |
| Atorvastatin | FDA-approved | HMGCR inhibitor, autophagy enhancer |
| Azelastine | FDA-approved | Dopamine receptor antagonist |
| Chlorphenamine | FDA-approved | Histamine receptor antagonist |
| Clarithromycin | FDA-approved | Bacterial 50S ribosomal subunit inhibitor, |
| | | autophagy inhibitor |
| Fluphenazine | FDA-approved | Dopamine receptor antagonist, autophagy enhancer |
| Liothyronine | FDA-approved | Thyroid hormone stimulant |
| Loperamide | FDA-approved | Opioid receptor agonist, autophagy enhancer |
| Midodrine | FDA-approved | Adrenergic receptor agonist |
| Nifedipine | FDA-approved | Calcium channel blocker, autophagy enhancer |
| Olanzapine | FDA-approved | Dopamine receptor antagonist, autophagy enhancer |
| Pirfenidone | FDA-approved | TGFβ receptor inhibitor, anti-fibrotic, autophagy |
| | | enhancer |
| Rucaparib | FDA-approved | PARP inhibitor, autophagy enhancer |
| Trifluoperazine | FDA-approved | Dopamine receptor antagonist, autophagy dual- |
| | | modulator |
| AZD-8055 | Investigational | mTOR inhibitor, autophagy enhancer |
| Berbamine | Investigational | Calmodulin antagonist, autophagy inhibitor |
| CGP-60474 | Investigational | CDK inhibitor |
| Darinaparsin | Investigational | Apoptosis stimulant |
| Hexamethylene | Investigational | Sodium/hydrogen antiport inhibitor |
| Isoliquiritigenin | Investigational | Guanylate cyclase activator, autophagy enhancer |
| JAK3-Inhibitor-II | Investigational | JAK inhibitor |
| Saracatinib | Investigational | SRC inhibitor |
| Taurodeoxycholic | Investigational | Bile acid |
| acid | | |
| Triptolide | Investigational | RNA polymerase inhibitor |

TABLE 16
Complete list of drugs and compounds with potential antiviral activity against SARS-
CoV-2-infection.

| Drug/Compound | | |
|---|---|---|
| Name | CMap ID | Mechanism of Action |
| 2-aminopurine | BRD-K35128472 | Serine/threonine kinase inhibitor |
| 5-nonyloxytryptamine | BRD-K08219523 | Serotonin receptor agonist |
| Abiraterone | BRD-K55301415 | 17,20 lyase inhibitor, Androgen biosynthesis |
| | | inhibitor, Cytochrome P450 inhibitor, Steroid |
| | | sulfatase inhibitor |
| AG-879 | BRD-K59469039 | Angiogenesis inhibitor, Tyrosine kinase |
| | | inhibitor, VEGFR inhibitor |
| Alaproclate | BRD-A14966924 | Serotonin receptor antagonist |
| Alfacalcidol | BRD-K93433262 | Vitamin D receptor agonist |
| Alisertib | BRD-K75295174 | Aurora kinase inhibitor |
| Altrenogest | BRD-A27554692 | Progestogen hormone |
| ALW-II-38-3 | BRD-K68191783 | Ephrin inhibitor |
| AM-281 | BRD-K59419204 | Cannabinoid receptor antagonist |
| Amiodarone | BRD-K17561142 | Potassium channel blocker |
| Anandamide | BRD-K78280988 | Cannabinoid receptor agonist |
| APHA-compound-8 | BRD-K74733595 | HDAC inhibitor |
| Apicidin | BRD-K64606589 | HDAC inhibitor |
| Arcyriaflavin-a | BRD-K72726508 | CDK inhibitor |
| Arecaidine | BRD-K63792901 | Acetylcholine receptor agonist |
| Arecaidine | BRD-K23922020 | Acetylcholine receptor agonist |
| AS-605240 | BRD-K41895714 | PI3K inhibitor |
| AS-703026 | BRD-K89014967 | MEK inhibitor |
| Aspirin | BRD-K11433652 | Cyclooxygenase inhibitor |
| Avrainvillamide- | BRD-A70731303 | nucleophosmin inhibitor |
| analog-5 | | |
| AY-9944 | BRD-K03642198 | Hedgehog pathway modulator |
| Azathioprine | BRD-K32821942 | Dehydrogenase inhibitor |
| AZD-8055 | BRD-K69932463 | MTOR inhibitor |
| Barasertib | BRD-K63923597 | Aurora kinase inhibitor |
| BAY-36-7620 | BRD-K54704028 | Glutamate receptor antagonist |
| Benidipine | BRD-A35519318 | Calcium channel blocker |
| Bergenin | BRD-A15034104 | Interleukin inhibitor |
| BH31-1 | BRD-A38913120 | BCL inhibitor |
| BIBX-1382 | BRD-K70914287 | EGFR inhibitor, Tyrosine kinase inhibitor |
| BIX-01338 | BRD-K26863634 | Histone lysine methyltransferase inhibitor |
| BML-ST330 | BRD-A77118605 | Phospholipase inhibitor |
| BMS-191011 | BRD-K95609758 | Potassium channel activator |
| BMY-14802 | BRD-A15435692 | Sigma receptor antagonist |
| BMY-45778 | BRD-K84895041 | IP1 prostacyclin receptor agonist |
| Bosutinib | BRD-K99964838 | ABL inhibitor, BCR-ABL kinase inhibitor, SRC |
| | | inhibitor |
| Brazilin | BRD-A83326220 | Nitric oxide production inhibitor |
| BRD-K64835161 | BRD-K64835161 | — |
| Brompheniramine | BRD-A68723818 | Histamine receptor antagonist |
| Calmidazolium | BRD-A98283014 | Calcium channel blocker, Calmodulin |
| | | antagonist |
| Camptothecin | BRD-A30437061 | Topoisomerase inhibitor |
| Ceforanide | BRD-K37848908 | Penicillin binding protein inhibitor |
| Cetraxate | BRD-K48932581 | Mucus protecting agent |
| CGP-7930 | BRD-K65786282 | GABA receptor positive allosteric modulator |
| CHEMBL-374350 | BRD-K59962020 | NFKB pathway inhibitor |
| Chenodeoxycholic- | BRD-K18135438 | 11-β-HSD1 inhibitor, FXR agonist |
| acid | | |
| Chlorpromazine | BRD-K89997465 | Dopamine receptor antagonist |
| Ciclacillin | BRD-K89046952 | Bacterial cell wall synthesis inhibitor |
| Cimaterol | BRD-A65440446 | Adrenergic receptor agonist |
| Cisapride | BRD-K06895174 | Serotonin receptor agonist |
| Clobenpropit | BRD-K71430621 | Histamine receptor antagonist |
| Cortisone | BRD-A54487287 | Glucocorticoid receptor agonist |
| Corynanthine | BRD-K06467078 | Adrenergic receptor antagonist |
| Dactolisib | BRD-K12184916 | MTOR inhibitor, PI3K inhibitor, Protein kinase |
| | | inhibitor |
| Dapsone | BRD-K62363391 | Bacterial antifolate |
| Decitabine | BRD-K79254416 | DNA methyltransferase inhibitor |
| Demeclocycline | BRD-A75368507 | Bacterial 30S ribosomal subunit inhibitor |
| Dephostatin | BRD-K60274257 | Tyrosine phosphatase inhibitor |
| Desipramine | BRD-K60762818 | Tricyclic antidepressant |
| Desmethylclozapine | BRD-K10042277 | Acetylcholine receptor agonist |
| Desoxycorticosterone | BRD-A75402480 | Mineralocorticoid receptor agonist |
| Dichlorobenzamil | BRD-K12906962 | Sodium/calcium exchange inhibitor |
| Dihydrosamidin | BRD-K63945320 | Phospholipase inhibitor, Nitric oxide production |
| | | inhibitor, platelet activating factor receptor |
| | | antagonist |
| Dipyridamole | BRD-K86301799 | Phosphodiesterase inhibitor |
| Droxinostat | BRD-K11558771 | HDAC inhibitor |
| Duloxetine | BRD-K71103788 | Serotonin and norepinephrine reuptake inhibitor |
| Dydrogesterone | BRD-K68620903 | Progesterone receptor agonist |
| E-4031 | BRD-K41713976 | Potassium channel blocker |
| Edrophonium | BRD-K81128206 | Acetylcholinesterase inhibitor |
| Eicosatrienoic-acid | BRD-K63913457 | Vasodilator |
| Elesclomol | BRD-K82135108 | Oxidative stress inducer |
| Emetine | BRD-A25687296 | Protein synthesis inhibitor |
| ENMD-2076 | BRD-K68488863 | FLT3 inhibitor, VEGFR inhibitor, Aurora |
| | | kinase inhibitor |
| Entacapone | BRD-K83636919 | Catechol O methyltransferase inhibitor |
| Entinostat | BRD-K77908580 | HDAC inhibitor |
| Epicatechin | BRD-K50660797 | Bacterial DNA gyrase inhibitor, |
| | | Cyclooxygenase inhibitor, DNA polymerase |
| | | inhibitor |
| Equilin | BRD-K04046242 | Estrogen receptor agonist |
| Eugenol | BRD-K32977963 | Androgen receptor antagonist |
| Ezetimibe | BRD-A41519720 | Niemann-Pick C1-like 1 protein antagonist, |
| | | Cholesterol inhibitor |
| Fenoldopam | BRD-A50684349 | Dopamine receptor agonist |
| FGIN-1-27 | BRD-K09778810 | Inositol monophosphatase inhibitor |
| Flavanone | BRD-A07824748 | 11-β-HSD1 inhibitor |
| Fludarabine | BRD-K66788707 | DNA synthesis inhibitor, DNA repair enzyme |
| | | inhibitor, Purine antagonist |
| Formestane | BRD-A31801025 | Aromatase inhibitor |
| Fostamatinib | BRD-K20285085 | SYK inhibitor |
| FR-122047 | BRD-K30990140 | Cyclooxygenase inhibitor |
| GBR-12783 | BRD-K92015269 | Dopamine uptake inhibitor |
| GBR-12935 | BRD-K50135270 | Dopamine uptake inhibitor |
| Gemcitabine | BRD-K15108141 | Ribonucleotide reductase inhibitor |
| Glipizide | BRD-K12219985 | Sulfonylurea |
| GR-127935 | BRD-K11911061 | Serotonin receptor antagonist |
| GS-39783 | BRD-K75478907 | GABA receptor modulator |
| GSK-1059615 | BRD-K06750613 | PI3K inhibitor |
| GSK-1904529A | BRD-K04833372 | IGF-1 inhibitor, IGF-1R inhibitor, Insulin |
| | | receptor ligand |
| GW-9662 | BRD-K93258693 | PPAR receptor antagonist |
| H-7 | BRD-A55756846 | PKA inhibitor |
| HDAC3-selective | BRD-K29313308 | HDAC inhibitor |
| Heliomycin | BRD-K64517075 | ATP synthase inhibitor |
| Heraclenol | BRD-A77050075 | Vitamin K antagonist |
| Hexylresorcinol | BRD-K99946902 | Local anesthetic |
| HG-6-64-01 | BRD-U37049823 | RAF inhibitor |
| Homoharringtonine | BRD-K76674262 | Protein synthesis inhibitor |
| Homosalate | BRD-A34751532 | HSP inducer |
| Hydroxycholesterol | BRD-A36707673 | LXR agonist |
| Hyoscyamine | BRD-K40530731 | Acetylcholine receptor antagonist |
| Ibuprofen | BRD-A17655518 | Cyclooxygenase inhibitor, NFkB pathway |
| | | inhibitor |
| Imipramine | BRD-K38436528 | Norepinephrine and Serotonin transporter |
| | | inhibitor |
| Immethridine | BRD-K49519092 | Histamine receptor agonist |
| Iodophenpropit | BRD-K51918615 | Histamine receptor antagonist |
| I-OMe-AG-538 | BRD-K35377380 | IGF-1 inhibitor |
| Ioxaglic-acid | BRD-K79124250 | Radiopaque medium |
| Ipratropium | BRD-A05352148 | Acetylcholine receptor antagonist |
| Isotretinoin | BRD-K76723084 | Retinoid receptor agonist |
| JAK3-Inhibitor-II | BRD-K52850071 | JAK inhibitor |
| JNJ-16259685 | BRD-K64670467 | Glutamate receptor antagonist |
| Kavain | BRD-A75455249 | Calcium channel modulator, Sodium channel |
| | | blocker |
| KIN001-127 | BRD-A29901043 | ITK inhibitor |
| KU-0063794 | BRD-K67566344 | MTOR inhibitor |
| KU-55933 | BRD-K25311561 | ATM kinase inhibitor |
| KU-C103443N | BRD-A81402010 | CDC inhibitor, Rho associated kinase inhibitor |
| KUC104502N | BRD-K24538644 | — |
| L-165041 | BRD-K40656405 | PPAR receptor agonist |
| L-733060 | BRD-K15791587 | Tachykinin antagonist |
| L-741626 | BRD-K05181463 | Dopamine receptor antagonist |
| Leflunomide | BRD-K78692225 | Dihydroorotate dehydrogenase inhibitor, |
| | | PDGFR receptor inhibitor |
| Linsitinib | BRD-K08589866 | IGF-1 inhibitor |
| Liothyronine | BRD-K89152108 | Thyroid hormone stimulant |
| Lonidamine | BRD-K96670504 | Glucokinase inhibitor |
| LY-2140023 | BRD-K49519144 | Glutamate receptor agonist |
| LY-288513 | BRD-K24675965 | CCK receptor antagonist |
| Lypressin | BRD-K93331255 | Vasopressin receptor agonist |
| M2-PK-activator | BRD-K80672993 | — |
| m- | BRD-K36965586 | Serotonin receptor agonist |
| chlorophenylbiguanide | | |
| Meclozine | BRD-A50311610 | CAR agonist |
| Mefenamic-acid | BRD-K92778217 | Cyclooxygenase inhibitor |
| Mepacrine | BRD-A45889380 | Cytokine production inhibitor, NFkB pathway |
| | | inhibitor, TP53 activator |
| Mephenytoin | BRD-A83937277 | Hydantoin antiepileptic |
| Mepireserpate | BRD-A71765365 | Catecholamine depleting sympatholytic |
| Mercaptopurine | BRD-K91601245 | Immunosuppressant, Protein synthesis inhibitor, |
| | | Purine antagonist |
| Mesna | BRD-M40783228 | Antioxidant |
| Mesoridazine | BRD-A14395271 | Dopamine receptor antagonist |
| Metergoline | BRD-A30435184 | Dopamine receptor agonist, Serotonin receptor |
| | | antagonist |
| Metformin | BRD-K79602928 | Insulin sensitizer |
| Methimazole | BRD-K54416256 | Antithyroid |
| Metixene | BRD-A33711280 | Acetylcholine receptor antagonist |
| Metrizamide | BRD-A45543382 | Radiopaque medium |
| Midodrine | BRD-A79981887 | Adrenergic receptor agonist |
| Mirtazapine | BRD-A64977602 | Adrenergic receptor antagonist, Serotonin |
| | | receptor antagonist |
| MK-5108 | BRD-K53665955 | Aurora kinase inhibitor |
| ML-7 | BRD-K93201660 | Myosin light chain kinase inhibitor |
| ML-9 | BRD-K68402494 | Myosin light chain kinase inhibitor |
| Molsidomine | BRD-K35531059 | Guanylyl cyclase activator |
| Moracizine | BRD-K21548250 | Sodium channel blocker |
| Mosapride | BRD-A39052811 | Serotonin receptor agonist |
| MR-16728 | BRD-A30590053 | Acetylcholine release enhancer, Acetylcholine |
| | | release stimulant |
| MRS-1845 | BRD-A32949107 | Calcium channel blocker |
| Mycophenolate- | BRD-K92428153 | Dehydrogenase inhibitor, Hydroxycarboxylic |
| mofetil | | acid receptor agonist, Immunosuppressant, |
| | | Inosine monophosphate dehydrogenase |
| | | inhibitor, Inositol monophosphatase inhibitor |
| n-arachidonyl-GABA | BRD-K06024458 | cannabinoid receptor agonist |
| Narciclasine | BRD-K06792661 | Coflilin signaling pathway activator, LIM |
| | | kinase activator, Rho associated kinase activator |
| NBI-27914 | BRD-K61177364 | CRF receptor antagonist |
| NGB-2904 | BRD-K05181084 | Dopamine receptor antagonist |
| Niacin | BRD-K61993165 | NAD precursor with lipid lowering effect, |
| | | vitamin B |
| Nilotinib | BRD-K81528515 | ABL inhibitor, BCR-ABL kinase inhibitor |
| NNC-55-0396 | BRD-K78122587 | T-type calcium channel blocker |
| Norgestrel | BRD-A50928468 | Progesterone receptor agonist |
| NSC-663284 | BRD-K03109492 | CDC inhibitor |
| nTZDpa | BRD-K54708045 | PPAR receptor agonist |
| NU-7026 | BRD-K09537769 | DNA dependent protein kinase inhibitor, MTOR |
| | | inhibitor, PI3K inhibitor |
| NU-7441 | BRD-K00337317 | DNA dependent protein kinase inhibitor, P- |
| | | glycoprotein inhibitor |
| NVP-TAE684 | BRD-K50140147 | ALK inhibitor |
| Ochratoxin-a | BRD-K39944607 | Phenylalanyl tRNA synthetase inhibitor |
| Oleoylethanolamide | BRD-K66956375 | Cannabinoid receptor agonist, Glucose |
| | | dependent insulinotropic receptor agonist, |
| | | Potassium channel blocker, PPAR receptor |
| | | agonist |
| OSI-027 | BRD-K94294671 | MTOR inhibitor |
| Oxfendazole | BRD-A33447119 | Anthelmintic |
| Oxiconazole | BRD-K23369905 | Bacterial cell wall synthesis inhibitor |
| Oxybutynin | BRD-A65013509 | Acetylcholine receptor antagonist |
| Panobinostat | BRD-K02130563 | HDAC inhibitor |
| PD-0325901 | BRD-K49865102 | MEK inhibitor, MAPK inhibitor, Protein kinase |
| | | inhibitor |
| PD-102807 | BRD-A89337244 | Acetylcholine receptor antagonist |
| PD-184352 | BRD-K05104363 | MEK inhibitor |
| Perospirone | BRD-K85503079 | Dopamine and serotonin receptors' antagonist |
| PG-9 | BRD-A70268693 | Acetylcholine receptor agonist |
| PHA-665752 | BRD-K95435023 | c-Met inhibitor |
| Phenytoin | BRD-K55930204 | Hydantoin antiepileptic |
| Phosphodiesterase-V- | BRD-K68873215 | Phosphodiesterase inhibitor |
| inhibitor-II | | |
| Phylloquinone | BRD-A55815733 | Vitamin K, Γ carboxylase enzyme |
| PI-103 | BRD-K67868012 | MTOR inhibitor, PI3K inhibitor |
| Pidorubicine | BRD-K04548931 | Topoisomerase inhibitor |
| PIK-90 | BRD-K99818283 | PI3K inhibitor |
| Pimozide | BRD-K01292756 | Dopamine receptor antagonist |
| Pizotifen | BRD-K75958195 | Serotonin receptor antagonist |
| PKCβ-inhibitor | BRD-K89687904 | PKC inhibitor |
| Ponalrestat | BRD-K68332390 | Aldose reductase inhibitor |
| PP-30 | BRD-K30677119 | RAF inhibitor |
| Procyclidine | BRD-A31800922 | Acetylcholine receptor antagonist |
| Prostaglandin-b2 | BRD-K82865713 | CAMP inhibitor |
| Proxymetacaine | BRD-K79116891 | Sodium channel blocker |
| PSB-36 | BRD-A70407468 | Adenosine receptor antagonist |
| Pterostilbene | BRD-K92870997 | Cyclooxygenase inhibitor, PPAR receptor |
| | | agonist |
| Puromycin | BRD-A28970875 | Protein synthesis inhibitor |
| QL-XII-47 | BRD-U86922168 | BTK inhibitor, Cytoplasmic tyrosine protein |
| | | kinase BMX inhibitor |
| Raloxifene | BRD-K63828191 | Estrogen receptor antagonist, Selective estrogen |
| | | receptor modulator (SERM) |
| Razoxane | BRD-K07265709 | Chelating agent, Topoisomerase inhibitor |
| Rescinnamine | BRD-K52930707 | ACE inhibitor |
| Reserpine | BRD-K95921201 | Vesicular monoamine transporter inhibitor |
| Ribavirin | BRD-A96255180 | Antiviral |
| Ropivacaine | BRD-K50938786 | Sodium channel blocker |
| Rottlerin | BRD-K03816923 | MAP kinase inhibitor, Protein kinase inhibitor |
| RS-17053 | BRD-K76840893 | Adrenergic receptor antagonist |
| RS-67333 | BRD-K46142322 | Serotonin receptor partial agonist |
| RU-28318 | BRD-A92585442 | Cytochrome P450 inhibitor |
| SA-792728 | BRD-K20755323 | Sphingosine kinase inhibitor |
| SA-94315 | BRD-K20197062 | Caspase inhibitor |
| Salicin | BRD-K64614248 | Anti-inflammatory |
| Salmeterol | BRD-A01320529 | Adrenergic receptor agonist |
| SB-216641 | BRD-K30867024 | Serotonin receptor antagonist |
| SB-590885 | BRD-K01253243 | RAF inhibitor |
| SCH-23390 | BRD-K45435259 | Dopamine receptor antagonist |
| SDZ-205-557 | BRD-K15868788 | Serotonin receptor antagonist |
| Semaxanib | BRD-K63504947 | VEGFR inhibitor |
| Sertraline | BRD-K82036761 | Serotonin receptor antagonist |
| SKF-81297 | BRD-A09828896 | Dopamine receptor agonist |
| SN-38 | BRD-A36630025 | Topoisomerase inhibitor |
| Somatostatin | BRD-K14681867 | Somatostatin receptor agonist |
| Sphingosine | BRD-K62959606 | Ceramidase inhibitor |
| Splitomycin | BRD-K27710560 | SIRT inhibitor |
| SR-27897 | BRD-K35629949 | CCK receptor antagonist |
| Stavudine | BRD-K93880783 | DNA directed DNA polymerase inhibitor, |
| | | Reverse transcriptase inhibitor |
| STO-609 | BRD-K52620403 | Calmodulin antagonist |
| Sulfafurazole | BRD-K50859149 | Bacterial antifolate |
| Sumatriptan | BRD-K50938287 | Serotonin receptor agonist |
| TC-2559 | BRD-K67352070 | Acetylcholine receptor agonist |
| Telmisartan | BRD-K73999723 | Angiotensin receptor antagonist |
| Temsirolimus | BRD-A62025033 | MTOR inhibitor |
| Terbinafine | BRD-K68132782 | Fungal squalene epoxidase inhibitor |
| Terbutaline | BRD-A50157456 | Adrenergic receptor agonist |
| Terfenadine | BRD-A06352418 | Histamine receptor antagonist |
| Tetrahydrobiopterin | BRD-A67605442 | Nitric oxide (NO) stimulant, NO synthase |
| | | stimulant, Phenylalanine 4-hydroxylase |
| | | stimulant |
| Tetrahydropalmatine | BRD-A43940795 | Serotonin release inhibitor |
| TGX-221 | BRD-A41692738 | PI3K inhibitor |
| Thiotepa | BRD-K09631521 | Cytochrome P450 inhibitor |
| Tolazoline | BRD-K46211610 | Adrenergic receptor antagonist |
| Torin-1 | BRD-K40175214 | MTOR inhibitor, PI3K inhibitor |
| TPCA-1 | BRD-K51575138 | IKK inhibitor |
| Tranylcypromine | BRD-A43974575 | Monoamine oxidase inhibitor |
| Tretinoin | BRD-K06926592 | Retinoid receptor agonist, Retinoid receptor |
| | | ligand |
| Tribenoside | BRD-A60294240 | Anti-inflammatory, Capillary stabilizing agent |
| Trichostatin-a | BRD-K68202742 | HDAC inhibitor, CDK activator, ID1 inhibitor |
| Tyrphostin-AG-556 | BRD-K14441456 | EGFR inhibitor |
| UNC-0321 | BRD-K74236984 | Histone lysine methyltransferase inhibitor |
| VER-155008 | BRD-K32330832 | HSP inhibitor |
| Vorinostat | BRD-K81418486 | HDAC inhibitor |
| VU-0366037-2 | BRD-K39823328 | Glutamate receptor modulator |
| VU-0404997-2 | BRD-A34208323 | Glutamate receptor modulator |
| VU-0415374-1 | BRD-K83010055 | Glutamate receptor modulator |
| Wiskostatin | BRD-A18579359 | Neural Wiskott-Aldrich syndrome protein |
| | | inhibitor |
| Wortmannin | BRD-A11678676 | PI3K inhibitor |
| WZ-3146 | BRD-K73293050 | EGFR inhibitor |
| WZ-4-145 | BRD-U25771771 | EGFR inhibitor |
| Y-134 | BRD-K94832621 | Estrogen receptor antagonist |
| YM-976 | BRD-K12932420 | Phosphodiesterase inhibitor |
| Zamifenacin | BRD-K80451230 | Acetylcholine receptor antagonist |
| ZM-447439 | BRD-K72703948 | Aurora kinase inhibitor |

TABLE 17
Complete list of drugs and compounds which can potentially elicit anti-cytokine
activity against hyperinflammation in SARS-COV-2-infected cells.

| Drug/Compound Name | CMap ID | Mechanism of Action |
|---|---|---|
| 3-matida | BRD-A87125127 | Glutamate receptor antagonist |
| 9-methyl-5H-6-thia-4,5- | BRD-K14696368 | NFkB pathway inhibitor |
| diaza-chrysene-6,6-dioxide | | |
| AC-55649 | BRD-K93176058 | Retinoid receptor agonist |
| Acadesine | BRD-A95696820 | AMPK activator |
| Acetyl-geranyl-cysteine | BRD-U01690642 | Isoprenylated protein methylation |
| | | inhibitor |
| AICA-ribonucleotide | BRD-A67373739 | AMPK activator |
| Alfuzosin | BRD-A09056319 | Adrenergic receptor antagonist |
| Alosetron | BRD-K46742498 | Serotonin receptor antagonist |
| Alprenolol | BRD-A00993607 | Adrenergic receptor antagonist |
| Alverine | BRD-K89055274 | Muscle relaxant |
| ALW-II-38-3 | BRD-K68191783 | Ephrin inhibitor |
| Aminomethyltransferase | BRD-A28318179 | Nitric oxide synthase inhibitor |
| Amoxapine | BRD-K02265150 | Norepinephrine reuptake inhibitor |
| Amylocaine | BRD-A09062839 | Local anesthetic |
| Anagrelide | BRD-K62200014 | Phosphodiesterase inhibitor |
| AQ-RA741 | BRD-K81729199 | Acetylcholine receptor antagonist |
| AR-C133057XX | BRD-K40892394 | Nitric oxide synthase inhibitor |
| Atorvastatin | BRD-U88459701 | HMGCR inhibitor |
| Auraptene | BRD-K85013741 | Nitric oxide production inhibitor |
| AY-9944 | BRD-K03642198 | Hedgehog pathway modulator |
| AZD-6482 | BRD-K58772419 | PI3K inhibitor |
| AZD-8055 | BRD-K69932463 | MTOR inhibitor |
| Azelastine | BRD-A68888262 | Histamine receptor antagonist |
| Benzydamine | BRD-K76133116 | Membrane integrity inhibitor, Prostanoid |
| | | receptor antagonist, Prostanoid receptor |
| | | inhibitor |
| Bepridil | BRD-A91008255 | Calcium channel or L-type Ca⁺⁺ channel |
| | | blocker |
| Berbamine | BRD-K50464341 | Calmodulin antagonist |
| BI-78D3 | BRD-K73982490 | JNK inhibitor |
| BIIB021 | BRD-K51967704 | HSP inhibitor |
| Bisoprolol | BRD-A89175223 | Adrenergic receptor antagonist |
| bis-tyrphostin | BRD-K32906660 | EGFR inhibitor |
| BMS-299897 | BRD-K02950022 | γ secretase inhibitor |
| BP-554 | BRD-K45479396 | Serotonin receptor agonist |
| BRD-A80383043 | BRD-A80383043 | Glutamate receptor agonist and/or |
| | | antagonist |
| BRD-K34437622 | BRD-K34437622 | Thymidylate synthase inhibitor |
| BRD-K63784565 | BRD-K63784565 | Topoisomerase inhibitor |
| BRD-K64835161 | BRD-K64835161 | NA |
| BRL-52537 | BRD-A37347161 | Opioid receptor agonist |
| Bromfenac | BRD-K47679368 | Cyclooxygenase inhibitor |
| Bromocriptine | BRD-A69960130 | Dopamine receptor agonist |
| Buphenine | BRD-A36267905 | Adrenergic receptor agonist |
| Bupropion | BRD-A05186015 | Dopamine uptake inhibitor |
| Butylparaben | BRD-K08287586 | DNA synthesis inhibitor |
| Carmoxirole | BRD-K82484965 | Dopamine receptor agonist |
| Carpindolol | BRD-A15530910 | Adrenergic receptor antagonist, serotonin |
| | | receptor antagonist |
| Carteolol | BRD-A42167015 | Adrenergic receptor antagonist |
| CDK1-5-inhibitor | BRD-K87932577 | CDK inhibitor, Glycogen synthase kinase |
| | | inhibitor |
| CGP-54626 | BRD-A55369275 | GABA receptor antagonist |
| CGP-60474 | BRD-K79090631 | CDK inhibitor |
| CGS-20625 | BRD-K68103045 | Benzodiazepine receptor agonist, GABA |
| | | benzodiazepine site receptor partial |
| | | agonist |
| Chlordiazepoxide | BRD-K86595100 | Benzodiazepine receptor agonist |
| Chlorphenamine | BRD-A04553218 | Histamine receptor antagonist |
| Chlorprothixene | BRD-K59058766 | Dopamine receptor antagonist |
| Cisapride | BRD-K06895174 | Serotonin receptor agonist |
| CITCO | BRD-K53263234 | CAR agonist |
| CL-82198 | BRD-K00675675 | Metalloproteinase inhibitor |
| Clarithromycin | BRD-K49668410 | Bacterial 50S ribosomal subunit inhibitor |
| Clebopride | BRD-K17294426 | Dopamine receptor antagonist |
| Clonidine | BRD-K98530306 | Adrenergic receptor agonist |
| CNQX | BRD-K53545112 | Glutamate receptor antagonist |
| Cotinine | BRD-K94144010 | Nicotine metabolite |
| Cyclazosin | BRD-A37837077 | Adrenergic receptor antagonist |
| Cycloserine | BRD-K87226815 | Bacterial cell wall synthesis inhibitor |
| D-64406 | BRD-K27665173 | PDGFR receptor inhibitor |
| Danazol | BRD-A92537424 | Estrogen receptor antagonist, |
| | | Progesterone receptor agonist |
| Darinaparsin | BRD-K35723520 | Apoptosis stimulant |
| Dasatinib | BRD-K49328571 | BCR-ABL kinase inhibitor, Ephrin |
| | | inhibitor, KIT inhibitor, PDGFR receptor |
| | | inhibitor, SRC inhibitor, Tyrosine kinase |
| | | inhibitor |
| Daunorubicin | BRD-K43389675 | RNA synthesis inhibitor, Topoisomerase |
| | | inhibitor |
| Dephostatin | BRD-K60274257 | Tyrosine phosphatase inhibitor |
| Desipramine | BRD-K60762818 | Tricyclic antidepressant |
| Dexketoprofen | BRD-K43764301 | Cyclooxygenase inhibitor |
| Dichloroacetic-acid | BRD-K13664374 | Pyruvate dehydrogenase kinase inhibitor |
| Dicyclohexylurea | BRD-K81521265 | Epoxide hydolase inhibitor |
| Dicycloverine | BRD-K68507560 | Acetylcholine receptor antagonist |
| Diethylstilbestrol | BRD-K45330754 | Estrogen receptor agonist |
| Dinoprostone | BRD-K26521938 | Prostanoid receptor agonist |
| Dipropyl-5ct | BRD-K32645441 | Serotonin receptor agonist |
| DMBI | BRD-K96084870 | PDGFR receptor inhibitor, VEGFR |
| | | inhibitor |
| Dopamine | BRD-K43887077 | Dopamine receptor agonist |
| DUP-697 | BRD-K06221026 | Cyclooxygenase inhibitor |
| Edaravone | BRD-K35458079 | Nootropic agent |
| EHNA | BRD-K27450477 | Adenosine deaminase inhibitor |
| EI-247 | BRD-K32710582 | IGF-1 inhibitor |
| Eicosatetraynoic-acid | BRD-K06080977 | Cyclooxygenase inhibitor, Lipoxygenase |
| | | inhibitor |
| EMD-386088 | BRD-K47659338 | Serotonin receptor agonist |
| EMF-bca1-60 | BRD-K68437527 | caspase inhibitor |
| Enrofloxacin | BRD-K76534306 | Bacterial DNA gyrase inhibitor |
| Estradiol | BRD-A18917088 | Contraceptive agent, Estrogen receptor |
| | | agonist |
| Etifenin | BRD-K63979671 | Compound used in hepatobiliary scans of |
| | | the liver |
| Etilefrine | BRD-A09925278 | Adrenergic receptor agonist |
| Exemestane | BRD-A73741725 | Aromatase inhibitor |
| Felbamate | BRD-K99107520 | Glutamate receptor antagonist |
| Fipronil | BRD-A50675702 | GABA gated chloride channel blocker |
| FIT | BRD-K17896185 | Opioid receptor agonist |
| Fluphenazine | BRD-K55127134 | Dopamine receptor antagonist |
| Flutamide | BRD-K28307902 | Androgen receptor antagonist |
| Foliosidine | BRD-A49734948 | Plant alkaloid |
| Formestane | BRD-A31801025 | Aromatase inhibitor |
| Fostamatinib | BRD-K20285085 | SYK inhibitor |
| Fraxidin | BRD-K66944906 | Carbonic anhydrase inhibitor |
| Fursultiamine | BRD-A71157293 | Vitamin B |
| Gabazine | BRD-K93280214 | GABA receptor antagonist |
| GANT-58 | BRD-K64451768 | GLI antagonist |
| Gavestinel | BRD-K49890030 | Glutamate receptor antagonist |
| GDC-0941 | BRD-K52911425 | PI3K inhibitor |
| Geldanamycin | BRD-A19500257 | HSP inhibitor |
| GR-144053 | BRD-K12120659 | Integrin antagonist |
| GR-206 | BRD-K00184207 | Aryl hydrocarbon receptor ligand |
| GR-235 | BRD-K26674531 | Estrogen receptor agonist, FXR |
| | | antagonist, Progesterone receptor agonist |
| Hexamethyleneamiloride | BRD-K40990712 | Sodium/hydrogen antiport inhibitor |
| HLI-373 | BRD-K17349619 | MDM inhibitor |
| HY-11007 | BRD-K97056771 | BCR-ABL kinase inhibitor |
| ICI-199441 | BRD-K73290745 | Opioid receptor agonist |
| ICI-89406 | BRD-A03359064 | Adrenergic receptor antagonist |
| Ilomastat | BRD-K51662849 | Matrix metalloprotease inhibitor |
| Indatraline | BRD-K01649396 | Norepinephrine transporter inhibitor |
| Iodophenpropit | BRD-K51918615 | Histamine receptor antagonist |
| Iproniazid | BRD-K88568253 | Monoamine oxidase inhibitor |
| Ipsapirone | BRD-K90574421 | Serotonin receptor agonist |
| Isoliquiritigenin | BRD-K33583600 | Guanylate cyclase activator |
| ITE | BRD-K60298136 | Aryl hydrocarbon receptor agonist |
| JAK3-Inhibitor-II | BRD-K52850071 | JAK inhibitor |
| Ketanserin | BRD-K49671696 | Serotonin receptor antagonist |
| KI-16425 | BRD-A25569250 | Lysophosphatidic acid receptor antagonist |
| KIN001-127 | BRD-A29901043 | ITK inhibitor |
| KIN001-220 | BRD-K53561341 | Aurora kinase inhibitor |
| KIN001-244 | BRD-K09186807 | Phosphoinositide dependent kinase |
| | | inhibitor |
| L-655240 | BRD-K89402695 | Thromboxane receptor antagonist |
| L-750667 | BRD-K28806945 | Dopamine receptor antagonist |
| Larixinic-acid | BRD-K40619305 | Compound that interacts with metal |
| | | centers |
| Latrepirdine | BRD-K55703048 | Glutamate receptor antagonist |
| Latrunculin-b | BRD-A19248578 | Actin polymerization inhibitor, |
| | | Unidentified pharmacological activity |
| L-BSO | BRD-A47706533 | Glutathione transferase inhibitor |
| Linsitinib | BRD-K08589866 | IGF-1 inhibitor, insulin inhibitor, ARF6 |
| | | and TBK1 activator |
| Liothyronine | BRD-K89152108 | Thyroid hormone stimulant |
| Lisuride | BRD-K88871508 | Dopamine receptor agonist |
| Loperamide | BRD-K61250553 | Opioid receptor agonist |
| Loratadine | BRD-K82795137 | Histamine receptor antagonist |
| Mafenide | BRD-K30649484 | Carbonic anhydrase inhibitor |
| Maprotiline | BRD-K03319035 | Norepinephrine reuptake inhibitor, |
| | | Tricyclic antidepressant |
| m-chlorophenylbiguanide | BRD-K36965586 | Serotonin receptor agonist |
| MDM2-inhibitor | BRD-K84987553 | MDM inhibitor |
| Mead-ethanolamide | BRD-K09764130 | Cannabinoid receptor agonist |
| Mebeverine | BRD-A09467419 | Acetylcholine receptor antagonist |
| Medetomidine | BRD-A66563878 | Adrenergic receptor agonist |
| Mepacrine | BRD-A45889380 | Cytokine production inhibitor, NFkB |
| | | pathway inhibitor, TP53 activator |
| Meprylcaine | BRD-K65417056 | Local anesthetic |
| Mepyramine | BRD-K97564742 | Histamine receptor antagonist |
| Metoclopramide | BRD-K75641298 | Dopamine receptor and serotonin receptor |
| | | antagonist |
| Midodrine | BRD-A79981887 | Adrenergic receptor agonist |
| Milrinone | BRD-K67080878 | Phosphodiesterase inhibitor |
| MK-2206 | BRD-K68065987 | AKT inhibitor |
| MLN-4924 | BRD-K67844266 | Nedd activating enzyme inhibitor |
| MR-16728 | BRD-A30590053 | Acetylcholine release enhancer or |
| | | stimulant |
| MW-STK33-3B | BRD-K64310881 | Potassium channel activator |
| n-(3-acetamidophenyl)-3- | BRD-K61217870 | Glutamate receptor antagonist |
| chlorobenzamide | | |
| Naftopidil | BRD-A01787639 | Adrenergic receptor antagonist |
| NAS-181 | BRD-A23683907 | Serotonin receptor antagonist |
| Navitoclax | BRD-K82746043 | BCL inhibitor |
| Nefazodone | BRD-K90789829 | Adrenergic inhibitor, Norepinephrine |
| | | reuptake inhibitor, Serotonin receptor |
| | | antagonist, Serotonin reuptake inhibitor |
| Nevirapine | BRD-K15502390 | Reverse transcriptase inhibitor |
| Nicorandil | BRD-K97752965 | Nitric oxide donor, Potassium channel |
| | | activator |
| Nicotine | BRD-K05395900 | Acetylcholine receptor agonist |
| Nifedipine | BRD-K96354014 | Calcium channel blocker |
| Nifurtimox | BRD-A00100033 | DNA inhibitor |
| Nikkomycin | BRD-A74771556 | Chitin inhibitor |
| Nimodipine | BRD-A58048407 | Calcium channel blocker |
| NNC-05-2090 | BRD-K85015012 | GAT inhibitor, GABA uptake inhibitor |
| Nor-binaltorphimine | BRD-A11135865 | Opioid receptor antagonist |
| Norgestimate | BRD-A04756508 | Progesterone receptor agonist |
| NVP-AUY922 | BRD-K41859756 | HSP inhibitor |
| O-2050 | BRD-K02590140 | Cannabinoid receptor antagonist |
| o-3M3FBS | BRD-K46384212 | phospholipase activator |
| Olanzapine | BRD-K18895904 | Dopamine receptor/serotonin receptor |
| | | antagonist |
| Orantinib | BRD-K91696562 | FGFR, VEGFR, PDGFR inhibitor |
| Ornidazole | BRD-A42759514 | Antiprotozoal |
| Otenzepad | BRD-A00520476 | Acetylcholine receptor antagonist |
| Oxantel | BRD-K66019333 | Anthelmintic |
| Oxaprozin | BRD-K25394294 | Cyclooxygenase inhibitor |
| Oxybenzone | BRD-K59037100 | Lipase inhibitor |
| Oxybutynin | BRD-A65013509 | Acetylcholine receptor antagonist |
| Ozagrel | BRD-K19525698 | Thromboxane synthase inhibitor |
| Palonosetron | BRD-K08924299 | Serotonin receptor antagonist |
| Pantoprazole | BRD-A22380646 | ATPase inhibitor |
| PCA-4248 | BRD-A29289453 | Platelet activating factor receptor |
| | | antagonist |
| PF-04217903 | BRD-K73319509 | c-Met inhibitor |
| Phenelzine | BRD-K87024524 | Monoamine oxidase inhibitor |
| Phenothiazine | BRD-K59597909 | Dopamine receptor antagonist |
| PI-103 | BRD-K67868012 | MTOR inhibitor, PI3K inhibitor |
| PIK-75 | BRD-M16762496 | DNA protein kinase inhibitor, PI3K |
| | | inhibitor |
| PIK-90 | BRD-K99818283 | PI3K inhibitor |
| Piperacetazine | BRD-K16277217 | Dopamine receptor antagonist |
| Piperine | BRD-K59522102 | Monoamine oxidase inhibitor |
| Pirenperone | BRD-K25224017 | Serotonin receptor antagonist |
| Pirfenidone | BRD-K96862998 | TGF β receptor inhibitor |
| Piribedil | BRD-K47936004 | Dopamine receptor agonist |
| PLX-4720 | BRD-K16478699 | RAF inhibitor |
| PNU-22394 | BRD-K16551401 | Serotonin receptor agonist |
| PP-2 | BRD-K95785537 | SRC inhibitor |
| PP-30 | BRD-K30677119 | RAF inhibitor |
| Pravastatin | BRD-K60511616 | HMGCR inhibitor |
| Prima-1-met | BRD-K49456190 | thioredoxin inhibitor |
| Profenamine | BRD-A16311756 | Butyrylcholinesterase inhibitor, |
| | | Cholinergic receptor antagonist |
| Promazine | BRD-K06980535 | Dopamine receptor antagonist |
| Prostaglandin | BRD-K09436313 | Prostanoid receptor antagonist |
| Prostaglandin-a1 | BRD-K04010869 | HSP inducer, NFkB pathway inhibitor |
| PSB-11 | BRD-K10177585 | Adenosine receptor antagonist |
| PTB1 | BRD-K16554956 | AMPK activator |
| PU-H71 | BRD-K36529613 | HSP inhibitor |
| Pyrazinamide | BRD-K28667793 | Fatty acid synthase inhibitor |
| Pyroxamide | BRD-K11663430 | HDAC inhibitor |
| Quinpirole | BRD-A85280935 | Dopamine receptor agonist |
| Raltegravir | BRD-K05658747 | HIV integrase inhibitor |
| Reserpic-acid | BRD-K32755366 | Norepinephrine transporter inhibitor |
| Retinol | BRD-K13927029 | Retinoid receptor ligand |
| Rhamnetin | BRD-K37206356 | HDAC inhibitor |
| RITA | BRD-K00317371 | MDM inhibitor |
| RO-08-2750 | BRD-K00486786 | NGF binding inhibitor |
| RO-25-6981 | BRD-K51541829 | Ionotropic glutamate receptor antagonist, |
| | | Monamine transporter modulator |
| Roscovitine | BRD-K07691486 | CDK inhibitor |
| Rotenonic-acid | BRD-K34330170 | Retinoid receptor antagonist |
| RS-67506 | BRD-K50018155 | Serotonin receptor partial agonist |
| Rucaparib | BRD-K88560311 | PARP inhibitor |
| SA-792541 | BRD-K68143200 | CDC inhibitor |
| SAL-1 | BRD-K40213712 | Adenosine receptor antagonist |
| Salsolinol | BRD-K99595596 | Monoamine oxidase inhibitor, Tyrosine |
| | | hydroxylase inhibitor |
| Saracatinib | BRD-K19540840 | SRC inhibitor |
| SB-216763 | BRD-K59184148 | Glycogen synthase kinase inhibitor |
| SCH-28080 | BRD-K55748775 | ATPase inhibitor |
| SCH-442416 | BRD-K46469693 | Adenosine receptor antagonist |
| SD-169 | BRD-K91904471 | p38 MAPK inhibitor |
| SDZ-WAG-994 | BRD-A31007383 | Adenosine receptor agonist |
| Secoisolariciresinol | BRD-K91733562 | Antioxidant |
| Securinine | BRD-A25775766 | GABA receptor antagonist, TP53 |
| | | activator |
| Selegiline | BRD-K86434416 | Monoamine oxidase inhibitor |
| Sertraline | BRD-K82036761 | Serotonin receptor antagonist |
| SID-26681509 | BRD-K08417745 | Cathepsin inhibitor |
| Sildenafil | BRD-K50128260 | Phosphodiesterase inhibitor |
| Spironolactone | BRD-K90027355 | Mineralocorticoid receptor antagonist |
| STO-609 | BRD-K52620403 | Calmodulin antagonist |
| Syrosingopine | BRD-K14200658 | Vesicular monoamine transporter inhibitor |
| Taurodeoxycholic-acid | BRD-K33572481 | Bile acid |
| Temefos | BRD-K51805276 | Cholinesterase inhibitor |
| Temozolomide | BRD-K32107296 | DNA alkylating agent |
| TER-14687 | BRD-A33833419 | Inhibitor of translocation of PKCq in T |
| | | cells |
| Testosterone | BRD-A48720949 | androgen receptor agonist |
| TG-101348 | BRD-K12502280 | FLT3 inhibitor, JAK inhibitor |
| TGX-221 | BRD-A41692738 | PI3K inhibitor |
| Thenoyltrifluoroacetone | BRD-K00959089 | Chelating agent |
| Thioproperazine | BRD-K08619574 | Dopamine receptor antagonist |
| Ticlopidine | BRD-K00603606 | Purinergic receptor antagonist |
| Tolterodine | BRD-K54316499 | Acetylcholine receptor antagonist |
| Toltrazuril | BRD-K64514229 | Antiprotozoal |
| Topiramate | BRD-K29653726 | Carbonic anhydrase inhibitor, Glutamate |
| | | receptor antagonist, Kainate receptor |
| | | antagonist |
| Tosyllysyl-chloromethyl- | BRD-K10136726 | Chymotrypsin inhibitor |
| ketone | | |
| TPCA-1 | BRD-K51575138 | IKK inhibitor |
| Trazodone | BRD-K70778732 | Adrenergic receptor antagonist, Serotonin |
| | | receptor antagonist, Serotonin reuptake |
| | | inhibitor |
| Trifluoperazine | BRD-K89732114 | Dopamine receptor antagonist |
| Triptolide | BRD-A13122391 | RNA polymerase inhibitor |
| Tyrphostin-46 | BRD-K60184833 | Tyrosine kinase inhibitor |
| Tyrphostin-AG-82 | BRD-K03670461 | EGFR inhibitor |
| U-99194 | BRD-K70281171 | Dopamine receptor antagonist |
| UNC-0321 | BRD-K74236984 | Histone lysine methyltransferase inhibitor |
| Valproic-acid | BRD-K41260949 | HDAC inhibitor |
| Verapamil | BRD-A09533288 | Calcium channel blocker |
| VU-0420363-1 | BRD-K59633790 | SARS coronavirus 3C-like protease |
| | | inhibitor |
| WZ-4002 | BRD-K72420232 | EGFR inhibitor |
| Xaliproden | BRD-K88358234 | Serotonin receptor agonist |
| XAV-939 | BRD-K12762134 | Tankyrase inhibitor |
| Y-27632 | BRD-K44084986 | Rho associated kinase inhibitor |
| YC-1 | BRD-K60476892 | Guanylyl cyclase activator |
| YS-035 | BRD-K06208435 | Calcium channel blocker |
| Zacopride | BRD-A65615053 | Serotonin receptor antagonist |
| z-prolyl-prolinal | BRD-K60174629 | Prolyl endopeptidase inhibitor |
| Zuclopenthixol | BRD-K28761384 | Dopamine receptor antagonist |
| α-estradiol | BRD-A60070924 | Estrogen receptor agonist |

### Example 3

Highly safe approved drugs repurposed for an antiviral indication, whose tissue distribution and mode of action overlap with the tropism of SARS-CoV-2 infection (e.g., airways), have the potential to complement and enhance the efficacy of drugs that are designed to specifically target virus-expressed proteins.

A recent study (Example 1) has shown that the long acting beta2-adrenoreceptor agonist bronchodilator, salmeterol, can block in vitro SARS-CoV-2 replication at clinically relevant concentrations without apparent host cell toxicity. The systems-level analyses are consistent with either salmeterol acting to enhance autophagy as previously suggested for Dengue virus infection (Medigeshi G et al. Antimicrob Agents Chemother. 2016, 60(11), 6709-9718) or alternatively, by acting to stimulate the innate immune response (Example 1).

A second compound, linisitinib (known to be an insulin-like growth factor 1 receptor (IGF1R) inhibitor, currently under investigation, not FDA-approved), also computationally predicted and experimentally verified (Example 1) to be a potent inhibitor of SARS-CoV-2 viral entry in a dose-dependent manner showed the highest inhibitory activity without overt cytotoxicity in spike-induced syncytia formation assays. It is further noted that this compound may have multiple modes of action: it interacts with ADP ribosylation factor 6 (ARF6), a binding partner of SARS-CoV-2 endonuclease nsp15 and it promotes autophagy through activation of TANK-binding kinase 1 (TBK1) mediated by the ubiquitination of the ARF domain TRIM23.

A third compound that deserves special attention among those proposed herein is imipramine, an FDA-approved tricyclic antidepressant known to act as an inhibitor of serotonin transporter (SERT). It is proposed that imipramine targets the amino acid transporter, BOAT1 (that is structurally homologous to SERT) and supports the host cell ACE2 receptor to which the SARS-CoV-2 spike protein binds. Notably, a serotonin transporter inhibitor (fluvoxamine) has recently been reported to decrease Covid-19 deaths by 90% (Sidik S. Nature, 2021, doi: 10.1038/d41586-021-02988-4; Reis G. et al. Lancet, 2022, 10(1), E42-E51). The derivatives of imipramine can likely serve as important antiviral drugs for alleviating, if not curing, Covid-10 effects.

The repurposing of approved (or investigational) drugs predicted to block virus replication-dependent host cell machinery creates both a high barrier to viral induced drug resistance and a low barrier to risk-averse regulatory approval relative to drugs specifically targeting virus-expressed proteins. Importantly, the simultaneous targeting of diverse viral vulnerabilities involving both host cell and viral encoded proteins can result in effective synergistic drug combinations that include salmeterol in combination with either the SARS-CoV-2 RNA-dependent RNA polymerase inhibitor molnupiravir or the 3CL protease inhibitor paxlovid or with both as a triple drug combination. This strategy could potentially be applied to the predicted drugs as indicated herein this application in combination with any drugs in development targeting any essential viral-encoded protein. The same type of combination therapies could be adopted using linsitinib and imipramine-derivatives. Finally, the use of drugs targeting viral proteins essential for evading the innate immune response (nsp16/nsp10) in combination with those, repurposed, that enhance the immune response is a promising strategy, that could be facilitated by utilizing the QuartataWeb interface (Li H et al. Bioinformatics, 2020, 36(12), 3935-3937).

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. The method of claim 3, wherein:
the antiviral compound is selected from the group consisting of: imipramine, salmeterol, hexylresorcinol, brompheniramine, ezetimibe, temsirolimus, linsitinib, torin-1, rottlerin, semaxanib, ipratropium, AS-605240, mefenamic acid, JNJ16259685, QL-XII-47; derivatives thereof; and combinations thereof; and
the anti-hyperinflammatory compound is selected from the group consisting of: midodrine, olanzapine, trifluoperazine, fluphenazine, azelastine, chlorphenamine, clarithromycin, saracatinib, JAK3-Inhibitor-II, AZD-8055, CGP-60474, hexamethylene, loperamide, nifedipine, liothyronine, atorvastatin, triptolide, pirfenidone, isoliquiritigenin, rucaparib, berbamine, darinaparsin, taurodeoxycholic acid; derivatives thereof; and combinations thereof.

2. (canceled)

3. A method of treating or preventing a coronavirus infection in a subject in need thereof, comprising administering to the subject a therapeutically effective amount of a composition comprising an antiviral compound and an anti-hyperinflammatory compound.

4. The method of claim 2, wherein the antiviral compound inhibits cell fusion or viral entry.

5. The method of claim 3, wherein the antiviral compound comprises a histamine receptor antagonist, an acetylcholine receptor antagonist, a norepinephrine and serotonin reuptake inhibitor, an autophagy enhancer, a mTOR inhibitor, a PI3K inhibitor, an IGF-1- and insulin receptor inhibitor, a TBK1 activator through ARF1, an adrenergic receptor agonist, a VEGFR inhibitor, a local anesthetic, a cyclooxygenase inhibitor, a glutamate receptor antagonist, a Niemann-Pick Cl-like 1 protein antagonist, a cholesterol inhibitor, a cytoplasmic tyrosine protein kinase BMX inhibitor, a MAPK and protein kinase inhibitor, or a combination thereof.

6. The method of claim 3, wherein the antiviral compound comprises: imipramine, salmeterol, hexylresorcinol, brompheniramine, ezetimibe, temsirolimus, linsitinib, torin-1, rottlerin, semaxanib, ipratropium, AS-605240, mefenamic acid, JNJ16259685, QL-XII-47; derivatives thereof; or a combination thereof.

7. The method of claim 3, wherein the antiviral compound comprises: salmeterol, rottlerin, imipramine, linsitinib, hexylresorcinol, ezetimibe, brompheniramine; derivatives thereof; or a combination thereof.

8. The method of claim 3, wherein the antiviral compound comprises salmeterol, linisitinib, imipramine, fluvoxamine, derivatives thereof, or a combination thereof.

9. (canceled)

10. The method of claim 3, wherein the antiviral compound is selected from an IGF-1R and insulin receptor inhibitor, an adrenergic receptor agonist, or a combination thereof.

11. (canceled)

12. The method of claim 3, wherein the anti-hyperinflammatory compound is selected from an adrenergic receptor agonist, a dopamine receptor antagonist, an autophagy enhancer, an autophagy dual modulator, a histamine receptor antagonist, a bacterial 50S ribosomal subunit inhibitor, an autophagy inhibitor, a SRC inhibitor, a JAK inhibitor, a mTOR inhibitor, a CDK inhibitor, a sodium/hydrogen antiport inhibitor, an opioid receptor agonist, a calcium channel blocker, a thyroid hormone stimulant, a HMGCR inhibitor, a RNA polymerase inhibitor, a TGFβ receptor inhibitor, an anti-fibrotic, a guanylate cyclase activator, a PARP inhibitor, a calmodulin antagonist, an apoptosis stimulant, a bile acid, or a combination thereof.

13. The method of claim 3, wherein the anti-hyperinflammatory compound is selected from midodrine, olanzapine, trifluoperazine, fluphenazine, azelastine, chlorphenamine, clarithromycin, saracatinib, JAK3-Inhibitor-II, AZD-8055, CGP-60474, hexamethylene, loperamide, nifedipine, liothyronine, atorvastatin, triptolide, pirfenidone, isoliquiritigenin, rucaparib, berbamine, darinaparsin, taurodeoxycholic acid, or a combination thereof.

14. The method of claim 3, wherein the anti-hyperinflammatory compound elevates IFN signaling, suppresses cytokine pathways, or a combination thereof.

15. The method of claim 3, wherein the composition comprises salmeterol, linsitinib, impramine, derivatives thereof, or a combination thereof, optionally in combination with one or more additional agents.

16. (canceled)

17. (canceled)

18. The method of claim 3, wherein the composition comprises salmeterol in combination with molnupiravir, paxlovid, or a combination thereof.

19. (canceled)

20. (canceled)

21. (canceled)

22. (canceled)

23. (canceled)

24. (canceled)

25. The pharmaceutical composition of claim 27, wherein:
the antiviral compound is selected from the group consisting of: imipramine, salmeterol, hexylresorcinol, brompheniramine, ezetimibe, temsirolimus, linsitinib, torin-1, rottlerin, semaxanib, ipratropium, AS-605240, mefenamic acid, JNJ16259685, QL-XII-47; derivatives thereof; and combinations thereof; and
the anti-hyperinflammatory compound is selected from the group consisting of: midodrine, olanzapine, trifluoperazine, fluphenazine, azelastine, chlorphenamine, clarithromycin, saracatinib, JAK3-Inhibitor-II, AZD-8055, CGP-60474, hexamethylene, loperamide, nifedipine, liothyronine, atorvastatin, triptolide, pirfenidone, isoliquiritigenin, rucaparib, berbamine, darinaparsin, taurodeoxycholic acid; derivatives thereof; and combinations thereof.

26. (canceled)

27. A pharmaceutical composition for the treatment of coronavirus comprising a pharmaceutically acceptable excipient and a therapeutically effective amount of a composition comprising an antiviral compound and an anti-hyperinflammatory compound.

28. The pharmaceutical composition of claim 27, further comprising a propellant.

29. The pharmaceutical composition of claim 28, wherein the propellant comprises compressed air, ethanol, nitrogen, carbon dioxide, nitrous oxide, hydrofluoroalkanes (HFA), 1,1,1,2,-tetrafluoroethane, 1,1,1,2,3,3,3-heptafluoropropane, or a combination thereof.

30. A pressurized container comprising the pharmaceutical composition of claim 27, wherein the pressurized container comprises a manual pump spray, inhaler, meter-dosed inhaler, dry powder inhaler, nebulizer, vibrating mesh nebulizer, jet nebulizer, or ultrasonic wave nebulizer.

31. (canceled)

32. A method of identifying a compound for treating or preventing an infection with an infectious microbe in a subject in need thereof, the method comprising:
a) obtaining transcriptomic data from cells infected with the infectious microbe,
b) identifying differentially expressed genes (DEGs),
c) characterizing host-targeted antimicrobial or anticytokine signature,
d) identifying compounds that stimulate the anti-microbial or -cytokine signature,
e) evaluating known and predicted targets of compounds identified in step d),
f) constructing an infection host response protein-protein interaction (PPI) network and modules,
g) prioritizing compounds based on network proximity analysis,
h) clustering of prioritized compounds associated with selected disease modules,
i) selecting representative compounds from each cluster for in vitro assays, and
j) analyzing the results of steps a-i to thereby identify the compound for treating or preventing the infection.

33. The method of claim 32, wherein the infectious microbe comprises a coronavirus.

34-56. (canceled)
